(19) United States
(12) Patent Application Publication
(10) Pub. No.: US 2024/0327613 A1
(43) Pub. Date: Oct. 3, 2024

(54) RESIN COMPOSITION, CURED SUBSTANCE, OPTICAL MEMBER, ULTRAVIOLET ABSORBER, COMPOUND, PRODUCTION METHOD OF COMPOUND, AND POLYMER

(71) Applicant: FUJIFILM Corporation, Tokyo (JP)

(72) Inventors: Atsushi AZUMA, Shizuoka (JP); Daisuke SASAKI, Shizuoka (JP); Yusuke SAKAI, Shizuoka (JP)

(73) Assignee: FUJIFILM Corporation, Tokyo (JP)

(21) Appl. No.: 18/660,214

(22) Filed: May 9, 2024

Related U.S. Application Data
(63) Continuation of application No. PCT/JP2022/043145, filed on Nov. 22, 2022.

(30) Foreign Application Priority Data
Dec. 2, 2021 (JP) 2021-196054
Apr. 21, 2022 (JP) 2022-070074
Aug. 31, 2022 (JP) 2022-137638

(51) Int. Cl. C08K 5/45; C07D 495/04; C08F 220/14; G02B 5/00

(52) U.S. Cl. CPC C08K 5/45; C07D 495/04; C08F 220/14; G02B 5/003

(57) ABSTRACT

Provided is a resin composition containing a compound represented by Formula (1) and a resin, in which, in Formula (1), Q¹ represents a group represented by Formula (Q-1), Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹, R¹ and R² each independently represent a hydrogen atom or a substituent, and X¹ to X⁴ each independently represent —S— or the like. The present invention also relates to a cured substance, an optical member, an ultraviolet absorber, a compound, a production method of a compound, and a polymer.

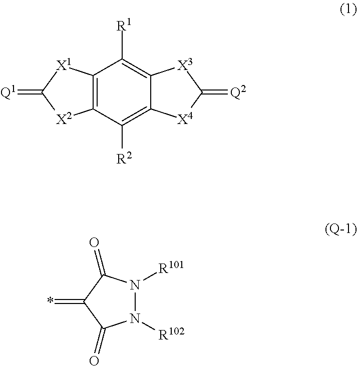

### CROSS-REFERENCE TO RELATED APPLICATIONS

This application is a Continuation of PCT International Application No. PCT/JP2022/043145 filed on Nov. 22, 2022, which claims priority under 35 U.S.C § 119(a) to Japanese Patent Application No. 2021-196054 filed on Dec. 2, 2021, Japanese Patent Application No. 2022-070074 filed on Apr. 21, 2022, and Japanese Patent Application No. 2022-137638 filed on Aug. 31, 2022. Each of the above application(s) is hereby expressly incorporated by reference, in its entirety, into the present application.

### BACKGROUND OF THE INVENTION

### 1. Field of the Invention

The present invention relates to a resin composition containing an ultraviolet absorber. In addition, the present invention relates to a cured substance and an optical member, which are formed of the resin composition. In addition, the present invention relates to an ultraviolet absorber, a compound, a production method of a compound, and a polymer.

### 2. Description of the Related Art

A benzobisdithiol compound has excellent absorption of ultraviolet rays, and has been used as an ultraviolet absorber or the like. For example, WO2009/022736A discloses use of a specific benzobisdithiol as an ultraviolet absorber.

### SUMMARY OF THE INVENTION

In the ultraviolet absorber, as one of required characteristics, it is required that the ultraviolet absorber has little coloration. In addition, in recent years, there has been a demand for having a high absorption ability for ultraviolet rays having a longer wavelength in the vicinity of a wavelength of 400 nm.

In addition, the ultraviolet absorption performance of the ultraviolet absorber may be degraded with time due to irradiation with light. In particular, an ultraviolet absorber having a maximal absorption wavelength on a longer wavelength side in an ultraviolet region has a tendency that light resistance is poor and the ultraviolet absorption ability thereof is likely to be degraded with time. Therefore, in recent years, there has been a demand for further improvement of performance in light resistance of the ultraviolet absorber.

Therefore, an object of the present invention is to provide a resin composition with which a cured substance having excellent absorption ability of ultraviolet rays in a vicinity of a wavelength of 400 nm, little coloration, and excellent light resistance can be produced. Another object of the present invention is to provide a cured substance, an optical member, an ultraviolet absorber, a compound, a production method of a compound, and a polymer.

As a result of intensive studies on a compound having a skeleton represented by Formula (1), the present inventor has found that a compound having a structure in which Q¹ and Q² in Formula (1) are in a specific combination is a compound having excellent absorption ability of ultraviolet rays in a vicinity of a wavelength of 400 nm, little coloration, and excellent light resistance, and has completed the present invention. Therefore, the present invention provides the following.
<1> A resin composition comprising:
   a compound represented by Formula (1); and
   a resin,

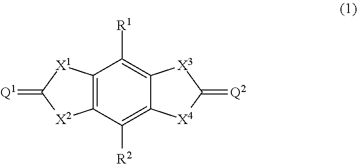

in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group,

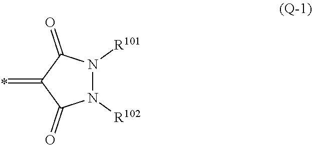

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.
<2> The resin composition according to <1>,
in which R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
   in a case where any one of R¹⁰¹ or R¹⁰² is a methyl group, the other is a hydrogen atom, an alkyl group having 2 or more carbon atoms, an aralkyl group, an aryl group, or a heterocyclic group, and
   in a case where any one of R¹⁰¹ or R¹⁰² is a phenyl group, the other is a hydrogen atom, an alkyl group, an aralkyl group, an aryl group having a substituent, or a heterocyclic group.
<3> The resin composition according to <1> or <2>,
in which the compound represented by Formula (1) is a compound represented by Formula (3),

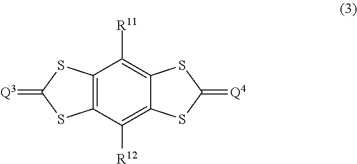

in Formula (3), Q³ represents the group represented by Formula (Q-1),
Q⁴ represents ═O, ═S, ═NR^{q11}, or ═CR^{q12}R^{q13}, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)O—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.
<4> The resin composition according to <3>,
in which R¹¹ and R¹² in Formula (3) each independently represent —OH, —O—Y¹¹, —OC(═O)—Y¹¹, —OC(═O)O—Y¹¹, —OC(═O)NR^{y11}—Y¹¹, or —OSO₂—Y¹¹, where R^{y11} represents a hydrogen atom, an alkyl group, an aralkyl group, or an aryl group, and Y¹¹ represents an alkyl group, an aralkyl group, or an aryl group.
<5> The resin composition according to <3>,
in which at least one of R¹¹, R¹², Q³, or Q⁴ in Formula (3) includes a group including a polymerizable group having an ethylenically unsaturated bond.
<6> The resin composition according to any one of <1> to <5>,
in which the resin is at least one selected from a (meth)acrylic resin, a polystyrene resin, a polyester resin, a polyurethane resin, a thiourethane resin, a polyimide resin, a polyamide resin, an epoxy resin, a polycarbonate resin, a phthalate resin, a cellulose acylate resin, or a cyclic olefin resin.
<7> A cured substance formed of the resin composition according to any one of <1> to <6>.
<8> An optical member comprising:
the cured substance according to <7>.
<9> An ultraviolet absorber comprising:
a compound represented by Formula (1),

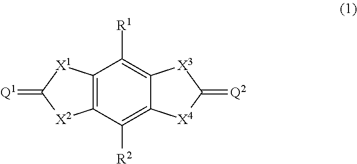

in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group,

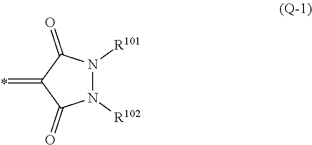

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
   in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
   in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.
<10> The ultraviolet absorber according to <9>,
in which R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
   in a case where any one of R¹⁰¹ or R¹⁰² is a methyl group, the other is a hydrogen atom, an alkyl group having 2 or more carbon atoms, an aralkyl group, an aryl group, or a heterocyclic group, and
   in a case where any one of R¹⁰¹ or R¹⁰² is a phenyl group, the other is a hydrogen atom, an alkyl group, an aralkyl group, an aryl group having a substituent, or a heterocyclic group.
<11> A compound represented by Formula (3),

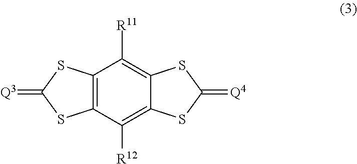

in Formula (3), Q³ represents a group represented by Formula (Q-1),
Q⁴ represents ═O, ═S, ═NR^{q11}, or ═CR^{q12}R^{q13}, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group,

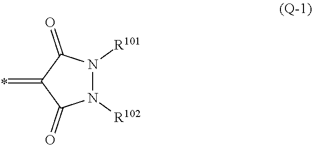

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
   in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
   in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.
<12> The compound according to <11>,
in which R¹¹ and R¹² in Formula (3) each independently represent —OH, —O—Y¹¹, —OC(═O)—Y¹¹, —OC(═O)O—Y¹¹, —OC(═O)NR^{y11}—Y¹¹, or —OSO₂—Y¹¹, where R^{y11} represents a hydrogen atom, an alkyl group, an aralkyl group, or an aryl group, and Y¹¹ represents an alkyl group, an aralkyl group, or an aryl group, and
R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
   in a case where any one of R¹⁰¹ or R¹⁰² is a methyl group, the other is a hydrogen atom, an alkyl group having 2 or more carbon atoms, an aralkyl group, an aryl group, or a heterocyclic group, and
   in a case where any one of R¹⁰¹ or R¹⁰² is a phenyl group, the other is a hydrogen atom, an alkyl group, an aralkyl group, an aryl group having a substituent, or a heterocyclic group.
<13> The compound according to <11>,
in which at least one of R¹¹, R¹², Q³, or Q⁴ in Formula (3) includes a group including a polymerizable group having an ethylenically unsaturated bond.
<14> A production method of a compound, comprising:
producing a compound represented by Formula (6) by reacting a compound represented by Formula (4) with a compound represented by Formula (5),

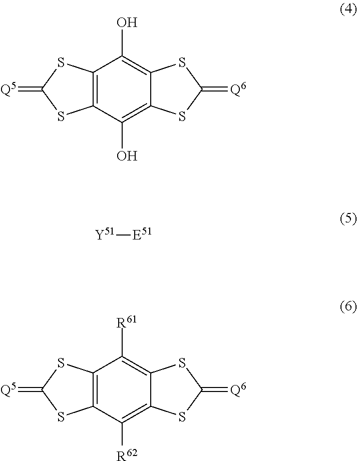

in Formula (4), Q⁵ represents a group represented by Formula (Q-1), Q⁶ represents ═O, ═S, ═NR^{q21}, or ═CR^{q22}R^{q23}, R^{q21} to R^{q23} each independently represent a hydrogen atom or a substituent, and R^{q22} and R^{q23} may be bonded to each other to form a ring, provided that, in a case where R^{q22} and R^{q23} are bonded to each other to form a ring, ═CR^{q22}R^{q23} does not have the same structure as Q⁵,
in Formula (5), E⁵¹ represents —COCl, —O(C═O)Cl, —NR^{e51}(C═O)Cl, —NCO, —Cl, —Br, —I, or —SO₂R^{e52}, R^{e51} represents a hydrogen atom, an alkyl group, an aralkyl group, or an aryl group, R^{e52} represents —Cl or an alkoxy group, and Y⁵¹ represents an alkyl group, an aralkyl group, or an aryl group,
   in Formula (6), Q⁵ represents a group represented by Formula (Q-1), Q⁶ represents ═O, ═S, ═NR^{q21}, or ═CR^{q22}R^{q23}, R^{q21} to R^{q23} each independently represent a hydrogen atom or a substituent, R^{q22} and R^{q23} may be bonded to each other to form a ring, provided that, in a case where R^{q22} and R²³ are bonded to each other to form a ring, ═CR^{q22}R^{q23} does not have the same structure as Q⁵, and
R⁶¹ and R⁶² each independently represent —O—Y⁶¹, —OC(═O)—Y⁶¹, —OC(═O)O—Y⁶¹, —OC(═O)NR^{y61}—Y⁶¹, or —OSO₂—Y⁶¹, where R^{y61} represents a hydrogen atom, an alkyl group, an aralkyl group, or an aryl group, and Y⁶¹ represents an alkyl group, an aralkyl group, or an aryl group,

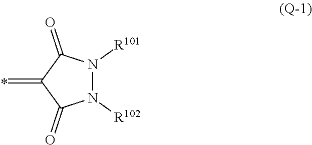

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
   in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
   in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.
<15> The production method of a compound according to <14>,
in which R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
   in a case where any one of R¹⁰¹ or R¹⁰² is a methyl group, the other is a hydrogen atom, an alkyl group having 2 or more carbon atoms, an aralkyl group, an aryl group, or a heterocyclic group, and
   in a case where any one of R¹⁰¹ or R¹⁰² is a phenyl group, the other is a hydrogen atom, an alkyl group, an aralkyl group, an aryl group having a substituent, or a heterocyclic group.
<16> A polymer including a structure derived from the compound according to <13>.

According to the present invention, it is possible to provide a resin composition with which a cured substance having excellent absorption ability of ultraviolet rays in a vicinity of a wavelength of 400 nm, little coloration, and excellent light resistance can be produced. In addition, according to the present invention, it is possible to provide a cured substance, an optical member, an ultraviolet absorber, a compound, a production method of a compound, and a polymer.

### DESCRIPTION OF THE PREFERRED EMBODIMENTS

Hereinafter, the details of the present invention will be described.

In citations for a group (atomic group) in the present specification, in a case where the group is denoted without specifying whether it is substituted or unsubstituted, the group includes both a group having no substituent and a group having a substituent. For example, “alkyl group” denotes not only an alkyl group having no substituent (unsubstituted alkyl group) but also an alkyl group having a substituent (substituted alkyl group).

In the present specification, a numerical range represented by “to” means a range including numerical values before and after “to” as a lower limit value and an upper limit value.

In the present specification, a total solid content denotes the total amount of all components of the resin composition, excluding a solvent.

In the present specification, “(meth)acrylate” represents either or both of acrylate and methacrylate, “(meth)acryl” represents either or both of acryl and methacryl, “(meth)allyl” represents either or both of allyl and methallyl, and “(meth)acryloyl” represents either or both of acryloyl and methacryloyl.

In the present specification, the term “step” not only means an independent step, but also includes a step which is not clearly distinguished from other steps in a case where an intended action of the step is obtained.

In the present specification, a weight-average molecular weight (Mw) and a number-average molecular weight (Mn) are each defined as a value in terms of polystyrene through measurement by means of gel permeation chromatography (GPC).

### <Resin Composition>

The resin composition according to the embodiment of the present invention contains a compound represented by Formula (1) and a resin.

The compound represented by Formula (1) is a compound which has excellent absorption ability of ultraviolet rays in the vicinity of a wavelength of 400 nm, has little coloration, and has excellent light resistance in which decomposition or the like due to irradiation with light is less likely to occur. Therefore, with the resin composition according to the embodiment of the present invention, it is possible to manufacture a cured substance having excellent absorption ability of ultraviolet rays in the vicinity of a wavelength of 400 nm, little coloration, and excellent light resistance.

The resin composition according to the embodiment of the present invention may be a composition in a solution state containing a solvent.

In addition, the resin composition according to the embodiment of the present invention may be a kneaded material. In the present specification, the kneaded material is obtained by kneading the compound represented by Formula (1) and the resin. That is, the kneaded material in the present specification is a kneaded material in which the compound represented by Formula (1) is mixed and dispersed in the resin, and is different from a solution in which the compound represented by Formula (1) and the resin are dissolved or dispersed in a solvent.

The kneaded material is also preferably in a form of pellet. In the present specification, the pellet is a material obtained by granulating (pelletizing) the kneaded material into a certain shape such as a spherical shape, an ellipsoidal shape, a cylindrical shape, and a prismatic shape. In addition, it is also preferable that the pellet is a master pellet (masterbatch). In addition, the master pellet (the masterbatch) is a material obtained by dispersing an additive such as an ultraviolet absorber having a high concentration in a resin, and is used by being mixed with the resin or the like at a specified magnification in a case of forming a molded body.

Hereinafter, each component contained in the resin composition will be described.

### <<Compound Represented by Formula (1) (Specific Compound)>>

The resin composition according to the embodiment of the present invention contains a compound represented by Formula (1) (hereinafter, also referred to as a specific compound).

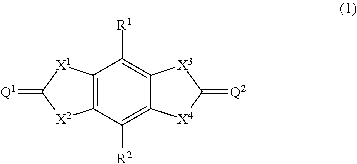

In Formula (1), Q¹ represents a group represented by Formula (Q-1);
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹;
R¹ and R² each independently represent a hydrogen atom or a substituent; and
X¹ to X⁴ each independently represent —S—, —NR^{X1}—, or —SO₂—, and R^{X1} represents a hydrogen atom or an alkyl group;

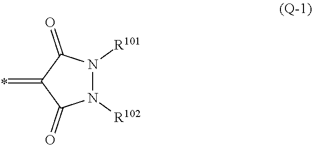

in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.
   —Regarding R¹ and R²—

Examples of the substituent represented by R¹ and R² in Formula (1) include an alkyl group, an aryl group, an aralkyl group, a heterocyclic group, a group including a polymerizable group having an ethylenically unsaturated bond, —OH, —O—Y¹¹, —OC(═O)—Y¹¹, —OC(═O)O—Y¹¹, —OC(═O)NR^{y11}—Y¹¹, —OSO₂—Y¹¹, a cyano group, a halogen atom, and a nitro group. R^{y11} represents a hydrogen atom, an alkyl group, an aralkyl group, or an aryl group, and Y¹¹ represents an alkyl group, an aralkyl group, or an aryl group.

The number of carbon atoms in the above-described alkyl group is preferably 1 to 30, more preferably 1 to 20, still more preferably 1 to 15, particularly preferably 1 to 10, and most preferably 1 to 8. The alkyl group may be linear, branched, or cyclic, and preferably linear or branched. The alkyl group may have a substituent. Examples of the substituent include groups described in a substituent T later.

The number of carbon atoms in the above-described aryl group is preferably 6 to 30, more preferably 6 to 20, still more preferably 6 to 15, particularly preferably 6 to 10, and most preferably 6 to 8. The aryl group may have a substituent. Examples of the substituent include groups described in a substituent T later.

The number of carbon atoms in an alkyl moiety of the above-described aralkyl group is preferably 1 to 10, more preferably 1 to 5, and still more preferably 1 to 3. The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent. Examples of the substituent include groups described in a substituent T later. Specific examples of the aralkyl group include a benzyl group.

A heterocyclic ring in the above-described heterocyclic group preferably includes a 5-membered or 6-membered saturated or unsaturated heterocyclic ring. The heterocyclic ring may be fused with an aliphatic ring, an aromatic ring, or another heterocyclic ring. Examples of a heteroatom constituting the ring of the heterocyclic ring include B, N, O, S, Se, and Te; and N, O, or S is preferable. It is preferable that the carbon atom of the heterocyclic ring has a free valence (monovalent) (the heterocyclic group is bonded at the carbon atom). The number of carbon atoms in the heterocyclic group is preferably 1 to 40, more preferably 1 to 30, and still more preferably 1 to 20. Examples of the saturated heterocyclic ring in the heterocyclic group include a pyrrolidine ring, a morpholine ring, a 2-bora-1,3-dioxolane ring, and a 1,3-thiazolidine ring. Examples of the unsaturated heterocyclic ring in the heterocyclic group include an imidazole ring, a thiazole ring, a benzothiazole ring, a benzoxazole ring, a benzotriazole ring, a benzoselenazole ring, a pyridine ring, a pyrimidine ring, and a quinoline ring.

Examples of the above-described halogen atom include a chlorine atom, a bromine atom, and an iodine atom.

Examples of the polymerizable group having an ethylenically unsaturated bond in the above-described group including a polymerizable group having an ethylenically unsaturated bond include a vinyl group, a (meth)allyl group, a (meth)acryloyl group, a (meth)acryloyloxy group, a (meth)acryloylamino group, and a vinylphenyl group; and a (meth)acryloyloxy group or a vinylphenyl group is preferable.

Examples of the group including a polymerizable group having an ethylenically unsaturated bond include a group represented by Formula (T1).

*—X^{T1}—Y^{T1}—Z^{T1} (T1)

In Formula (T1), X^{T1} represents a single bond, —O—, —OC(═O)—, —OC(═O)O—, or —OC(═O)NRx¹-, Rx¹ represents a hydrogen atom, an alkyl group, or an aryl group,
Y^{T1} represents a single bond or a divalent linking group, and
Z^{T1} represents a polymerizable group having an ethylenically unsaturated bond.

As the alkyl group represented by Rx¹, an alkyl group having 1 to 30 carbon atoms is preferable. Specific examples thereof include a methyl group, an ethyl group, an n-propyl group, an isopropyl group, and an n-butyl group. The aryl group represented by Rx¹ is preferably a substituted or unsubstituted aryl group having 6 to 30 carbon atoms. Specific examples thereof include a phenyl group, a p-tolyl group, and a naphthyl group. Rx¹ is preferably a hydrogen atom.

X^{T1} is preferably —O—, —OC(═O)—, or —OC(═O)NH—; and from the viewpoint of synthesis, more preferably —OC(═O)—.

Examples of the divalent linking group represented by Y^{T1} include a hydrocarbon group and a group in which two or more hydrocarbon groups are bonded to each other by a single bond or a linking group. Examples of the hydrocarbon group include an aliphatic hydrocarbon group and an aromatic hydrocarbon group, and an aliphatic hydrocarbon group is preferable. The number of carbon atoms in the aliphatic hydrocarbon group is preferably 1 to 30, more preferably 1 to 20, and still more preferably 1 to 15. The aliphatic hydrocarbon group may be linear, branched, or cyclic. In addition, the cyclic aliphatic hydrocarbon group may be a monocycle or a fused ring. In addition, the cyclic aliphatic hydrocarbon group may have a crosslinking structure. The number of carbon atoms in the aromatic hydrocarbon group is preferably 6 to 30, more preferably 6 to 20, and still more preferably 6 to 10. The hydrocarbon group may have a substituent. Examples of the substituent include a substituent T described later. Examples of the substituent include a hydroxy group.

Examples of the linking group which links two or more of the above-described hydrocarbon groups include —NH—, —S(═O)₂—, —O—, —C(═O)—, —OC(═O)—, —C(═O)O—, —NHC(═O)—, and —C(═O)NH—. Among these, —O—, —C(═O)—, —OC(═O)—, —C(═O)O—, —NHC(═O)—, or —C(═O)NH— is preferable.

Examples of the polymerizable group having an ethylenically unsaturated bond, represented by Z^{T1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (T1) include groups represented by T-1 to T-28. In the following structural formulae, Me represents a methyl group and * represents a bonding site.

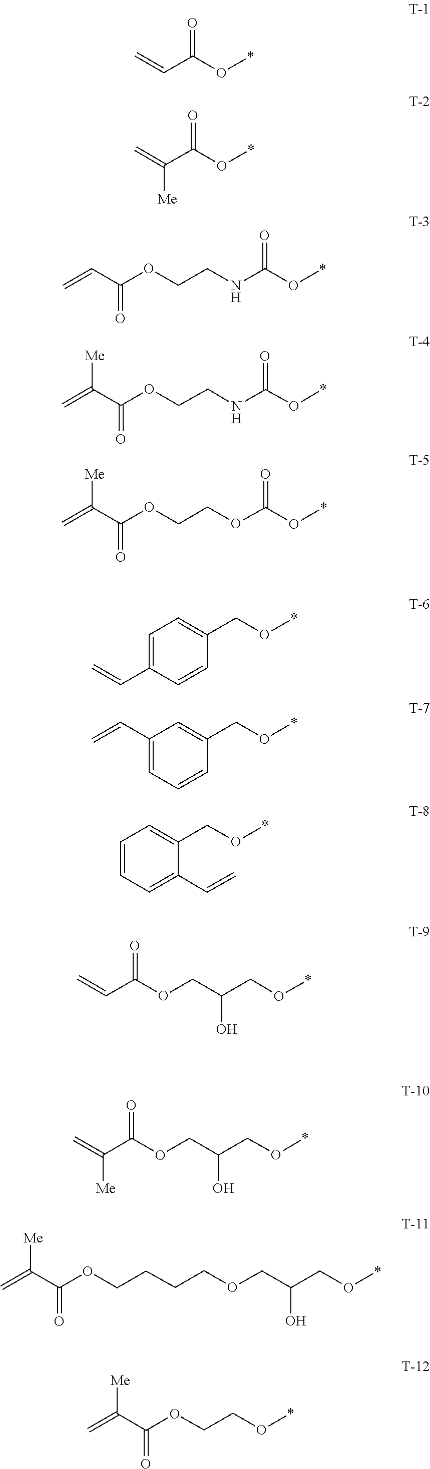

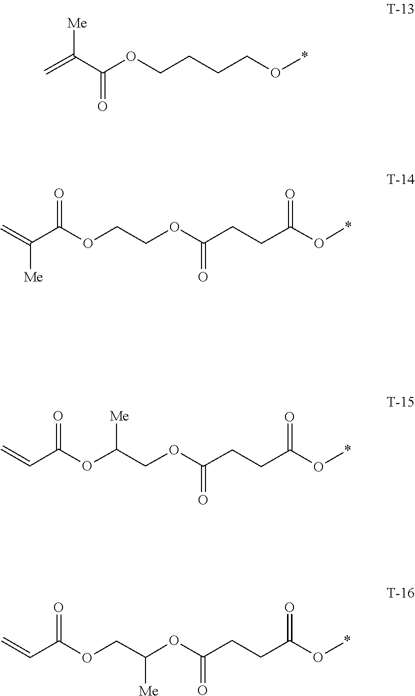 [SMILES: *OCCCCOC(=O)C(=C)C ; *OC(=O)CCC(=O)OCCOC(=O)C(=C)C ; *OC(=O)CCC(=O)OCC(C)OC(=O)C=C ; *OC(=O)CCC(=O)OC(C)COC(=O)C=C]

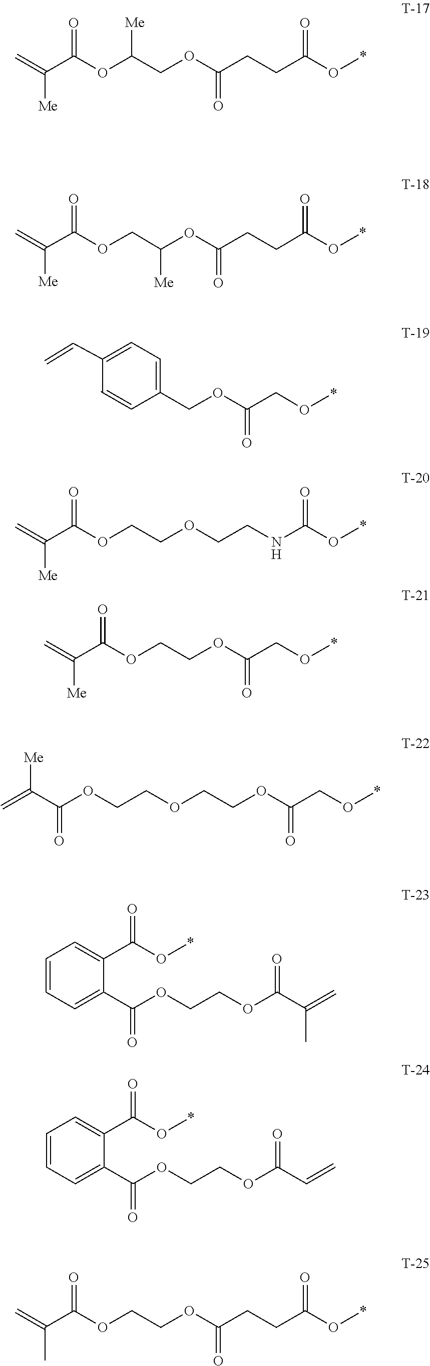

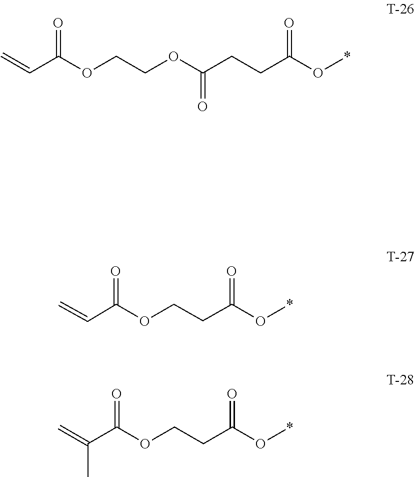

It is preferable that R¹ and R² in Formula (1) each independently represent —OH, —O—Y¹¹, —OC(═O)—Y¹¹, —OC(═O)O—Y¹¹, —OC(═O)NR^{y11}—Y¹¹, —OSO₂—Y¹¹, or the group including a polymerizable group having an ethylenically unsaturated bond.

Examples of one aspect thereof include an aspect in which R¹ and R² in Formula (1) are each independently —OH, —O—Y¹¹, —OC(═O)—Y¹¹, —OC(═O)O—Yu, —OC(═O)NR^{y11}—Y¹¹, or —OSO₂—Y¹¹. In this aspect, from the reason that stability is excellent, it is more preferable that R¹ and R² are each independently —OC(═O)—Y¹¹, —O—Y¹¹, or —OC(═O)NR^{y11}—Y¹¹. R^{y11} is preferably a hydrogen atom or an alkyl group and more preferably a hydrogen atom. From the reason of excellent solubility, Y¹¹ is preferably an alkyl group, more preferably a linear or branched alkyl group, and still more preferably a branched alkyl group.

Examples of another aspect thereof include an aspect in which at least one of R¹ or R² is a group including the group including a polymerizable group having an ethylenically unsaturated bond. According to this aspect, the effect of being capable of suppressing bleed-out in the resin is obtained.

—Regarding X¹ to X⁴—

X¹ to X⁴ in Formula (1) each independently represent —S—, —NR^{X1}—, or —SO₂—, and R^{X1} represents a hydrogen atom or an alkyl group. A preferred range of the alkyl group represented by R^{X1} is the same as that for the above-described alkyl group. R^{X1} is preferably a hydrogen atom.

From the reason that the effect of the present invention is more remarkable, X¹ to X⁴ in Formula (1) are preferably —S—.

### —Regarding Q¹—

Q¹ in Formula (1) represents a group represented by Formula (Q-1).

R¹⁰¹ and R¹⁰² in Formula (Q-1) each independently represent a hydrogen atom, an alkyl group, an aralkyl group, an aryl group, a heterocyclic group, or a group including a polymerizable group having an ethylenically unsaturated bond.

However, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond; in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond; and in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

The number of carbon atoms in the alkyl group represented by R¹⁰¹ and R¹⁰² is preferably 1 to 30. The upper limit thereof is preferably 20 or less, more preferably 15 or less, still more preferably 10 or less, and even more preferably 8 or less. The lower limit thereof is preferably 2 or more and more preferably 3 or more. The alkyl group may be linear, branched, or cyclic, and preferably linear or branched. The alkyl group may have a substituent. Examples of the substituent include groups described in a substituent T later.

The number of carbon atoms in the aryl group represented by R¹⁰¹ and R¹⁰² is preferably 6 to 30, more preferably 6 to 20, still more preferably 6 to 15, particularly preferably 6 to 10, and most preferably 6 to 8. The aryl group may have a substituent. Examples of the substituent include groups described in a substituent T later.

The number of carbon atoms in an alkyl moiety of the aralkyl group represented by R¹⁰¹ and R¹⁰² is preferably 1 to 10, more preferably 1 to 5, and still more preferably 1 to 3. The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent. Examples of the substituent include groups described in a substituent T later.

Examples of the heterocyclic group represented by R¹⁰¹ and R¹⁰² include the above-described heterocyclic groups.

Examples of the group including a polymerizable group having an ethylenically unsaturated bond, represented by as R¹⁰¹ and R¹⁰², include a group represented by Formula (V1).

*—X^{V1}—Y^{V1}—Z^{V1} (V1)

In Formula (V1), X^{V1} represents a single bond, —O—, —C(═O)—, —C(═O)O—, or —C(═O)NRx²-, Rx² represents a hydrogen atom, an alkyl group, or an aryl group,
Y^{V1} represents a single bond or a divalent linking group, and
Z^{V1} represents a polymerizable group having an ethylenically unsaturated bond.

The alkyl group and aryl group represented by Rx² have the same meaning as the alkyl group and aryl group represented by Rx¹ of the group represented by Formula (T1), and preferred ranges thereof are also the same. Rx² is preferably a hydrogen atom.

X^{V1} is preferably a single bond or —C(═O)—, and more preferably a single bond.

Examples of the divalent linking group represented by Y^{V1} include the groups described as the divalent linking group represented by Y^{T1} of the group represented by Formula (T1), and a preferred range thereof is also the same.

Examples of the polymerizable group having an ethylenically unsaturated bond, represented by Z^{V1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (V1) include groups represented by V-1 to V-12. In the following structural formulae, * is a bonding site.

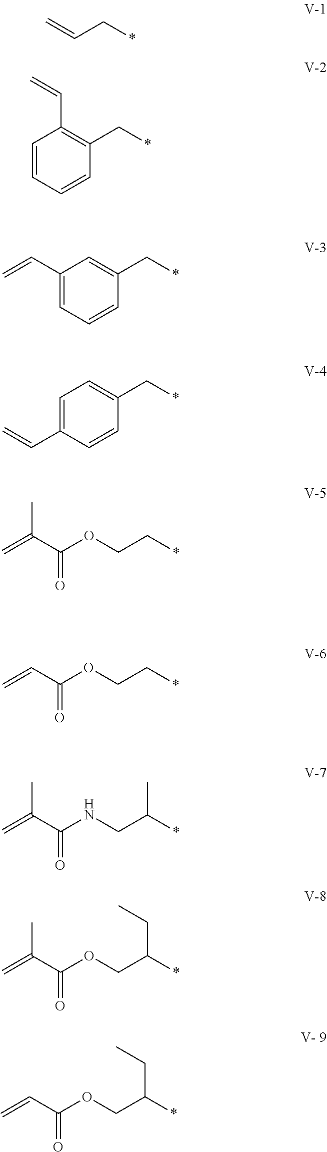

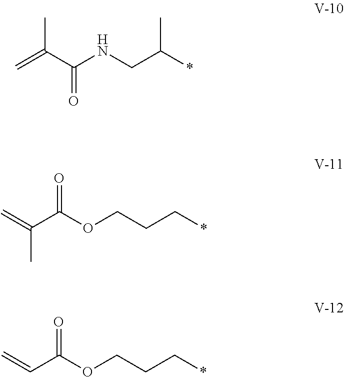

Examples of one aspect of R¹⁰¹ and R¹⁰² in Formula (Q-1) include an aspect that R¹⁰¹ and R¹⁰² each independently represent a hydrogen atom, an alkyl group, an aralkyl group, an aryl group, or a heterocyclic group, in which, in a case where any one of R¹⁰¹ or R¹⁰² is a hydrogen atom, the other is an alkyl group, an aralkyl group, an aryl group, or a heterocyclic group; in a case where any one of R¹⁰¹ or R¹⁰² is a methyl group, the other is a hydrogen atom, an alkyl group having 2 or more carbon atoms, an aralkyl group, an aryl group, or a heterocyclic group; and in a case where any one of R¹⁰¹ or R¹⁰² is a phenyl group, the other is a hydrogen atom, an alkyl group, an aralkyl group, an aryl group having a substituent, or a heterocyclic group.

R¹⁰¹ and R¹⁰² in Formula (Q-1) are each independently preferably an alkyl group or an aralkyl group, and more preferably an aralkyl group.

In a case where R¹⁰¹ and R¹⁰² are alkyl groups, it is preferable that the alkyl groups represented by R¹⁰¹ and R¹⁰² are each independently an alkyl group having 2 or more carbon atoms.

Examples of another aspect of R¹⁰¹ and R¹⁰² in Formula (Q-1) include an aspect in which at least one of R¹⁰¹ or R¹⁰² in Formula (Q-1) is the group including a polymerizable group having an ethylenically unsaturated bond. According to this aspect, the effect of being capable of suppressing bleed-out in the resin is obtained.

### —Regarding Q²—

Q² in Formula (1) represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring. However, in a case where R^{q2} and R^{q3} are bonded to each other to form a ring, ═CR^{q2}R^{q3} does not have the same structure as Q¹.

Examples of the substituent represented by R^{q1} to R^{q3} include a cyano group, a carbamoyl group, a sulfamoyl group, a nitro group, an acyl group, an alkylsulfonyl group, an arylsulfonyl group, an alkylsulfinyl group, an arylsulfinyl group, an alkoxycarbonyl group, an aryloxycarbonyl group, an alkyl group, an aryl group, a heterocyclic group, and a group including a polymerizable group having an ethylenically unsaturated bond. These groups may further have a substituent. Examples of the substituent include groups exemplified in the substituent T described later.

Examples of the carbamoyl group include a carbamoyl group having 1 to 10 carbon atoms; and a carbamoyl group having 2 to 8 carbon atoms is preferable, and a carbamoyl group having 2 to 5 carbon atoms is more preferable.

Examples of the sulfamoyl group include a sulfamoyl group having 0 to 10 carbon atoms; and a sulfamoyl group having 2 to 8 carbon atoms is preferable, and a sulfamoyl group having 2 to 5 carbon atoms is more preferable.

Examples of the acyl group include an acyl group having 1 to 20 carbon atoms; and an acyl group having 1 to 12 carbon atoms is preferable, and an acyl group having 1 to 8 carbon atoms is more preferable.

Examples of the alkylsulfonyl group include an alkylsulfonyl group having 1 to 20 carbon atoms; and an alkylsulfonyl group having 1 to 10 carbon atoms is preferable, and an alkylsulfonyl group having 1 to 8 carbon atoms is more preferable.

Examples of the arylsulfonyl group include an arylsulfonyl group having 6 to 20 carbon atoms, and an arylsulfonyl group having 6 to 10 carbon atoms is preferable.

Examples of the alkylsulfinyl group include an alkylsulfinyl group having 1 to 20 carbon atoms; and an alkylsulfinyl group having 1 to 10 carbon atoms is preferable, and an alkylsulfinyl group having 1 to 8 carbon atoms is more preferable.

Examples of the arylsulfinyl group include an arylsulfinyl group having 6 to 20 carbon atoms, and an arylsulfinyl group having 6 to 10 carbon atoms is preferable.

Examples of the alkoxycarbonyl group include an alkoxycarbonyl group having 2 to 20 carbon atoms; and an alkoxycarbonyl group having 2 to 12 carbon atoms is preferable, and an alkoxycarbonyl group having 2 to 8 carbon atoms is more preferable.

Examples of the aryloxycarbonyl group include an aryloxycarbonyl group having 6 to 20 carbon atoms; and an aryloxycarbonyl group having 6 to 12 carbon atoms is preferable, and an aryloxycarbonyl group having 6 to 8 carbon atoms is more preferable.

Examples of the alkyl group include an alkyl group having 1 to 18 carbon atoms; and an alkyl group having 1 to 10 carbon atoms is preferable and an alkyl group having 1 to 5 carbon atoms is more preferable.

Examples of the aryl group include an aryl group having 6 to 20 carbon atoms; and an aryl group having 6 to 15 carbon atoms is preferable, and an aryl group having 6 to 10 carbon atoms is more preferable.

A heterocyclic ring in the heterocyclic group preferably includes a 5-membered or 6-membered saturated or unsaturated heterocyclic ring. The heterocyclic ring may be fused with an aliphatic ring, an aromatic ring, or another heterocyclic ring. Examples of a heteroatom constituting the ring of the heterocyclic ring include B, N, O, S, Se, and Te; and N, O, or S is preferable. It is preferable that the carbon atom of the heterocyclic ring has a free valence (monovalent) (the heterocyclic group is bonded at the carbon atom). The number of carbon atoms in the heterocyclic group is preferably 1 to 40, more preferably 1 to 30, and still more preferably 1 to 20.

Examples of the group including a polymerizable group having an ethylenically unsaturated bond include a group represented by Formula (U1).

*—X^{U1}—Y^{U1}—Z^{U1} (U1)

In Formula (U1), X^{U1} represents a single bond, —C(═O)—, —C(═O)O—, or —C(═O)NRx³-, Rx³ represents a hydrogen atom, an alkyl group, or an aryl group,
Y^{U1} represents a single bond or a divalent linking group, and
Z^{U1} represents a polymerizable group having an ethylenically unsaturated bond.

The alkyl group and aryl group represented by Rx³ have the same meaning as the alkyl group and aryl group represented by Rx¹ of the group represented by Formula (T1), and preferred ranges thereof are also the same. Rx³ is preferably a hydrogen atom.

X^{U1} is preferably —C(═O)O— or —C(═O)NRx³-; and from the viewpoint of synthesis, more preferably —C(═O)O—.

Examples of the divalent linking group represented by Y^{U1} include the groups described as the divalent linking group represented by Y^{T1} of the group represented by Formula (T1), and a preferred range thereof is also the same.

Examples of the polymerizable group having an ethylenically unsaturated bond, represented by Z^{U1}, include a vinyl group, an allyl group, a (meth)acryloyl group, a (meth)acryloyloxy group, a (meth)acryloylamino group, and a vinylphenyl group; and a (meth)acryloyloxy group or a vinylphenyl group is preferable.

Specific examples of the group represented by Formula (U1) include groups represented by U-1 to U-11. In the following structural formulae, * is a bonding site.

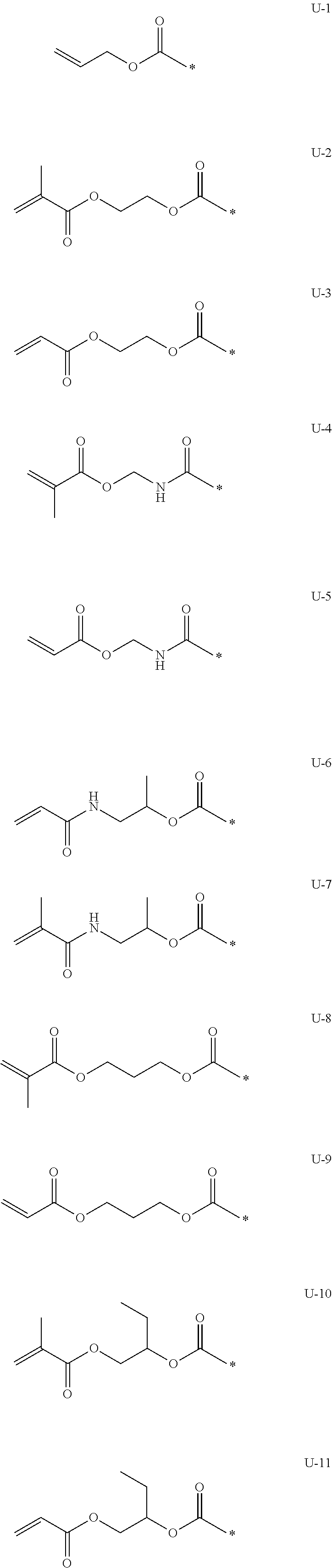

From the reason that the effect of the present invention is more remarkable, Q² in Formula (1) is preferably ═CR^{q2}R^{q3}. In addition, it is preferable that at least one of R^{q2} or R^{q3} is an electron withdrawing group, and it is more preferable that R^{q2} and R^{q3} are electron withdrawing groups.

It is also preferable that at least one of R^{q2} or R^{q3} is the group including a polymerizable group having an ethylenically unsaturated bond. In this aspect, it is also preferable that one of R^{q2} or R^{q3} is the group including a polymerizable group having an ethylenically unsaturated bond, and the other is an electron withdrawing group.

Examples of the electron withdrawing group include a substituent having the Hammett's substituent constant σp value of 0.2 or more. The Hammett's substituent constant σp value will be described. The Hammett's rule is an empirical rule advocated by L. P. Hammett in 1935 so as to quantitatively discuss the effect of substituent on the reaction or equilibrium of benzene derivatives and its propriety is widely admitted at present. Substituent constants obtained by the Hammett's rule are an σp value and an am value, and these values can be found in many general books. For example, these values are specifically described in “Lange's Handbook of Chemistry”, edited by J. A. Dean, 12th edition, 1979 (McGraw-Hill), “Chemistry Region”, extra edition, No. 122, pp. 96 to 103, 1979 (Nankodo, Co., Ltd.), and Chem. Rev., 1991, Vol. 91, pp. 165 to 195. In the present specification, a substituent having a Hammett's substituent constant σp value of 0.2 or more is denoted as the electron withdrawing group. The electron withdrawing group is preferably a group having a Hammett's substituent constant σp value of 0.25 or more, more preferably a group having a Hammett's substituent constant σp value of 0.3 or more, and still more preferably a group having a Hammett's substituent constant σp value of 0.35 or more.

Specific examples of the group having a Hammett's substituent constant σp value of 0.2 or more include a cyano group (σp value=0.66), a carboxyl group (—COOH; σp value=0.45), an alkoxycarbonyl group (—COOMe; σp value=0.45), an aryloxycarbonyl group (—COOPh; σp value=0.44), a carbamoyl group (—CONH₂; σp value=0.36), an alkylcarbonyl group (—COMe; σp value=0.50), an arylcarbonyl group (—COPh; σp value=0.43), an alkylsulfonyl group (—SO₂Me; σp value=0.72), and an arylsulfonyl group (—SO₂Ph; σp value=0.68). Me represents a methyl group and Ph represents a phenyl group. The values in the parentheses are representative σp values of the substituents extracted from “Chem. Rev.” vol. 91, pp. 165 to 195, 1991.

It is preferable that R^{q2} and R^{q3} are each independently a hydrogen atom, a cyano group, a carbamoyl group, a sulfamoyl group, an acyl group, an alkylsulfonyl group, an arylsulfonyl group, an alkylsulfinyl group, an arylsulfinyl group, a nitro group, an alkoxycarbonyl group, an aryloxycarbonyl group, or a group including a polymerizable group having an ethylenically unsaturated bond.

Examples of one aspect thereof include an aspect in which R^{q2} and R^{q3} are each independently a hydrogen atom, a cyano group, a carbamoyl group, a sulfamoyl group, an acyl group, an alkylsulfonyl group, an arylsulfonyl group, an alkylsulfinyl group, an arylsulfinyl group, a nitro group, an alkoxycarbonyl group, or an aryloxycarbonyl group. Among these, it is preferable that at least one of R^{q2} or R^{q3} is a cyano group, an alkoxycarbonyl group, a nitro group, or an alkylsulfonyl group, and it is more preferable that R^{q2} and R^{q3} are each independently a cyano group or an alkoxycarbonyl group. Examples of a preferred aspect thereof include an aspect in which R^{q2} and R^{q3} are cyano groups. Examples of another preferred aspect include an aspect in which one of R^{q2} or R^{q3} is a cyano group and the other is an alkoxycarbonyl group.

As another aspect, it is also preferable that at least one of R^{q2} or R^{q3} is the group including a polymerizable group having an ethylenically unsaturated bond. R^{q2} and R^{q3} may be each independently the group including a polymerizable group having an ethylenically unsaturated bond, and one of R^{q2} or R^{q3} may be the group including a polymerizable group having an ethylenically unsaturated bond and the other may be the electron withdrawing group.

In the present specification, the expression “in a case where R^{q2} and R^{q3} are bonded to each other to form a ring, ═CR^{q2}R^{q3} does not have the same structure as Q¹” includes not only a case of forming a ring other than the structure represented by Formula (Q-1), but also a case of forming a ring having a structure which is the structure represented by Formula (Q-1), in which the types of Rq¹ and Rq² in Formula (Q-1) are different from the type of Q¹. That is, Q² is a group having a structure different from that of Q¹.

In a case where R^{q2} and R^{q3} of ═CR^{q2}R^{q3} are bonded to each other to form a ring, from the reason that the effect of the present invention is more remarkable, the formed ring is preferably a ring other than the structure represented by Formula (Q-1). Examples of the ring other than the structure represented by Formula (Q-1) include a cyclopropane ring, a cyclobutane ring, a cyclopentane ring, a cyclohexane ring, a cycloheptane ring, a pyrrolidine ring, a tetrahydrofuran ring, a tetrahydrothiophene ring, an oxazoline ring, a thiazoline ring, a pyrroline ring, a pyrazoline ring, an imidazoline ring, an imidazolidine ring, a piperidine ring, a piperazine ring, and a pyran ring. These rings may have a substituent at any position.

It is preferable that at least one of R¹, R², Q¹, or Q² in Formula (1) includes the group including a polymerizable group having an ethylenically unsaturated bond, and it is more preferable that one or two of R¹, R², Q¹, and Q² include the group including a polymerizable group having an ethylenically unsaturated bond.

In addition, it is preferable that, in Formula (1), Q² is ═CR^{q2}R^{q3} and at least one of R¹, R², R^{q2}, R^{q3}, R¹⁰¹, or R¹⁰² is the group including a polymerizable group having an ethylenically unsaturated bond, and it is more preferable that one or two of R¹, R², R^{q2}, R^{q3}, R¹⁰¹, or R¹⁰² are the group including a polymerizable group having an ethylenically unsaturated bond.

In addition, the number of the polymerizable groups having an ethylenically unsaturated bond included in Formula (1) is preferably 1 or 2.

The specific compound is preferably a compound represented by Formula (3). The compound represented by Formula (3) is the compound according to the embodiment of the present invention.

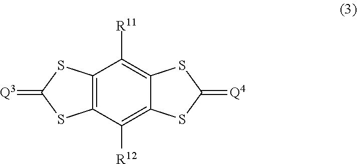

In Formula (3), Q³ represents the group represented by Formula (Q-1);
Q⁴ represents ═O, ═S, ═NR^{q11}, or ═CR^{q12}R^{q13}, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³; and
R¹¹ and R¹² each independently represent —OH, —O—Y¹¹, —OC(═O)—Y¹¹, —OC(═O)O—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.

Q³ and Q⁴ in Formula (3) have the same meaning as Q¹ and Q² in Formula (1), and preferred ranges thereof are also the same. In addition, preferred ranges of R^{y11} and Y¹¹ in Formula (3) are the same as those for R^{y11} and Y¹¹ described in Formula (1).

Examples of one aspect thereof include an aspect in which R¹¹ and R¹² in Formula (3) are each independently —OH, —O—Y¹¹, —OC(═O)—Y¹¹, —OC(═O)O—Y¹¹, —OC(═O)NR^{y11}—Y¹¹, or —OSO₂—Y¹¹. In this aspect, it is preferable that R¹¹ and R¹² are each independently —OC(═O)—Y¹¹, —O—Y¹¹, or —OC(═O)NR^{y11}—Y¹¹.

Examples of another aspect thereof include an aspect in which at least one of R¹¹ or R¹² is a group including the group including a polymerizable group having an ethylenically unsaturated bond.

It is preferable that at least one of R¹¹, R¹², Q³, or Q⁴ in Formula (3) includes the group including a polymerizable group having an ethylenically unsaturated bond, and it is more preferable that one or two of R¹¹, R¹², Q³, and Q⁴ include the group including a polymerizable group having an ethylenically unsaturated bond.

In addition, it is preferable that, in Formula (3), Q⁴ is ═CR^{q12}R^{q13} and at least one of R¹¹, R¹², R^{q12}, R^{q13}, R¹⁰¹, or R¹⁰² is the group including a polymerizable group having an ethylenically unsaturated bond, and it is more preferable that one or two of R¹¹, R¹², R^{q12}, R^{q13}, R¹⁰¹, or R¹⁰² are the group including a polymerizable group having an ethylenically unsaturated bond.

The number of the polymerizable groups having an ethylenically unsaturated bond included in Formula (3) is preferably 1 or 2.

The specific compound is preferably a compound represented by Formula (6).

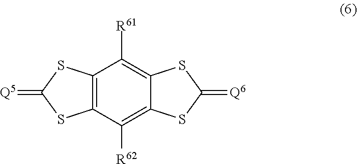

In Formula (6), Q⁵ represents a group represented by Formula (Q-1) described above, Q⁶ represents ═O, ═S, ═NR^{q21}, or ═CR^{q22}R^{q23}, R^{q21} to R^{q23} each independently represent a hydrogen atom or a substituent, R^{q22} and R^{q23} may be bonded to each other to form a ring, provided that, in a case where R^{q22} and R^{q23} are bonded to each other to form a ring, ═CR^{q22}R^{q23} does not have the same structure as Q⁵; and
R⁶¹ and R⁶² each independently represent —O—Y⁶¹, —OC(═O)—Y⁶¹, —OC(═O)O—Y⁶¹, —OC(═O)NR^{y61}—Y⁶¹, or —OSO₂—Y⁶¹, where R^{y61} represents a hydrogen atom, an alkyl group, an aralkyl group, or an aryl group, and Y⁶¹ represents an alkyl group, an aralkyl group, or an aryl group.

Q⁵ and Q⁶ in Formula (6) have the same meaning as Q¹ and Q² in Formula (1), and preferred ranges thereof are also the same. In addition, preferred ranges of R^{y61} and Y⁶¹ in Formula (6) are the same as those for R^{y11} and Y¹¹ described in Formula (1).

It is preferable that R⁶¹ and R⁶² in Formula (6) are each independently —OC(═O)—Y¹¹, —O—Y¹¹, or —OC(═O)NR^{y11}—Y¹¹.

### (Substituent T)

Examples of the substituent T include the following groups:
a halogen atom (for example, a chlorine atom, a bromine atom, or an iodine atom);
an alkyl group [a linear, branched, or cyclic alkyl group; including a linear or branched alkyl group (preferably a linear or branched alkyl group having 1 to 30 carbon atoms; for example, a methyl group, an ethyl group, an n-propyl group, an isopropyl group, a t-butyl group, an n-octyl group, an eicosyl group, a 2-chloroethyl group, a 2-cyanoethyl group, and a 2-ethylhexyl group), a cycloalkyl group (preferably a cycloalkyl group having 3 to 30 carbon atoms; for example, a cyclohexyl group, a cyclopentyl group, and a 4-n-dodecylcyclohexyl group), a bicycloalkyl group (preferably a bicycloalkyl group having 5 to 30 carbon atoms, that is, a monovalent group obtained by removing one hydrogen atom from a bicycloalkane having 5 to 30 carbon atoms; for example, a bicyclo[1,2,2]heptan-2-yl group and a bicyclo[2,2,2]octan-3-yl group), a tricyclo structure having many ring structures, and the like; an alkyl group in the substituent described below (for example, an alkyl group in the alkylthio group) also represents the alkyl group of this concept];
an alkenyl group [a linear, branched, or cyclic alkenyl group; including a linear or branched alkenyl group (preferably a linear or branched alkenyl group having 2 to 30 carbon atoms; for example, a vinyl group, an allyl group, a prenyl group, a geranyl group, and an oleyl group), a cycloalkenyl group (preferably a cycloalkenyl group having 3 to 30 carbon atoms, that is, a monovalent group obtained by removing one hydrogen atom from a cycloalkene having 3 to 30 carbon atoms; for example, a 2-cyclopenten-1-yl group and a 2-cyclohexen-1-yl group), and a bicycloalkenyl group (preferably a bicycloalkenyl group having 5 to 30 carbon atoms, that is, a monovalent group obtained by removing one hydrogen atom from a bicycloalkene with one double bond; for example, a bicyclo[2,2,1]hept-2-en-1-yl group and a bicyclo[2,2,2]oct-2-en-4-yl group);
an alkynyl group (preferably a linear or branched alkynyl group having 2 to 30 carbon atoms; for example, an ethynyl group and a propargyl group);
an aryl group (preferably an aryl group having 6 to 30 carbon atoms; for example, a phenyl group, a p-tolyl group, a naphthyl group, an m-chlorophenyl group, and an o-hexadecanoylaminophenyl group);
a heterocyclic group (preferably a monovalent group obtained by removing one hydrogen atom from an aromatic or non-aromatic heterocyclic compound having 5 or 6 ring members, and more preferably a 5- or 6-membered aromatic heterocyclic group having 3 to 30 carbon atoms; for example, a 2-furyl group, a 2-thienyl group, a 2-pyrimidinyl group, and a 2-benzothiazolyl group);
a cyano group;
a hydroxy group;
a nitro group;
a carboxyl group;
an alkoxy group (preferably a linear or branched alkoxy group having 1 to 30 carbon atoms; for example, a methoxy group, an ethoxy group, an isopropoxy group, a t-butoxy group, an n-octyloxy group, and a 2-methoxyethoxy group);
an aryloxy group (preferably an aryloxy group having 6 to 30 carbon atoms; for example, a phenoxy group, a 2-methylphenoxy group, a 4-t-butylphenoxy group, a 3-nitrophenoxy group, and a 2-tetradecanoylaminophenoxy group);
a heterocyclic oxy group (preferably a heterocyclic oxy group having 2 to 30 carbon atoms; for example, a 1-phenyltetrazol-5-oxy group and a 2-tetrahydropyranyloxy group);
an acyloxy group (preferably a formyloxy group, an alkylcarbonyloxy group having 2 to 30 carbon atoms, or an arylcarbonyloxy group having 6 to 30 carbon atoms; for example, a formyloxy group, an acetyloxy group, a pivaloyloxy group, a stearoyloxy group, a benzoyloxy group, and a p-methoxyphenylcarbonyloxy group);
a carbamoyloxy group (preferably a carbamoyloxy group having 1 to 30 carbon atoms; for example, an N,N-dimethylcarbamoyloxy group, an N,N-diethylcarbamoyloxy group, a morpholinocarbonyloxy group, an N,N-di-n-octylaminocarbonyloxy group, and an N-n-octylcarbamoyloxy group);
an alkoxycarbonyloxy group (preferably an alkoxycarbonyloxy group having 2 to 30 carbon atoms; for example, a methoxycarbonyloxy group, an ethoxycarbonyloxy group, a t-butoxycarbonyloxy group, and an n-octylcarbonyloxy group);
an aryloxycarbonyloxy group (preferably an aryloxycarbonyloxy group having 7 to 30 carbon atoms; for example, a phenoxycarbonyloxy group, a p-methoxyphenoxycarbonyloxy group, and a p-n-hexadecyloxyphenoxycarbonyloxy group);
an amino group (preferably an amino group, an alkylamino group having 1 to 30 carbon atoms, or an anilino group having 6 to 30 carbon atoms; for example, an amino group, a methylamino group, a dimethylamino group, an anilino group, an N-methyl-anilino group, and a diphenylamino group);
an acylamino group (preferably a formylamino group, an alkylcarbonylamino group having 2 to 30 carbon atoms, or an arylcarbonylamino group having 6 to 30 carbon atoms; for example, a formylamino group, an acetylamino group, a pivaloylamino group, a lauroylamino group, a benzoylamino group, and a 3,4,5-tri-n-octyloxyphenylcarbonylamino group);
an aminocarbonylamino group (preferably an aminocarbonylamino group having 1 to 30 carbon atoms; for example, a carbamoylamino group, an N,N-dimethylaminocarbonylamino group, an N,N-diethylaminocarbonylamino group, and a morpholinocarbonylamino group);
an alkoxycarbonylamino group (preferably an alkoxycarbonylamino group having 2 to 30 carbon atoms; for example, a methoxycarbonylamino group, an ethoxycarbonylamino group, a t-butoxycarbonylamino group, an n-octadecyloxycarbonylamino group, and an N-methyl-methoxycarbonylamino group);
an aryloxycarbonylamino group (preferably an aryloxycarbonylamino group having 7 to 30 carbon atoms; for example, a phenoxycarbonylamino group, a p-chlorophenoxycarbonylamino group, and an m-n-octyloxyphenoxycarbonylamino group);
a sulfamoylamino group (preferably a sulfamoylamino group having 0 to 30 carbon atoms; for example, a sulfamoylamino group, an N,N-dimethylaminosulfonylamino group, and an N-n-octylaminosulfonylamino group);
an alkylsulfonylamino group or arylsulfonylamino group (preferably an alkylsulfonylamino group having 1 to 30 carbon atoms or an arylsulfonylamino group having 6 to 30 carbon atoms; for example, a methylsulfonylamino group, a butylsulfonylamino group, a phenylsulfonylamino group, a 2,3,5-trichlorophenylsulfonylamino group, and a p-methylphenylsulfonylamino group);
a mercapto group;
an alkylthio group (preferably an alkylthio group having 1 to 30 carbon atoms; for example, a methylthio group, an ethylthio group, and an n-hexadecylthio group);
an arylthio group (preferably an arylthio group having 6 to 30 carbon atoms; for example, a phenylthio group, a p-chlorophenylthio group, and an m-methoxyphenylthio group);
a heterocyclic thio group (preferably a heterocyclic thio group having 2 to 30 carbon atoms; for example a 2-benzothiazolylthio group and a 1-phenyltetrazol-5-ylthio group);
a sulfamoyl group (preferably a sulfamoyl group having 0 to 30 carbon atoms; for example, an N-ethylsulfamoyl group, an N-(3-dodecyloxypropyl)sulfamoyl group, an N,N-dimethylsulfamoyl group, an N-acetylsulfamoyl group, an N-benzoylsulfamoyl group, and an N—(N′-phenylcarbamoyl)sulfamoyl group);
a sulfo group;
an alkylsulfinyl group or arylsulfinyl group (preferably an alkylsulfinyl group having 1 to 30 carbon atoms or an arylsulfinyl group having 6 to 30 carbon atoms; for example, a methylsulfinyl group, an ethylsulfinyl group, a phenylsulfinyl group, and a p-methylphenylsulfinyl group);
an alkylsulfonyl group or arylsulfonyl group (preferably an alkylsulfonyl group having 1 to 30 carbon atoms or an arylsulfonyl group having 6 to 30 carbon atoms; for example, a methylsulfonyl group, an ethylsulfonyl group, a phenylsulfonyl group, and a p-methylphenylsulfonyl group);
an acyl group (preferably a formyl group, an alkylcarbonyl group having 2 to 30 carbon atoms, an arylcarbonyl group having 7 to 30 carbon atoms, or a heterocyclic carbonyl group having 4 to 30 carbon atoms, in which a heterocycle is bonded to a carbonyl group at a carbon atom; for example, an acetyl group, a pivaloyl group, a 2-chloroacetyl group, a stearoyl group, a benzoyl group, a p-n-octyloxyphenylcarbonyl group, a 2-pyridyl carbonyl group, and a 2-furylcarbonyl group);
an aryloxycarbonyl group (preferably an aryloxycarbonyl group having 7 to 30 carbon atoms; for example, a phenoxycarbonyl group, an o-chlorophenoxycarbonyl group, an m-nitrophenoxycarbonyl group, and a p-t-butylphenoxycarbonyl group);
an alkoxycarbonyl group (preferably an alkoxycarbonyl group having 2 to 30 carbon atoms; for example, a methoxycarbonyl group, an ethoxycarbonyl group, a t-butoxycarbonyl group, and an n-octadecyloxycarbonyl group);
a carbamoyl group (preferably a carbamoyl group having 1 to 30 carbon atoms; for example, a carbamoyl group, an N-methylcarbamoyl group, an N,N-dimethylcarbamoyl group, an N,N-di-n-octylcarbamoyl group, and an N-(methylsulfonyl)carbamoyl group);
an arylazo group or heterocyclic azo group (preferably an arylazo group having 6 to 30 carbon atoms or a heterocyclic azo group having 3 to 30 carbon atoms; for example, a phenylazo group, a p-chlorophenylazo group, and a 5-ethylthio-1,3,4-thiadiazol-2-ylazo group);
an imide group (preferably an N-succinimide group or an N-phthalimide group);
a phosphino group (preferably a phosphino group having 2 to 30 carbon atoms; for example, a dimethylphosphino group, a diphenylphosphino group, and a methylphenoxyphosphino group);
a phosphinyl group (preferably a phosphinyl group having 2 to 30 carbon atoms; for example, a phosphinyl group, a dioctyloxyphosphinyl group, and a diethoxyphosphinyl group);
a phosphinyloxy group (preferably a phosphinyloxy group having 2 to 30 carbon atoms; for example, a diphenoxyphosphinyloxy group and a dioctyloxyphosphinyloxy group); and
a phosphinylamino group (preferably a phosphinylamino group having 2 to 30 carbon atoms; for example a dimethoxyphosphinylamino group and a dimethylaminophosphinylamino group).

Among the groups described above, one or more hydrogen atoms of a group having hydrogen atoms may be substituted with the above-described substituents T. Examples of such substituents include an alkylcarbonylaminosulfonyl group, an arylcarbonylaminosulfonyl group, an alkylsulfonylaminocarbonyl group, and an arylsulfonylaminocarbonyl group. Specific examples thereof include a methylsulfonylaminocarbonyl group, a p-methylphenylsulfonylaminocarbonyl group, an acetylaminosulfonyl group, and a benzoylaminosulfonyl group.

Specific examples of the specific compound include compounds having the following structures. In the structural formulae shown below, Et is an ethyl group, Me is a methyl group, ⁿBu is an n-butyl group, ^{t}Bu is a tert-butyl group, and Ph is a phenyl group.

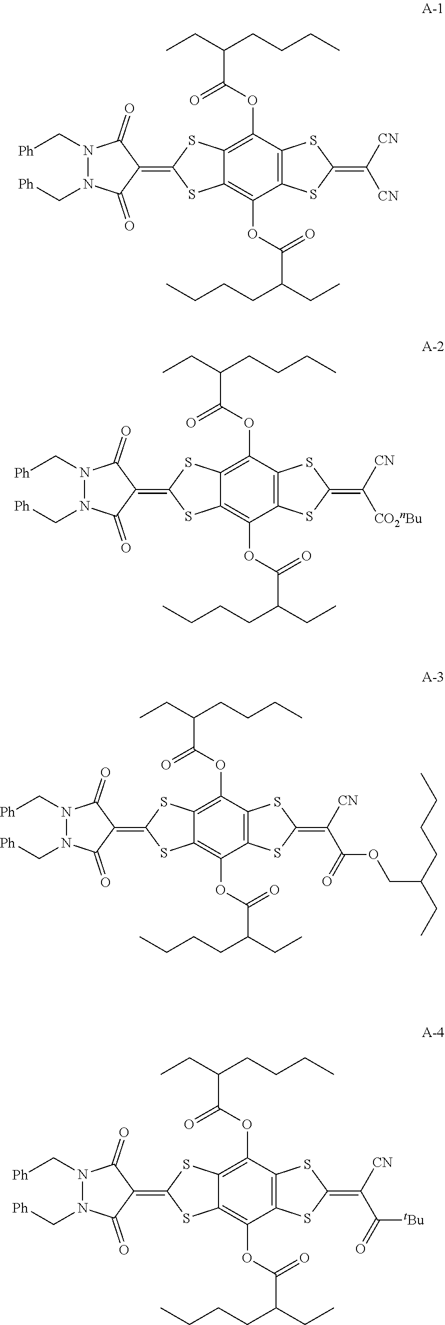

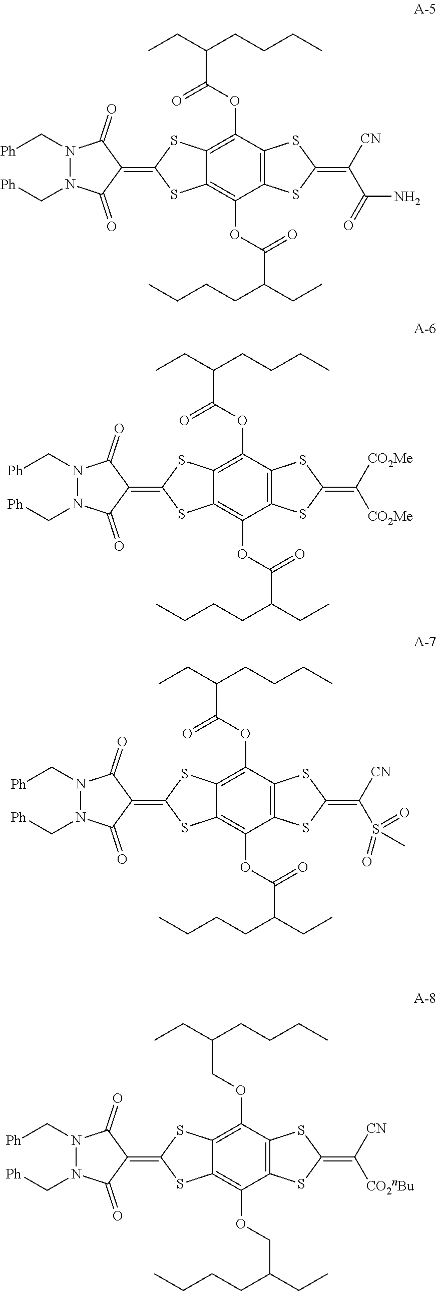

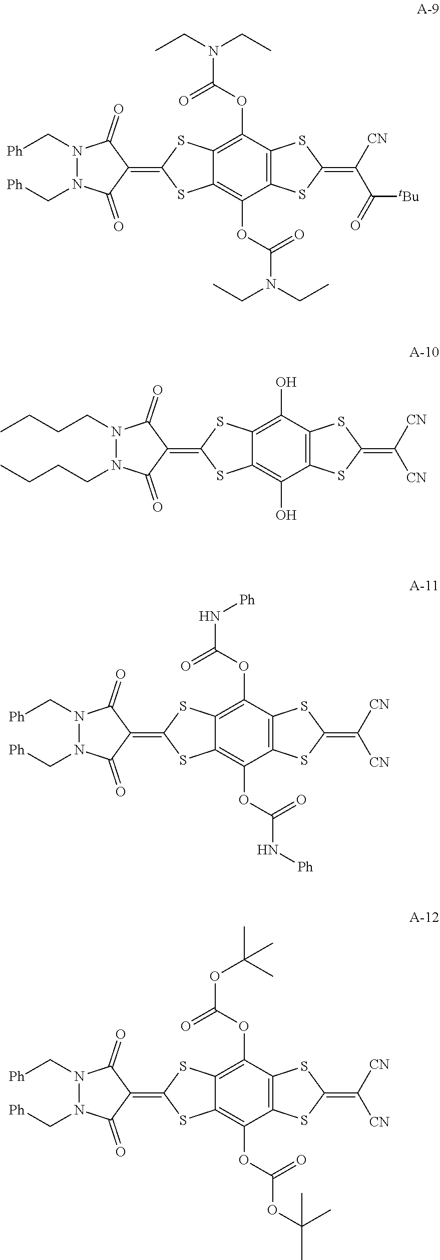 [SMILES: CCN(CC)C(=O)Oc1c2c(c(OC(=O)N(CC)CC)c3c1SC(=C1C(=O)N(Cc4ccccc4)N(Cc4ccccc4)C1=O)S3)SC(=C(C#N)C(=O)C(C)(C)C)S2 ; CCCCN1C(=O)C(=C2Sc3c(O)c4c(c(O)c3S2)SC(=C(C#N)C#N)S4)C(=O)N1CCCC ; N#CC(C#N)=C1Sc2c(OC(=O)Nc3ccccc3)c3c(c(OC(=O)Nc4ccccc4)c2S1)SC(=C1C(=O)N(Cc2ccccc2)N(Cc2ccccc2)C1=O)S3 ; CC(C)(C)OC(=O)Oc1c2c(c(OC(=O)OC(C)(C)C)c3c1SC(=C1C(=O)N(Cc4ccccc4)N(Cc4ccccc4)C1=O)S3)SC(=C(C#N)C#N)S2]

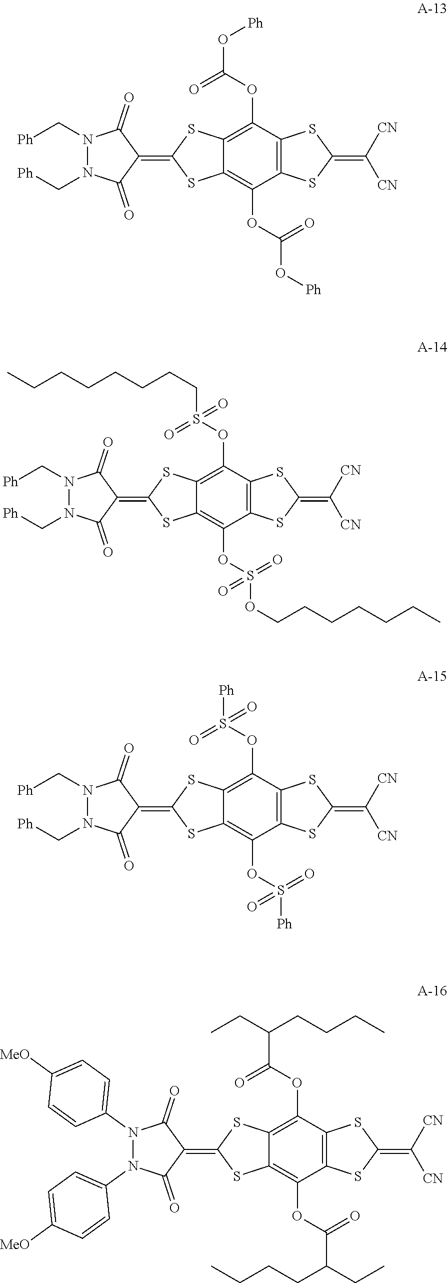 [SMILES: N#CC(C#N)=C1Sc2c(OC(=O)Oc3ccccc3)c3c(c(OC(=O)Oc4ccccc4)c2S1)SC(=C1C(=O)N(Cc2ccccc2)N(Cc2ccccc2)C1=O)S3 ; CCCCCCCCS(=O)(=O)Oc1c2c(c(OS(=O)(=O)OCCCCCCC)c3c1SC(=C1C(=O)N(Cc4ccccc4)N(Cc4ccccc4)C1=O)S3)SC(=C(C#N)C#N)S2 ; N#CC(C#N)=C1Sc2c(OS(=O)(=O)c3ccccc3)c3c(c(OS(=O)(=O)c4ccccc4)c2S1)SC(=C1C(=O)N(Cc2ccccc2)N(Cc2ccccc2)C1=O)S3 ; CCCCC(CC)C(=O)Oc1c2c(c(OC(=O)C(CC)CCCC)c3c1SC(=C1C(=O)N(c4ccc(OC)cc4)N(c4ccc(OC)cc4)C1=O)S3)SC(=C(C#N)C#N)S2]

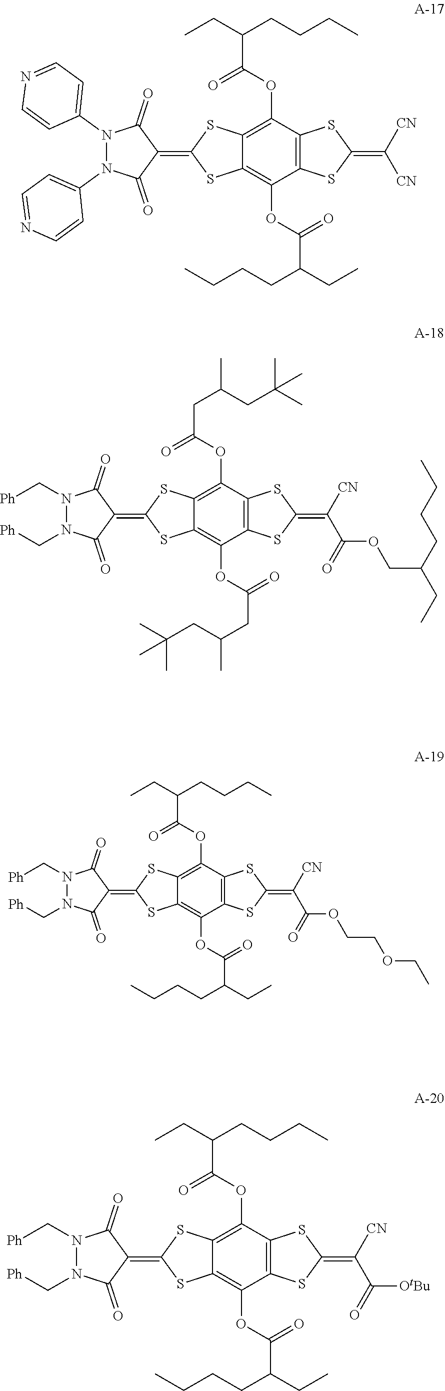 [SMILES: CCCCC(CC)C(=O)Oc1c2c(c(OC(=O)C(CC)CCCC)c3c1SC(=C1C(=O)N(c4ccncc4)N(c4ccncc4)C1=O)S3)SC(=C(C#N)C#N)S2 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C#N)C(=O)OCCOCC)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C#N)C(=O)OC(C)(C)C)S2]

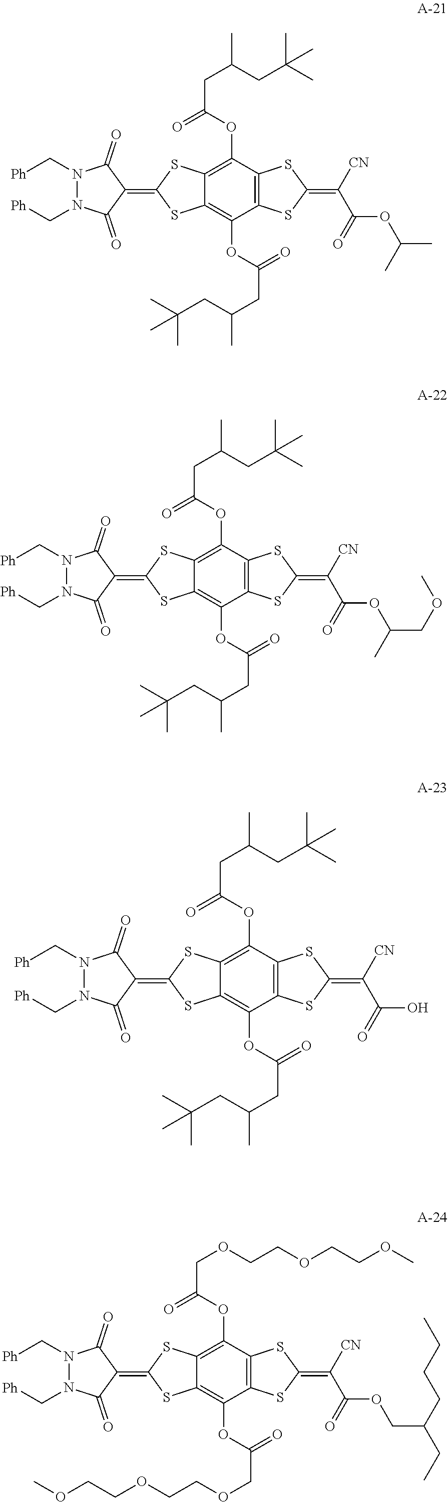 [SMILES: CC(CC(=O)Oc1c2c(c(OC(=O)CC(C)CC(C)(C)C)c3c1SC(=C1C(=O)N(Cc4ccccc4)N(Cc4ccccc4)C1=O)S3)SC(=C(C#N)C(=O)OC(C)C)S2)CC(C)(C)C ; COCC(C)OC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; CC(CC(=O)Oc1c2c(c(OC(=O)CC(C)CC(C)(C)C)c3c1SC(=C1C(=O)N(Cc4ccccc4)N(Cc4ccccc4)C1=O)S3)SC(=C(C#N)C(=O)O)S2)CC(C)(C)C ; CCCCC(CC)COC(=O)C(C#N)=C1Sc2c(OC(=O)COCCOCCOC)c3c(c(OC(=O)COCCOCCOC)c2S1)SC(=C1C(=O)N(Cc2ccccc2)N(Cc2ccccc2)C1=O)S3]

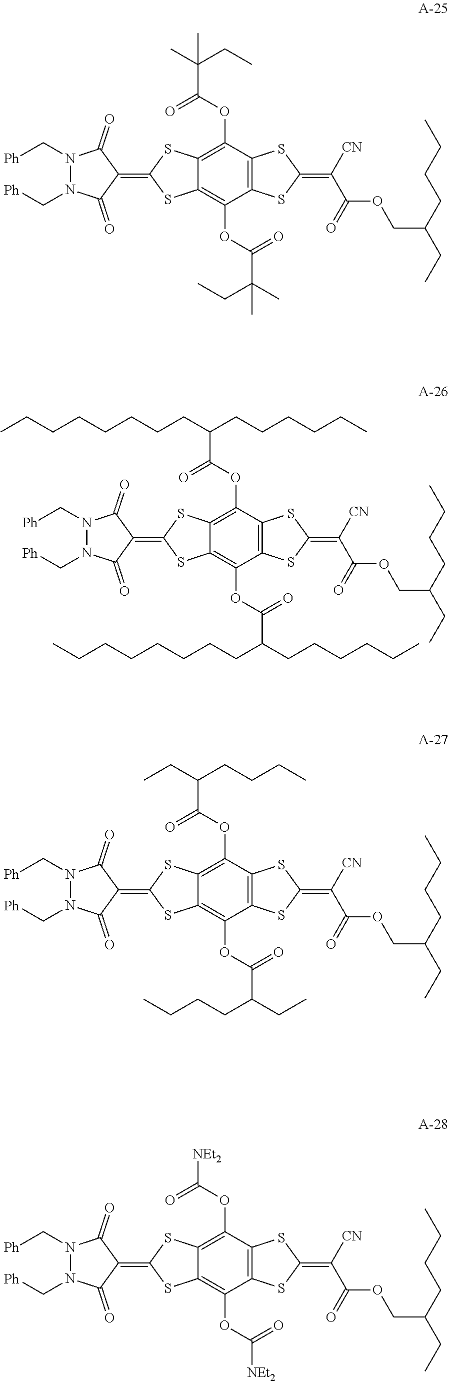

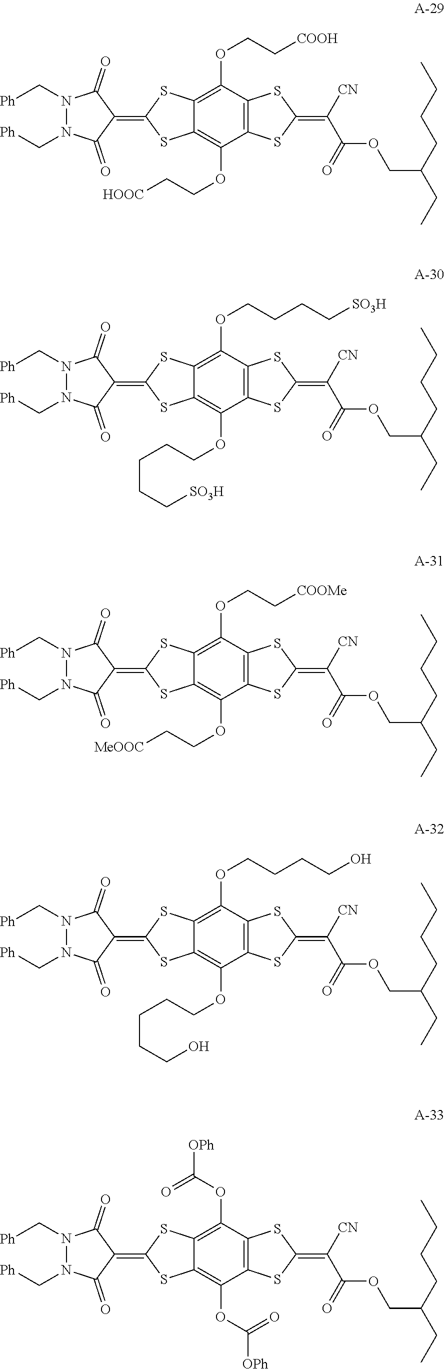

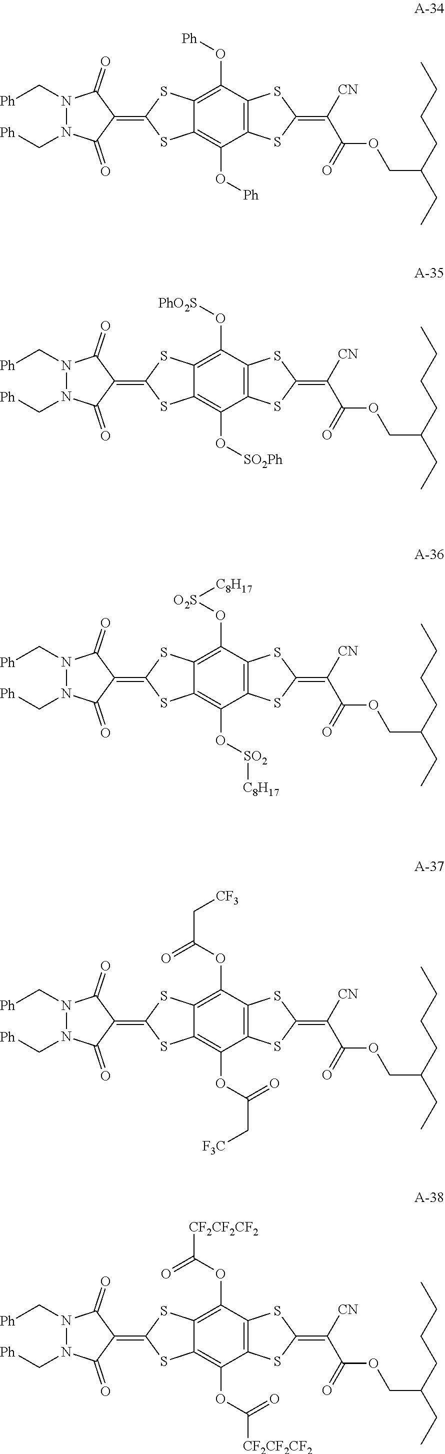

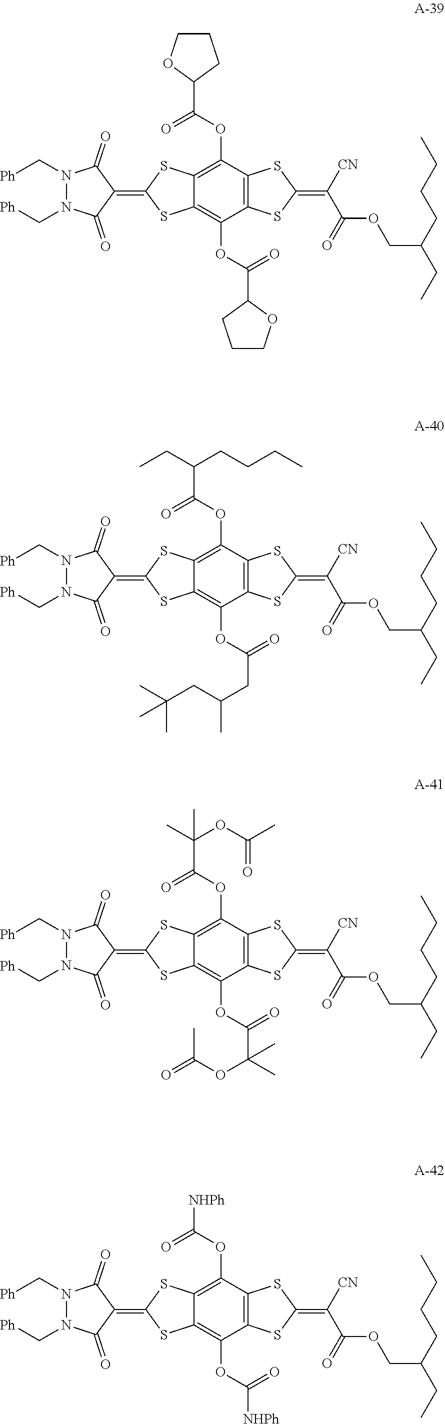

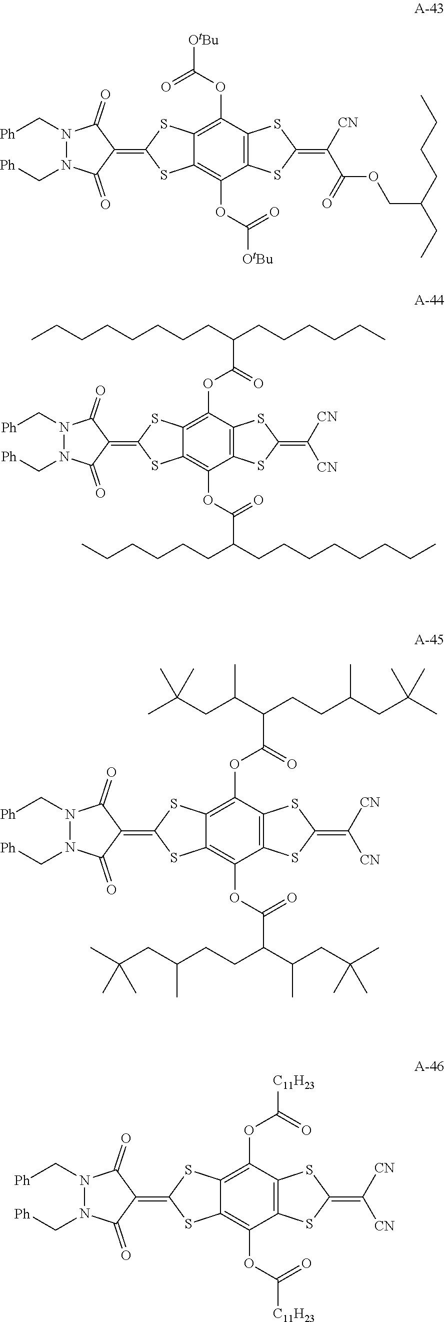

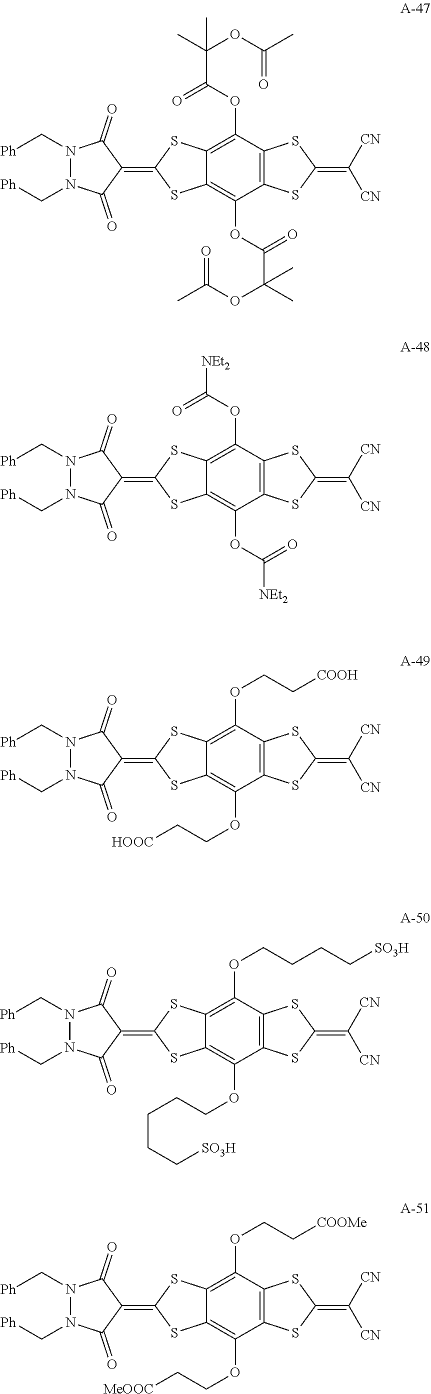

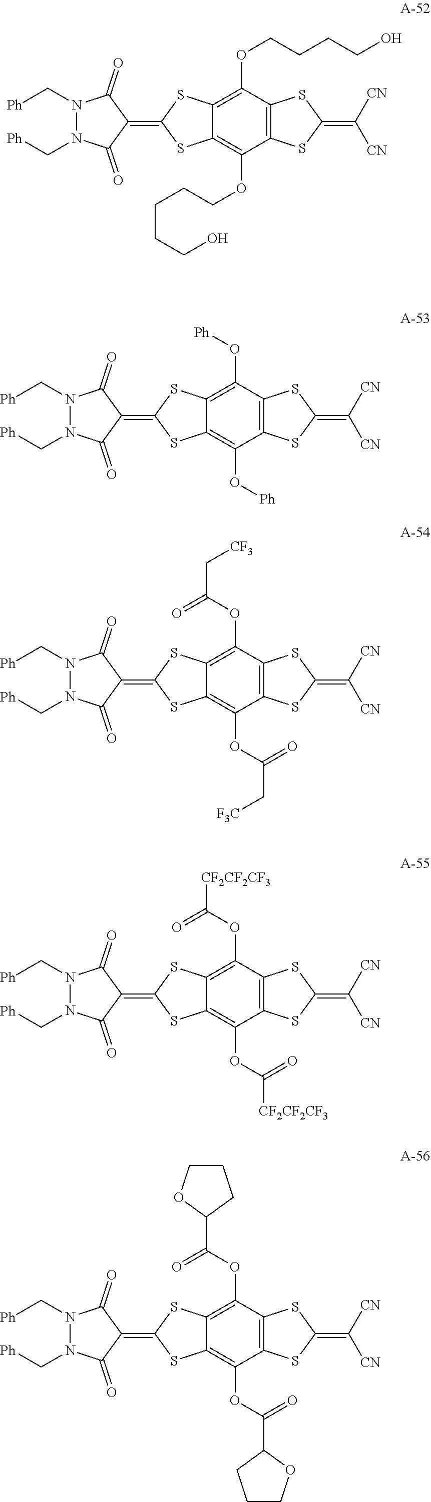

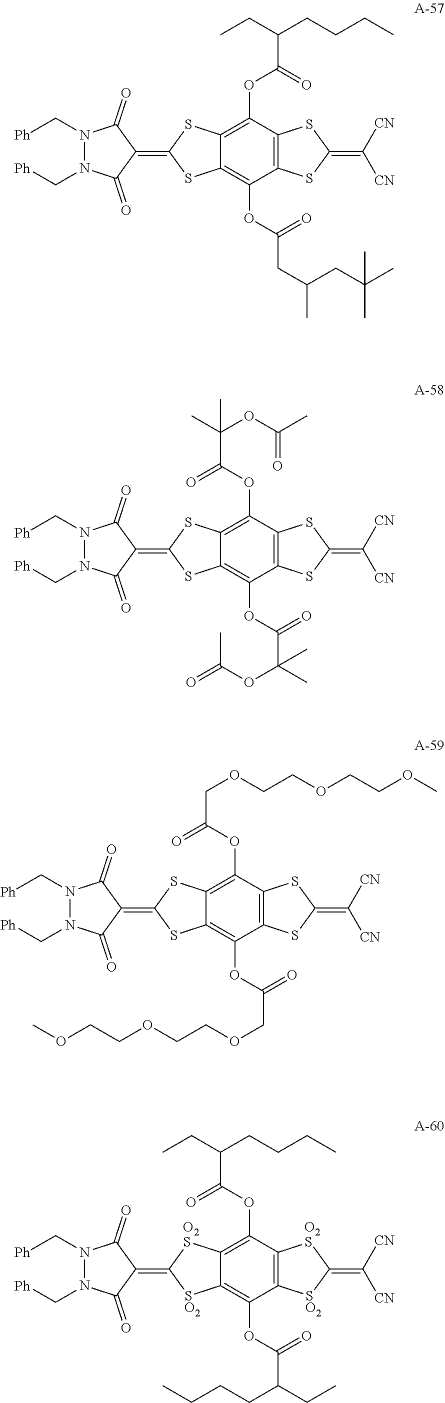 [SMILES: CCCCC(CC)C(=O)Oc1c2c(c(OC(=O)CC(C)CC(C)(C)C)c3c1SC(=C1C(=O)N(Cc4ccccc4)N(Cc4ccccc4)C1=O)S3)SC(=C(C#N)C#N)S2 ; CC(=O)OC(C)(C)C(=O)Oc1c2c(c(OC(=O)C(C)(C)OC(C)=O)c3c1SC(=C1C(=O)N(Cc4ccccc4)N(Cc4ccccc4)C1=O)S3)SC(=C(C#N)C#N)S2 ; COCCOCCOCC(=O)Oc1c2c(c(OC(=O)COCCOCCOC)c3c1SC(=C1C(=O)N(Cc4ccccc4)N(Cc4ccccc4)C1=O)S3)SC(=C(C#N)C#N)S2 ; CCCCC(CC)C(=O)Oc1c2c(c(OC(=O)C(CC)CCCC)c3c1S(=O)(=O)C(=C1C(=O)N(Cc4ccccc4)N(Cc4ccccc4)C1=O)S3(=O)=O)S(=O)(=O)C(=C(C#N)C#N)S2(=O)=O]

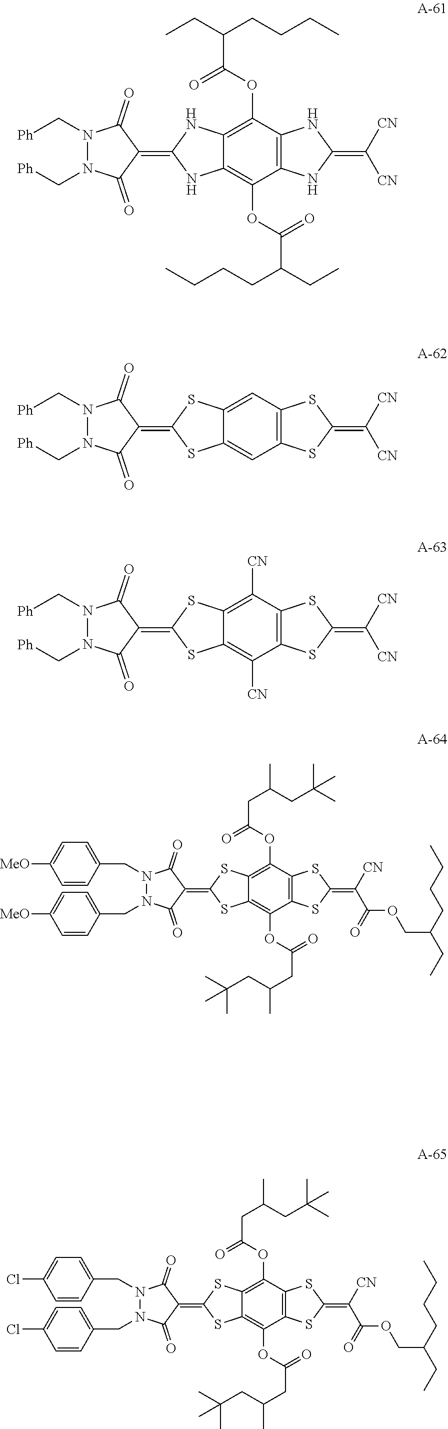

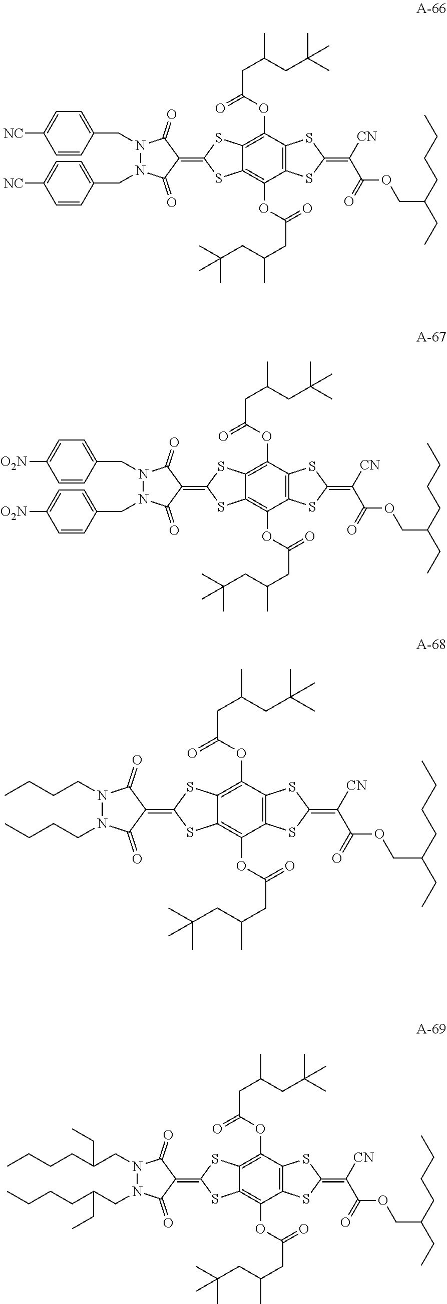

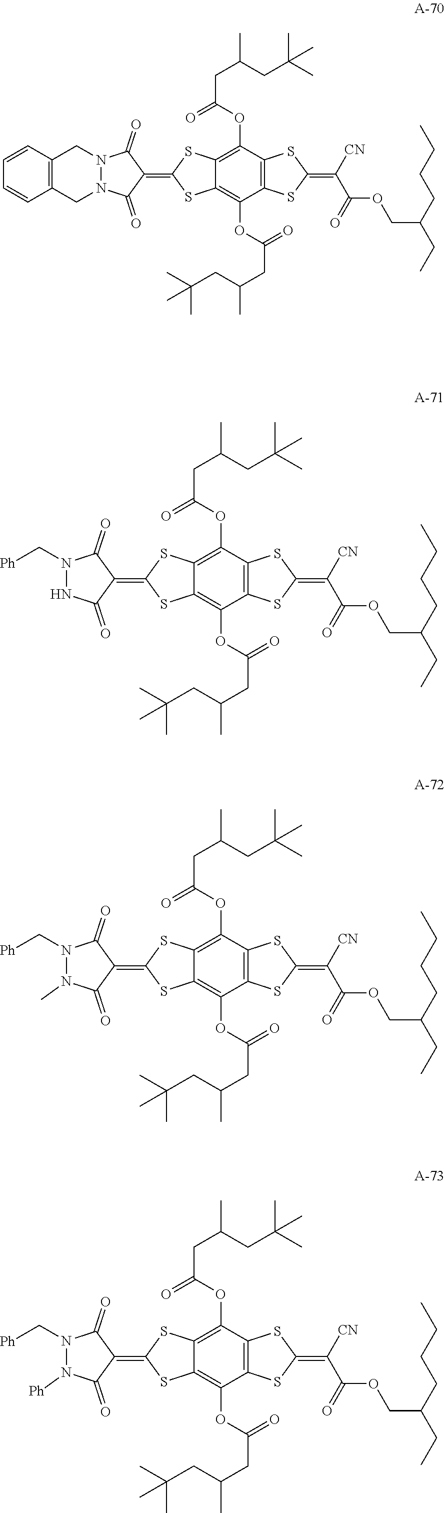 [SMILES: CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N2Cc4ccccc4CN2C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)NN(Cc2ccccc2)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(C)N(Cc2ccccc2)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(c2ccccc2)C1=O)S3]

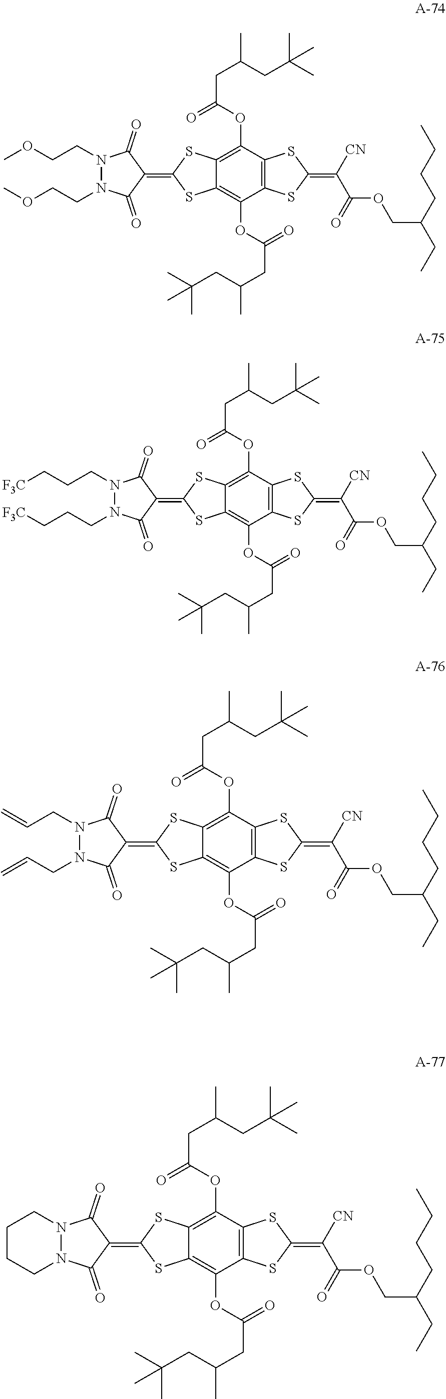 [SMILES: CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(CCOC)N(CCOC)C1=O)S3 ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(CCCC(F)(F)F)N(CCCC(F)(F)F)C1=O)S3 ; C=CCN1C(=O)C(=C2Sc3c(OC(=O)CC(C)CC(C)(C)C)c4c(c(OC(=O)CC(C)CC(C)(C)C)c3S2)SC(=C(C#N)C(=O)OCC(CC)CCCC)S4)C(=O)N1CC=C ; CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N2CCCCN2C1=O)S3]

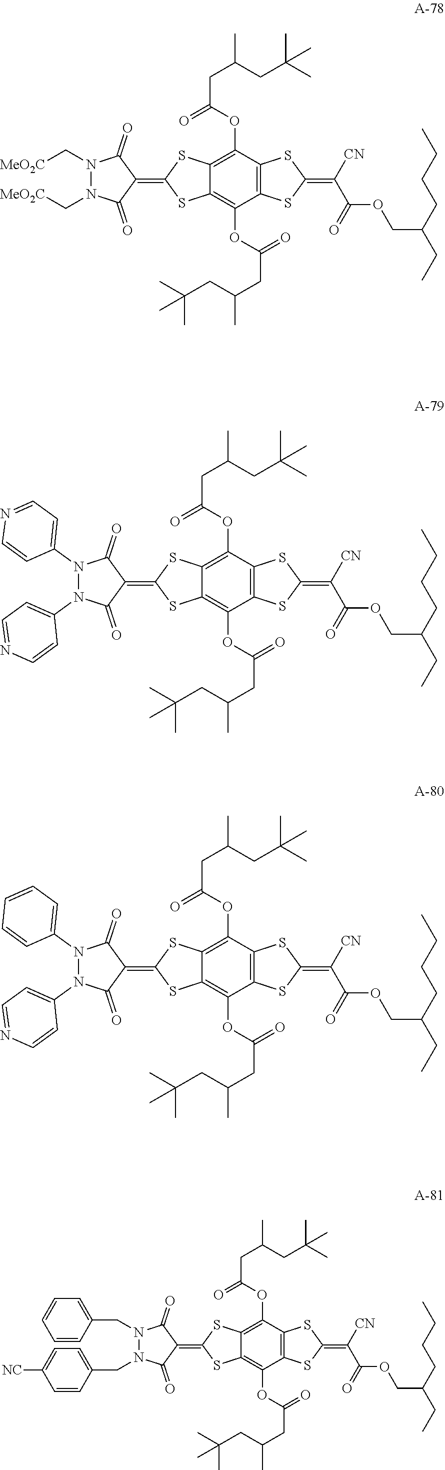

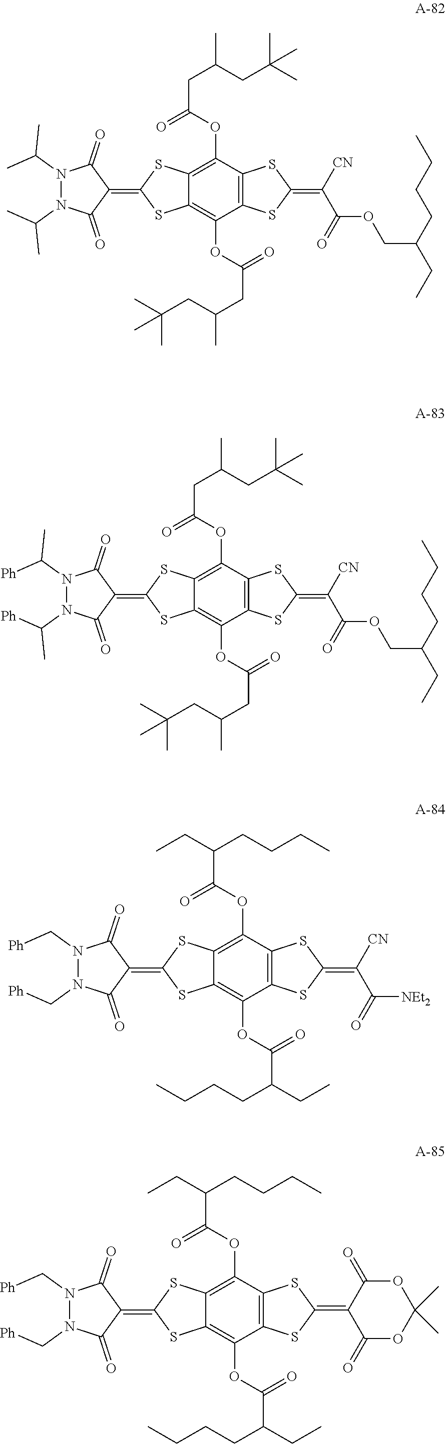

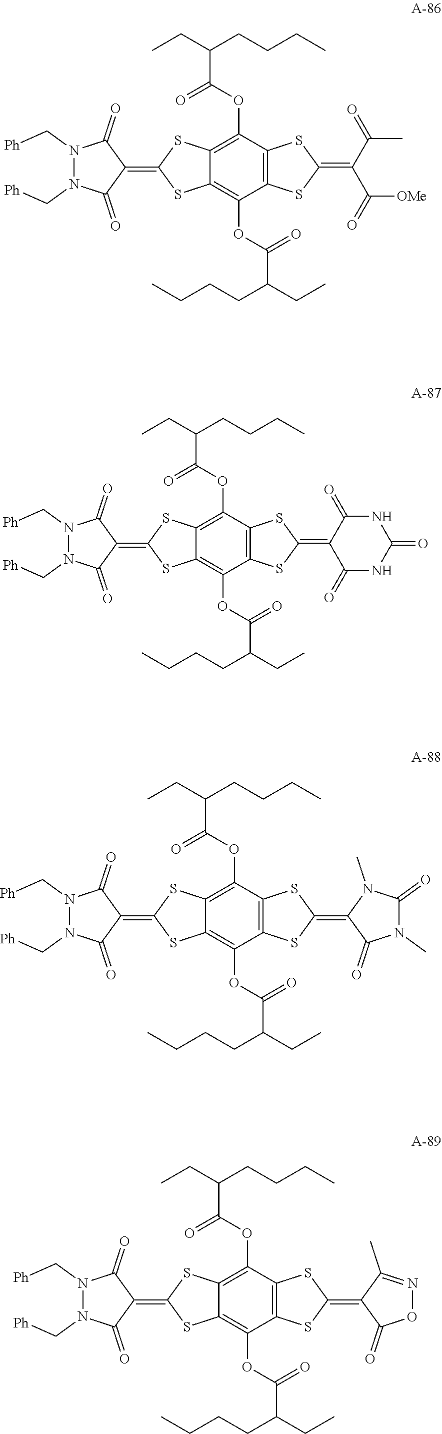 [SMILES: CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C(C)=O)C(=O)OC)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C1C(=O)NC(=O)NC1=O)S2 ; CCCCC(CC)C(=O)Oc1c2c(c(OC(=O)C(CC)CCCC)c3c1SC(=C1C(=O)N(C)C(=O)N1C)S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3c1SC(=C1C(=O)ON=C1C)S3)SC(=C1C(=O)N(Cc3ccccc3)N(Cc3ccccc3)C1=O)S2]

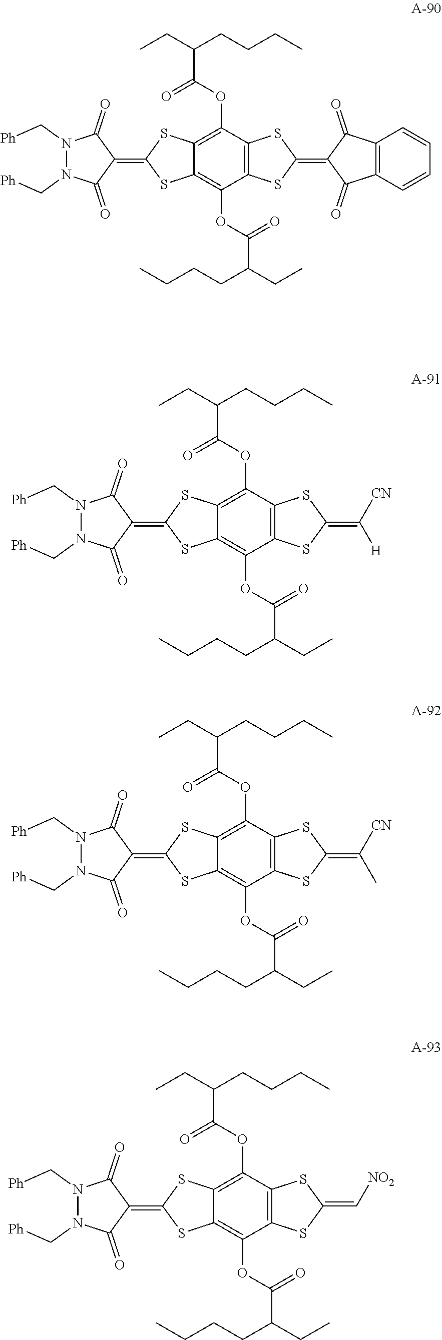 [SMILES: CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C1C(=O)c3ccccc3C1=O)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=CC#N)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C)C#N)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C[N+](=O)[O-])S2]

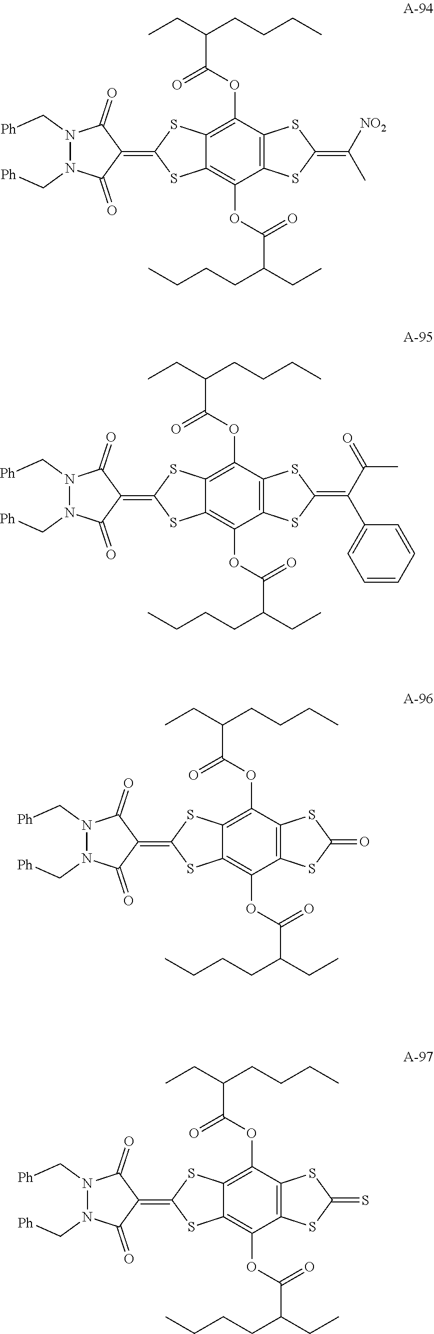 [SMILES: CCCCC(CC)C(=O)Oc1c2c(c(OC(=O)C(CC)CCCC)c3c1SC(=C(C)[N+](=O)[O-])S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3c1SC(=C(C(C)=O)c1ccccc1)S3)SC(=C1C(=O)N(Cc3ccccc3)N(Cc3ccccc3)C1=O)S2 ; CCCCC(CC)C(=O)Oc1c2c(c(OC(=O)C(CC)CCCC)c3sc(=O)sc13)SC(=C1C(=O)N(Cc3ccccc3)N(Cc3ccccc3)C1=O)S2 ; CCCCC(CC)C(=O)Oc1c2c(c(OC(=O)C(CC)CCCC)c3sc(=S)sc13)SC(=C1C(=O)N(Cc3ccccc3)N(Cc3ccccc3)C1=O)S2]

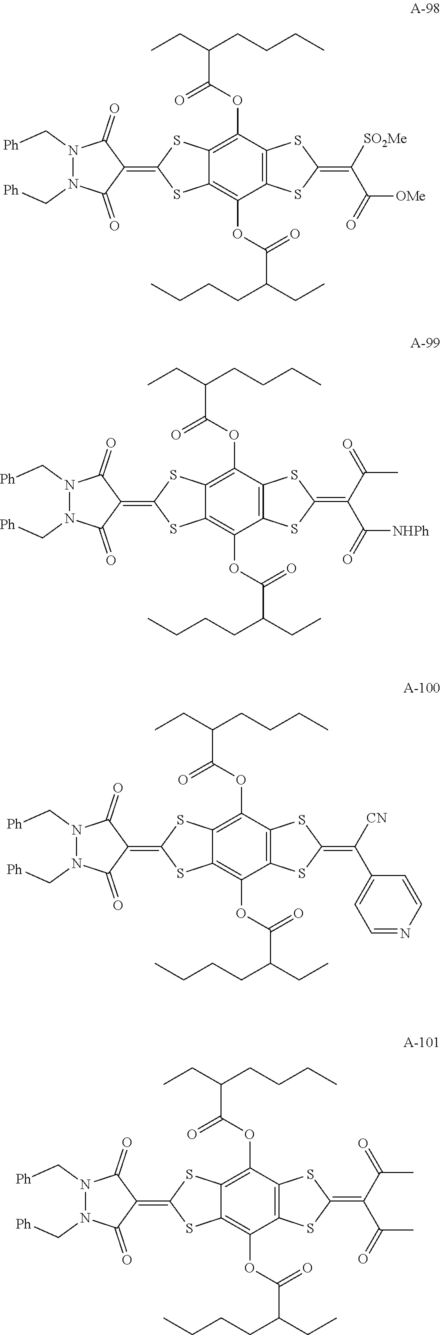

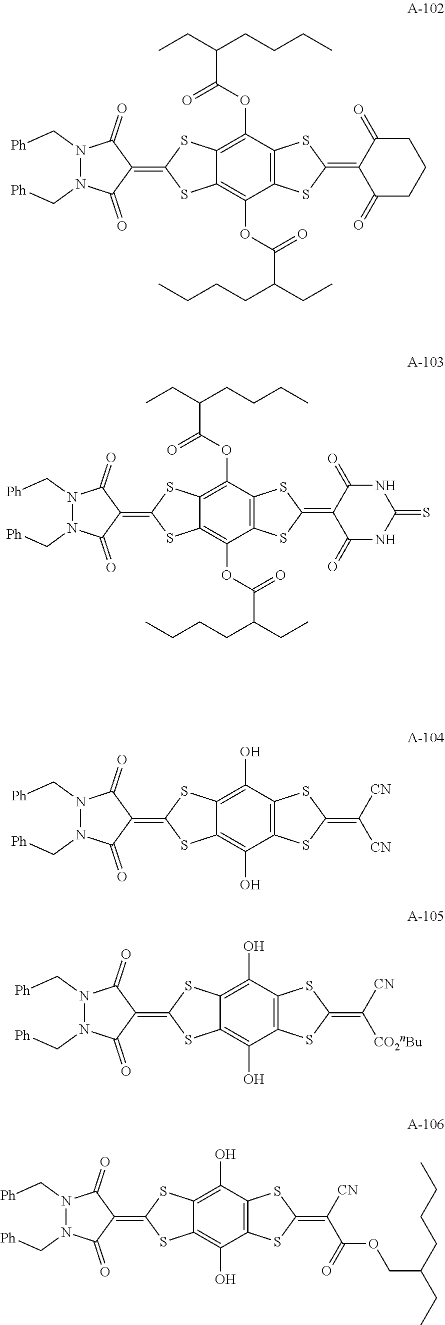

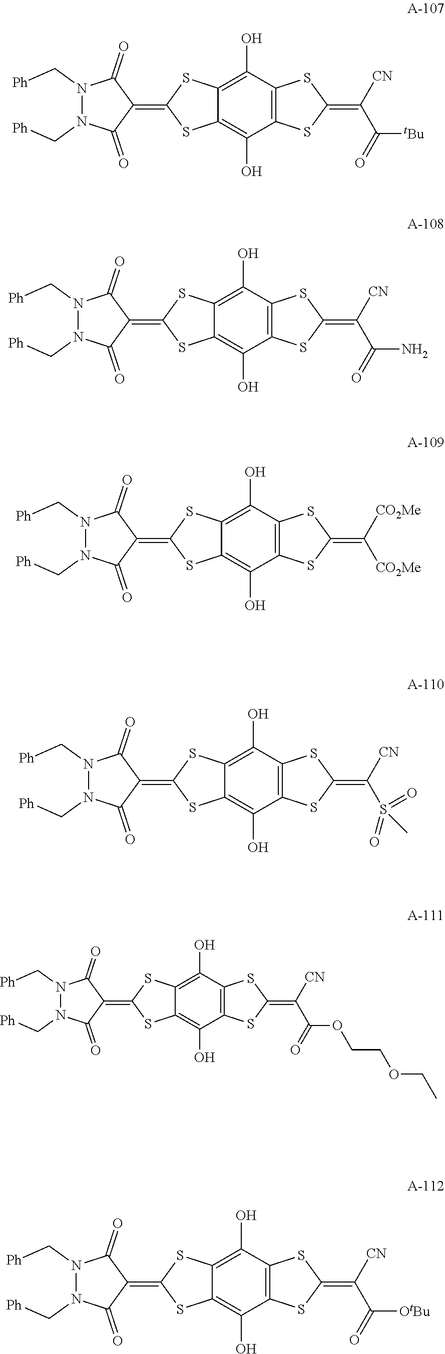

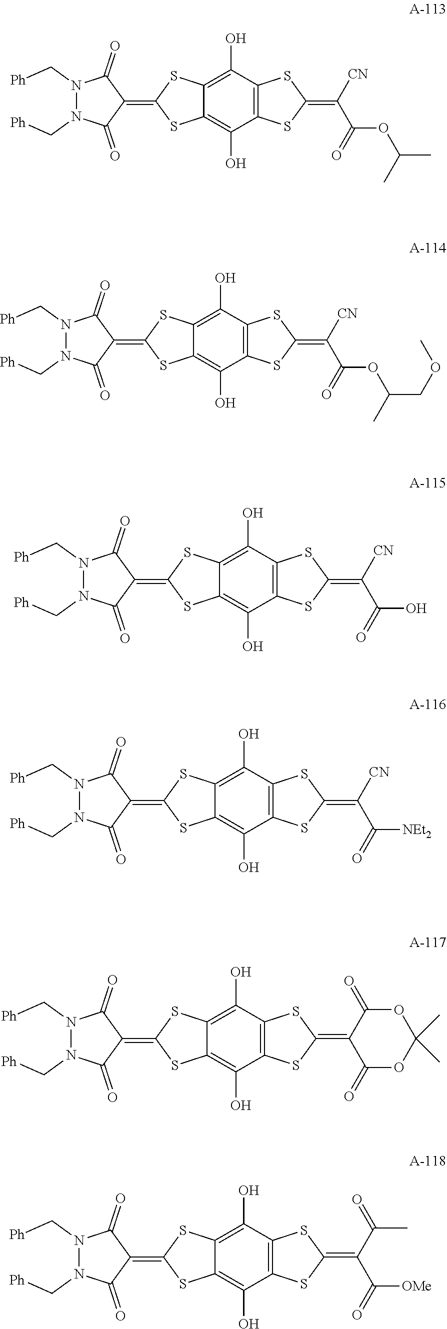

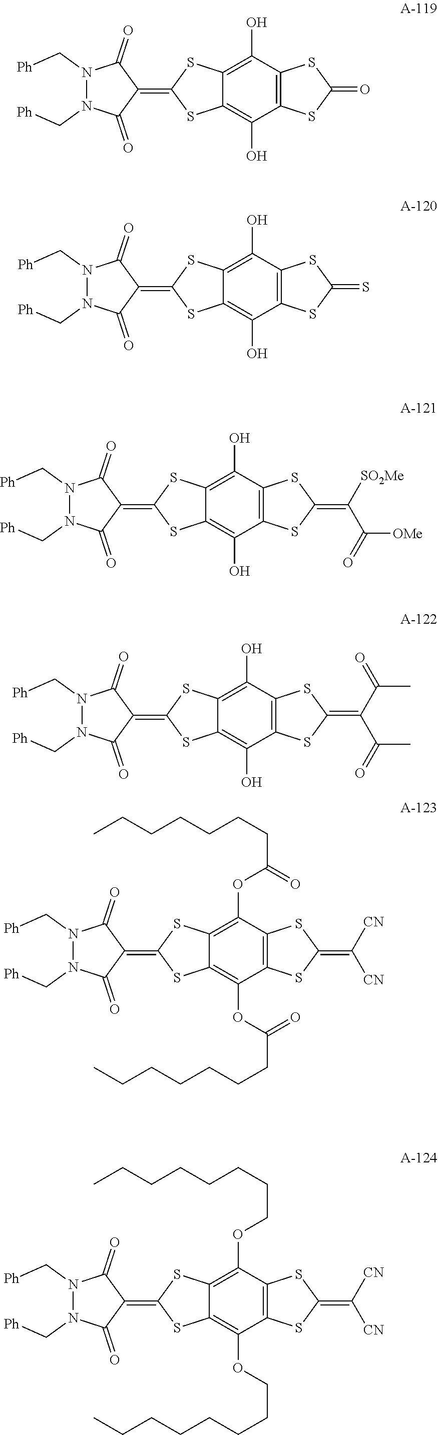

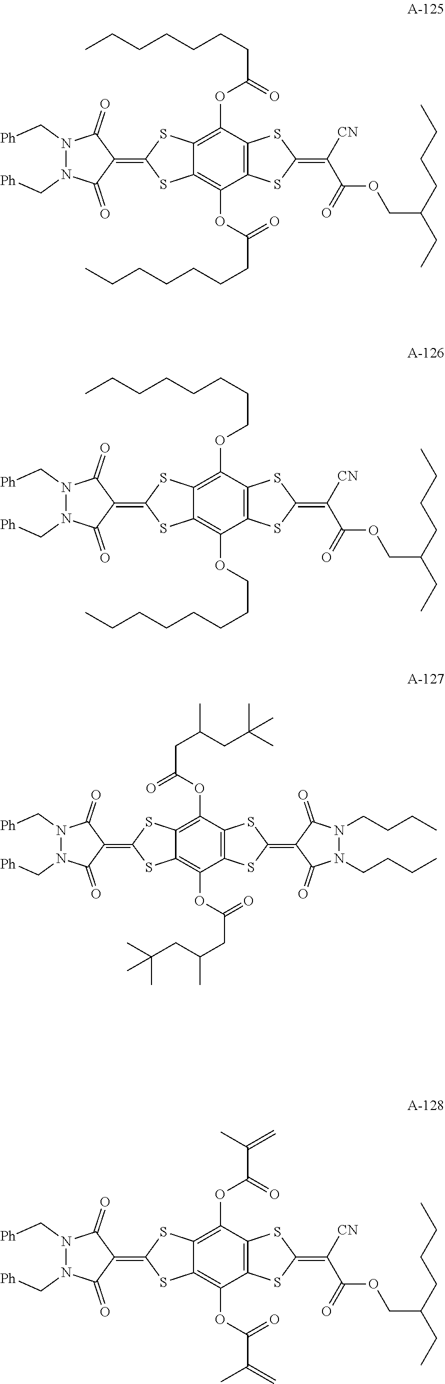 [SMILES: CCCCCCCC(=O)Oc1c2c(c(OC(=O)CCCCCCC)c3c1SC(=C1C(=O)N(Cc4ccccc4)N(Cc4ccccc4)C1=O)S3)SC(=C(C#N)C(=O)OCC(CC)CCCC)S2 ; CCCCCCCCOc1c2c(c(OCCCCCCCC)c3c1SC(=C1C(=O)N(Cc4ccccc4)N(Cc4ccccc4)C1=O)S3)SC(=C(C#N)C(=O)OCC(CC)CCCC)S2 ; CCCCN1C(=O)C(=C2Sc3c(OC(=O)CC(C)CC(C)(C)C)c4c(c(OC(=O)CC(C)CC(C)(C)C)c3S2)SC(=C2C(=O)N(Cc3ccccc3)N(Cc3ccccc3)C2=O)S4)C(=O)N1CCCC ; C=C(C)C(=O)Oc1c2c(c(OC(=O)C(=C)C)c3c1SC(=C1C(=O)N(Cc4ccccc4)N(Cc4ccccc4)C1=O)S3)SC(=C(C#N)C(=O)OCC(CC)CCCC)S2]

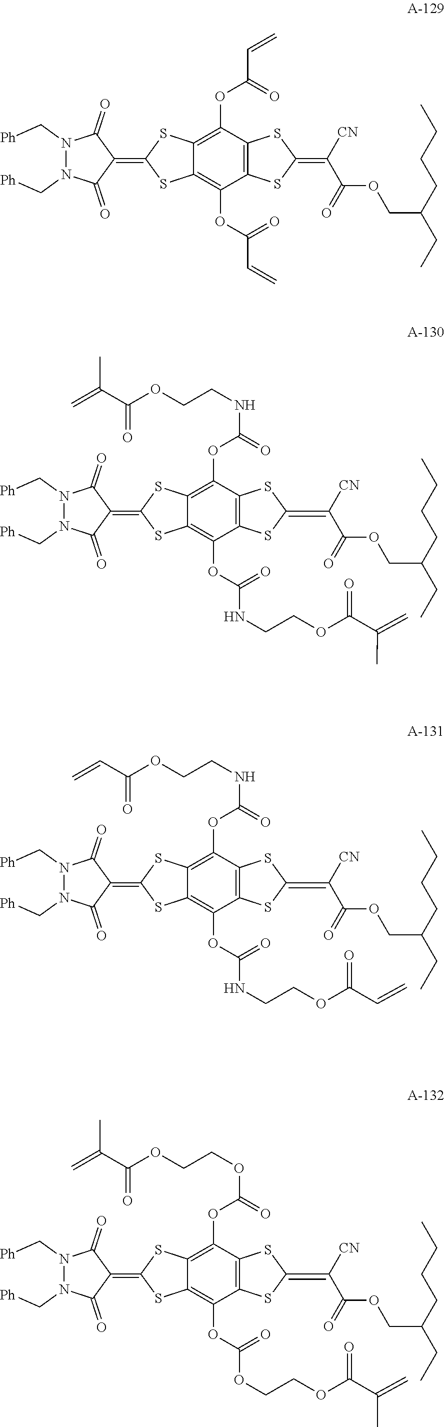 [SMILES: C=CC(=O)Oc1c2c(c(OC(=O)C=C)c3c1SC(=C1C(=O)N(Cc4ccccc4)N(Cc4ccccc4)C1=O)S3)SC(=C(C#N)C(=O)OCC(CC)CCCC)S2 ; C=C(C)C(=O)OCCNC(=O)Oc1c2c(c(OC(=O)NCCOC(=O)C(=C)C)c3c1SC(=C1C(=O)N(Cc4ccccc4)N(Cc4ccccc4)C1=O)S3)SC(=C(C#N)C(=O)OCC(CC)CCCC)S2 ; C=CC(=O)OCCNC(=O)Oc1c2c(c(OC(=O)NCCOC(=O)C=C)c3c1SC(=C1C(=O)N(Cc4ccccc4)N(Cc4ccccc4)C1=O)S3)SC(=C(C#N)C(=O)OCC(CC)CCCC)S2 ; C=C(C)C(=O)OCCOC(=O)Oc1c2c(c(OC(=O)OCCOC(=O)C(=C)C)c3c1SC(=C1C(=O)N(Cc4ccccc4)N(Cc4ccccc4)C1=O)S3)SC(=C(C#N)C(=O)OCC(CC)CCCC)S2]

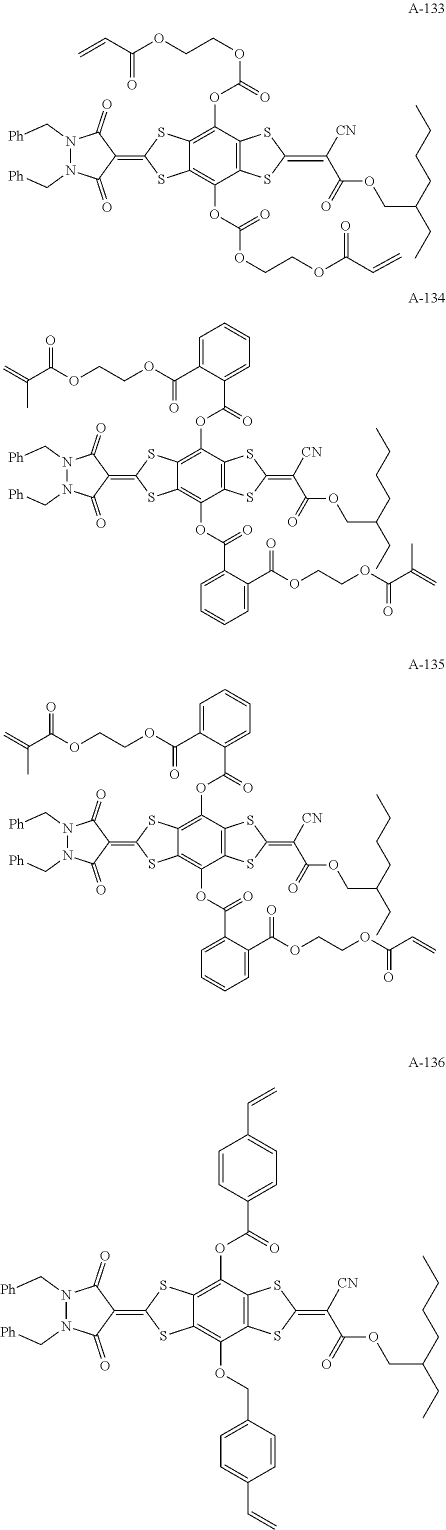

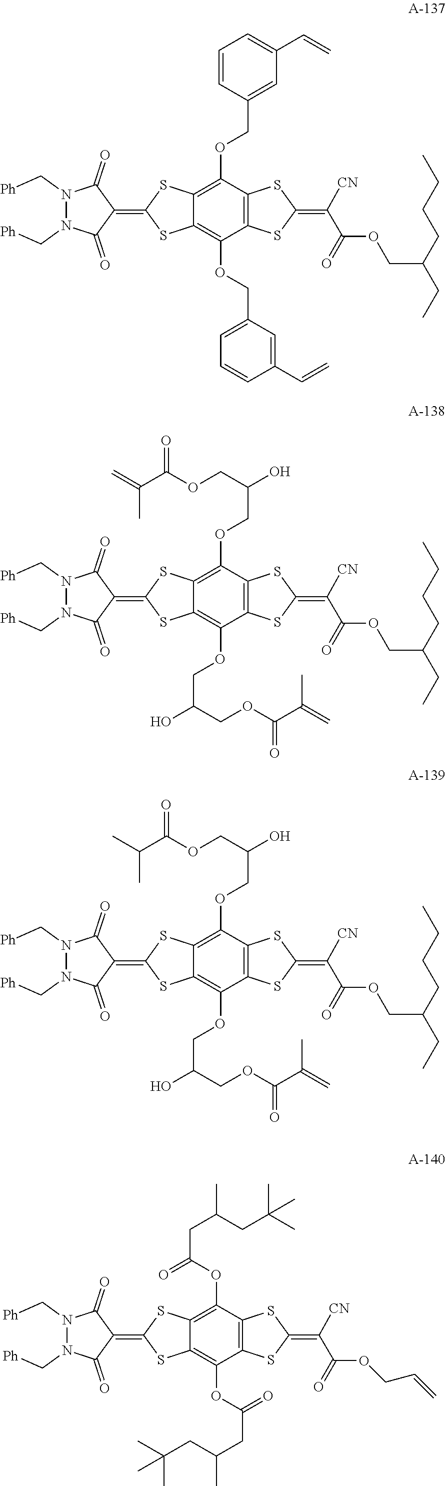 [SMILES: C=Cc1cccc(COc2c3c(c(OCc4cccc(C=C)c4)c4c2SC(=C2C(=O)N(Cc5ccccc5)N(Cc5ccccc5)C2=O)S4)SC(=C(C#N)C(=O)OCC(CC)CCCC)S3)c1 ; C=C(C)C(=O)OCC(O)COc1c2c(c(OCC(O)COC(=O)C(=C)C)c3c1SC(=C1C(=O)N(Cc4ccccc4)N(Cc4ccccc4)C1=O)S3)SC(=C(C#N)C(=O)OCC(CC)CCCC)S2 ; C=C(C)C(=O)OCC(O)COc1c2c(c(OCC(O)COC(=O)C(C)C)c3c1S/C(=C(/C#N)C(=O)OCC(CC)CCCC)S3)SC(=C1C(=O)N(Cc3ccccc3)N(Cc3ccccc3)C1=O)S2 ; C=CCOC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3]

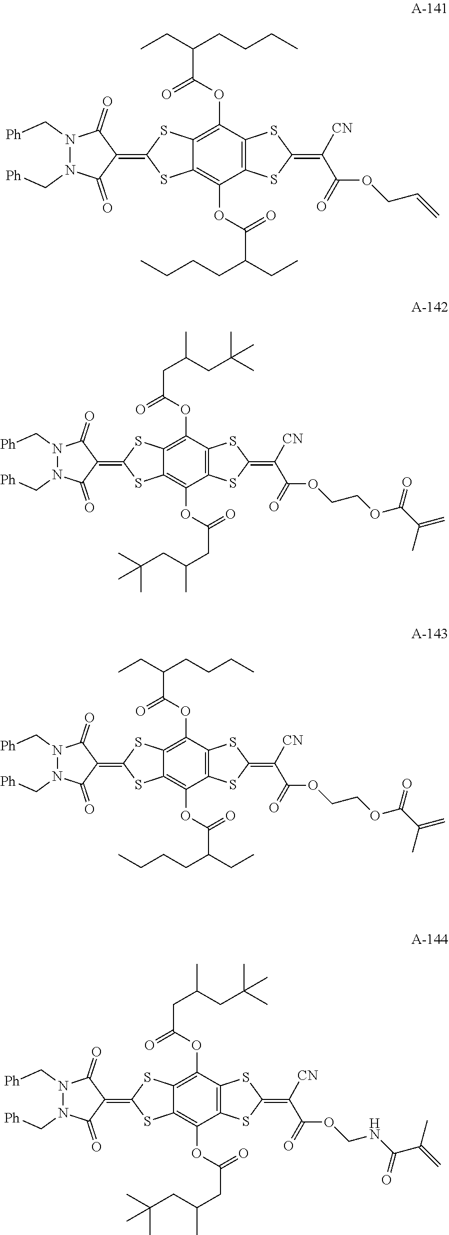 [SMILES: C=CCOC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3 ; C=C(C)C(=O)OCCOC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; C=C(C)C(=O)OCCOC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3 ; C=C(C)C(=O)NCOC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3]

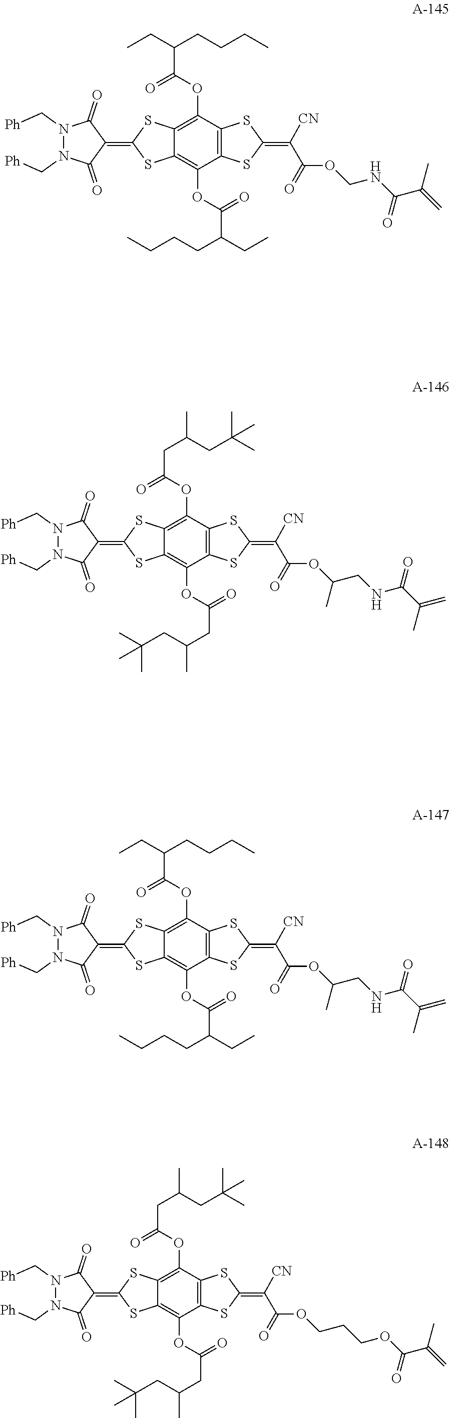

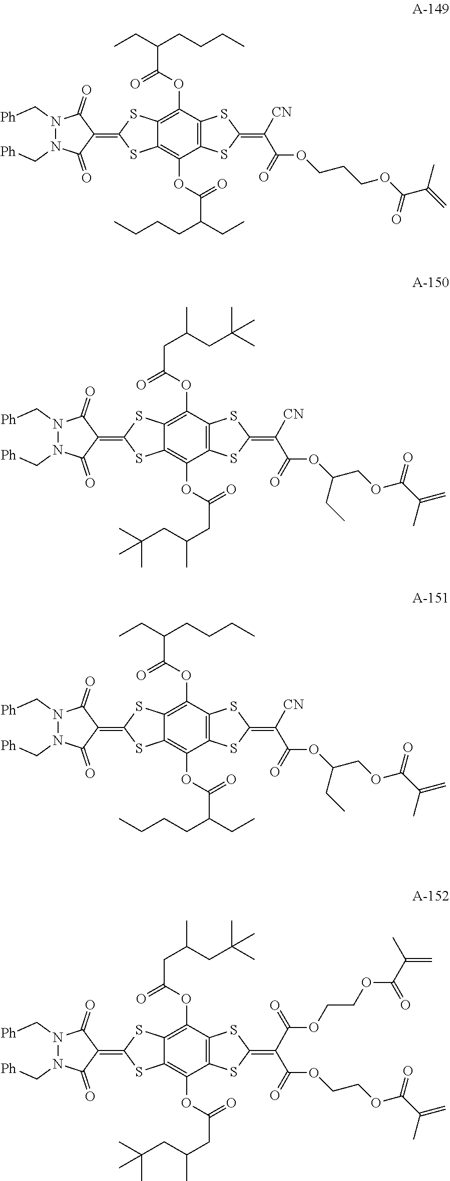

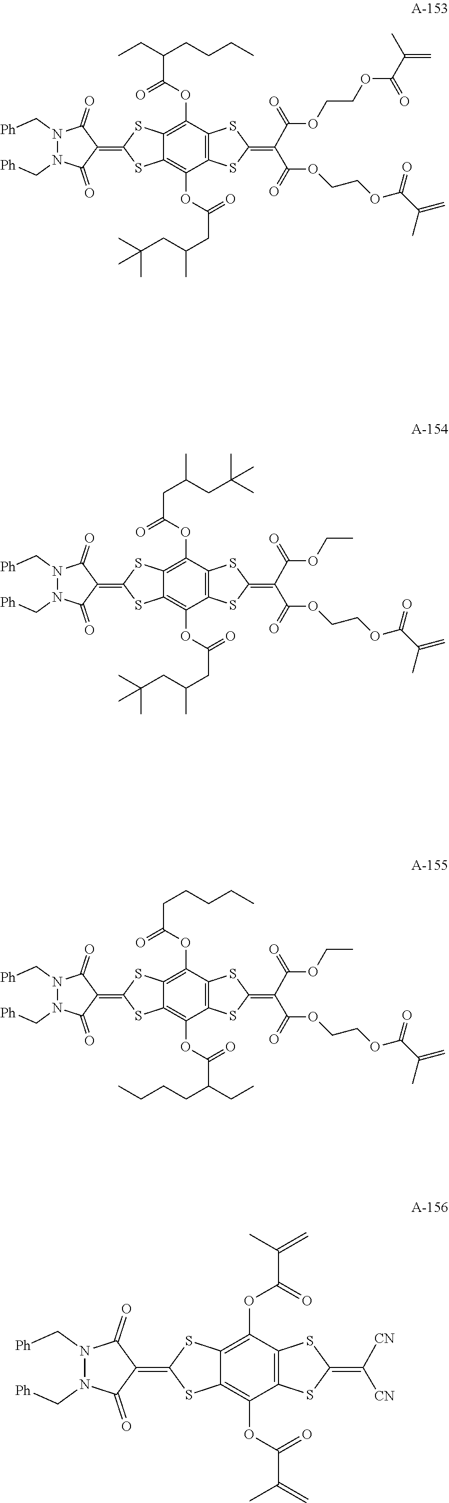

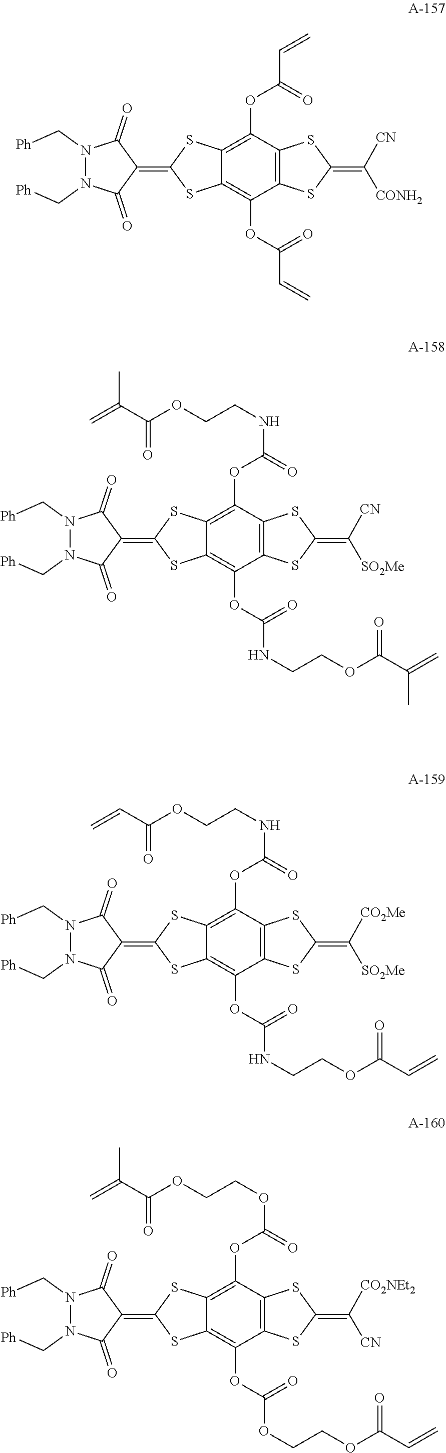

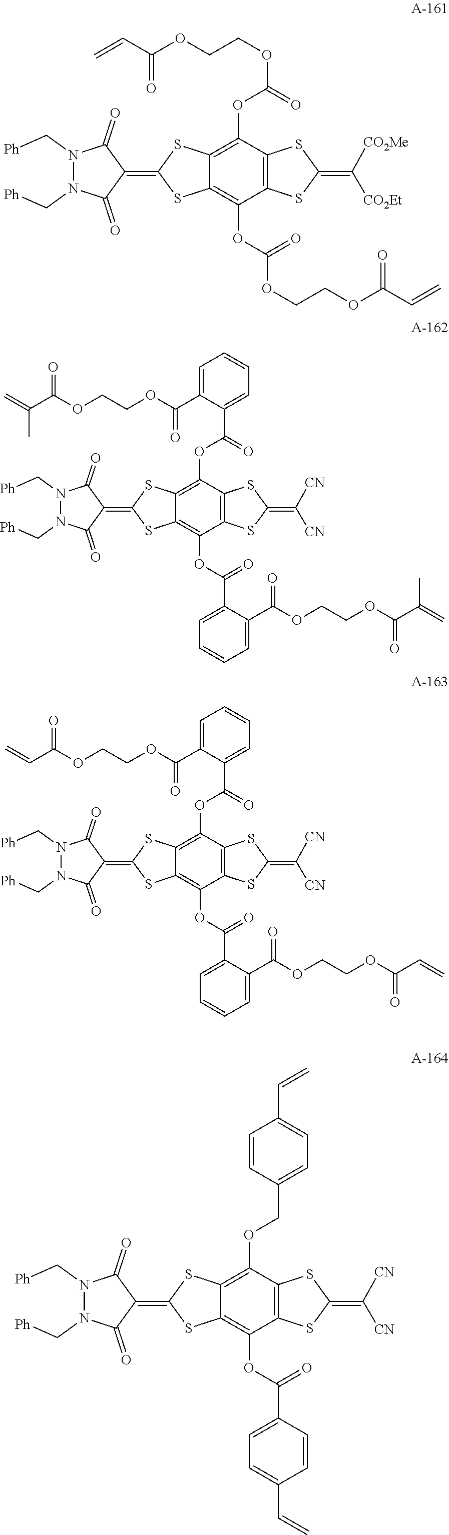

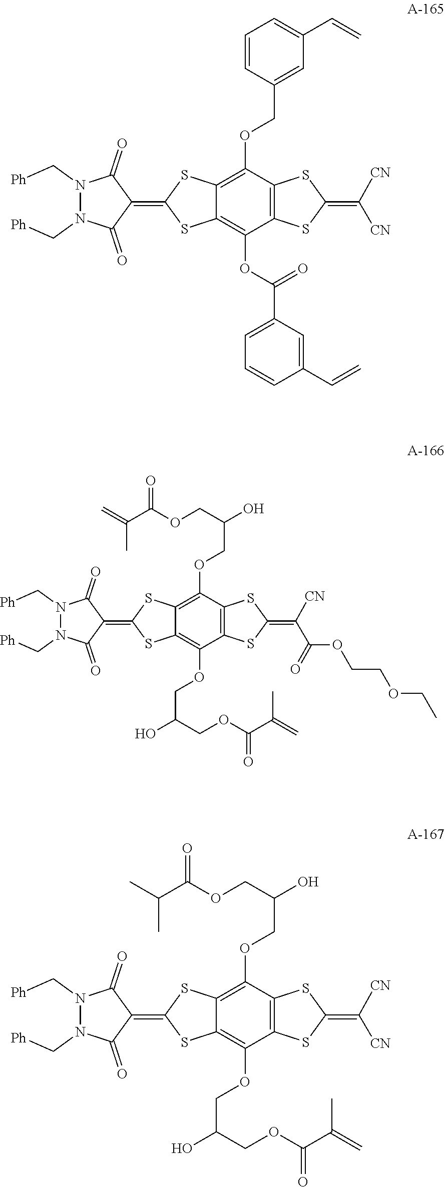 [SMILES: C=Cc1cccc(COc2c3c(c(OC(=O)c4cccc(C=C)c4)c4c2SC(=C2C(=O)N(Cc5ccccc5)N(Cc5ccccc5)C2=O)S4)SC(=C(C#N)C#N)S3)c1 ; C=C(C)C(=O)OCC(O)COc1c2c(c(OCC(O)COC(=O)C(=C)C)c3c1SC(=C1C(=O)N(Cc4ccccc4)N(Cc4ccccc4)C1=O)S3)SC(=C(C#N)C(=O)OCCOCC)S2 ; C=C(C)C(=O)OCC(O)COc1c2c(c(OCC(O)COC(=O)C(C)C)c3c1SC(=C1C(=O)N(Cc4ccccc4)N(Cc4ccccc4)C1=O)S3)SC(=C(C#N)C#N)S2]

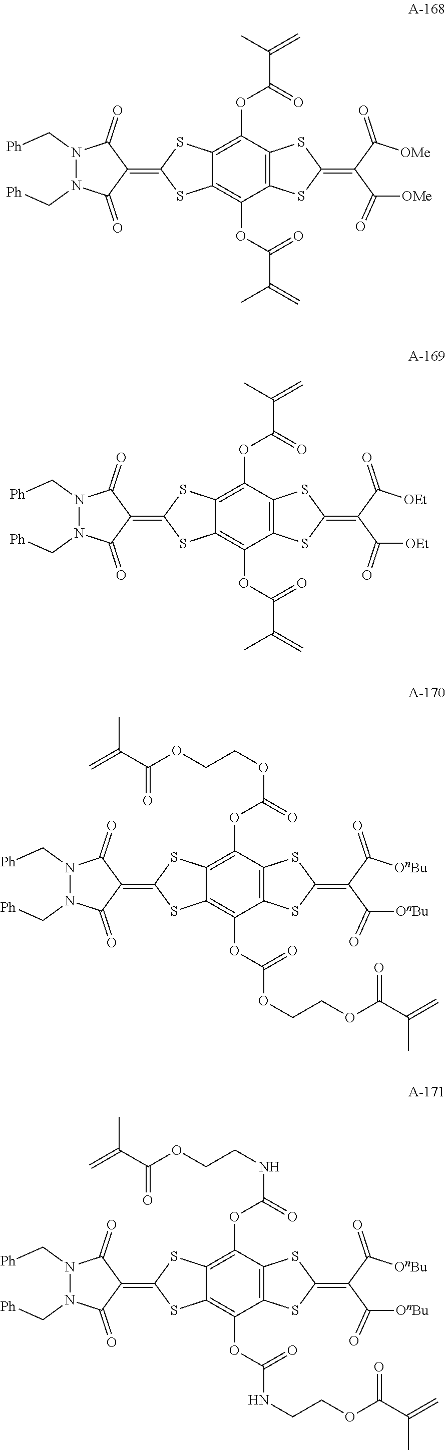

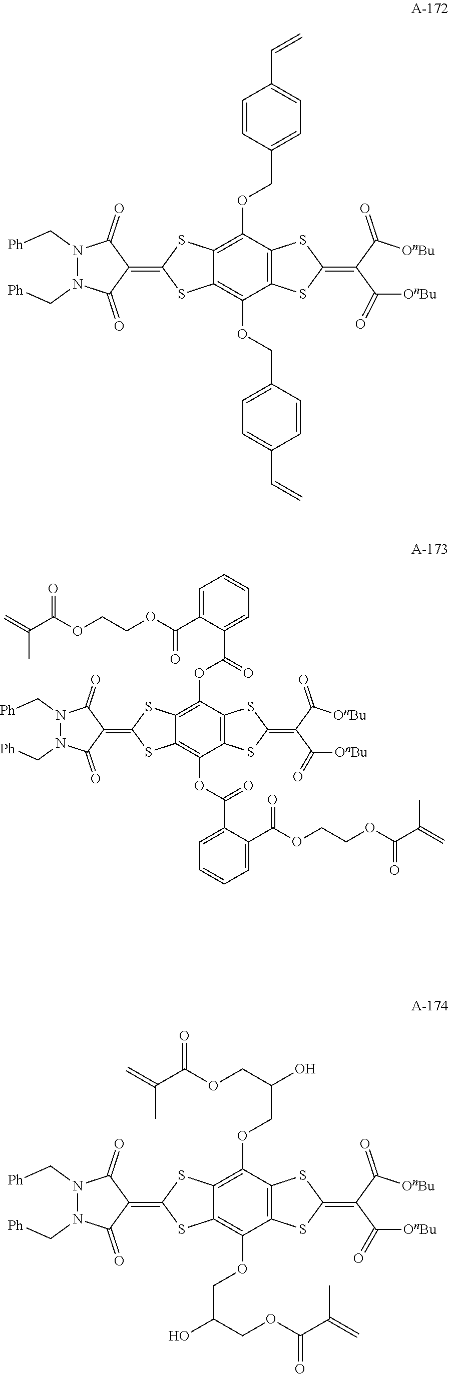

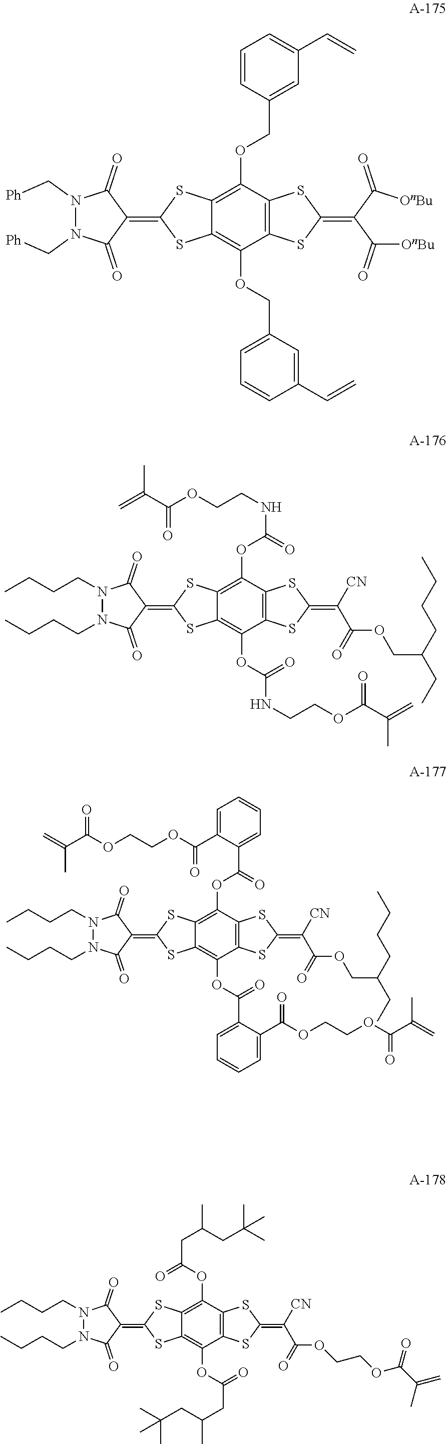

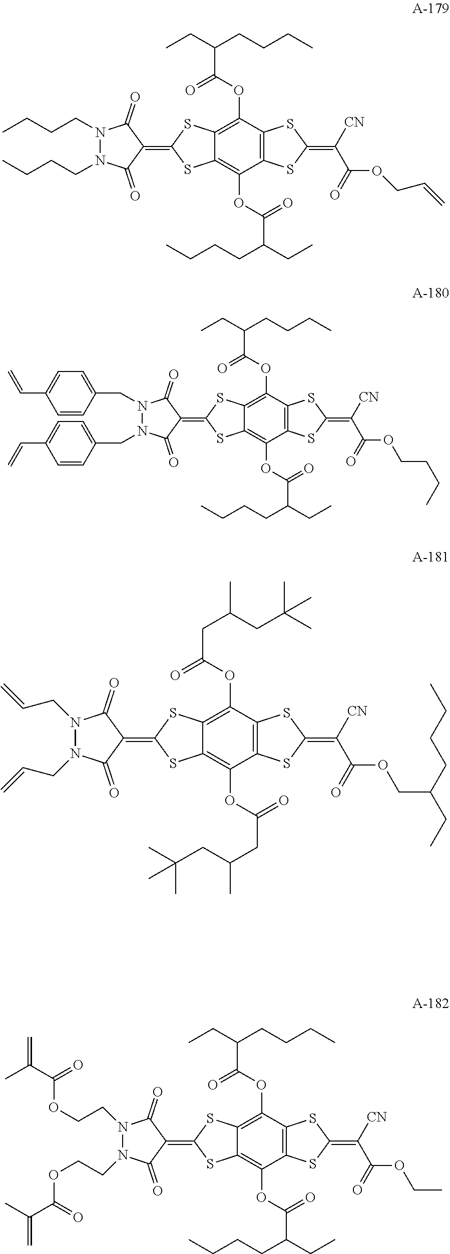

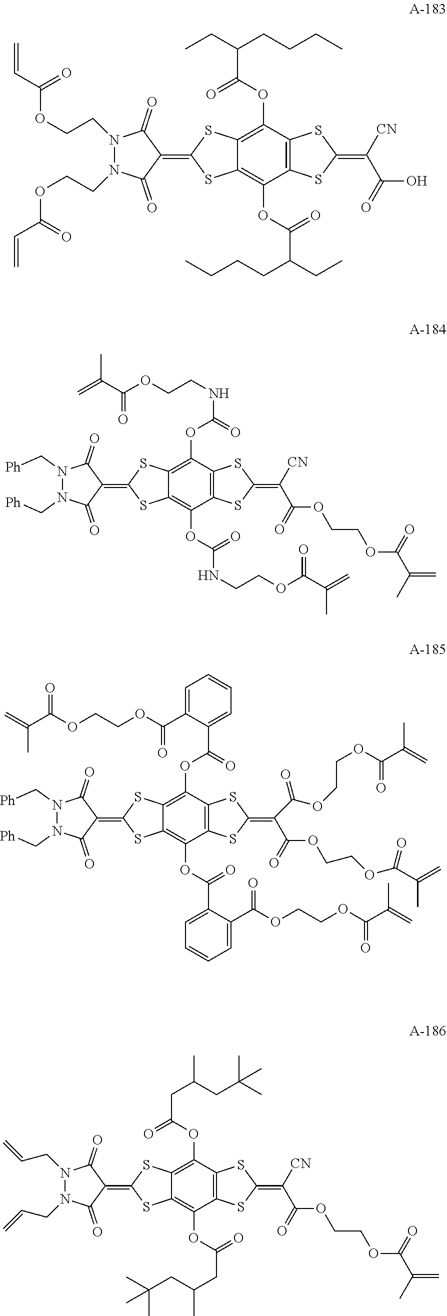

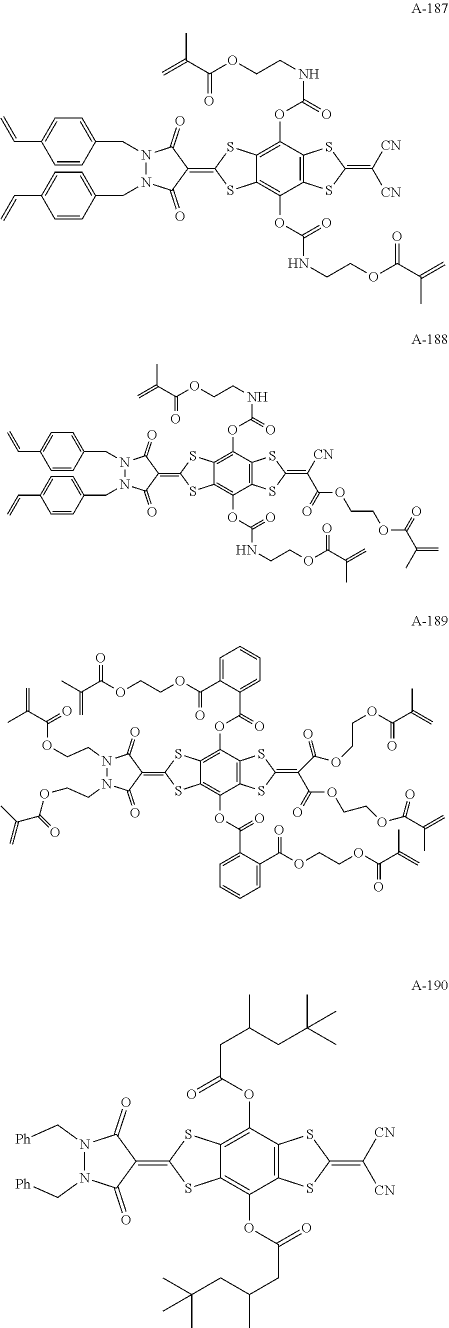

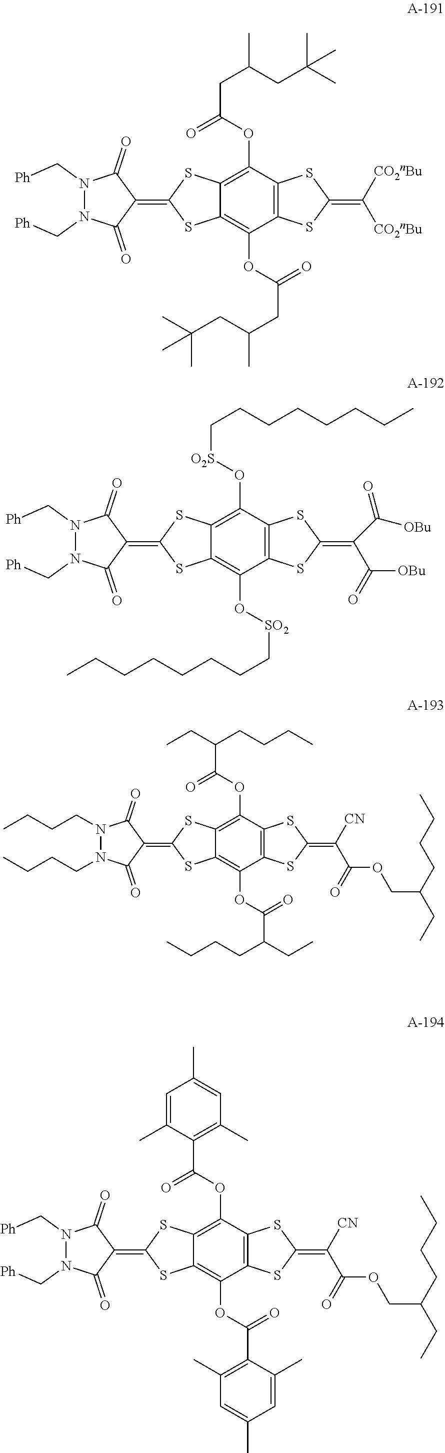

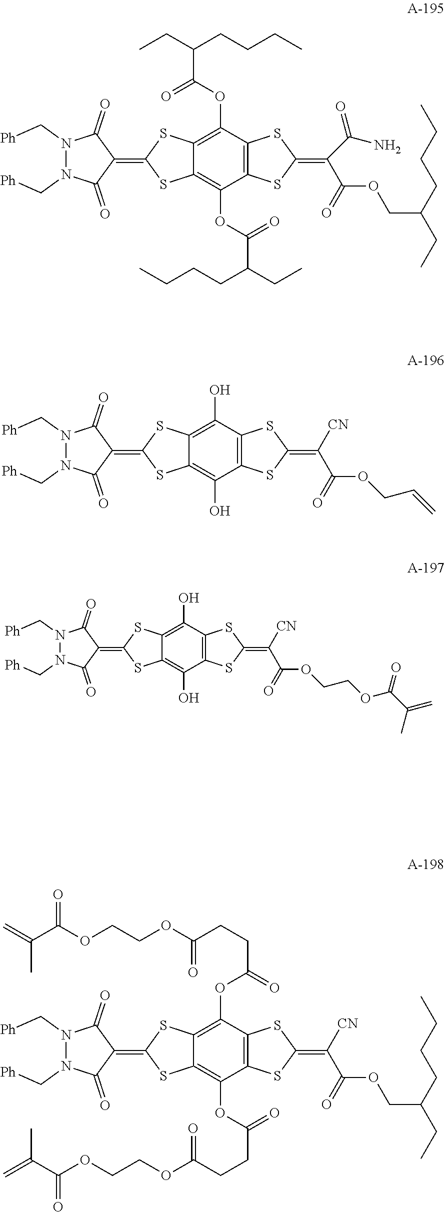

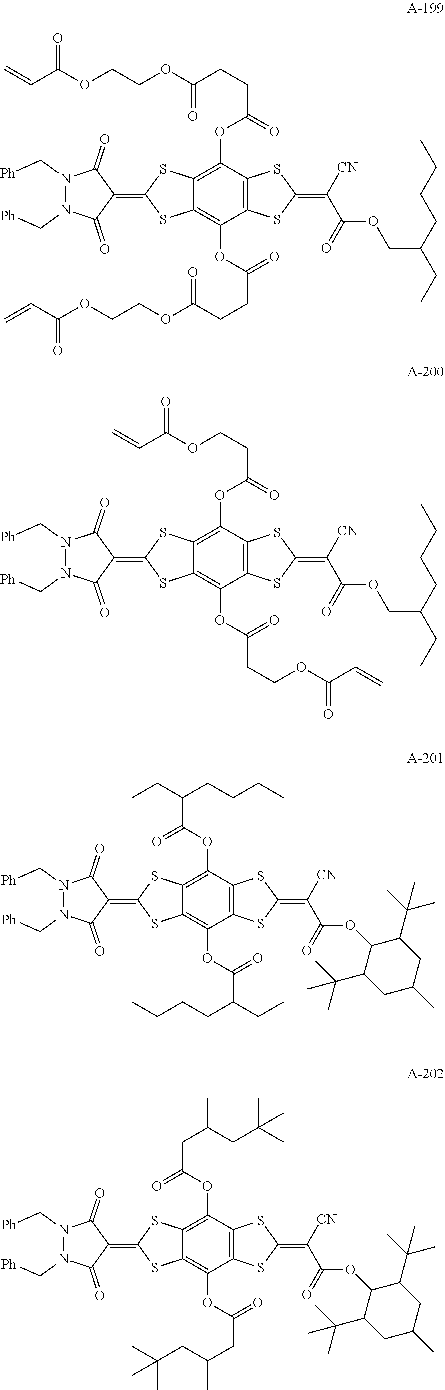

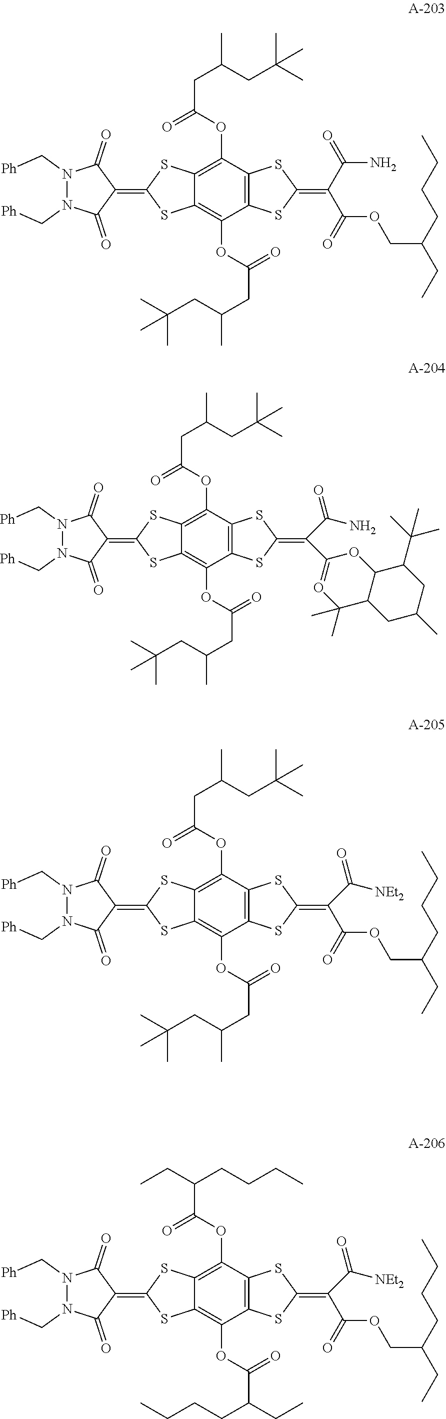

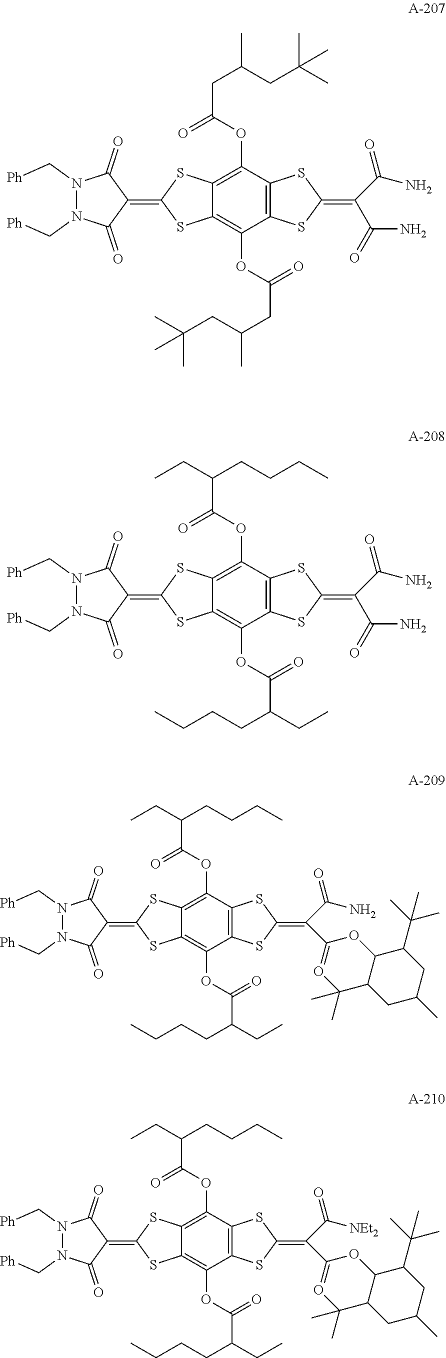

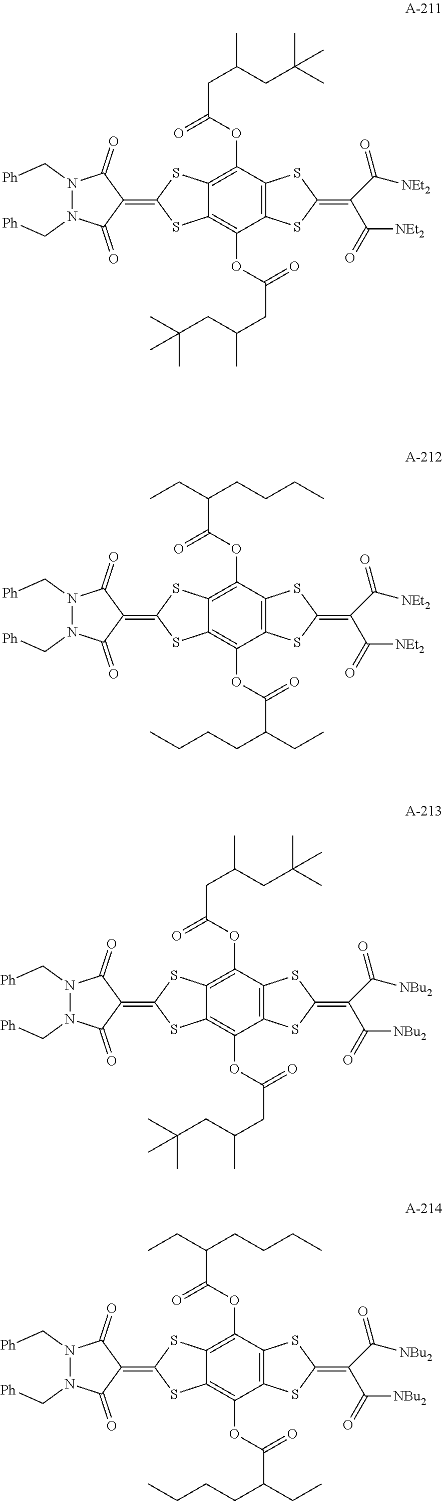

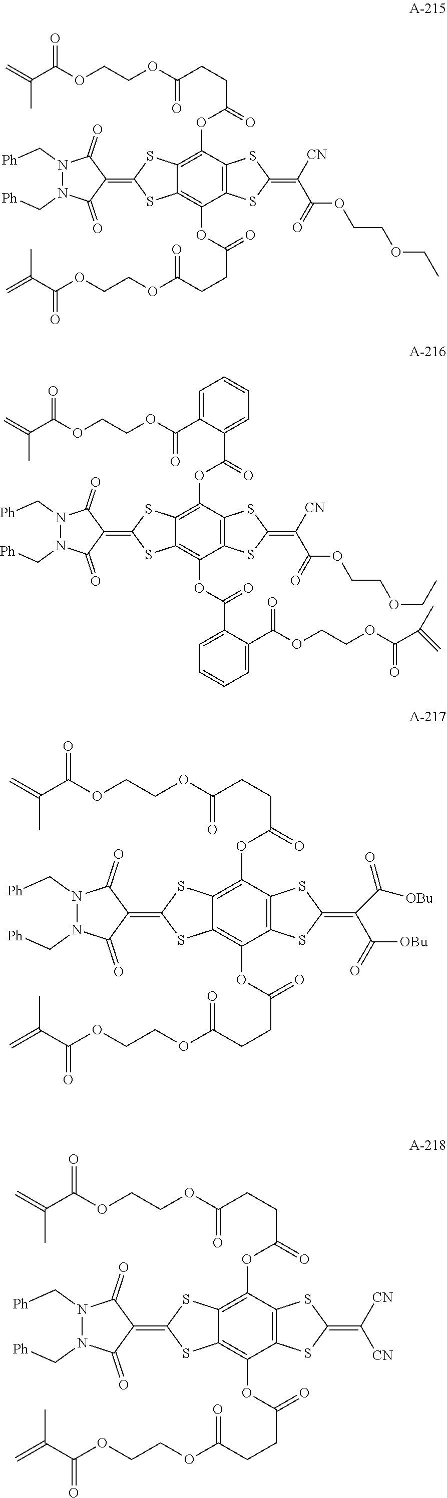

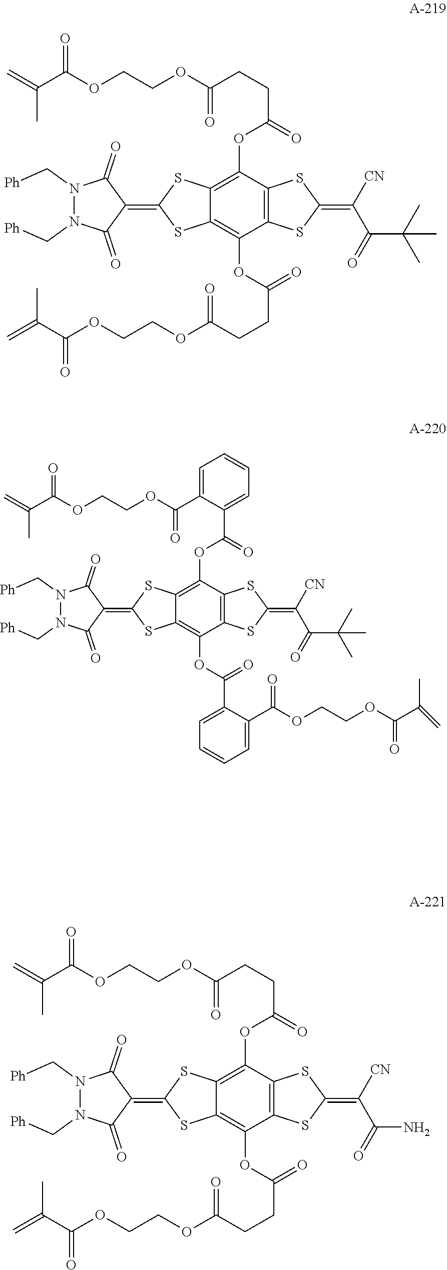

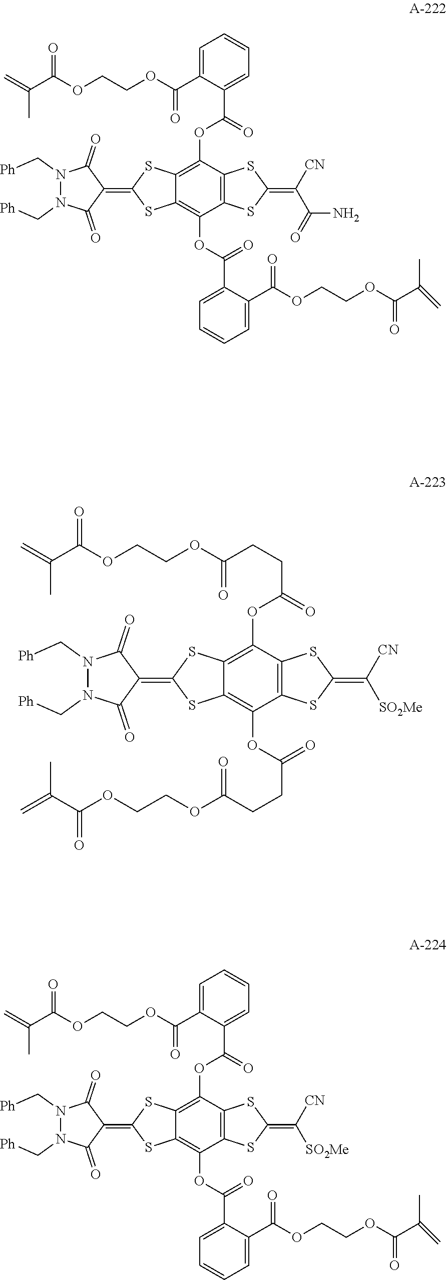

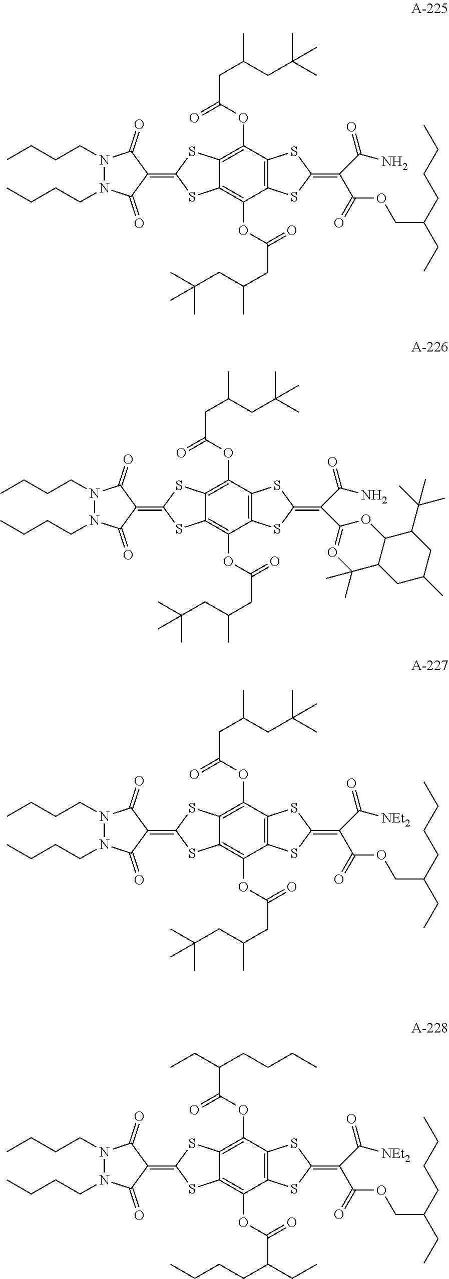

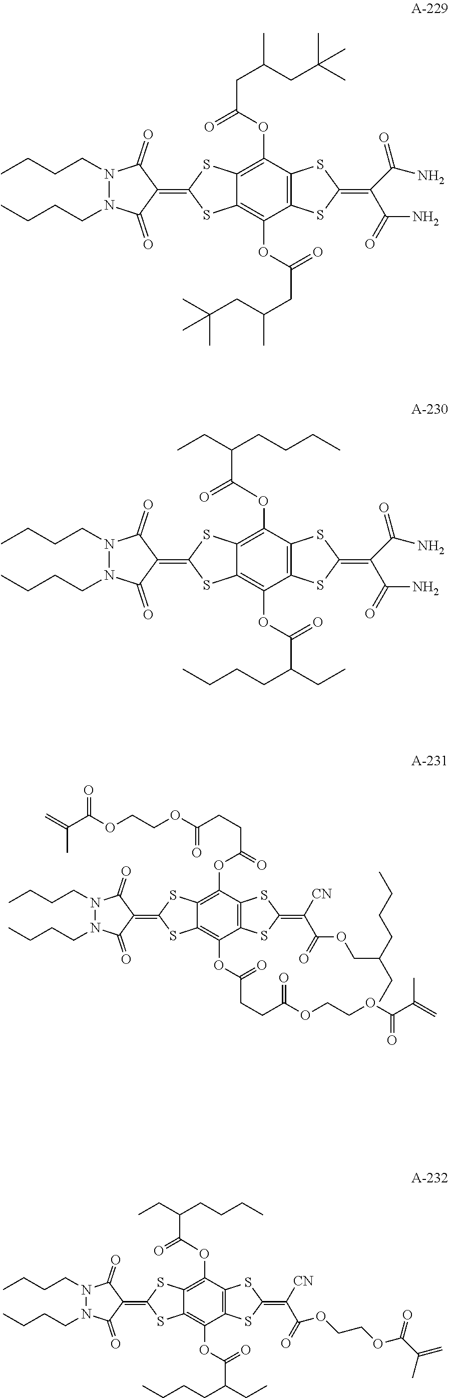

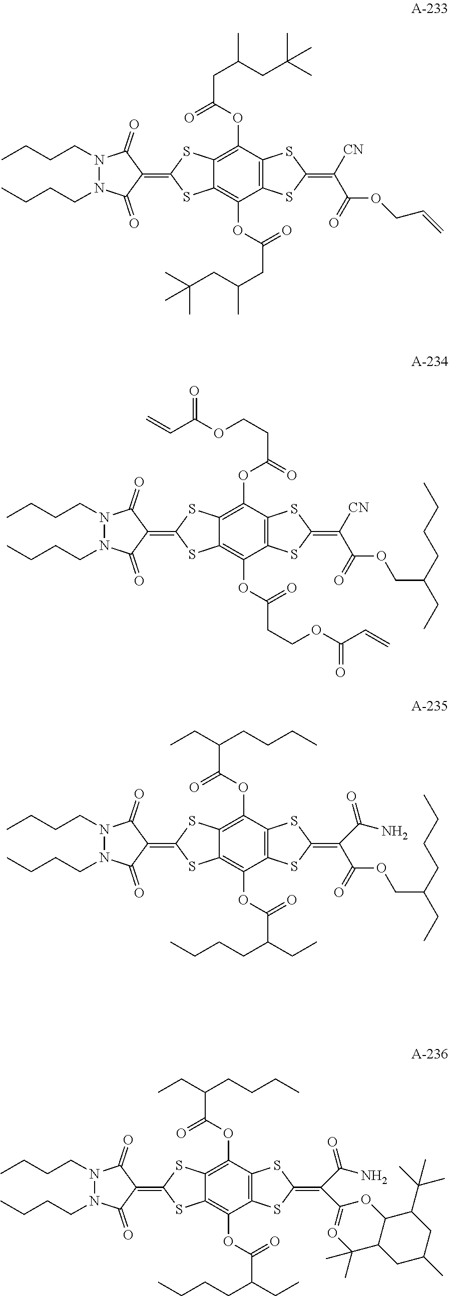

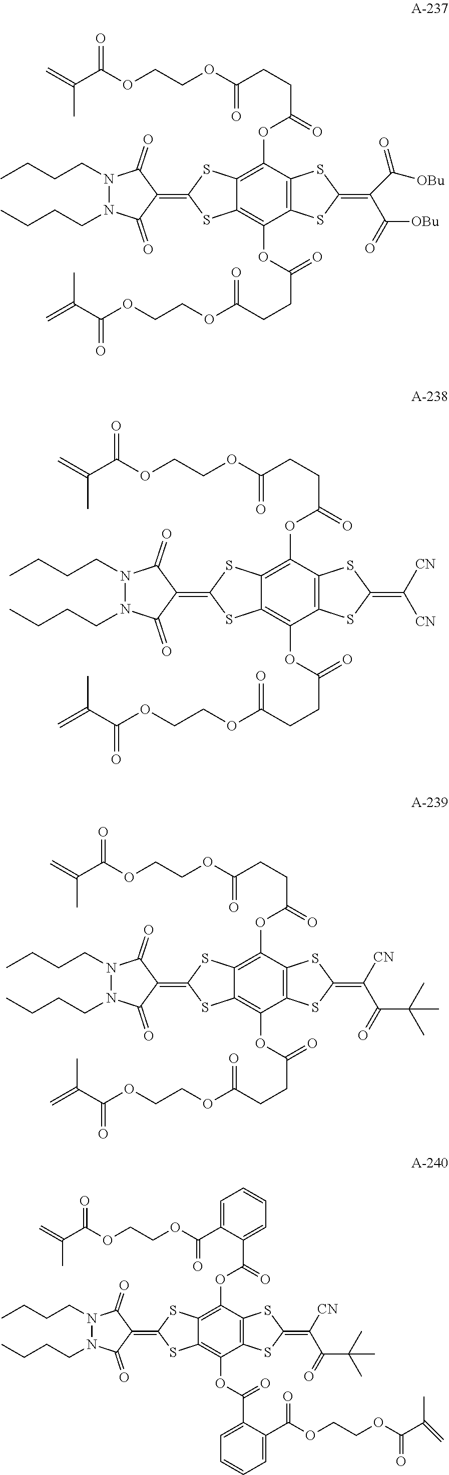

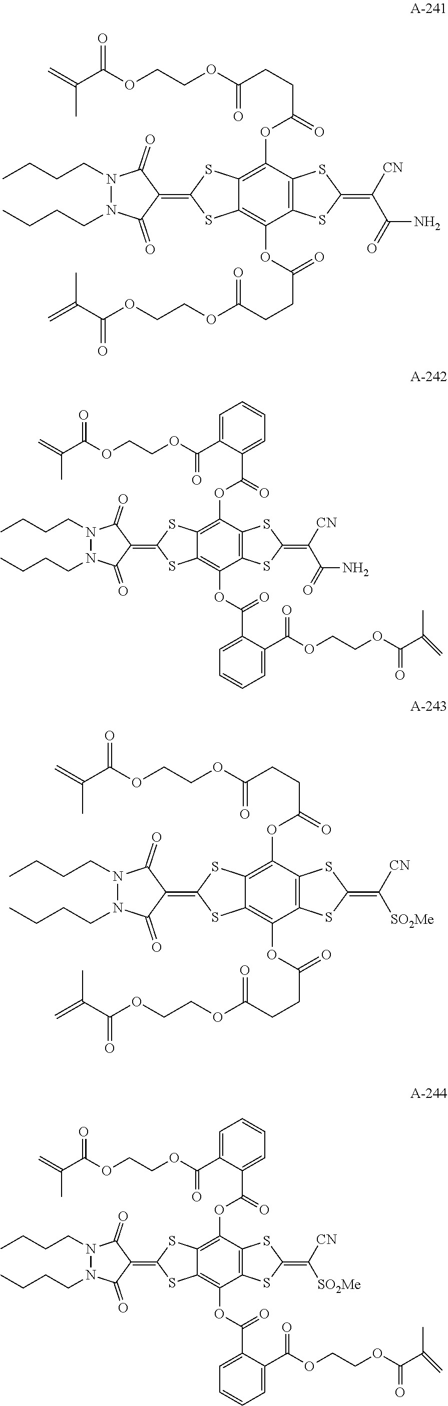

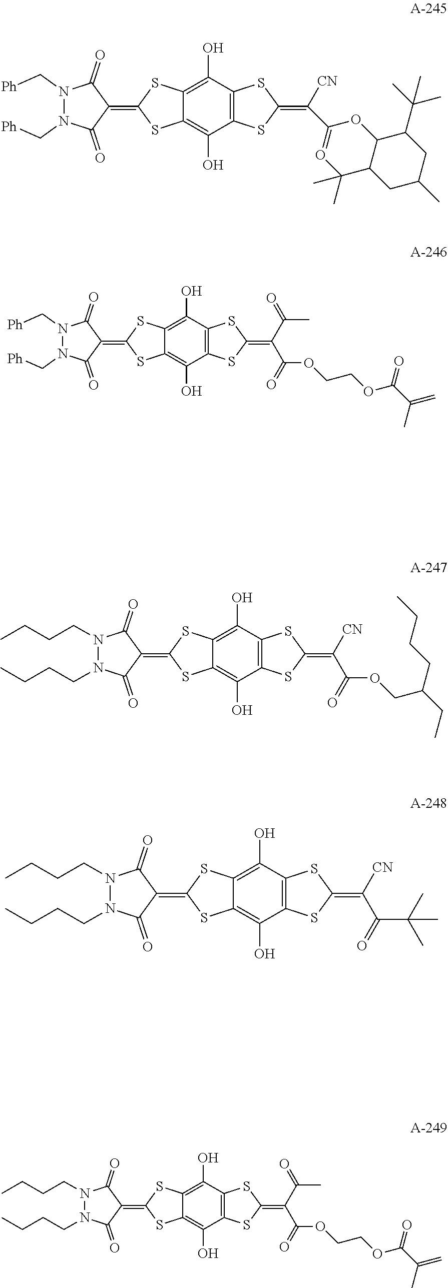

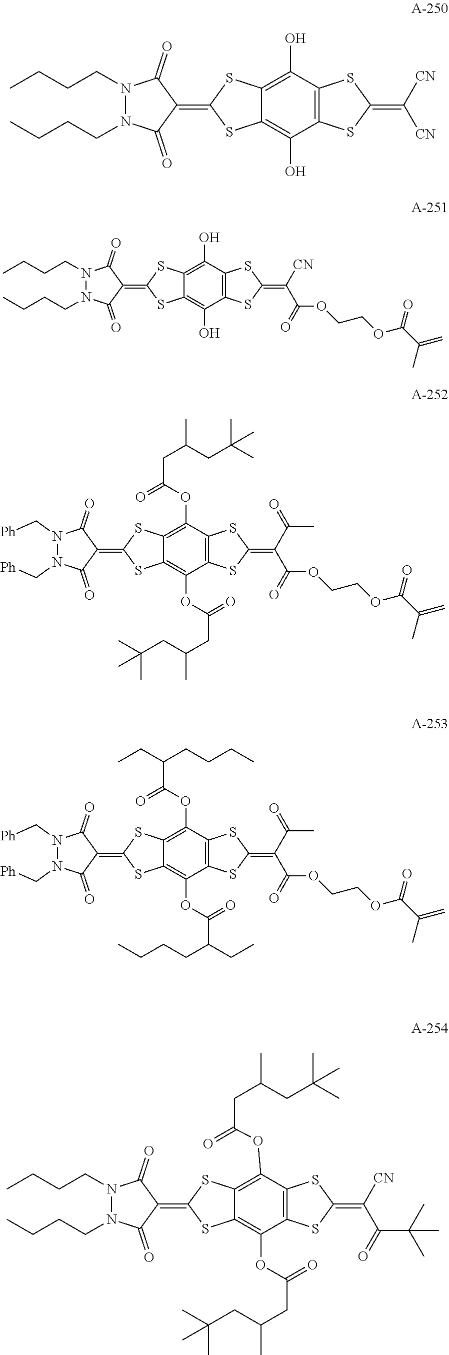

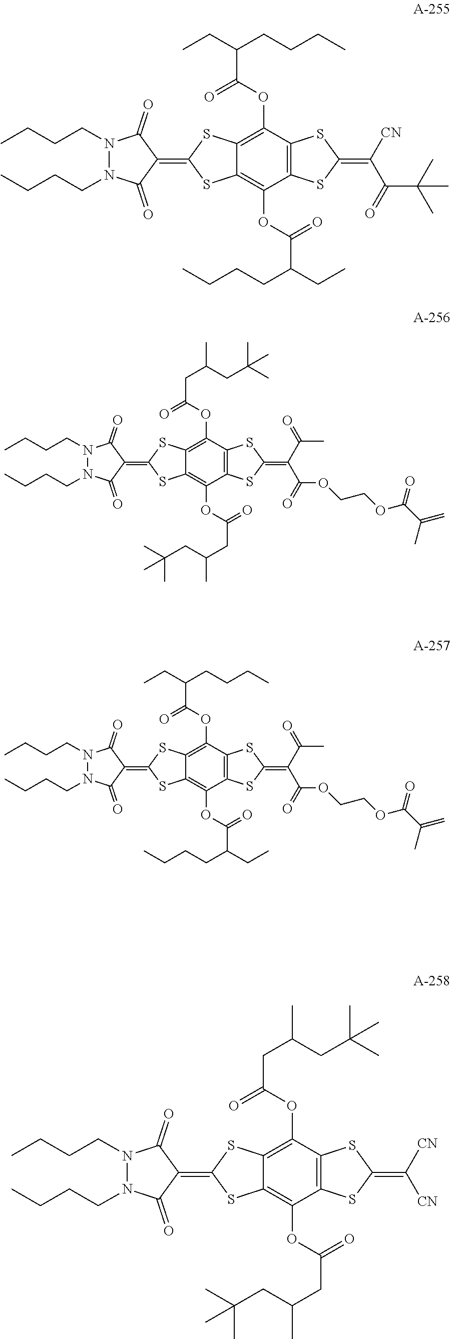

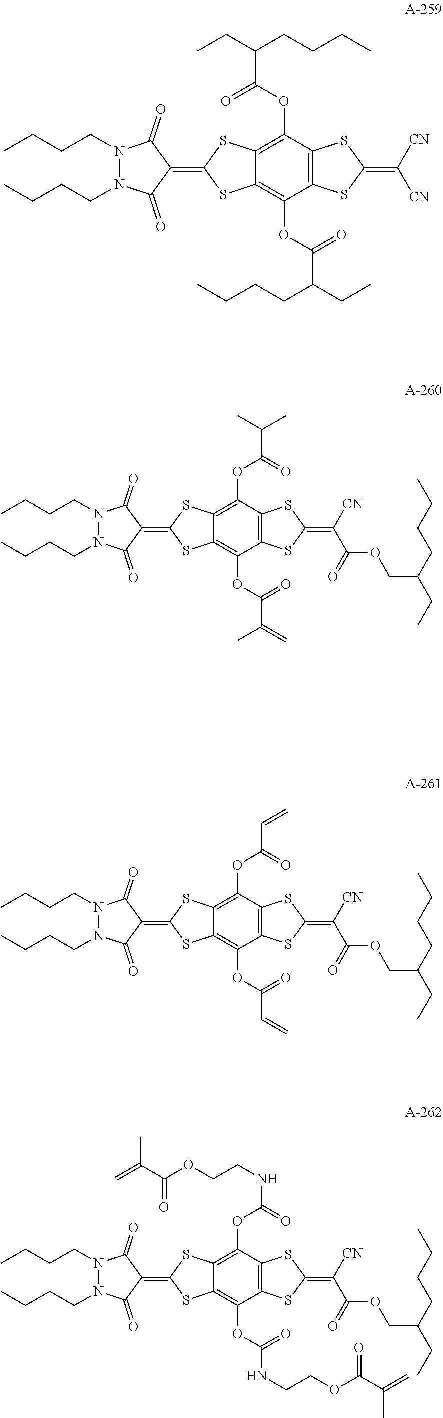 [SMILES: CCCCC(CC)C(=O)Oc1c2c(c(OC(=O)C(CC)CCCC)c3c1SC(=C1C(=O)N(CCCC)N(CCCC)C1=O)S3)SC(=C(C#N)C#N)S2 ; C=C(C)C(=O)Oc1c2c(c(OC(=O)C(C)C)c3c1S/C(=C(/C#N)C(=O)OCC(CC)CCCC)S3)SC(=C1C(=O)N(CCCC)N(CCCC)C1=O)S2 ; C=CC(=O)Oc1c2c(c(OC(=O)C=C)c3c1SC(=C1C(=O)N(CCCC)N(CCCC)C1=O)S3)SC(=C(C#N)C(=O)OCC(CC)CCCC)S2 ; C=C(C)C(=O)OCCNC(=O)Oc1c2c(c(OC(=O)NCCOC(=O)C(=C)C)c3c1SC(=C1C(=O)N(CCCC)N(CCCC)C1=O)S3)SC(=C(C#N)C(=O)OCC(CC)CCCC)S2]

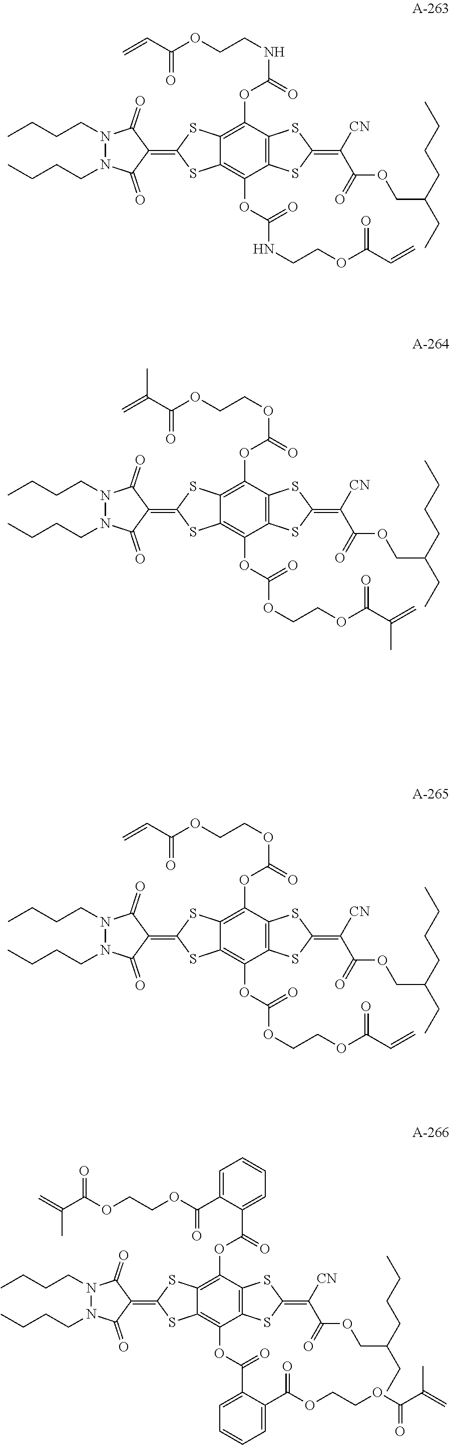 [SMILES: C=CC(=O)OCCNC(=O)Oc1c2c(c(OC(=O)NCCOC(=O)C=C)c3c1SC(=C1C(=O)N(CCCC)N(CCCC)C1=O)S3)SC(=C(C#N)C(=O)OCC(CC)CCCC)S2 ; C=C(C)C(=O)OCCOC(=O)Oc1c2c(c(OC(=O)OCCOC(=O)C(=C)C)c3c1SC(=C1C(=O)N(CCCC)N(CCCC)C1=O)S3)SC(=C(C#N)C(=O)OCC(CC)CCCC)S2 ; C=CC(=O)OCCOC(=O)Oc1c2c(c(OC(=O)OCCOC(=O)C=C)c3c1SC(=C1C(=O)N(CCCC)N(CCCC)C1=O)S3)SC(=C(C#N)C(=O)OCC(CC)CCCC)S2 ; C=C(C)C(=O)OCCOC(=O)c1ccccc1C(=O)Oc1c2c(c(OC(=O)c3ccccc3C(=O)OCCOC(=O)C(=C)C)c3c1SC(=C1C(=O)N(CCCC)N(CCCC)C1=O)S3)SC(=C(C#N)C(=O)OCC(CC)CCCC)S2]

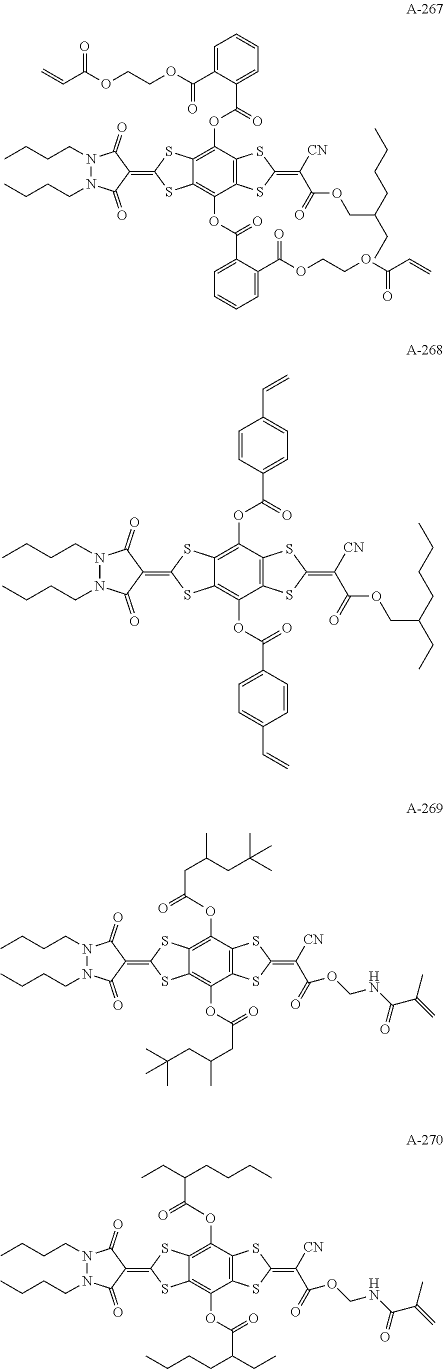

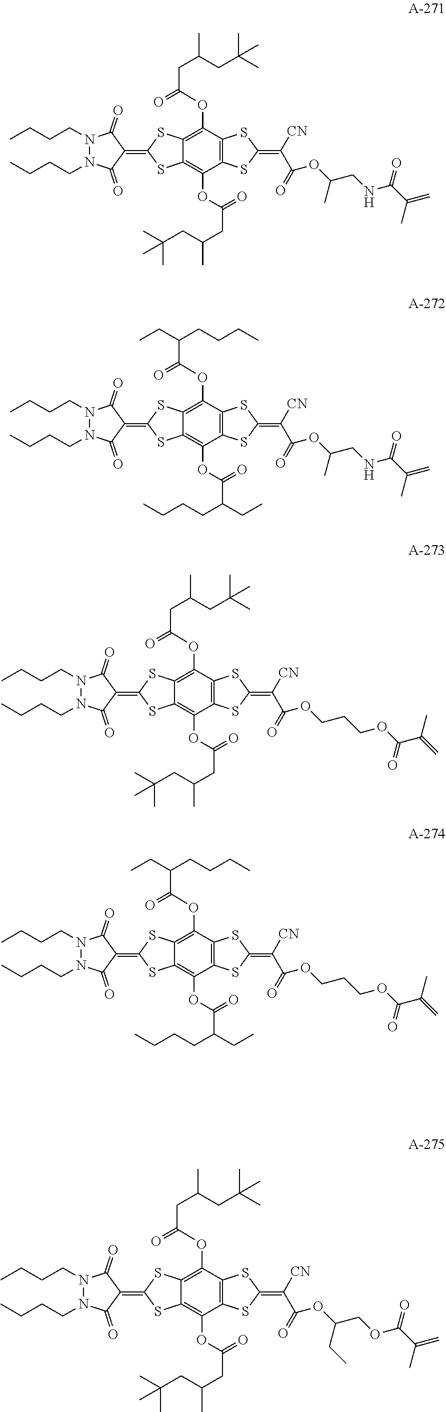

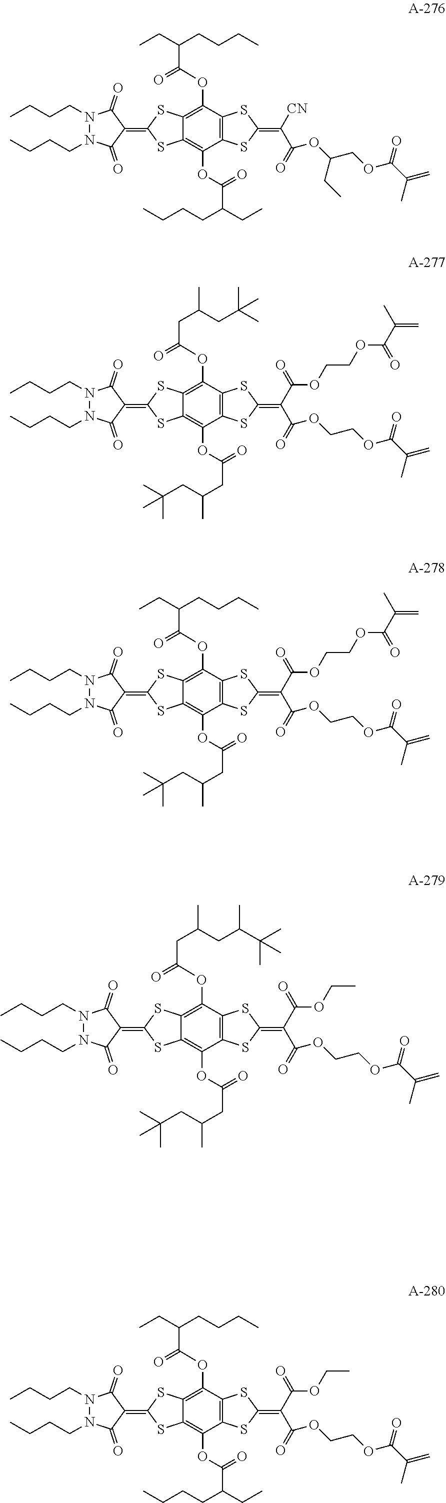

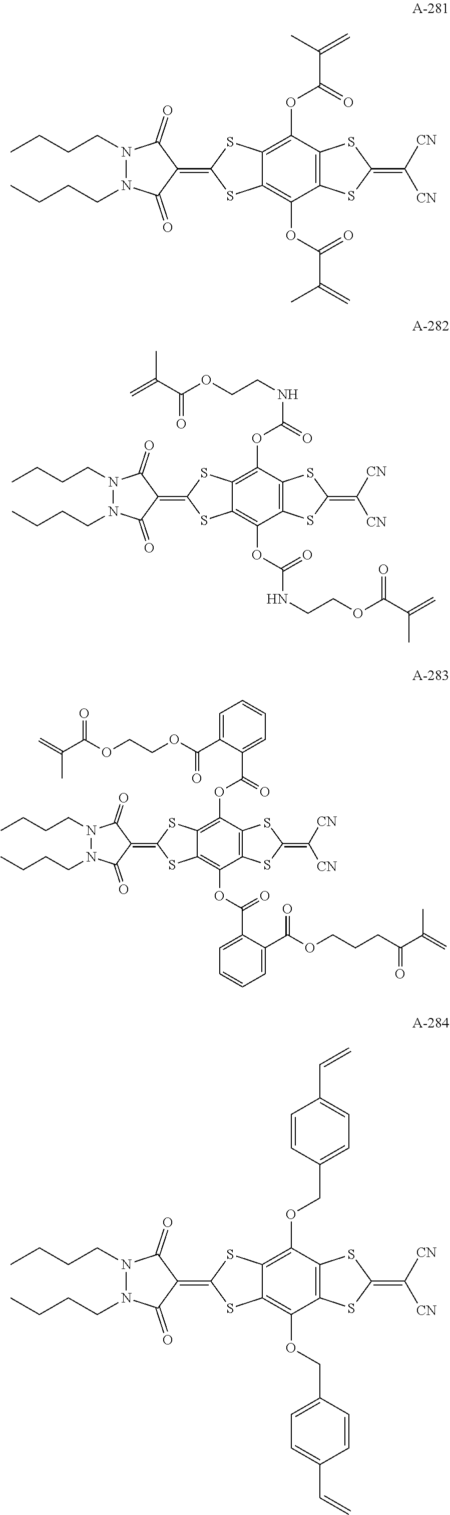

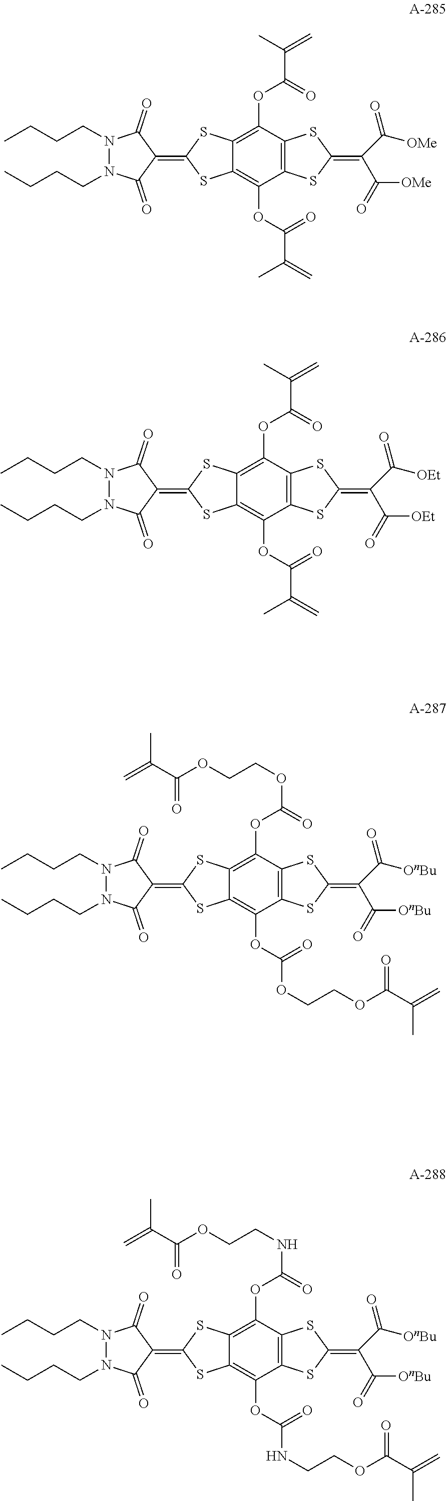

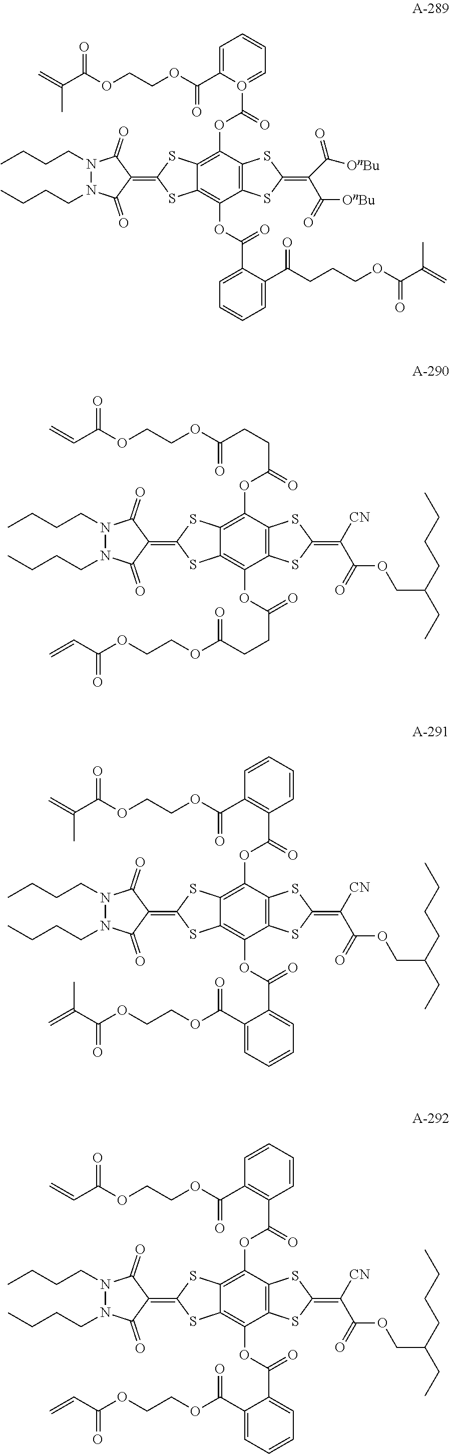

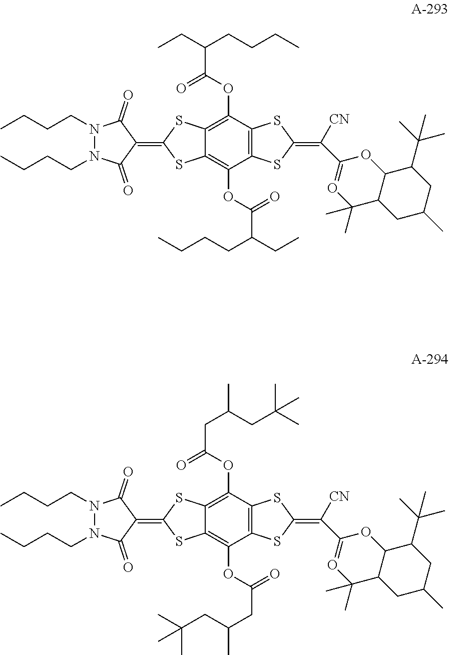

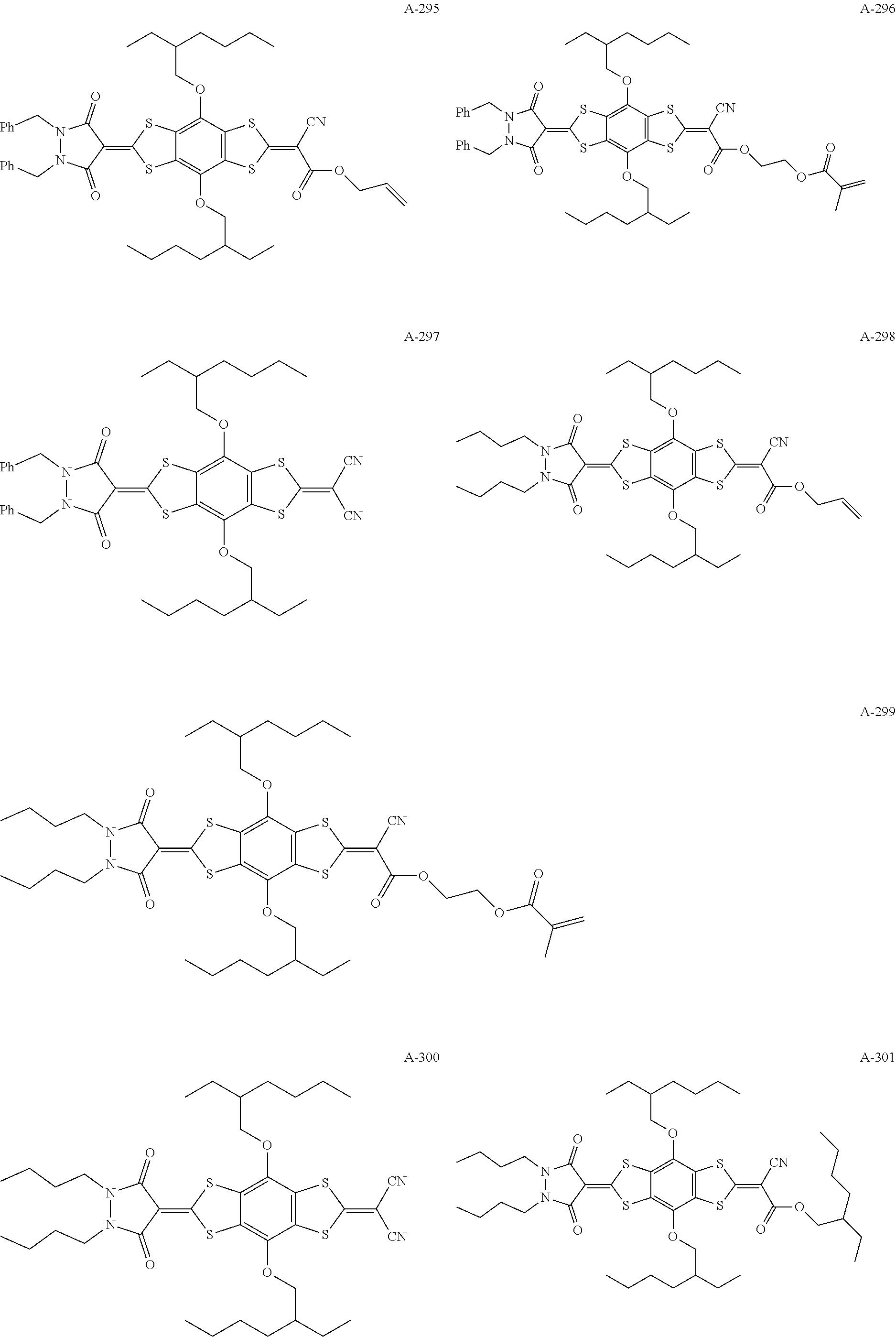

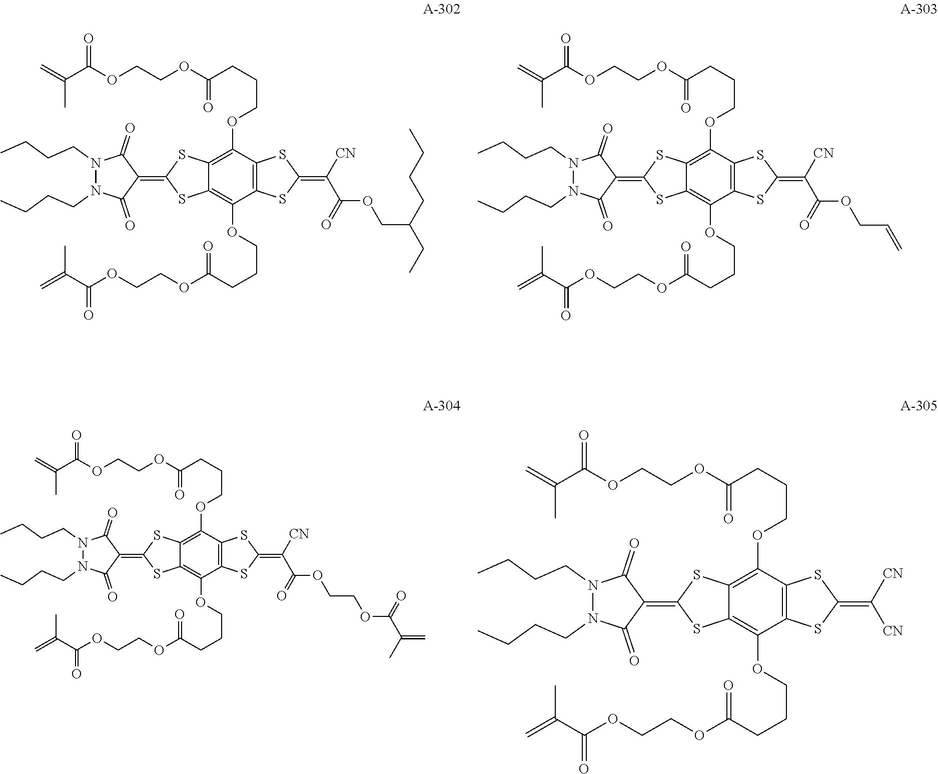 [SMILES: C=C(C)C(=O)OCCOC(=O)CCCOc1c2c(c(OCCCC(=O)OCCOC(=O)C(=C)C)c3c1SC(=C1C(=O)N(CCCC)N(CCCC)C1=O)S3)SC(=C(C#N)C(=O)OCC(CC)CCCC)S2 ; C=CCOC(=O)C(C#N)=C1Sc2c(OCCCC(=O)OCCOC(=O)C(=C)C)c3c(c(OCCCC(=O)OCCOC(=O)C(=C)C)c2S1)SC(=C1C(=O)N(CCCC)N(CCCC)C1=O)S3 ; C=C(C)C(=O)OCCOC(=O)CCCOc1c2c(c(OCCCC(=O)OCCOC(=O)C(=C)C)c3c1SC(=C1C(=O)N(CCCC)N(CCCC)C1=O)S3)SC(=C(C#N)C(=O)OCCOC(=O)C(=C)C)S2 ; C=C(C)C(=O)OCCOC(=O)CCCOc1c2c(c(OCCCC(=O)OCCOC(=O)C(=C)C)c3c1SC(=C1C(=O)N(CCCC)N(CCCC)C1=O)S3)SC(=C(C#N)C#N)S2]

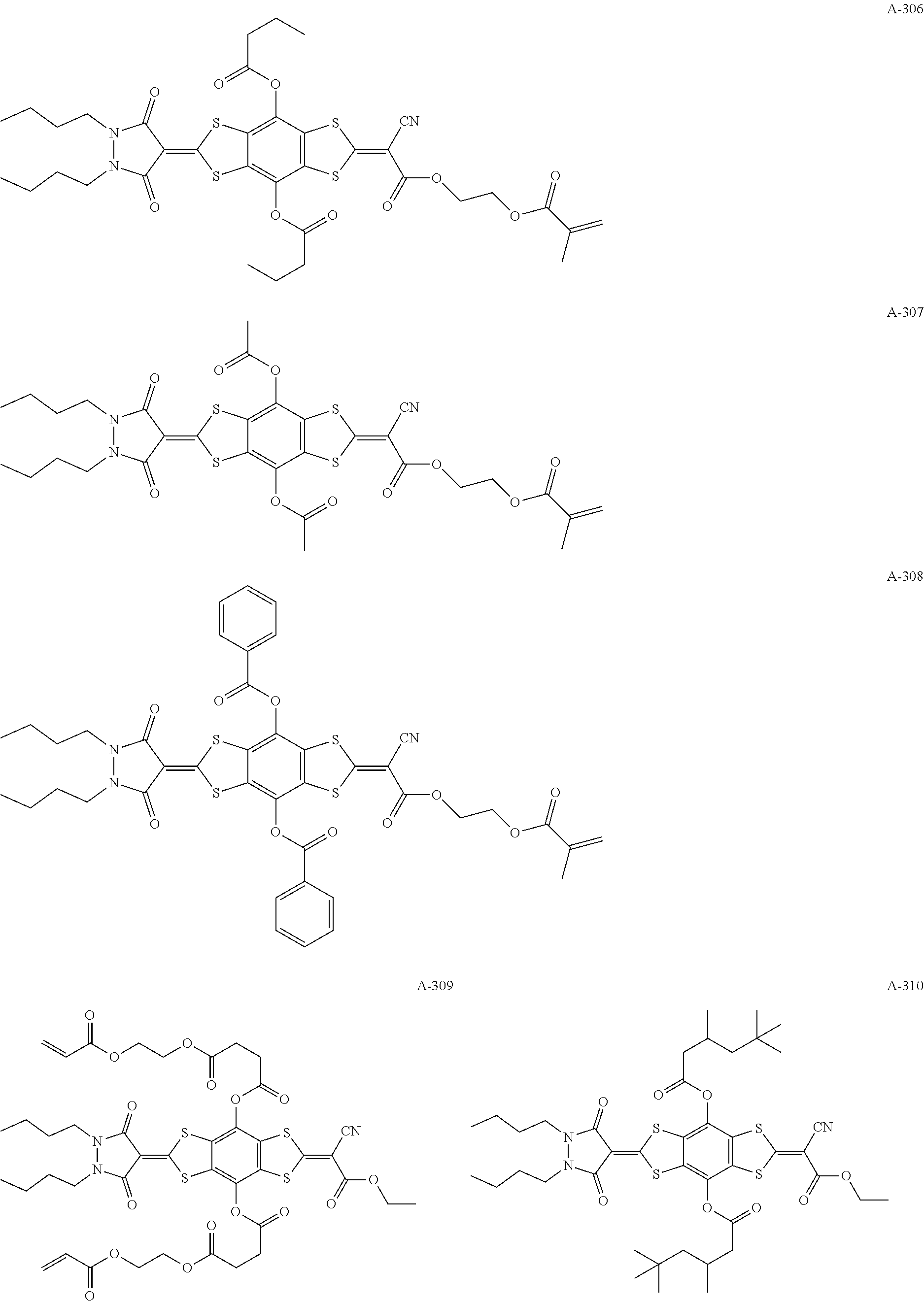

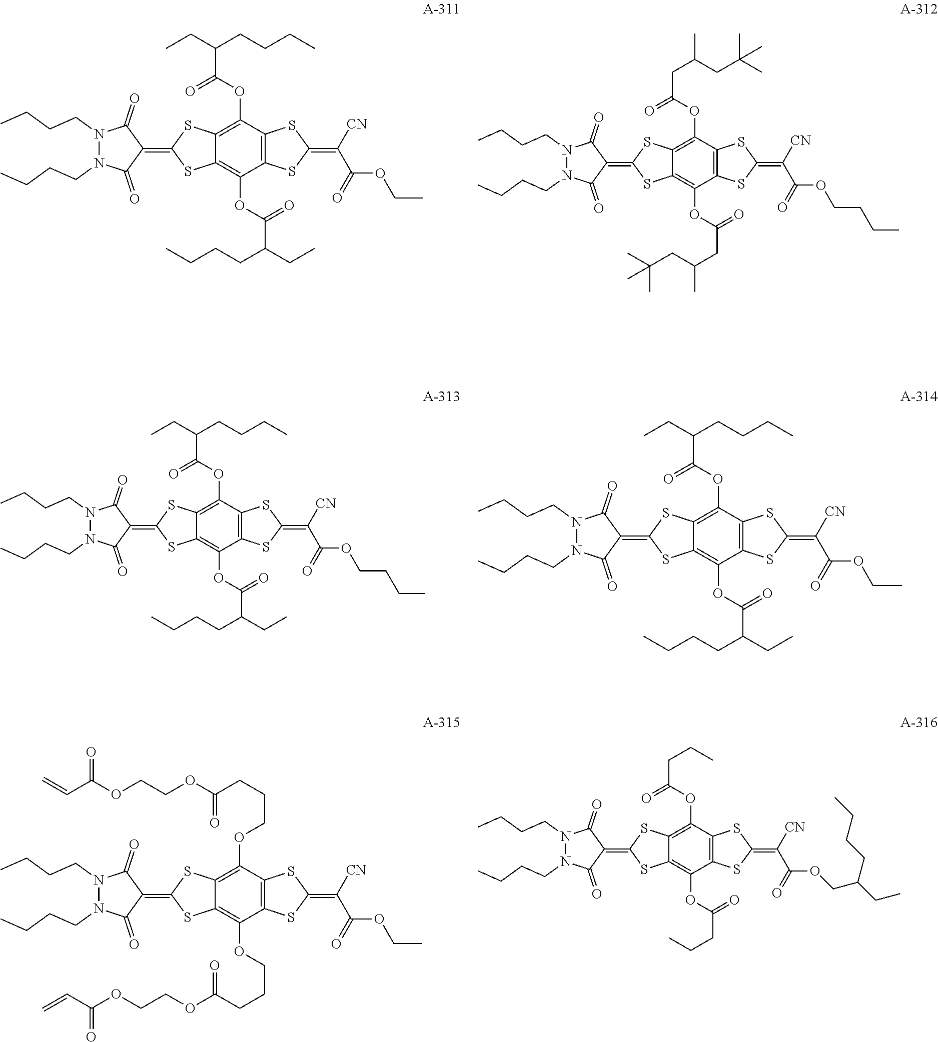

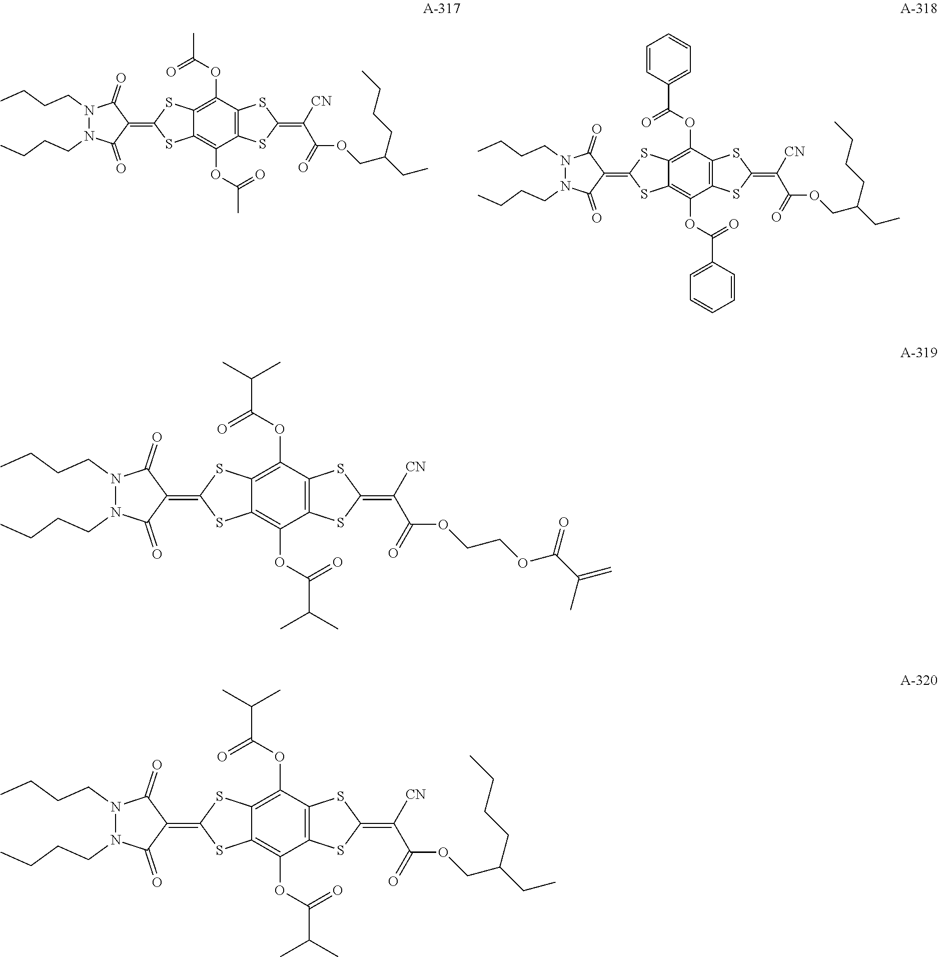

The specific compound is preferably used as an ultraviolet absorber.

The maximal absorption wavelength of the specific compound is preferably in a wavelength range of 380 to 420 nm, and more preferably in a wavelength range of 390 to 410 nm.

In the specific compound, it is preferable that a value of a ratio of an absorbance at a wavelength of 440 nm to an absorbance at a wavelength of 400 nm in a case where the absorbance at the wavelength of 400 nm is set to 1 is less than 0.02.

A molar absorption coefficient of the specific compound at the maximal absorption wavelength is preferably 80,000 L/mol·cm or more, more preferably 85,000 L/mol·cm or more, and still more preferably 90,000 L/mol·cm or more.

In addition, a molar absorption coefficient at a wavelength of 400 nm is preferably 30,000 L/mol·cm or more, more preferably 40,000 L/mol·cm or more, and still more preferably 50,000 L/mol·cm or more.

In addition, a molar absorption coefficient at a wavelength of 440 nm is preferably 1,000 L/mol·cm or less, more preferably 800 L/mol·cm or less, and still more preferably 600 L/mol·cm or less.

The absorbance, the maximal absorption wavelength, the molar absorption coefficient of the specific compound can be determined by measuring spectral spectrum of a solution which is prepared by dissolving the specific compound in ethyl acetate at room temperature (25° C.), using a 1 cm quartz cell. Examples of a measuring device include a spectrophotometer (UV-1800PC, manufactured by Shimadzu Corporation).

The specific compound can be produced according to a method disclosed in WO2009/022736A.

In addition, among the specific compounds, the compound represented by Formula (6) can also be produced by reacting a compound represented by Formula (4) with a compound represented by Formula (5).

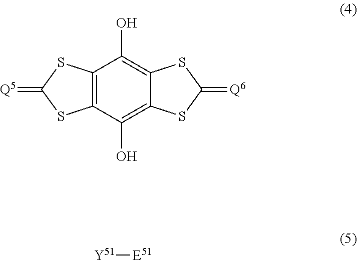

In Formula (4), Q⁵ represents a group represented by Formula (Q-1), Q⁶ represents ═O, ═S, ═NR^{q21}, or ═CR^{q22}R^{q23}, R^{q21} to R^{q23} each independently represent a hydrogen atom or a substituent, R^{q22} and R^{q23} may be bonded to each other to form a ring, provided that, in a case where R^{q22} and R^{q23} are bonded to each other to form a ring, ═CR^{q22}R^{q23} does not have the same structure as Q⁵.

In Formula (5), E⁵¹ represents —COCl, —O(C═O)Cl, —NR^{e51}(C═O)Cl, —NCO, —Cl, —Br, —I, or —SO₂R^{e52}, R^{e51} represents a hydrogen atom, an alkyl group, an aralkyl group, or an aryl group, R^{e52} represents —Cl or an alkoxy group, and Y⁵¹ represents an alkyl group, an aralkyl group, or an aryl group.

Q⁵ and Q⁶ in Formula (4) have the same meaning as Q⁵ and Q⁶ in Formula (6), and preferred ranges thereof are also the same.

The number of carbon atoms in the alkyl group represented by Y⁵¹ of Formula (5) is preferably 1 to 30, more preferably 1 to 20, still more preferably 1 to 15, particularly preferably 1 to 10, and most preferably 1 to 8. The alkyl group may be linear, branched, or cyclic, and preferably linear or branched. The alkyl group may have a substituent. Examples of the substituent include the groups described in the substituent T above.

The number of carbon atoms in the aryl group represented by Y⁵¹ of Formula (5) is preferably 6 to 30, more preferably 6 to 20, still more preferably 6 to 15, particularly preferably 6 to 10, and most preferably 6 to 8. The aryl group may have a substituent. Examples of the substituent include the groups described in the substituent T above.

The number of carbon atoms in an alkyl moiety of the aralkyl group represented by Y⁵¹ of Formula (5) is preferably 1 to 10, more preferably 1 to 5, and still more preferably 1 to 3. The number of carbon atoms in an aryl moiety of the above-described aralkyl group is preferably 6 to 30, more preferably 6 to 20, still more preferably 6 to 15, particularly preferably 6 to 10, and most preferably 6 to 8. The aralkyl group may have a substituent. Examples of the substituent include the groups described in the substituent T above.

Y⁵¹ in Formula (5) is preferably an alkyl group.

The alkyl group, the aralkyl group, and the aryl group, represented by R^{e51} in E⁵¹ of Formula (5), are the same as the groups described in Y⁵¹ of Formula (5).

The number of carbon atoms in the alkoxy group represented by R^{e52} in E⁵¹ of Formula (5) is preferably 1 to 30, more preferably 1 to 20, still more preferably 1 to 15, particularly preferably 1 to 10, and most preferably 1 to 8.

The reaction between the compound represented by Formula (4) and the compound represented by Formula (5) can be carried out in an organic solvent. The organic solvent is not particularly limited, but for example, it is preferably an amide-based solvent such as dimethylformamide, dimethylacetamide, and N-methyl-2-pyrrolidone, tetrahydrofuran, acetonitrile, toluene, methanol, ethanol, isopropyl alcohol, or a mixed solution thereof, and dimethylformamide or dimethylacetamide is particularly preferable. In addition, a reaction ratio of the compound represented by Formula (4) and the compound represented by Formula (5) can be appropriately set according to a structure of the desired compound represented by Formula (6). A reaction temperature is not particularly limited, but is preferably 0° C. to a boiling point of the reaction solvent. A reaction time is not particularly limited, but for example, can be set to 1 hour to 48 hours.

A content of the specific compound in the total solid content of the resin composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

The content of the specific compound is preferably 0.01 to 50 parts by mass with respect to 100 parts by mass of the resin. The lower limit thereof is preferably 0.05 parts by mass or more and more preferably 0.1 parts by mass or more. The upper limit thereof is preferably 40 parts by mass or less, more preferably 30 parts by mass or less, and still more preferably 20 parts by mass or less.

The resin composition may contain only one kind of the specific compound or two or more kinds thereof. In a case where two or more kinds of specific compounds are contained, it is preferable that the total amount thereof is within the above-described range.

### <<Resin>>

The resin composition according to the embodiment of the present invention contains a resin. The resin can be appropriately selected from resins which satisfy various physical properties such as transparency, refractive index, and workability, which are required according to the intended use or purpose.

Examples of the resin include a (meth)acrylic resin, an ene-thiol resin, a polyester resin, a polycarbonate resin, a vinyl polymer [for example, a polydiene resin, a polyalkene resin, a polystyrene resin, a polyvinyl ether resin, a polyvinyl alcohol resin, a polyvinyl ketone resin, a polyfluorovinyl resin, a polyvinyl bromide resin, and the like], a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polyquinoxaline resin, a polybenzoimidazoline resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, a polyamide imide resin, an olefin resin, a cyclic olefin resin, an epoxy resin, and a cellulose acylate resin.

Examples of the (meth)acrylic resin include polymers including a constitutional unit derived from (meth)acrylic acid and/or an ester thereof. Specific examples thereof include polymers obtained by polymerizing at least one compound selected from the group consisting of (meth)acrylic acid, (meth)acrylic acid ester, (meth)acrylamide, and (meth)acrylonitrile.

Examples of the polyester resin include polymers obtained by reacting a polyol (such as ethylene glycol, propylene glycol, glycerin, and trimethylolpropane) with a polybasic acid (such as aromatic dicarboxylic acid (for example, terephthalic acid, isophthalic acid, naphthalenedicarboxylic acid, and dicarboxylic acid in which a hydrogen atom of these aromatic rings is replaced with a methyl group, an ethyl group, or a phenyl group), aliphatic dicarboxylic acid having 2 to 20 carbon atoms (for example, adipic acid, sebacic acid, and dodecanedicarboxylic acid), and alicyclic dicarboxylic acid (for example, cyclohexanedicarboxylic acid)); and polymers obtained by ring-opening polymerization of a cyclic ester compound such as caprolactone monomers (for example, polycaprolactone).

Specific examples of the polyester resin include polyethylene terephthalate and polyethylene naphthalate.

Examples of the epoxy resin include bisphenol A-type epoxy resin, bisphenol F-type epoxy resin, phenol novolac-type epoxy resin, cresol novolac-type epoxy resin, and aliphatic epoxy resin. As the epoxy resin, a commercially available product on the market may be used, and examples of the commercially available product include the following.

Examples of a commercially available product of the bisphenol A-type epoxy resin include jER825, jER827, jER828, jER834, jER1001, jER1002, jER1003, jER1055, jER1007, jER1009, and jER1010 (all manufactured by Mitsubishi Chemical Corporation); and EPICLON 860, EPICLON 1050, EPICLON 1051, and EPICLON 1055 (all manufactured by DIC Corporation). Examples of a commercially available product of the bisphenol F-type epoxy resin include jER806, jER807, jER4004, jER4005, jER4007, and jER4010 (all manufactured by Mitsubishi Chemical Corporation); EPICLON 830 and EPICLON 835 (both manufactured by DIC Corporation); and LCE-21 and RE-602S (both manufactured by Nippon Kayaku Co., Ltd.). Examples of a commercially available product of the phenol novolac-type epoxy resin include jER152, jER154, jER157S70, and jER157S65 (all manufactured by Mitsubishi Chemical Corporation); and EPICLON N-740, EPICLON N-770, and EPICLON N-775 (all manufactured by DIC Corporation). Examples of a commercially available product of the cresol novolac-type epoxy resin include EPICLON N-660, EPICLON N-665, EPICLON N-670, EPICLON N-673, EPICLON N-680, EPICLON N-690, and EPICLON N-695 (all manufactured by DIC Corporation); and EOCN-1020 (manufactured by Nippon Kayaku Co., Ltd.). Examples of a commercially available product of the aliphatic epoxy resin include ADEKA RESIN EP series (for example, EP-4080S, EP-4085S, and EP-4088S; manufactured by ADEKA Corporation); CELLOXIDE 2021P, CELLOXIDE 2081, CELLOXIDE 2083, CELLOXIDE 2085, EHPE 3150, EPOLEAD PB 3600, and EPOLEAD PB 4700 (all manufactured by Daicel Corporation); DENACOL EX-212L, EX-214L, EX-216L, EX-321L, and EX-850L (all manufactured by Nagase ChemteX Corporation); ADEKA RESIN EP series (for example, EP-4000S, EP-4003S, EP-4010S, and EP-4011S; manufactured by ADEKA Corporation); NC-2000, NC-3000, NC-7300, XD-1000, EPPN-501, and EPPN-502 (all manufactured by ADEKA Corporation); and jER1031S (manufactured by Mitsubishi Chemical Corporation). Other examples of the commercially available product of the epoxy resin include MARPROOF G-0150M, G-0105SA, G-0130SP, G-0250SP, G-1005S, G-1005SA, G-1010S, G-2050M, G-01100, and G-01758 (all manufactured by NOF Corporation, epoxy group-containing polymer).

As the cellulose acylate resin, cellulose acylate described in paragraphs 0016 to 0021 of JP2012-215689A is preferably used. As the polyester resin, a commercially available product such as the VYLON Series (for example, VYLON 500) manufactured by Toyobo Co., Ltd. can also be used. As a commercially available product of the (meth)acrylic resin, SK Dyne Series (for example, SK Dyne-SF2147) manufactured by Soken Chemical & Engineering Co., Ltd. can also be used.

As the polystyrene resin, a resin including 50% by mass or more of a repeating unit derived from a styrene-based monomer is preferable; a resin including 70% by mass or more of a repeating unit derived from a styrene-based monomer is more preferable; and a resin including 85% by mass or more of a repeating unit derived from a styrene-based monomer is still more preferable.

Specific examples of the styrene-based monomer include styrene and derivatives thereof. Here, the styrene derivative is a compound in which another group is bonded to styrene, and examples thereof include alkylstyrene such as o-methylstyrene, m-methylstyrene, p-methylstyrene, 2,4-dimethylstyrene, o-ethylstyrene, or p-ethylstyrene, and substituted styrene in which a hydroxyl group, an alkoxy group, a carboxyl group, or halogen is introduced to a benzene nucleus of styrene such as hydroxystyrene, tert-butoxystyrene, vinyl benzoic acid, o-chlorostyrene, or p-chlorostyrene.

In addition, the polystyrene resin may include a repeating unit derived from a monomer other than the styrene-based monomer. Examples of other monomers include alkyl (meth)acrylate such as methyl (meth)acrylate, cyclohexyl (meth)acrylate, methylphenyl (meth)acrylate, or isopropyl (meth)acrylate; an unsaturated carboxylic acid monomer such as methacrylic acid, acrylic acid, itaconic acid, maleic acid, fumaric acid, and cinnamic acid; an unsaturated dicarboxylic acid anhydride monomer which is an anhydride of maleic acid anhydride, itaconic acid, ethylmaleic acid, methylitaconic acid, or chloromaleic acid; an unsaturated nitrile monomer such as acrylonitrile and methacrylonitrile; and a conjugated diene such as 1,3-butadiene, 2-methyl-1,3-butadiene (isoprene), 2,3-dimethyl-1,3-butadiene, 1,3-pentadiene, and 1,3-hexadiene.

Examples of a commercially available product of the polystyrene resin include AS-70 (acrylonitrile-styrene copolymer resin) manufactured by NIPPON STEEL & SUMITOMO METAL CORPORATION; SMA2000P (styrene-maleic acid copolymer) manufactured by KAWAHARA PETROCHEMICAL CO., LTD.; CLEAREN 530L and CLEAREN 730L manufactured by Denka Company Limited; TUFPRENE 126S and ASAPRENE T411 manufactured by Asahi Kasei Corporation; KRATON D1102A and KRATON D1116A manufactured by Kraton Corporation; STYROLUX S and STYROLUX T manufactured by INEOS Styrolution Group GmbH; ASAFLEX 840 and ASAFLEX 860 manufactured by Asahi Kasei Corporation; 679, HF77, SGP-10, 475D, H0103, and HT478 manufactured by PS Japan Corporation; and DICSTYRENE XC-515, DICSTYRENE XC-535, and DICSTYRENE GH-8300-5 manufactured by DIC Corporation. In addition, examples of a commercially available product of the hydrogenated polystyrene resin include TUFTEC H Series manufactured by Asahi Kasei Corporation, KRATON G Series manufactured by Shell Japan Limited, DYNARON (hydrogenated styrene-butadiene random copolymer) manufactured by JSR Corporation, and SEPTON manufactured by Kuraray Co., Ltd. In addition, examples of a commercially available product of the modified polystyrene resin include TUFTEC M Series manufactured by Asahi Kasei Corporation, EPOFRIEND manufactured by Daicel Corporation, polar group-modified DYNARON manufactured by JSR Corporation, and RESEDA manufactured by TOAGOSEI CO., LTD.

Examples of the cyclic olefin resin include (R1) polymers including a structural unit derived from a norbornene compound, (R2) polymers including a structural unit derived from a monocyclic olefin compound which is not a norbornene compound, (R3) polymers including a structural unit derived from a cyclic conjugated diene compound, (R4) polymers including a structural unit derived from a vinyl alicyclic hydrocarbon compound, and hydrides of polymers including a structural unit derived from each of the compounds (R1) to (R4). In the present specification, the polymer including a structural unit derived from a norbornene compound and the polymer including a structural unit derived from a monocyclic olefin compound include ring-opening polymers of the respective compounds.

The cyclic olefin resin is not particularly limited, but is preferably a polymer having a structural unit derived from a norbornene compound, which is represented by Formula (A-II) or Formula (A-III). The polymer having the structural unit represented by Formula (A-II) is an addition polymer of a norbornene compound, and the polymer having the structural unit represented by Formula (A-III) is a ring-opening polymer of a norbornene compound.

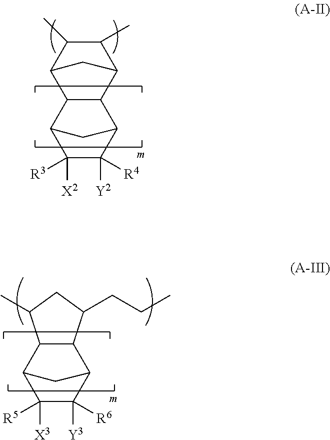

In Formulae (A-II) and (A-III), m represents an integer of 0 to 4 and is preferably 0 or 1.

R³ to R⁶ in Formulae (A-II) and (A-III) each independently represent a hydrogen atom or a hydrocarbon group having 1 to 10 carbon atoms.

Examples of the hydrocarbon group represented by R³ to R⁶ include an alkyl group, an alkenyl group, an alkynyl group, and an aryl group, and an alkyl group or an aryl group is preferable.

X² and X³, Y² and Y³ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)ₙCOOR¹¹, —(CH₂)ₙOCOR¹², —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (—CO)₂O or (—CO)₂NR¹⁵ which is formed by bonding X² and Y², or X³ and Y³.

Here, R¹¹ to R¹⁵ in the above-described groups which can be adopted as X², X³, Y², and Y³ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10, and is preferably 0 to 8 and more preferably 0 to 6.

In Formulae (A-II) and (A-III), R³ to R⁶ are each independently preferably a hydrogen atom or —CH₃, and from the viewpoint of moisture permeability, still more preferably a hydrogen atom.

Each of X² and X³ is preferably a hydrogen atom, —CH₃, or —C₂H₅, and from the viewpoint of moisture permeability, still more preferably a hydrogen atom.

Y² and Y³ are each independently preferably a hydrogen atom, a halogen atom (particularly a chlorine atom), or —(CH₂)ₙCOOR¹¹ (particularly —COOCH₃), and from the viewpoint of moisture permeability, still more preferably a hydrogen atom.

Other groups are appropriately selected.

The polymer having the structural unit represented by Formula (A-II) or Formula (A-III) may further include one or more of a structural unit represented by Formula (A-I).

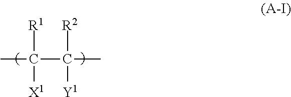

In Formula (A-I), R¹ and R² each independently represent a hydrogen atom or a hydrocarbon group having 1 to 10 carbon atoms, and X¹ and Y¹ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)ₙCOOR¹¹, —(CH₂)ₙOCOR¹², —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (—CO)₂O or (—CO)₂NR¹⁵ formed by X² and Y² or X³ and Y³ being bonded to each other. R¹¹ to R¹⁵ in the above-described groups which can be adopted as X¹ and Y¹ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10.

A content of the structural unit represented by Formula (A-II) or Formula (A-III) in the cyclic polyolefin resin is preferably 90% by mass or less, more preferably 30% to 85% by mass, still more preferably 50% to 79% by mass, and even more preferably 60% to 75% by mass.

The cyclic olefin resin is described in JP1998-007732A (JP-H10-007732A), JP2002-504184A, WO2004/070463A, and the like, the contents of which can be referred to as appropriate.

The cyclic olefin resin is obtained by performing an addition polymerization of a norbornene compound (for example, a polycyclic unsaturated compound of norbornene) with each other.

Examples of a commercially available product of the cyclic olefin resin include ARTON series (for example, ARTON G, ARTON F, and ARTON RX4500) manufactured by JSR Corporation, and Zeonor ZF14, Zeonor ZF16, Zeonex 250, and Zeonex 280 (manufactured by Zeon Corporation).

In addition, examples of the cyclic olefin resin include copolymers obtained by an addition copolymerization of, as necessary, a norbornene compound, and olefin such as ethylene, propylene, and butene, conjugated diene such as butadiene and isoprene, unconjugated diene such as ethylidene norbornene, or an ethylenically unsaturated compound such as acrylonitrile, acrylic acid, methacrylic acid, maleic acid anhydride, acrylic acid ester, methacrylic acid ester, maleimide, vinyl acetate, and vinyl chloride. Among these, a copolymer with ethylene is preferable. Examples of the above-described addition (co)polymers of a norbornene compound include APL8008T (Tg: 70° C.), APL6011T (Tg: 105° C.), APL6013T (Tg: 125° C.), and APL6015T (Tg: 145° C.) which are sold by Mitsui Chemicals, Inc. under a trade name of APL and have different glass transition temperatures (Tg). In addition, pellets such as TOPAS8007, TOPAS6013, and TOPAS6015 are commercially available from Polyplastics Co., Ltd. Furthermore, Appear3000 is commercially available from Film Ferrania S. R. L.

In addition, the hydride of the cyclic olefin resin can be synthesized by an addition polymerization or a ring-opening metathesis polymerization of a norbornene compound or the like and then an addition of hydrogen. The synthesis method is described, for example, in JP1989-240517A (JP-H01-240517A), JP1995-196736A (JP-H07-196736A), JP1985-026024A (JP-S60-026024A), JP1987-019801A (JP-S62-019801A), JP2003-159767A, and JP2004-309979A.

A weight-average molecular weight of the cyclic olefin resin is preferably 5,000 to 500,000, more preferably 8,000 to 200,000, and still more preferably 10,000 to 100,000.

Examples of the polycarbonate resin include a reaction product of a polyhydric phenol compound and phosgene or a carbonic ester compound.

Examples of the polyhydric phenol compound include hydroquinone, resorcinol, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenyl sulfone, 4,4′-dihydroxydiphenyl sulfoxide, 4,4′-dihydroxydiphenyl sulfide, 3,3′-dimethyl-4,4′-dihydroxydiphenyl sulfide, and 4,4′-dihydroxydiphenyl oxide. Among these, hydroquinone, resorcinol, 4,4′-dihydroxydiphenyl, or bisphenol A is preferable.

Examples of the carbonic ester compound include phosgene, diphenyl carbonate, bis(chlorophenyl) carbonate, dinaphthyl carbonate, bis(diphenyl) carbonate, dimethyl carbonate, diethyl carbonate, and dibutyl carbonate; and bis(diphenyl) carbonate, dimethyl carbonate, or diethyl carbonate is preferable.

Examples of a commercially available product of the polycarbonate resin include PANLITE L-1250WP and PANLITE SP-1516 manufactured by Teijin Limited; Iupizeta EP-5000 and Iupizeta EP-4000 manufactured by Mitsubishi Gas Chemical Company Inc.; and Calibre 301-30 manufactured by Sumika Polycarbonate Ltd.

Examples of the thiourethane resin include a reaction product of an isocyanate compound and a polythiol compound and a reaction product of a thiourethane resin precursor. Examples of a commercially available product of the thiourethane resin precursor include MR-7, MR-8, MR-10, and MR-174 manufactured by Mitsui Chemicals, Inc.

Examples of the polyamide resin include an aliphatic polyamide resin and an aromatic polyamide resin. Examples of the aliphatic polyamide resin include Nylon 6, Nylon 11, Nylon 12, Nylon 46, Nylon 66, Nylon 666, Nylon 610, and Nylon 612. Examples of the aromatic polyamide resin include a resin which is polymerized by dehydration condensation of a diamine and a dicarboxylic acid, in which at least one of the diamine or the dicarboxylic acid includes an aromatic ring. Specific examples of the aromatic polyamide resin include a condensation polymer of m-xylylenediamine and adipic acid or an adipic acid halide.

The resin may have an acid group. Examples of the acid group include a carboxyl group, a phosphoric acid group, a sulfonic acid group, and a phenolic hydroxy group. As the acid group, one kind may be used alone, or two or more kinds may be used in combination. The resin having an acid group can be used as an alkali-soluble resin or as a dispersant.

As the resin having an acid group, reference can be made to the description in paragraphs 0558 to 0571 of JP2012-208494A (paragraphs 0685 to 0700 of the corresponding US2012/0235099A) and the description in paragraphs 0076 to 0099 of JP2012-198408A, the contents of which are incorporated herein by reference. In addition, as the resin having an acid group, ACRYBASE FF-426 (manufactured by NIPPON SHOKUBAI CO., LTD.) can also be used.

The acid value of the resin having an acid group is preferably 30 to 200 mgKOH/g. The lower limit or the acid value is preferably 50 mgKOH/g or more and more preferably 70 mgKOH/g or more. In addition, the upper limit of the acid value is preferably 150 mgKOH/g or less and more preferably 120 mgKOH/g or less. The acid value of the resin is a value calculated by measuring in accordance with JIS K0070: 1992 and converting as 1 mmol/g=56.1 mgKOH/g.

The resin may have a curable group. Examples of the curable group include an ethylenically unsaturated bond-containing group, an epoxy group, a methylol group, and an alkoxysilyl group. Examples of the ethylenically unsaturated bond-containing group include a vinyl group, a styrene group, an allyl group, a methallyl group, and a (meth)acryloyl group. Examples of the alkoxysilyl group include a monoalkoxysilyl group, a dialkoxysilyl group, and a trialkoxysilyl group.

Examples of a commercially available product of the resin containing a curable group include Dianal BR Series (poly(methyl methacrylate)(PMMA), for example Dianal BR-80, BR-83, and BR-87, manufactured by Mitsubishi Chemical Corporation); Photomer 6173 (COOH-containing polyurethane acrylic oligomer, Diamond Shamrock Co., Ltd.); Viscoat R-264 and KS Resist 106 (both manufactured by Osaka Organic Chemical Industry Ltd.); Cyclomer P series (for example, ACA230AA) and Placcel CF 200 series (all manufactured by Daicel Corporation); Ebecryl 3800 (manufactured by Daicel UCB Company, Ltd.); and Acrycure RD-F8 (manufactured by Nippon Shokubai Co., Ltd.). In addition, examples thereof also include commercially available products such as the products described in the section of the epoxy resin described above.

In a case where the resin composition according to the embodiment of the present invention is used for a lens (for example, an eyeglass lens), as the resin, a thermoplastic resin such as a carbonate resin and a (meth)acrylic resin, or a thermosetting resin such as a urethane resin is suitable.

As the resin, a pressure sensitive adhesive or an adhesive can also be used. Examples of the pressure sensitive adhesive include an acrylic pressure sensitive adhesive, a rubber-based pressure sensitive adhesive, and a silicone-based pressure sensitive adhesive. The acrylic pressure sensitive adhesive refers to a pressure sensitive adhesive including a polymer ((meth)acrylic polymer) of a (meth)acrylic monomer. Examples of the adhesive include a urethane resin adhesive, a polyester adhesive, an acrylic resin adhesive, an ethylene vinyl acetate resin adhesive, a polyvinyl alcohol adhesive, a polyamide adhesive, and a silicone adhesive. Among these, as the adhesive, from the viewpoint of high adhesive force, a urethane resin adhesive or a silicone adhesive is preferable. As the adhesive, a commercially available product on the market may be used, and examples of the commercially available product thereof include a urethane resin adhesive (LIS-073-50U: trade name, manufactured by of Toyo Ink Co., Ltd.) and an acrylic pressure sensitive adhesive (SK Dyne-SF2147: trade name, manufactured by Soken Chemical & Engineering Co., Ltd.).

The resin is preferably at least one selected from a (meth)acrylic resin, a polystyrene resin, a polyester resin, a polyurethane resin, a thiourethane resin, a polyimide resin, a polyamide resin, an epoxy resin, a polycarbonate resin, a phthalate resin, a cellulose acylate resin, or a cyclic olefin resin; and from the reason that compatibility with the specific compound is favorable and a cured substance in which surface unevenness is suppressed is easily obtained, it is more preferable to be at least one selected from a (meth)acrylic resin, a polystyrene resin, a polyester resin, a polyurethane resin, or a cyclic olefin resin.

A weight-average molecular weight (Mw) of the resin is preferably 2,000 to 2,000,000. The lower limit of Mw of the resin is preferably 5,000 or more, more preferably 10,000 or more, and still more preferably 50,000 or more. The upper limit of Mw of the resin is preferably 1,000,000 or less, more preferably 500,000 or less, and still more preferably 200,000 or less. In addition, in a case of using an epoxy resin, the weight-average molecular weight (Mw) of the epoxy resin is preferably 100 or more, and more preferably 200 to 2,000,000. The upper limit of Mw of the epoxy resin is preferably 1,000,000 or less and more preferably 500,000 or less. The lower limit of Mw of the epoxy resin is preferably 2000 or more.

The weight-average molecular weight (Mw) is a value measured by gel permeation chromatography (GPC). In the measurement by GPC, HLC (registered trademark)-8020GPC (manufactured by Tosoh Corporation) is used as a measuring device, three columns of TSKgel (registered trademark) Super Multipore HZ-H (4.6 mmID×15 cm, manufactured by Tosoh Corporation) are used as a column, and tetrahydrofuran (THF) is used as an eluent. In addition, as the measurement conditions, a sample concentration of 0.45% by mass, a flow rate of 0.35 ml/min, a sample injection amount of 10 μl, and a measurement temperature of 40° C. are set, and a RI detector is used. The calibration curve is created from eight samples of “Standard sample TSK standard, polystyrene” manufactured by Tosoh Corporation: “F-40”, “F-20”, “F-4”, “F-1”, “A-5000”, “A-2500”, “A-1000”, and “n-propylbenzene”.

The total light transmittance of the resin is preferably 80% or more, more preferably 85% or more, and still more preferably 90% or more. In the present specification, the total light transmittance of the resin is a value measured based on the contents described in “The Fourth Series of Experimental Chemistry 29 Polymer Material” (Maruzen, 1992), pp. 225 to 232, edited by the Chemical Society of Japan.

A content of the resin in the total solid content of the resin composition is preferably 1% to 99.9% by mass. The lower limit thereof is preferably 30% by mass or more, more preferably 50% by mass or more, and still more preferably 70% by mass or more. The upper limit thereof is preferably 95% by mass or less, more preferably 90% by mass or less, and still more preferably 80% by mass or less. The resin composition may contain only one kind of the resin or two or more kinds thereof. In a case where two or more kinds of resins are contained, it is preferable that the total amount thereof is within the above-described range.

### <<Other Ultraviolet Absorbers>>

The resin composition according to the embodiment of the present invention can contain an ultraviolet absorber other than the above-described specific compound (hereinafter, also referred to as other ultraviolet absorbers). According to this aspect, a cured substance capable of shielding light having a wavelength in the ultraviolet region over a wide range can be formed.

The maximal absorption wavelength of the other ultraviolet absorbers is preferably in a wavelength range of 300 to 380 nm, more preferably in a wavelength range of 300 to 370 nm, still more preferably in a wavelength range of 310 to 360 nm, and particularly preferably in a wavelength range of 310 to 350 nm.

It is also preferable that the other ultraviolet absorbers are compounds having a polymerizable group. Examples of the polymerizable group include a vinyl group, an allyl group, a (meth)acryloyl group, a (meth)acryloyloxy group, a (meth)acryloylamino group, and a vinylphenyl group.

Examples of the other ultraviolet absorbers include an aminobutadiene-based ultraviolet absorber, a dibenzoylmethane-based ultraviolet absorber, a benzotriazole-based ultraviolet absorber, a benzophenone-based ultraviolet absorber, a salicylic acid-based ultraviolet absorber, an acrylate-based ultraviolet absorber, and a triazine-based ultraviolet absorber. Among these, a benzotriazole-based ultraviolet absorber, a benzophenone-based ultraviolet absorber, or a triazine-based ultraviolet absorber is preferable, and a benzotriazole-based ultraviolet absorber or a triazine-based ultraviolet absorbers is more preferable. Specific examples of the other ultraviolet absorbers include compounds described in Examples later. In addition, as the other ultraviolet absorbers, compounds and the like described in paragraphs 0065 to 0070 of JP2009-263616A, paragraph 0065 of WO2017/122503A, JP2003-128730A, JP2003-129033A, JP2014-077076A, JP2015-164994A, JP2015-168822A, JP2018-135282A, JP2018-168089A, JP2018-168278A, JP2018-188589A, JP2019-001767A, JP2020-023697A, JP2020-041013A, JP5518613B, JP5868465B, JP6301526B, JP6354665B, JP2017-503905A, WO2015/064674A, WO2015/064675A, WO2017/102675A, WO2018/190281A, WO2018/216750A, WO2019/087983A, EP2379512B, and EP2951163B can be used.

In a case where the resin composition contains other ultraviolet absorbers, a content of the other ultraviolet absorbers in the total solid content of the resin composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

In addition, the total content of the above-described specific compound and the other ultraviolet absorbers in the total solid content of the resin composition is preferably 0.01% to 50% by mass. The lower limit thereof is preferably 0.05% by mass or more and more preferably 0.1% by mass or more. The upper limit thereof is preferably 40% by mass or less, more preferably 30% by mass or less, and still more preferably 20% by mass or less.

The resin composition may contain only one kind of the other ultraviolet absorbers or two or more kinds thereof. In a case where two or more kinds of other ultraviolet absorbers are contained, it is preferable that the total amount thereof is within the above-described range.

### <<Polymerizable Compound>>

The resin composition according to the embodiment of the present invention can contain a polymerizable compound. As the polymerizable compound, a compound which can be polymerized and cured by applying energy can be used without limitation. The polymerizable compound may be a radically polymerizable compound or a cationically polymerizable compound. Examples of the radically polymerizable compound include a compound having an ethylenically unsaturated bond-containing group. The polymerizable compound is preferably a compound having an ethylenically unsaturated bond-containing group, and more preferably a compound having two or more ethylenically unsaturated bond-containing groups. The upper limit of the number of ethylenically unsaturated bond-containing groups included in the polymerizable compound is preferably 15 or less, more preferably 10 or less, and still more preferably 6 or less. Examples of the ethylenically unsaturated bond-containing group included in the polymerizable compound include a vinyl group, an allyl group, and a (meth)acryloyl group.

It is preferable that the polymerizable compound may be any one of a monomer, a prepolymer (that is, a dimer, a trimer, or an oligomer), a mixture thereof, or a (co)polymer of a compound selected from the monomer and the prepolymer.

A molecular weight of the polymerizable compound is preferably 100 to 3,000. The upper limit thereof is preferably 2,000 or less and more preferably 1,500 or less. The lower limit thereof is preferably 150 or more and more preferably 250 or more.

### (Radically Polymerizable Compound)

Examples of the radically polymerizable compound include a compound having an ethylenically unsaturated bond-containing group.

Examples of the radically polymerizable compound include unsaturated carboxylic acids (for example, acrylic acid, methacrylic acid, itaconic acid, crotonic acid, isocrotonic acid, and maleic acid), esters of unsaturated carboxylic acid, amides of unsaturated carboxylic acid, and (co)polymers of unsaturated carboxylic acid, or an ester or amide thereof. Among these, an ester of unsaturated carboxylic acid and aliphatic polyhydric alcohol, an amide of unsaturated carboxylic acid and aliphatic polyvalent amine, or a homopolymer or copolymer thereof is preferable.

As the radically polymerizable compound, an addition reactant of an unsaturated carboxylic acid ester or unsaturated carboxylic acid amide having a nucleophilic substituent (for example, a hydroxy group, an amino group, a mercapto group, and the like) and a monofunctional or polyfunctional isocyanate compound or epoxy compound; a dehydration condensation reactant of an unsaturated carboxylic acid ester or unsaturated carboxylic acid amide having a nucleophilic substituent and a monofunctional or polyfunctional carboxylic acid; an addition reactant of an unsaturated carboxylic acid ester or unsaturated carboxylic acid amide having an electrophilic substituent (for example, an isocyanate group, an epoxy group, and the like) and a monofunctional or polyfunctional alcohol, amine, or thiol; and a substitution reactant of an unsaturated carboxylic acid ester or unsaturated carboxylic acid amide having a leaving substituent (for example, a halogen group, a tosyloxy group, and the like) and a monofunctional or polyfunctional alcohol, amine, or thiol can also be used. Furthermore, a compound obtained by substituting the above-described unsaturated carboxylic acid with an unsaturated phosphonic acid, styrene, vinyl ether, or the like can also be used.

A plurality of compounds with different numbers of functional groups or a plurality of compounds with different kinds of polymerizable groups (for example, acrylic acid ester, methacrylic acid ester, a styrene compound, a vinyl ether compound, and the like) may be used in combination as the radically polymerizable compound.

As the radically polymerizable compound, a (meth)acrylate compound is preferable; a bi- or higher functional (meth)acrylate compound is more preferable; a bi- to pentadecafunctional (meth)acrylate compound is still more preferable; a bi- to decafunctional (meth)acrylate compound is even more preferable; and a bi- to hexafunctional (meth)acrylate compound is particularly preferable.

Specific examples of the radically polymerizable compound include pentaerythritol tri(meth)acrylate, pentaerythritol tetra(meth)acrylate, dipentaerythritol penta(meth)acrylate, dipentaerythritol hexa(meth)acrylate, tri((meth)acryloyloxyethyl) isocyanurate, a pentaerythritol tetra(meth)acrylate ethylene oxide (EO)-modified product, a dipentaerythritol hexa(meth)acrylate ethylene oxide (EO)-modified product, and benzyl (meth)acrylate.

Examples of a commercially available product of the radically polymerizable compound include polyfunctional (meth)acrylate compounds such as KAYARAD Series (for example, D-330, D-320, D-310, PET-30, TPA-330, and DPHA) manufactured by Nippon Kayaku Co., Ltd.; NK Ester Series (for example, A-DPH-12E, A-TMMT, and A-TMM-3) manufactured by Shin-Nakamura Chemical Co., Ltd.; Light Acrylate Series (for example, DCP-A) manufactured by Kyoeisha Chemical Co., Ltd.; ARONIX Series (for example, M-305, M-306, M-309, M-450, M-402, and TO-1382) manufactured by TOAGOSEI CO., LTD.; and VISCOAT Series (for example, V #802) manufactured by Osaka Organic Chemical Industry Ltd.

As the radically polymerizable compound, the (meth)acrylate compounds described in JP1973-064183A (JP-S48-064183A), JP1974-043191B (JP-S49-043191B), and JP1977-030490B (JP-S52-030490B), and the compounds introduced as photocurable monomers and oligomers in The Adhesion Society of Japan, vol. 20, No. 7, pp. 300 to 308 (1984) can be used.

### (Cationically Polymerizable Compound)

Examples of the cationically polymerizable compound include a compound having a cationically polymerizable group. Examples of the cationically polymerizable group include a cyclic ether group such as an epoxy group and an oxetanyl group, and a vinyl ether group. Among these, a cyclic ether group is preferable. In addition, the cationically polymerizable compound is preferably a polyfunctional cationically polymerizable compound having two or more cationically polymerizable groups.

Examples of the cationically polymerizable compound include a polyfunctional alicyclic epoxy compound, a polyfunctional heterocyclic epoxy compound, a polyfunctional oxetane compound, an alkylene glycol diglycidyl ether, and an alkylene glycol monovinyl monoglycidyl ether.

Specific examples of the cationically polymerizable compound include 3′,4′-epoxycyclohexylmethyl 3,4-epoxycyclohexane carboxylate, bisphenol A diglycidyl ether, hydrogenated bisphenol A diglycidyl ether, 3,4-epoxycyclohexylmethyl-3′,4′-epoxycyclohexane carboxylate, a 1,2-epoxy-4-(2-oxiranyl)cyclohexane adduct of 2,2-bis(hydroxymethyl)-1-butanol, xylylene bisoxetane, 3-ethyl-3-hydroxymethyl oxetane, 3-ethyl-3-{[(3-ethyloxetane-3-yl)methoxy]methyl}oxetane, cyclohexanedimethanol divinyl ether, 2-ethylhexyl vinyl ether, cyclohexanedimethanol monovinyl ether, 4-hydroxybutyl vinyl ether, and compounds described in paragraphs 0029 to 0058 of JP2012-046577A.

As the cationically polymerizable compound, a (meth)acrylate compound having a cationically polymerizable group can also be used. Specific examples of the (meth)acrylate compound having a cationically polymerizable group include 3,4-epoxycyclohexylmethyl methacrylate. Examples of a commercially available product thereof include CYCLOMER M100 manufactured by Daicel Corporation.

As the cationically polymerizable compound, ARON OXETANE series (OXT-101, OXT-121, OXT-221, and the like) manufactured by TOAGOSEI CO., LTD.; CELLOXIDE series (2021P) manufactured by Daicel Corporation; and alkyl divinyl ether CHDVE, alkyl monovinyl ether EHVE, hydroxyalkyl vinyl ether CHMVE, and hydroxyalkyl vinyl ether HBVE manufactured by Nippon Carbide Industries Co., Inc. can also be used. In addition, those exemplified as specific examples of the epoxy resin described below can also be used.

In a case where the resin composition contains a polymerizable compound, a content of the polymerizable compound in the total solid content of the resin composition is preferably 0.1% to 90% by mass. The lower limit thereof is preferably 1% by mass or more and more preferably 5% by mass or more. The upper limit thereof is preferably 80% by mass or less and more preferably 70% by mass or less. The resin composition may contain only one kind of the polymerizable compound or two or more kinds thereof. In a case where two or more kinds of polymerizable compounds are contained, it is preferable that the total amount thereof is within the above-described range.

### <<Polymerization Initiator>>

The resin composition can contain a polymerization initiator. As the polymerization initiator, a compound capable of generating initiating species required for the polymerization reaction by applying energy can be used. Examples of the polymerization initiator include a radical polymerization initiator and a cationic polymerization initiator. In a case where the radically polymerizable compound is used as the polymerizable compound, the polymerization initiator is preferably a radical polymerization initiator. In a case where the cationically polymerizable compound is used as the polymerizable compound, the polymerization initiator is preferably a cationic polymerization initiator.

The polymerization initiator can be appropriately selected from, for example, a photopolymerization initiator and a thermal polymerization initiator, and a photopolymerization initiator is preferable. The photopolymerization initiator is a compound which is photosensitized by exposure light and initiates or promotes the polymerization of the polymerizable compound. Examples of the photopolymerization initiator include a photoradical polymerization initiator and a photocationic polymerization initiator, and a photoradical polymerization initiator is preferable. It is preferable that the photoradical polymerization initiator is a compound which is sensitive to actinic rays having a wavelength of 300 nm or more to generate radicals.

### (Photoradical Polymerization Initiator)

Examples of the photoradical polymerization initiator include an oxime compound, a halogenated hydrocarbon derivative (for example, a compound having a triazine skeleton, a compound having an oxadiazole skeleton, and the like), an oxadiazole compound, a carbonyl compound, a ketal compound, a benzoin compound, an acridine compound, an organic peroxide, an azo compound, a coumarin compound, an azide compound, a metallocene compound, a hexaarylbiimidazole compound, an organic borate compound, a disulfonate compound, an onium salt compound, an acetophenone compound, an acylphosphine compound, and a benzophenone compound.

Examples of the acetophenone compound include an aminoacetophenone compound and a hydroxyacetophenone compound. Examples of the acetophenone compound include acetophenone compounds described in JP2009-191179A and JP1998-291969A (JP-H10-291969A). Examples of a commercially available product of the aminoacetophenone compound include Omnirad 907, Omnirad 369, Omnirad 369E, and Omnirad 379EG (all manufactured by IGM Resins B. V.). Examples of a commercially available product of the hydroxyacetophenone compound include Omnirad 184, Omnirad 1173, Omnirad 2959, and Omnirad 127 (all manufactured by IGM Resins B. V.).

Examples of the acylphosphine compound include the acylphosphine compound described in JP4225898B. Examples of a commercially available product of the acylphosphine compound include Omnirad 819 and Omnirad TPO (both manufactured by IGM Resins B. V.).

Examples of the benzophenone compound include benzophenone, 2-methylbenzophenone, 3-methylbenzophenone, 4-methylbenzophenone, 4-methoxybenzophenone, 2-chlorobenzophenone, 4-chlorobenzophenone, 4-bromobenzophenone, 2-carboxybenzophenone, 2-ethoxycarbonylbenzophenone, benzophenone tetracarboxylic acid or tetramethyl ester thereof, 4,4′-bis(dialkylamino)benzophenones (for example, 4,4′-bis(dimethylamino)benzophenone, 4,4′-bis(dicyclohexylamino)benzophenone, 4,4′-bis(diethylamino)benzophenone, and 4,4′-bis(dihydroxyethylamino)benzophenone), 4-methoxy-4′-dimethylaminobenzophenone, 4,4′-dimethoxybenzophenone, 4-dimethylaminobenzophenone, and 4-dimethylaminoacetophenone. Among these, from the viewpoint of sensitivity and light resistance of the cured substance to be obtained, 4,4′-bis(diethylamino)benzophenone is preferable.

Examples of the oxime compound include compounds described in JP2001-233842A, compounds described in JP2000-080068A, compounds described in JP2006-342166A, and compounds described in paragraphs 0073 to 0075 of JP2016-006475A. Among the oxime compounds, an oxime ester compound is preferable. Examples of a commercially available product of the oxime compound include Irgacure OXE01 and Irgacure OXE02 (manufactured by BASF SE), and Irgacure OXE03 (manufactured by BASF SE).

Examples of the halogenated hydrocarbon derivative include compounds described in Wakabayashi et al., “Bull Chem. Soc. Japan” 42, 2924 (1969), U.S. Pat. No. 3,905,815A, JP1971-004605B (JP-S46-004605B), JP1973-036281A (JP-S48-036281A), JP1980-32070A (JP-S55-032070A), JP1985-239736A (JP-S60-239736A), JP1986-169835A (JP-S61-169835A), JP1986-169837A (JP-S61-169837A), JP1987-058241A (JP-S62-058241A), JP1987-212401A (JP-S62-212401A), JP1988-070243A (JP-S63-070243A), JP1988-298339A (JP-S63-298339A), and M. P. Hutt “Journal of Heterocyclic Chemistry” 1 (No. 3), (1970); and an oxazole compound or a triazine compound, substituted with a trihalomethyl group, is preferable.

Examples of the hexaarylbiimidazole compound include compounds described in JP1994-029285B (JP-H06-029285B), U.S. Pat. Nos. 3,479,185A, 4,311,783A, and 4,622,286A. Specific examples thereof include 2,2′-bis(o-chlorophenyl)-4,4′,5,5′-tetraphenylbiimidazole, 2,2′-bis(o-bromophenyl)-4,4′,5,5′-tetraphenylbiimidazole, 2,2′-bis(o,p-dichlorophenyl)4,4′,5,5′-tetraphenylbiimidazole, 2,2′-bis(o-chlorophenyl)-4,4′,5,5′-tetra(m-methoxyphenyl)biimidazole, 2,2′-bis(o,o′-dichlorophenyl)-4,4′,5,5′-tetraphenylbiimidazole, 2,2′-bis(o-nitrophenyl)-4,4′,5,5′-tetraphenylbiimidazole, 2,2′-bis(o-methylphenyl)-4,4′,5,5′-tetraphenylbiimidazole, and 2,2′-bis(o-trifluorophenyl)-4,4′,5,5′-tetraphenylbiimidazole.

### (Photocationic Polymerization Initiator)

The photocationic polymerization initiator is not particularly limited as long as it is a compound which generates a protonic acid or a Lewis acid by being irradiated with light. The photoacid generator is preferably a compound which is sensitive to actinic rays having a wavelength of 300 nm or more, preferably 300 to 450 nm, and generates an acid. The photoacid generator is preferably a compound which generates an acid having a pKa of 4 or less upon irradiation with light, more preferably a compound which generates an acid having a pKa of 3 or less upon irradiation with light, and still more preferably a compound which generates an acid having a pKa of 2 or less upon irradiation with light.

Examples of the photocationic polymerization initiator include an oxime sulfonate compound, a triazine compound, a sulfonium salt, an iodonium salt, a quaternary ammonium salt, a diazomethane compound, a sulfone compound, a sulfonic acid ester compound, an iminosulfonic acid ester compound, a carboxylic acid ester compound, and a sulfonimide compound.

Specific examples of the photocationic polymerization initiator include compounds described in paragraphs 0061 to 0108 of JP2012-046577A and paragraphs 0029 and 0030 of JP2002-122994A, compounds described in paragraphs 0037 to 0063 of JP2002-122994A, and oxime sulfonate compounds described in paragraphs 0081 to 0108 of JP2013-210616A. Examples of a commercially available product of the photocationic polymerization initiator include WPAG-469 (manufactured by FUJIFILM Wako Pure Chemical Corporation), CPI-100P (manufactured by San-Apro Ltd.), CPI-210S (manufactured by San-Apro Ltd.), and Irgacure 290 (manufactured by BASF SE).

### (Thermal Polymerization Initiator)

The thermal polymerization initiator is not particularly limited, and a known thermal polymerization initiator can be used. Examples thereof include azo-based compounds such as dimethyl 2,2′-azobis(isobutyrate), 2,2′-azobisisobutyronitrile, 2,2′-azobis(2,4-dimethyl-4-methoxyvaleronitrile), 2,2′-azobis(2,4-dimethylvaleronitrile), dimethyl-2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl-1,1′-azobis(1-cyclohexanecarboxylate), and 2,2′-azobis[2-(2-imidazolin-2-yl)propane]dihydrochloride;
organic peroxides such as 1,1-di(t-hexylperoxy)cyclohexane, 1,1-di(t-butylperoxy)cyclohexane, 2,2-di(4,4-di-(t-butylperoxy)cyclohexyl)propane, t-hexyl peroxy isopropyl monocarbonate, t-butyl peroxy-3,5,5-trimethyl hexanoate, t-butyl peroxy laurate, dicumyl peroxide, di-t-butyl peroxide, t-butyl peroxy-2-ethyl hexanoate, t-hexyl peroxy-2-ethyl hexanoate, cumene hydroperoxide, and t-butyl hydroperoxide; and
inorganic peroxides such as potassium persulfate, ammonium persulfate, and hydrogen peroxide.

In a case where the resin composition contains a polymerization initiator, a content of the polymerization initiator in the total solid content of the resin composition is preferably 0.1% to 20% by mass. The lower limit thereof is preferably 0.3% by mass or more and more preferably 0.4% by mass or more. The upper limit thereof is preferably 15% by mass or less and more preferably 10% by mass or less. The resin composition may contain only one kind of the polymerization initiator or two or more kinds thereof. In a case where two or more kinds of polymerization initiators are contained, it is preferable that the total amount thereof is within the above-described range.

### <<Catalyst>>

The resin composition according to the embodiment of the present invention can contain a catalyst. Examples of the catalyst include an acid catalyst such as hydrochloric acid, sulfuric acid, acetic acid, and propionic acid, and a base catalyst such as sodium hydroxide, potassium hydroxide, and triethylamine. In a case where the resin composition contains a catalyst, a content of the catalyst is preferably 0.1 to 100 parts by mass, more preferably 0.1 to 50 parts by mass, and still more preferably 0.1 to 20 parts by mass with respect to 100 parts by mass of the resin. The resin composition may contain only one kind of the catalyst or two or more kinds thereof. In a case of containing two or more kinds of catalysts, it is preferable that the total amount thereof is within the above-described range.

### <<Silane Coupling Agent>>

The resin composition according to the embodiment of the present invention can contain a silane coupling agent. According to this aspect, adhesiveness of a film to be obtained with a support can be further improved. In the present invention, the silane coupling agent means a silane compound having a hydrolyzable group and other functional groups. In addition, the hydrolyzable group refers to a substituent directly linked to a silicon atom and capable of forming a siloxane bond due to at least one of a hydrolysis reaction or a condensation reaction. Examples of the hydrolyzable group include a halogen atom, an alkoxy group, and an acyloxy group, and an alkoxy group is preferable. That is, it is preferable that the silane coupling agent is a compound having an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, an isocyanate group, and a phenyl group, and an amino group, a (meth)acryloyl group, or an epoxy group is preferable. Specific examples of the silane coupling agent include the compounds described in paragraphs 0018 to 0036 of JP2009-288703A and the compounds described in paragraphs 0056 to 0066 of JP2009-242604A, the contents of which are incorporated herein by reference. Examples of a commercially available product of the silane coupling agent include A-50 (organosilane) manufactured by Soken Chemical & Engineering Co., Ltd. A content of the silane coupling agent in the total solid content of the resin composition is preferably 0.1% to 5% by mass. The upper limit thereof is preferably 3% by mass or less and more preferably 2% by mass or less. The lower limit thereof is preferably 0.5% by mass or more and more preferably 1% by mass or more. The silane coupling agent may be used alone or in combination of two or more kinds thereof. In a case of using two or more kinds thereof, the total amount thereof is preferably within the above-described range.

### <<Surfactant>>

The resin composition according to the embodiment of the present invention can contain a surfactant. Examples of the surfactant include surfactants described in paragraph 0017 of JP4502784B and paragraphs 0060 to 0071 of JP2009-237362A.

As the surfactant, a nonionic surfactant, a fluorine-based surfactant, or a silicone-based surfactant is preferable.

Examples of a commercially available product of the fluorine-based surfactant include: MEGAFACE F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP, MFS-330, R-41, R-41-LM, R-01, R-40, R-40-LM, RS-43, TF-1956, RS-90, R-94, RS-72-K, and DS-21 (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC-1068, SC-381, SC-383, S-393, and KH-40 (all of which are manufactured by Asahi Glass Co., Ltd.); POLYFOX PF636, PF656, PF6320, PF6520, and PF7002 (all of which are manufactured by OMNOVA Solutions Inc.); and FTERGENT 710FM, 610FM, 601AD, 601ADH2, 602A, 215M, 245F, 251, 212M, 250, 209F, 222F, 208G, 710LA, 710FS, 730LM, 650AC, and 681 (all of which are manufactured by NEOS COMPANY LIMITED).

As the fluorine-based surfactant, an acrylic compound, which has a molecular structure having a functional group containing a fluorine atom and in which, by applying heat to the molecular structure, the functional group containing a fluorine atom is broken to volatilize a fluorine atom, can also be suitably used. Examples of such a fluorine-based surfactant include MEGAFACE DS series manufactured by DIC Corporation (The Chemical Daily (Feb. 22, 2016) and Nikkei Business Daily (Feb. 23, 2016)), for example, MEGAFACE DS-21.

It is also preferable that a polymer of a fluorine atom-containing vinyl ether compound having a fluorinated alkyl group or a fluorinated alkylene ether group, and a hydrophilic vinyl ether compound is used as the fluorine-based surfactant.

A block polymer can also be used as the fluorine-based surfactant.

As the fluorine-based surfactant, a fluorine-containing polymer compound including a repeating unit derived from a (meth)acrylate compound having a fluorine atom and a repeating unit derived from a (meth)acrylate compound having 2 or more (preferably 5 or more) alkyleneoxy groups (preferably ethyleneoxy groups or propyleneoxy groups) can also be used.

As the fluorine-based surfactant, a fluorine-containing polymer having an ethylenically unsaturated bond-containing group in the side chain can be used. Examples of a commercially available product thereof include MEGAFACE RS-101, RS-102, RS-718K, and RS-72-K (all manufactured by DIC Corporation).

Due to concerns of environmental suitability in a case of a compound having a linear perfluoroalkyl group having 7 or more carbon atoms, an alternative material for perfluorooctanoic acid (PFOA) or perfluorooctanesulfonic acid (PFOS) is preferably used as the fluorine-based surfactant.

Examples of the silicone-based surfactant include a linear polymer consisting of a siloxane bond and a modified siloxane polymer with an organic group introduced in the side chain or the terminal. Examples of a commercially available product of the silicone-based surfactant include DOWSIL 8032 ADDITIVE, TORAY SILICONE DC3PA, TORAY SILICONE SH7PA, TORAY SILICONE DC11PA, TORAY SILICONE SH21PA, TORAY SILICONE SH28PA, TORAY SILICONE SH29PA, TORAY SILICONE SH30PA, and TORAY SILICONE SH8400 (all of which are manufactured by Dow Corning Toray Co., Ltd.), X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 (all of which are manufactured by Shin-Etsu Chemical Co., Ltd.), F-4440, TSF-4300, TSF-4445, TSF-4460, and TSF-4452 (all of which are manufactured by Momentive Performance Materials Co., Ltd.), and BYK307, BYK323, and BYK330 (all of which are manufactured by BYK Chemie).

Examples of a nonionic surfactant include glycerol, trimethylolpropane, trimethylolethane, and ethoxylate and propoxylate thereof (for example, glycerol propoxylate, glycerol ethoxylate, and the like), polyoxyethylene lauryl ether, polyoxyethylene stearyl ether, polyoxyethylene oleyl ether, polyoxyethylene octyl phenyl ether, polyoxyethylene nonyl phenyl ether, polyethylene glycol dilaurate, polyethylene glycol distearate, and sorbitan fatty acid ester. Examples of a commercially available product of the nonionic surfactant include PLURONIC L10, L31, L61, L62, 10R5, 17R2, and 25R2 (all of which are manufactured by BASF SE), TETRONIC 304, 701, 704, 901, 904, and 150R1 (all of which are manufactured by BASF SE), SOLSPERSE 20000 (manufactured by Lubrizol Corporation), NCW-101, NCW-1001, and NCW-1002 (all of which are manufactured by FUJIFILM Wako Pure Chemical Corporation), PIONIN D-6112, D-6112-W, and D-6315 (all of which are manufactured by Takemoto Oil&Fat Co., Ltd.), and OLFINE E1010 and SURFYNOL 104, 400, and 440 (all of which are manufactured by Nissin Chemical Co., Ltd.).

A content of the surfactant in the total solid content of the resin composition is preferably 0.01% to 3.0% by mass, more preferably 0.05% to 1.0% by mass, and still more preferably 0.10% to 0.80% by mass. The surfactant may be used alone or in combination of two or more kinds thereof. In a case of using two or more kinds thereof, the total amount thereof is preferably within the above-described range.

### <<Solvent>>

It is preferable that the resin composition further contains a solvent. The solvent is not particularly limited, and examples thereof include water and an organic solvent. The solvent is preferably an organic solvent.

Examples of the organic solvent include an alcohol-based solvent, an ester-based solvent, an ether-based solvent, a ketone-based solvent, an amide-based solvent, a hydrocarbon-based solvent, and a halogen-based solvent.

Specific examples of the alcohol-based solvent include methanol, ethanol, 1-propanol, 2-propanol, 1-butanol, 2-butanol, 2-methyl-1-propanol, 1-methoxy-2-propanol, 2-ethoxyethanol, 2-butoxyethanol, ethylene glycol, propylene glycol, and glycerin.

Specific examples of the ester-based solvent include methyl acetate, ethyl acetate, n-butyl acetate, isobutyl acetate, amyl formate, isoamyl acetate, isobutyl acetate, butyl propionate, isopropyl butyrate, ethyl butyrate, butyl butyrate, methyl lactate, ethyl lactate, alkoxyacetic acid alkyl esters (such as methyl alkoxy acetate, ethyl alkoxy acetate, butyl alkoxy acetate (specific examples thereof include methyl methoxyacetate, ethyl methoxyacetate, butyl methoxyacetate, methyl ethoxyacetate, and ethyl ethoxyacetate)), 3-oxypropionic acid alkyl esters, 2-oxypropionic acid alkyl esters, methyl 2-oxy-2-methyl propionate, ethyl 2-oxy-2-methyl propionate, methyl pyruvate, ethyl pyruvate, propyl pyruvate, methyl acetoacetate, ethyl acetoacetate, methyl 2-oxobutanoate, ethyl 2-oxobutanoate, methyl cellosolve acetate, ethyl cellosolve acetate, propylene glycol monomethyl ether acetate, propylene glycol monoethyl ether acetate, propylene glycol monopropyl ether acetate, methyl 3-ethoxy propionate, ethyl 3-ethoxy propionate, and ethylene carbonate.

Specific examples of the ether-based solvent include diethylene glycol dimethyl ether, tetrahydrofuran, ethylene glycol monomethyl ether, ethylene glycol monoethyl ether, diethylene glycol monomethyl ether, diethylene glycol monoethyl ether, diethylene glycol monobutyl ether, propylene glycol monomethyl ether, polyethylene glycol monoalkyl ether, polypropylene glycol monoalkyl ether, polyethylene glycol, polypropylene glycol, ethylene glycol dialkyl ether, propylene glycol dialkyl ether, polyethylene glycol dialkyl ether, polypropylene glycol dialkyl ether, and dioxane.

Specific examples of the amide-based solvent include N-methylpyrrolidone, dimethylformamide, and dimethylacetamide.

Examples of the ketone-based solvent include methyl ethyl ketone, cyclohexanone, cyclopentanone, 2-heptanone, and 3-heptanone.

Specific examples of the hydrocarbon-based solvent include toluene and xylene.

Specific examples of the halogen-based solvent include chloroform and methylene chloride.

These organic solvents may be used in combination of two or more kinds thereof.

The organic solvent preferably includes at least one selected from methyl 3-ethoxy propionate, ethyl 3-ethoxy propionate, ethyl cellosolve acetate, ethyl lactate, diethylene glycol dimethyl ether, butyl acetate, methyl 3-methoxypropionate, 2-heptanone, cyclohexanone, cyclopentanone, ethyl carbitol acetate, butyl carbitol acetate, propylene glycol monomethyl ether, or propylene glycol monomethyl ether acetate.

A content of the solvent in the resin composition is preferably 10% to 90% by mass, more preferably 30% to 90% by mass, and still more preferably 50% to 90% by mass. The resin composition may contain only one kind of the solvent or two or more kinds thereof. In a case where two or more kinds of solvents are contained, it is preferable that the total amount thereof is within the above-described range.

In addition, in a case where the resin composition according to the embodiment of the present invention is used as a kneaded material, a content of the organic solvent in the resin composition is preferably 0.1% by mass or less and more preferably 0.01% by mass or less.

### <<Plasticizer>>

In a case where the resin composition according to the embodiment of the present invention is used as a kneaded material, the resin composition according to the embodiment of the present invention can contain a plasticizer. Examples of the plasticizer include a phthalic acid ester-based plasticizer, a phosphoric acid ester-based plasticizer, a trimellitic acid ester-based plasticizer, a fatty acid ester-based plasticizer, a polyester-based plasticizer, a glycerin-based plasticizer, and a polyalkylene glycol-based plasticizer; and a phthalic acid ester-based plasticizer or a phosphoric acid ester-based plasticizer is preferable.

Examples of the phthalic acid ester-based plasticizer include dimethyl phthalate, diethyl phthalate, diisopropyl phthalate, dibutyl phthalate, diisobutyl phthalate, dihexyl phthalate, dicyclohexyl phthalate, diphenyl phthalate, bis(2-ethylhexyl) phthalate, diisononyl phthalate, diisodecyl phthalate, and diundecyl phthalate.

Examples of the phosphoric acid ester-based plasticizer include trimethyl phosphate, triethyl phosphate, tributyl phosphate, triphenyl phosphate, and tricresyl phosphate.

Examples of the trimellitic acid ester-based plasticizer include tributyl trimellitate and tris(2-ethylhexyl) trimellitate.

Examples of the fatty acid ester-based plasticizer include dimethyl adipate, diethyl adipate, dipropyl adipate, diisopropyl adipate, dibutyl adipate, diisobutyl adipate, dimethyl dodecanoate, dibutyl maleate, and ethyl oleate.

Examples of the polyester-based plasticizer include polyester consisting of an acid component such as adipic acid, sebacic acid, terephthalic acid, isophthalic acid, naphthalenedicarboxylic acid, diphenyldicarboxylic acid, or rosin and a diol component such as propylene glycol, 1,3-butanediol, 1,4-butanediol, 1,6-hexanediol, ethylene glycol, or diethylene glycol, and polyester consisting of hydroxycarboxylic acid such as polycaprolactone. Terminals of these polyesters may be blocked with a monofunctional carboxylic acid or a monofunctional alcohol, or the terminals thereof may be blocked with an epoxy compound or the like.

Examples of the glycerin-based plasticizer include glycerin monoacetomonolaurate, glycerin diacetomonolaurate, glycerin monoacetomonostearate, glycerin diacetomonooleate, and glycerin monoacetomonomontanate.

Examples of the polyalkylene glycol-based plasticizer include polyalkylene glycol such as polyethylene glycol, polypropylene glycol, polytetramethylene glycol, an ethylene oxide addition polymer of bisphenols, a propylene oxide addition polymer of bisphenols, or a tetrahydrofuran addition polymer of bisphenols, and a terminal epoxy-modified compound, a terminal ester-modified compound, and a terminal ether-modified compound thereof.

A molecular weight of the plasticizer is preferably less than 3,000, more preferably 2,000 or less, and still more preferably 1,500 or less.

A content of the plasticizer in the resin composition is preferably 0.001% to 30% by mass. The lower limit thereof is preferably 0.005% by mass or more and more preferably 0.01% by mass or more. The upper limit thereof is preferably 20% by mass or less and more preferably 10% by mass or less.

The kneaded material may contain only one kind of the plasticizer or two or more kinds thereof. In a case where two or more kinds of plasticizers are contained, it is preferable that the total amount thereof is within the above-described range.

### <<Other Additives>>

The resin composition may appropriately contain optional additives such as an antioxidant, a light stabilizer, a processing stabilizer, an anti-aging agent, and a compatibilizer as necessary. By appropriately containing these components in the resin composition, various characteristics of the cured substance to be obtained can be appropriately adjusted.

### <Use of Resin Composition>

The resin composition according to the embodiment of the present invention can also be suitably used for applications, in a case where the resin composition may be exposed to sunlight or light including ultraviolet rays. Specific examples thereof include coating materials or films for window glass of houses, facilities, and transportation equipment; interior/exterior materials and interior/exterior paints of houses, facilities, and transportation equipment; members for light sources that emit ultraviolet rays, such as a fluorescent lamp and a mercury lamp; solar cells, precision machineries, electronic and electrical equipment, and members for a display device; containers or packaging materials for food, chemicals, and drugs; agricultural and industrial sheets; clothing textile products and fibers such as sportswear, stockings, and hats; lenses such as plastics lenses, contact lenses, glasses, and artificial eyes, or coating materials thereof, optical supplies such as optical filters, prisms, mirrors, and photographic materials; stationery such as tapes and inks; and marking boards, marking devices, and the surface coating materials thereof. For details thereof, reference can be made to paragraphs 0158 to 0218 of JP2009-263617A and paragraphs 0161 to 0194 of JP2009-096971A, the contents of which are incorporated herein by reference.

The resin composition according to the embodiment of the present invention can be preferably used for an optical member or the like. For example, the resin composition according to the embodiment of the present invention is preferably used for an ultraviolet cut filter, a lens, a protective material, or the like. The form of the protective material is not particularly limited, and examples thereof include a coating film, a film, and a sheet. In addition, the resin composition according to the embodiment of the present invention can also be used as a pressure sensitive adhesive or an adhesive.

In addition, the resin composition according to the embodiment of the present invention can also be used for various members of a display device. For example, in a case of a liquid crystal display device, the resin composition according to the embodiment of the present invention can be used for each member constituting the liquid crystal display device, such as an antireflection film, a polarizing plate protective film, an optical film, a retardation film, a pressure sensitive adhesive, and an adhesive. In addition, in a case of an organic electroluminescent display device, the resin composition according to the embodiment of the present invention can be used for each member constituting the organic electroluminescent display device, such as an optical film, a polarizing plate protective film in a circularly polarizing plate, a retardation film such as a ¼ wave plate, and an adhesive or a pressure sensitive adhesive.

### <Ultraviolet Absorber>

The ultraviolet absorber according to the embodiment of the present invention contains the above-described compound represented by Formula (1) (specific compound). The compound represented by Formula (1) is the same as the above description. The ultraviolet absorber can also be suitably used for applications in which there is a possibility of being exposed to sunlight or light including ultraviolet rays. Specific examples thereof include those described above. In addition, the ultraviolet absorber according to the embodiment of the present invention can also be used for packaging materials, containers, paints, coatings, inks, fibers, building materials, recording media, image display devices, covers for solar cells, glass coatings, and the like. In addition, the ultraviolet absorber according to the embodiment of the present invention can also be used for an optical member described later.

### <Cured Substance and Applications Thereof>

The cured substance according to the embodiment of the present invention is formed of the above-described resin composition according to the embodiment of the present invention. The “cured substance” in the present specification includes a dried substance obtained by drying and solidifying the resin composition, and a cured substance obtained by curing the resin composition in a case where the resin composition undergoes a curing reaction.

The cured substance according to the embodiment of the present invention may be obtained as a molded body formed by molding the resin composition into a desired form. The form of the molded body can be appropriately selected according to the intended use and purpose. Examples thereof include a coating film, a film, a sheet, a plate, a lens, a tube, and a fiber.

The cured substance according to the embodiment of the present invention is preferably used as an optical member. Examples of the optical member include an ultraviolet cut filter, a lens, and a protective material. In addition, the cured substance according to the embodiment of the present invention can also be used as a polarizing plate or the like.

The ultraviolet cut filter can be used for an article such as an optical filter, a display device, a solar cell, and window glass. The type of the display device is not particularly limited, and examples thereof include a liquid crystal display device and an organic electroluminescent display device.

In a case where the cured substance according to the embodiment of the present invention is used for a lens, the cured substance according to the embodiment of the present invention may be formed into a lens shape and used. In addition, the cured substance according to the embodiment of the present invention may be used for a coating film on a surface of a lens, an interlayer (adhesive layer) of a cemented lens, or the like. Examples of the cemented lens include those described in paragraphs 0094 to 0102 of WO2019/131572A, and the contents of which are incorporated herein by reference.

The type of protective material is not particularly limited, and examples thereof include a protective material for a display device, a protective material for a solar cell, a protective material for window glass, and an organic electroluminescent display device. The form of the protective material is not particularly limited, and examples thereof include a coating film, a film, and a sheet.

### <Optical Member>

The optical member according to the embodiment of the present invention includes a cured substance formed of the above-described resin composition according to the embodiment of the present invention. The cured substance according to the embodiment of the present invention may be obtained as a molded product formed by molding the above-described resin composition according to the embodiment of the present invention into a desired form. The form of the molded body can be appropriately selected according to the intended use and purpose. Examples thereof include a coating film, a film, a sheet, a plate, a lens, a tube, and a fiber.

Examples of the type of optical member include an ultraviolet cut filter, a lens, and a protective material.

The ultraviolet cut filter can be used for an article such as an optical filter, a display device, a solar cell, and window glass. The type of the display device is not particularly limited, and examples thereof include a liquid crystal display device and an organic electroluminescent display device.

Examples of the lens include those obtained by forming the cured substance according to the embodiment of the present invention into a lens shape, and those using the cured substance according to the embodiment of the present invention as a coating film on a surface of the lens or as an interlayer (an adhesive layer or a pressure-sensitive adhesive layer) of a cemented lens.

The type of protective material is not particularly limited, and examples thereof include a protective material for a display device, a protective material for a solar cell, and a protective material for window glass. The form of the protective material is not particularly limited, and examples thereof include a coating film, a film, and a sheet.

In addition, examples of one aspect of the optical member include a resin film. The resin film can be formed of the above-described resin composition according to the embodiment of the present invention. Examples of the resin used in a resin composition for forming the resin film include the above-described resins; and a (meth)acrylic resin, a polyester fiber, a cyclic olefin resin, or a cellulose acylate resin is preferable, and a cellulose acylate resin is more preferable. A resin composition containing the cellulose acylate resin can contain additives described in paragraphs 0022 to 0067 of JP2012-215689A. Examples of such additives include sugar esters. By adding a sugar ester compound to the resin composition containing the cellulose acylate resin, the total haze and inside haze can be decreased without impairing expression of optical properties even in a case where a heat treatment is not performed before a stretching step. In addition, the resin film formed of the resin composition containing the cellulose acylate resin (cellulose acylate film) can be produced by a method described in paragraphs 0068 to 0096 of JP2012-215689A. In addition, a hard coat layer described in paragraphs 0097 to 0113 of JP2012-215689A may be further laminated on the resin film.

In addition, examples of other forms of the optical member include an optical member including a laminate of a support and a resin layer. In the optical member, at least one of the support or the resin layer includes the above-described cured substance according to the embodiment of the present invention.

A thickness of the resin layer in the above-described laminate is preferably 1 μm to 2,500 μm and more preferably 10 μm to 500 μm.

As the support in the above-described laminate, a material having transparency within a range in which the optical performance is not impaired is preferable. The fact that the support has transparency means that the support is optically transparent, and specifically, means that the total light transmittance of the support is 85% or more. The total light transmittance of the support is preferably 90% or more, and more preferably 95% or more.

Suitable examples of the support include a resin film. Examples of a resin constituting the resin film include ester resins (for example, polyethylene terephthalate (PET), polyethylene naphthalate (PEN), polybutylene terephthalate (PBT), and polycyclohexanedimethylene terephthalate (PCT)), olefin resins (for example, polypropylene (PP) and polyethylene (PE)), polyvinyl chloride (PVA), and cellulose triacetate (TAC). Among these, PET is preferable in terms of general purpose properties.

A thickness of the support can be appropriately selected depending on the intended use or purpose. In general, the thickness thereof is preferably 5 μm to 2,500 μm and more preferably 20 μm to 500 μm.

In addition, a peelable support can also be used as the above-described support. Such a laminate is preferably used as a polarizing plate or the like. Here, the peelable support is a support from which the support can be peeled off from the resin film. A stress in a case of peeling the support from the resin film is preferably 0.05 N/25 mm or more and 2.00 N/25 mm or less, more preferably 0.08 N/25 mm or more and 0.50 N/25 mm or less, and still more preferably 0.11 N/25 mm or more and 0.20 N/25 mm or less. The stress in a case of peeling the support from the resin film is evaluated by bonding and fixing the surface of the laminate cut to have a size of a width of 25 mm and a length of 80 mm to a glass base material through an acrylic pressure sensitive adhesive sheet, grasping one end (one side with a width of 25 mm) of a test piece in the length direction using a tension tester (RTF-1210, manufactured by A & D Co., Ltd.), and performing a 90° peeling test (in conformity with Japanese Industrial Standards (JIS) K 6854-1: 1999 “Adhesive-Determination of peel strength of bonded assemblies-Part 1: 900 peeling”) in an atmosphere of a temperature of 23° C. and a relative humidity of 60% at a crosshead speed (grasping movement speed) of 200 mm/min.

A support containing polyethylene terephthalate (PET) as a main component (a component having the highest content in terms of mass among components constituting the support) is preferable as the peelable support. From the viewpoint of mechanical strength, a weight-average molecular weight of PET is preferably 20,000 or more, more preferably 30,000 or more, and still more preferably 40,000 or more. The weight-average molecular weight of PET can be determined by dissolving the support in hexafluoroisopropanol (HFIP) with the above-described GPC method. A thickness of the support is not particularly limited, but is preferably 0.1 to 100 μm, more preferably 0.1 to 75 μm, still more preferably 0.1 to 55 μm, and particularly preferably 0.1 to 10 μm. In addition, the support may be subjected to a corona treatment, a glow discharge treatment, undercoating, or the like as a known surface treatment.

In addition, examples of other forms of the optical member include a laminate obtained by laminating a hard coat layer, a transparent support, and a pressure-sensitive adhesive layer or an adhesive layer in this order. Such a laminate is preferably used as an ultraviolet cut filter or a protective material (a protective film or a protective sheet). The optical member in this form is not limited as long as any of the support, the hard coat layer, or the pressure-sensitive adhesive layer or the adhesive layer contains the above-described cured substance according to the embodiment of the present invention.

As the hard coat layer, for example, hard coat layers described in JP2013-045045A, JP2013-043352A, JP2012-232459A, JP2012-128157A, JP2011-131409A, JP2011-131404A, JP2011-126162A, JP2011-075705A, JP2009-286981A, JP2009-263567A, JP2009-075248A, JP2007-164206A, JP2006-096811A, JP2004-075970A, JP2002-156505A, JP2001-272503A, WO2012/018087A, WO2012/098967A, WO2012/086659A, and WO2011/105594A can be applied. From the viewpoint of further improving the scratch resistance, a thickness of the hardcoat layer is preferably 5 to 100 μm.

The optical member in this form has a pressure-sensitive adhesive layer or an adhesive layer on a side of the support opposite to a side where the hard coat layer is provided. The type of the pressure sensitive adhesive or the adhesive used for the pressure-sensitive adhesive layer or the adhesive layer is not particularly limited, and a known pressure sensitive adhesive or adhesive can be used. In addition, as the pressure sensitive adhesive or the adhesive, those containing an acrylic resin described in paragraphs 0056 to 0076 of JP2017-142412A and a crosslinking agent described in paragraphs 0077 to 0082 of JP2017-142412A are also preferably used. In addition, the pressure sensitive adhesive or the adhesive may contain an adhesiveness improver (silane compound) described in paragraphs 0088 to 0097 of JP2017-142412A and additives described in paragraph 0098 of JP2017-142412A. The pressure-sensitive adhesive layer or the adhesive layer can be formed by a method described in paragraphs 0099 and 0100 of JP2017-142412A. From the viewpoint of achieving both adhesive strength and handleability, a thickness of the pressure-sensitive adhesive layer or the adhesive layer is preferably 5 μm to 100 μm.

The optical member according to the embodiment of the present invention can be preferably used as a constituent member of a display such as a liquid crystal display device (LCD) or an organic electroluminescent display device (OLED).

Examples of the liquid crystal display device include a liquid crystal display device in which a member such as an antireflection film, a polarizing plate protective film, an optical film, a retardation film, a pressure sensitive adhesive, and an adhesive contains the cured substance according to the embodiment of the present invention. The optical member including the cured substance according to the embodiment of the present invention may be disposed on a visual observer side (front side) or a backlight side with respect to the liquid crystal cell, and may be disposed on an outer side or an inner side with respect to the polarizer.

Examples of the organic electroluminescent display device include an organic electroluminescent display device in which a member such as an optical film, a polarizing plate protective film in a circularly polarizing plate, a retardation film such as a ¼ wave plate, an adhesive, and a pressure sensitive adhesive contains the cured substance according to the embodiment of the present invention. In a case of using the cured substance according to the embodiment of the present invention with the above-described configuration, deterioration of the organic electroluminescent display device due to external light can be suppressed.

### <Polymer>

The polymer according to the embodiment of the present invention is a polymer including a structure (hereinafter, also referred to as a structure (3)) derived from a compound having a structure in which at least one of R¹¹, R¹², Q³, or Q⁴ is the group including a polymerizable group having an ethylenically unsaturated bond, among the compounds represented by Formula (3) described above. Hereinafter, among the compounds represented by Formula (3) described above, the compound having a structure in which at least one of R¹¹, R¹², Q³, or Q⁴ is the group including a polymerizable group having an ethylenically unsaturated bond is also referred to as a specific compound (3).

The polymer according to the embodiment of the present invention may include a structure derived from a compound (hereinafter, also referred to as other polymerizable compounds) having an ethylenically unsaturated bond-containing group, the compound being other than the specific compound (3), in addition to the above-described structure derived from the specific compound (3). That is, the polymer according to the embodiment of the present invention may form a copolymer of the specific compound (3) and other polymerizable compounds. Examples of the other polymerizable compounds include polymerizable compounds described as the material used in the resin composition according to the embodiment of the present invention, and compounds having a polymerizable group, which are described as the material used as the other ultraviolet absorbers.

A content of the structure derived from the specific compound (3) in the polymer according to the embodiment of the present invention is preferably 0.01% to 100% by mass. The upper limit thereof is more preferably 50% by mass or less and still more preferably 10% by mass or less. The lower limit thereof is more preferably 0.02% by mass or more and still more preferably 0.1% by mass or more.

A weight-average molecular weight of the polymer according to the embodiment of the present invention is preferably 5,000 to 80,000, more preferably 10,000 to 60,000, and still more preferably 10,000 to 40,000.

The polymer according to the embodiment of the present invention can be used for an ultraviolet absorber, an optical member, and the like.

The polymer according to the embodiment of the present invention can also be used by being mixed with a resin. Examples of the resin include the resins described in the section of the resin composition according to the embodiment of the present invention above.

### EXAMPLES

Hereinafter, the present invention will be described in detail using Examples. Materials, used amounts, proportions, treatment details, treatment procedures, and the like shown in the following examples can be appropriately changed within a range not departing from the scope of the present invention. Therefore, the scope of the present invention is not limited to the specific examples shown below. In addition, in the structural formulae shown below, Me is a methyl group, Et is an ethyl group, ⁿBu is an n-butyl group, Bu is a tert-butyl group, Ph is a phenyl group, and Ac is an acetyl group.

### SYNTHESIS EXAMPLE

### (Synthesis Example 1) Synthesis of Intermediate 1-3

An intermediate 1-1 was synthesized according to the following scheme. In the following scheme, the synthesis of the intermediate 1-1 was carried out with reference to a method described in paragraphs 0222 and 0223 of JP2009-263617A, using 1,2-dibenzylpyrazolidine-3,5-dione instead of 1,2-dibutylpyrazolidine-3,5-dione, thereby obtaining 77 g (yield: 78%) of the intermediate 1-1.

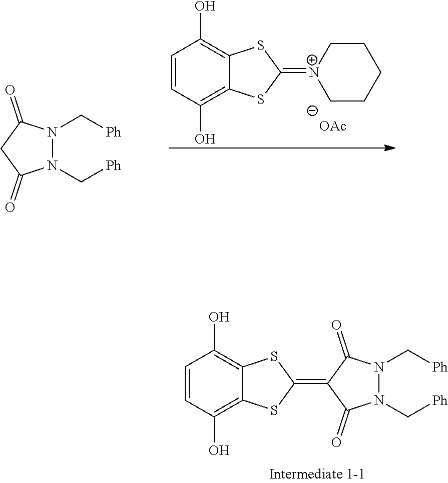 [SMILES: O=C1CC(=O)N(Cc2ccccc2)N1Cc1ccccc1 ; Oc1ccc(O)c2sc(=[N+]3CCCCC3)sc12 ; O=C1C(=C2Sc3c(O)ccc(O)c3S2)C(=O)N(Cc2ccccc2)N1Cc1ccccc1 ; "OAc"]

Next, an intermediate 1-2 was synthesized according to the following synthesis scheme. 50 g of the intermediate 1-1, 24.5 g of 2,3-dichloro-5,6-dicyano-p-benzoquinone, and 500 ml of tetrahydrofuran were added and mixed with each other, and the mixture was stirred at 20° C. for 1 hour. After completion of the reaction, 500 mL of hexane was added thereto, and the precipitated solid was collected by filtration and then washed with 150 mL of hexane to obtain 42 g (yield: 84%) of the intermediate 1-2.

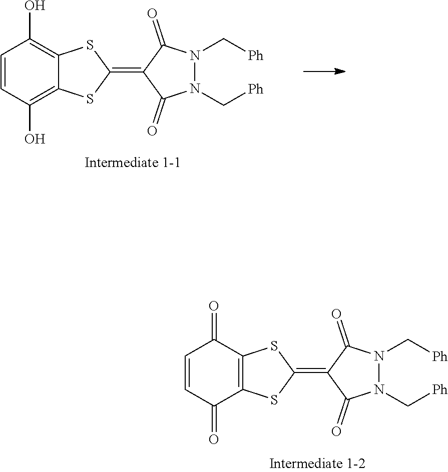 [SMILES: O=C1C(=C2Sc3c(O)ccc(O)c3S2)C(=O)N(Cc2ccccc2)N1Cc1ccccc1 ; O=C1C=CC(=O)C2=C1SC(=C1C(=O)N(Cc3ccccc3)N(Cc3ccccc3)C1=O)S2]

Next, an intermediate 1-3 was synthesized according to the following synthesis scheme. 30 g of the intermediate 1-2, 8 g of piperidinium pentamethylenedithiocarbamate, 360 mL of N-methyl-2-pyrrolidone, 160 mL of acetic acid, and 54 mL of acetone were added and mixed with each other, and the mixture was stirred at 60° C. for 1 hour. The precipitated solid was collected by filtration and then washed with 300 ml of acetone to obtain 8.0 g (yield: 36%) of the intermediate 1-3.

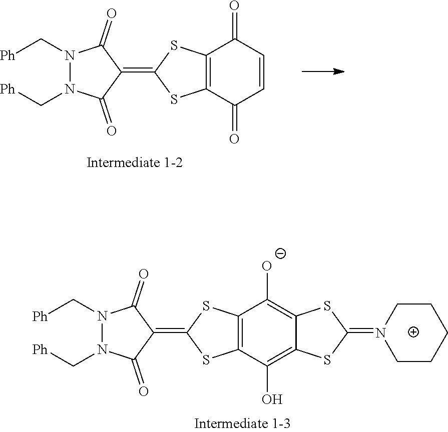

### (Synthesis Example 2) Synthesis of Compound A-104

A compound A-104 was synthesized according to the following synthesis scheme. 3.0 g of the intermediate 1-3, 0.58 g of malononitrile, and 150 ml of N-methyl-2-pyrrolidone were added and mixed with each other, and the mixture was stirred at 80° C. for 1 hour. After cooling to room temperature, 1 mL of hydrochloric acid and 150 ml of water were added thereto, and the mixture was stirred for 30 minutes. The precipitated solid was collected by filtration, 200 ml of acetonitrile was added thereto, and the mixture was heated and refluxed under a nitrogen atmosphere for 1 hour. After cooling to room temperature and stirring at room temperature for 1 hour, the solid was collected by filtration and then washed with 100 ml of acetonitrile to obtain 2.1 g (yield: 79%) of the compound A-104. In addition, in a proton nuclear magnetic resonance (¹H-NMR, solvent: deuterated dimethyl sulfoxide (dDMSO)) of the obtained compound A-104, chemical shifts δ were 11.6 (s, 2H), 7.29 (m, 10H), and 4.80 (s, 4H).

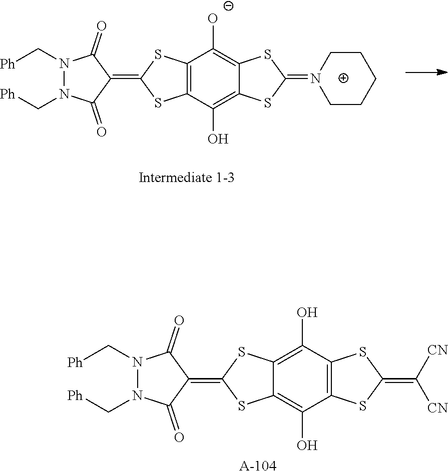

### (Synthesis Example 3) Synthesis of Compound A-1

A compound A-1 was synthesized according to the following synthesis scheme. 1.5 g of the compound A-104, 0.76 g of triethylamine, 1.0 g of 2-ethylhexanoyl chloride, and 30 ml of dimethylacetamide were added and mixed with each other, and the mixture was stirred at 20° C. for 1 hour. After completion of the reaction, 30 ml of water was added thereto, followed by stirring for 30 minutes. The precipitated solid was collected by filtration, washed with 30 ml of methanol, and then purified by silica gel column chromatography to obtain 1.6 g (yield: 75%) of the compound A-1. In addition, in a proton nuclear magnetic resonance (¹H-NMR, solvent: deuterated chloroform (CDCl₃)) of the obtained compound A-1, chemical shifts δ were 7.27 (m, 6H), 7.10 (m, 4H), 4.75 (s, 4H), 2.69 (m, 2H), 1.8 to 1.6 (m, 8H), 1.5 to 1.3 (m, 8H), 1.10 (m, 6H), and 0.94 (m, 6H).

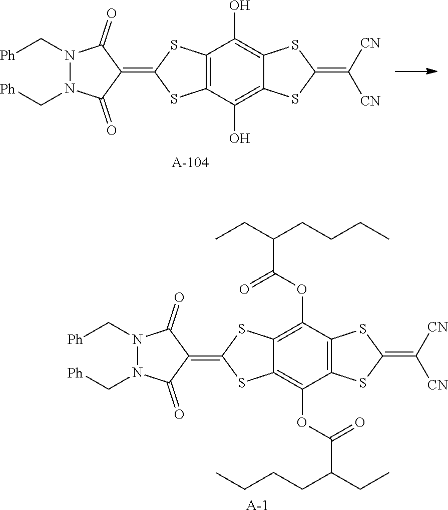

### (Synthesis Example 4) Synthesis of Compound A-105

0.71 g (yield: 60%) of a compound A-105 was obtained by the same method as in Synthesis Example 2, except that, in Synthesis Example 2, n-butyl cyanoacetate was used instead of malononitrile.

¹H-NMR (dDMSO): δ 11.5 (s, 2H), 7.28 (m, 10H), 4.80 (s, 4H), 4.24 (t, 2H), 1.62 (m, 2H), 1.37 (m, 2H), 0.92 (t, 3H)

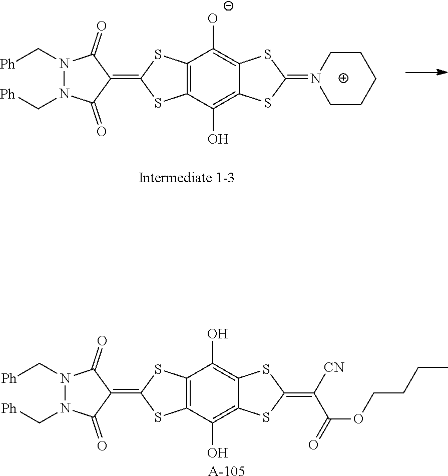

### (Synthesis Example 5) Synthesis of Compound A-2

0.61 g (yield: 74%) of a compound A-2 was obtained by the same method as in Synthesis Example 3, except that, in Synthesis Example 3, the compound A-105 was used instead of the compound A-104.

¹H-NMR (CDCl₃): δ 7.27 (m, 6H), 7.10 (m, 4H), 4.75 (s, 4H), 4.28 (t, 2H), 2.69 (m, 2H), 1.91 to 1.71 (m, 10H), 1.60 to 1.44 (m, 10H), 1.15 (m, 6H), 0.96 (m, 9H)

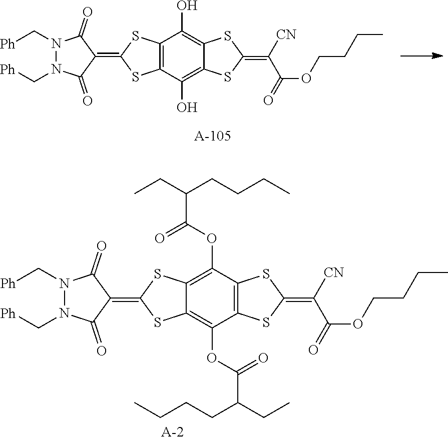

### (Synthesis Example 6) Synthesis of Compound A-106

1.0 g (yield: 78%) of a compound A-106 was obtained by the same method as in Synthesis Example 2, except that, in Synthesis Example 2, 2-ethylhexyl cyanoacetate was used instead of malononitrile.

¹H-NMR (dDMSO): δ 11.5 (s, 2H), 7.24 (m, 10H), 4.80 (s, 4H), 4.16 (d, 2H), 1.63 (m, 1H), 1.29 (m, 8H), 0.88 (m, 6H)

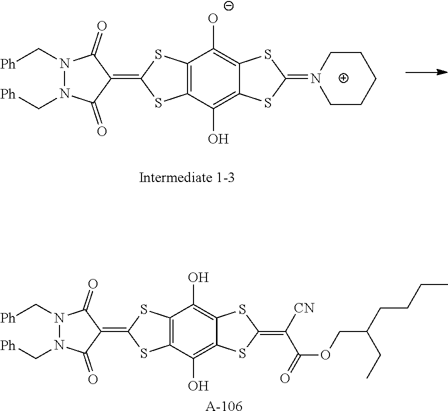

### (Synthesis Example 7) Synthesis of Compound A-3

0.42 g (yield: 68%) of a compound A-3 was obtained by the same method as in Synthesis Example 3, except that, in Synthesis Example 3, the compound A-106 was used instead of the compound A-104.

¹H-NMR (CDCl₃): δ 7.27 (m, 6H), 7.10 (m, 4H), 4.75 (s, 4H), 4.19 (m, 2H), 2.69 (m, 2H), 1.90 to 1.66 (m, 9H), 1.47 to 1.31 (m, 16H), 1.14 (m, 6H), 1.01 to 0.90 (m, 12H)

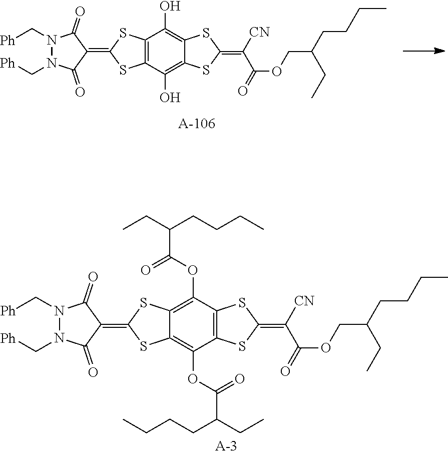

### (Synthesis Example 8) Synthesis of Compound A-18

0.33 g (yield: 79%) of a compound A-18 was obtained by the same method as in Synthesis Example 7, except that, in Synthesis Example 7, 3,5,5-trimethylhexanoyl chloride was used instead of 2-ethylhexanoyl chloride.

¹H-NMR (CDCl₃): δ 7.27 (m, 6H), 7.10 (m, 4H), 4.76 (s, 4H), 4.19 (m, 2H), 2.77 (m, 2H), 2.56 (m, 2H), 2.24 (m, 2H), 1.67 (m, 1H), 1.55 to 1.32 (m, 12H), 1.18 (m, 6H), 0.98 to 0.90 (m, 24H)

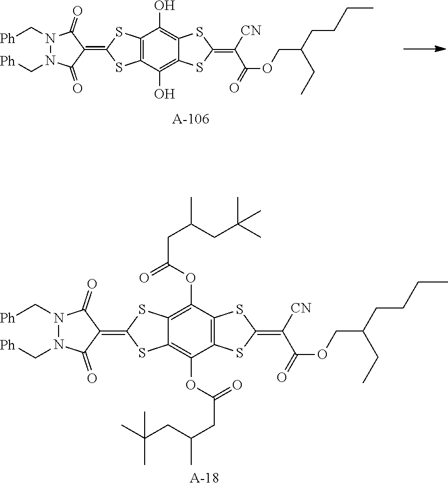

### (Synthesis Example 9) Synthesis of Compound A-107

1.1 g (yield: 76%) of a compound A-107 was obtained by the same method as in Synthesis Example 2, except that, in Synthesis Example 2, pivaloylacetonitrile was used instead of malononitrile.

¹H-NMR (dDMSO): δ 11.5 (s, 2H), 7.29 (m, 10H), 4.80 (s, 4H), 1.34 (s, 9H)

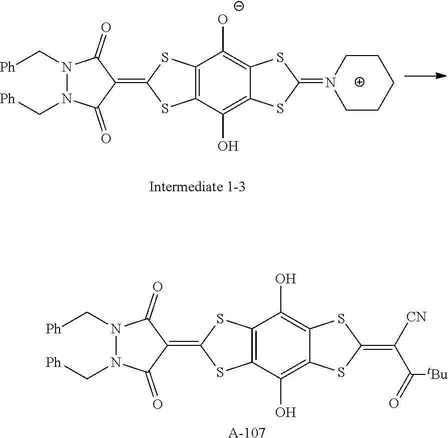

### (Synthesis Example 10) Synthesis of Compound A-4

0.43 g (yield: 62%) of a compound A-4 was obtained by the same method as in Synthesis Example 3, except that, in Synthesis Example 3, the compound A-107 was used instead of the compound A-104.

¹H-NMR (CDCl₃): δ 7.27 (m, 6H), 7.10 (m, 4H), 4.75 (s, 4H), 2.71 (m, 2H), 1.8 to 1.6 (m, 8H), 1.48 to 1.45 (m, 8H), 1.39 (s, 9H), 1.15 (m, 6H), 0.99 (m, 6H)

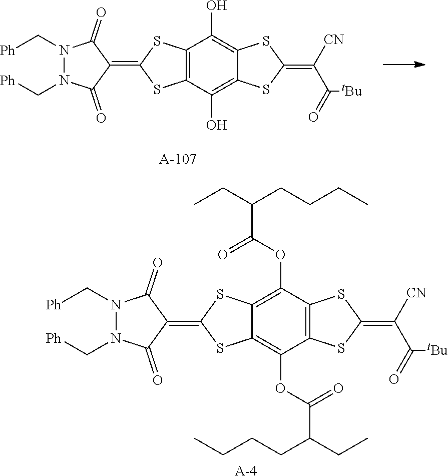

### (Synthesis Example 11) Synthesis of Compound A-108

0.68 g (yield: 75%) of a compound A-108 was obtained by the same method as in Synthesis Example 2, except that, in Synthesis Example 2, 2-cyanoacetamide was used instead of malononitrile.

¹H-NMR (dDMSO): δ 11.3 (s, 2H), 7.65 (m, 2H), 7.29 (m, 10H), 4.80 (s, 4H)

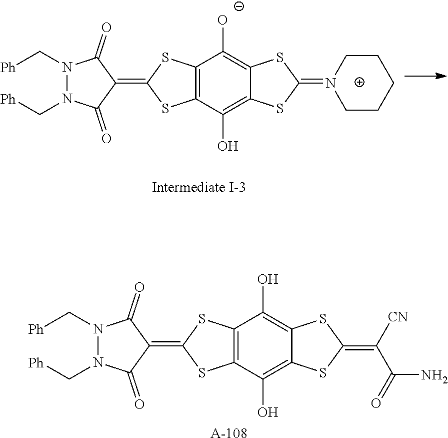

### (Synthesis Example 12) Synthesis of Compound A-5

0.41 g (yield: 58%) of a compound A-5 was obtained by the same method as in Synthesis Example 3, except that, in Synthesis Example 3, the compound A-108 was used instead of the compound A-104.

¹H-NMR (CDCl₃): δ 7.27 (m, 6H), 7.10 (m, 4H), 6.00 (br, 1H), 5.46 (br, 1H), 4.75 (s, 4H), 2.69 (m, 2H), 1.92 to 1.71 (m, 8H), 1.55 to 1.43 (m, 8H), 1.16 (m, 6H), 0.98 (m, 6H)

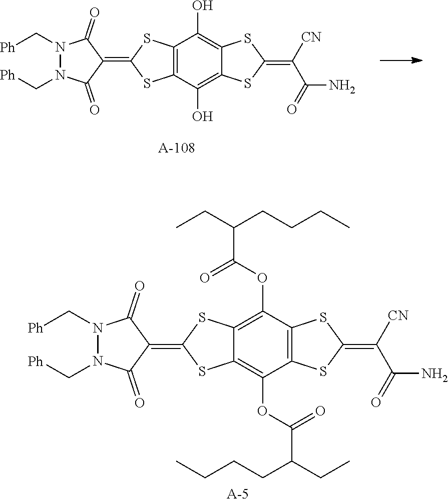

### (Synthesis Example 13) Synthesis of Compound A-109

An intermediate 2-3 was obtained by the same method as in Synthesis Example 1, except that, in Synthesis Example 1, dimethyl malonate was used instead of 1,2-dibenzylpyrazolidine-3,5-dione.

Next, a compound A-109 was synthesized according to the following synthesis scheme. 2.0 g of the intermediate 2-3, 1.1 g of 1,2-dibenzylpyrazolidine-3,5-dione, and 100 ml of N-methyl-2-pyrrolidone were added and mixed with each other, and the mixture was stirred at 100° C. for 1 hour. After cooling to room temperature, 1 mL of hydrochloric acid and 150 ml of water were added thereto, and the mixture was stirred for 30 minutes. The precipitated solid was collected by filtration, 100 ml of acetonitrile was added thereto, and the mixture was heated and refluxed under a nitrogen atmosphere for 1 hour. After cooling to room temperature and stirring at room temperature for 1 hour, the solid was collected by filtration and then washed with 50 ml of acetonitrile to obtain 1.3 g (yield: 52%) of the compound A-109.

¹H-NMR (dDMSO): δ 11.2 (s, 2H), 7.29 (m, 10H), 4.80 (s, 4H), 3.79 (s, 6H)

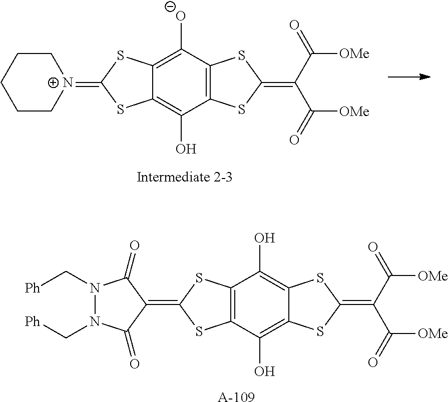

### (Synthesis Example 14) Synthesis of Compound A-6

0.45 g (yield: 65%) of a compound A-6 was obtained by the same method as in Synthesis Example 3, except that, in Synthesis Example 3, the compound A-109 was used instead of the compound A-104.

¹H-NMR (CDCl₃): δ 7.27 (m, 6H), 7.10 (m, 4H), 4.75 (s, 4H), 3.88 (s, 6H), 2.71 (m, 2H), 1.95 to 1.73 (m, 8H), 1.59 to 1.41 (m, 8H), 1.16 (m, 6H), 0.99 (m, 6H)

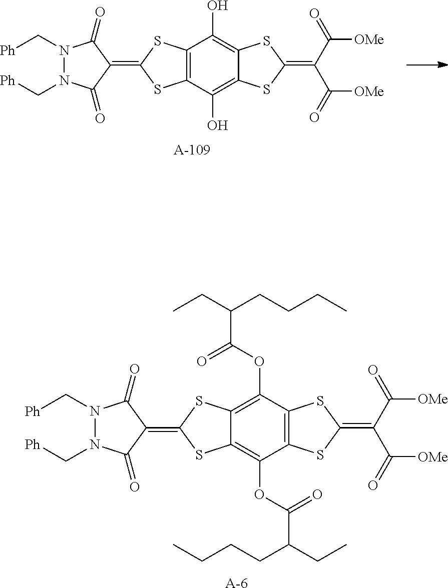

### (Synthesis Example 15) Synthesis of Compound A-110

1.2 g (yield: 84%) of a compound A-110 was obtained by the same method as in Synthesis Example 13, except that, in Synthesis Example 13, methanesulfonyl acetonitrile was used instead of dimethyl malonate, and an intermediate 3-3 was used instead of the intermediate 2-3.

¹H-NMR (dDMSO): δ 11.5 (s, 2H), 7.29 (m, 10H), 4.80 (s, 4H), 3.36 (s, 3H)

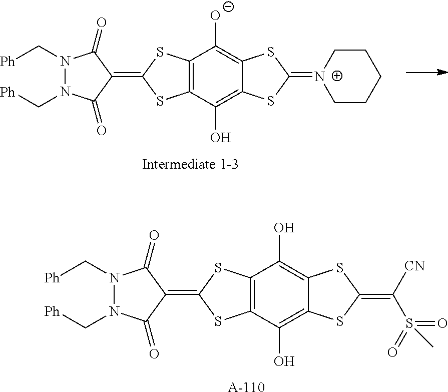

### (Synthesis Example 16) Synthesis of Compound A-7

0.63 g (yield: 57%) of a compound A-7 was obtained by the same method as in Synthesis Example 3, except that, in Synthesis Example 3, the compound A-110 was used instead of the compound A-104.

¹H-NMR (CDCl₃): δ 7.27 (m, 6H), 7.10 (m, 4H), 4.75 (s, 4H), 3.19 (s, 3H), 2.68 (m, 2H), 1.91 to 1.74 (m, 8H), 1.50 to 1.40 (m, 8H), 1.13 (m, 6H), 0.97 (m, 6H)

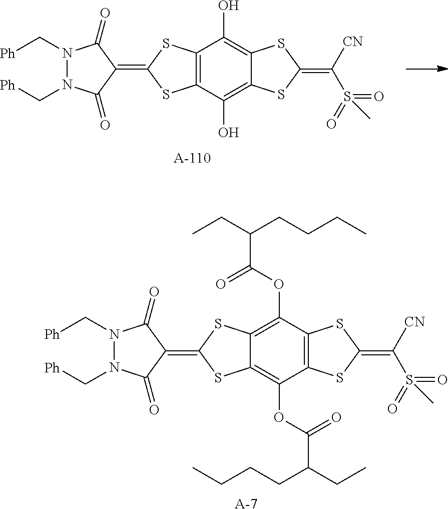

### (Synthesis Example 17) Synthesis of Compound A-46

0.48 g (yield: 60%) of a compound A-46 was obtained by the same method as in Synthesis Example 3, except that, in Synthesis Example 3, lauroyl chloride was used instead of 2-ethylhexanoyl chloride.

¹H-NMR (CDCl₃): δ 7.27 (m, 6H), 7.10 (m, 4H), 4.77 (s, 4H), 2.73 (t, 2H), 1.82 (dd, 4H), 1.53 to 1.22 (m, 32H), 0.88 (t, 6H)

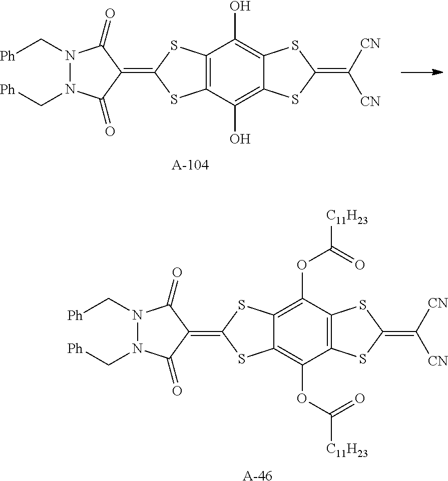

### (Synthesis Example 18) Synthesis of Compound A-47

0.35 g (yield: 61%) of a compound A-47 was obtained by the same method as in Synthesis Example 3, except that, in Synthesis Example 3, 2-acetoxyisobutyryl chloride was used instead of 2-ethylhexanoyl chloride.

¹H-NMR (CDCl₃): δ 7.27 (m, 6H), 7.10 (m, 4H), 4.75 (s, 4H), 2.23 (s, 6H), 1.78 (s, 12H)

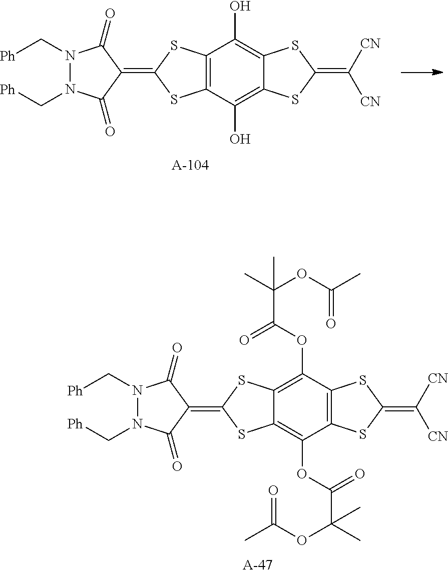

### (Synthesis Example 19) Synthesis of Compound A-44

A compound A-44 was synthesized according to the following synthesis scheme. 0.50 g of the compound A-104, 0.42 g of triethylamine, 0.60 g of 2-hexyldecanoic acid, 0.28 g of thionyl chloride, and 20 ml of dimethylacetamide were added and mixed with each other, and the mixture was stirred at 20° C. for 1 hour. After completion of the reaction, 30 ml of water was added thereto, and the mixture was stirred for 30 minutes. The precipitated solid was collected by filtration, washed with 30 ml of methanol, and then purified by column to obtain 0.60 g (yield: 67%) of the compound A-44.

¹H-NMR (CDCl₃): δ 7.27 (m, 6H), 7.10 (m, 4H), 4.75 (s, 4H), 2.74 (m, 2H), 1.81 to 1.60 (m, 8H), 1.52 to 1.26 (m, 40H), 0.86 (m, 12H)

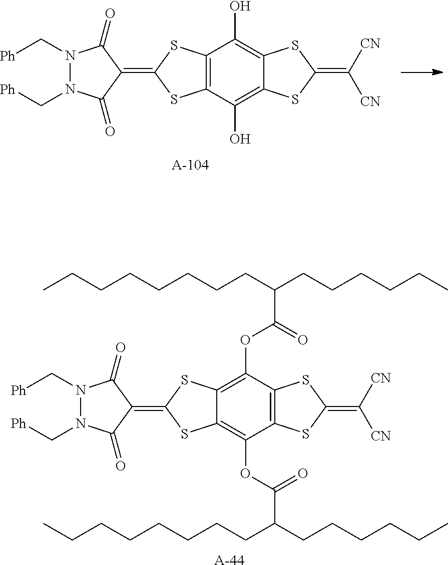

### (Synthesis Example 20) Synthesis of Compound A-45

0.50 g (yield: 44%) of a compound A-45 was obtained by the same method as in Synthesis Example 19, except that, in Synthesis Example 19, 2,2,4,8,10,10-hexamethylundecane-5-carboxylic acid was used instead of 2-hexyldecanoic acid.

¹H-NMR (CDCl₃): δ 7.27 (m, 6H), 7.10 (m, 4H), 4.74 (s, 4H), 2.64 (m, 2H), 2.15 (m, 1H), 1.96 (m, 1H), 1.83 to 1.68 (m, 4H), 1.52 to 1.15 (m, 22H), 1.13 to 1.05 (m, 22H), 0.98 to 0.89 (m, 18H)

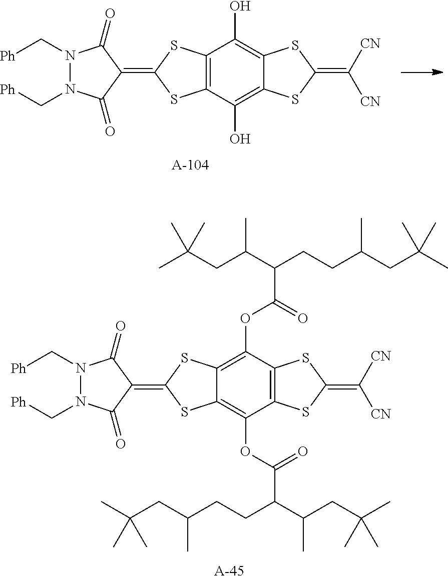

### (Synthesis Example 21) Synthesis of Compound A-26

0.38 g (yield: 58%) of a compound A-26 was obtained by the same method as in Synthesis Example 19, except that, in Synthesis Example 19, the compound A-106 was used instead of the compound A-104.

¹H-NMR (CDCl₃): δ 7.27 (m, 6H), 7.10 (m, 4H), 4.74 (s, 4H), 4.19 (m, 2H), 2.74 (m, 2H), 1.85 (m, 4H), 1.69 (m, 5H), 1.52 to 1.26 (m, 48H), 0.92 to 0.84 (m, 18H)

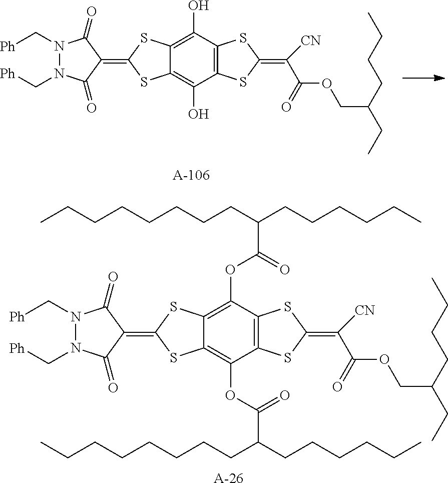

### (Synthesis Example 22) Synthesis of Compound A-111

0.97 g (yield: 80%) of a compound A-Ill was obtained by the same method as in Synthesis Example 2, except that, in Synthesis Example 2, 2-ethoxyethyl cyanoacetate was used instead of malononitrile.

¹H-NMR (dDMSO): δ 11.5 (s, 2H), 7.28 (m, 10H), 4.80 (s, 4H), 4.35 (t, 2H), 3.64 (t, 2H), 3.50 (q, 2H), 1.12 (t, 3H)

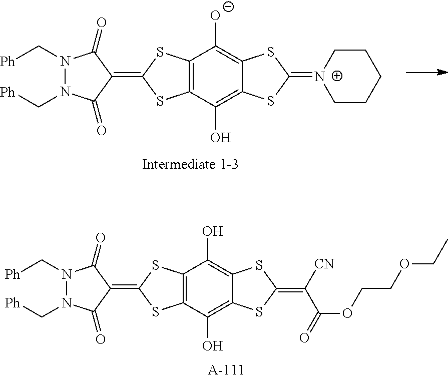

### (Synthesis Example 23) Synthesis of Compound A-19

0.32 g (yield: 78%) of a compound A-19 was obtained by the same method as in Synthesis Example 3, except that, in Synthesis Example 3, the compound A-111 was used instead of the compound A-104.

¹H-NMR (CDCl₃): δ 7.27 (m, 6H), 7.10 (m, 4H), 4.75 (s, 4H), 4.41 (t, 2H), 3.72 (t, 2H), 3.56 (q, 2H), 2.70 (m, 2H), 1.92 to 1.71 (m, 8H), 1.60 to 1.44 (m, 8H), 1.22 to 1.13 (m, 9H), 0.98 (m, 6H)

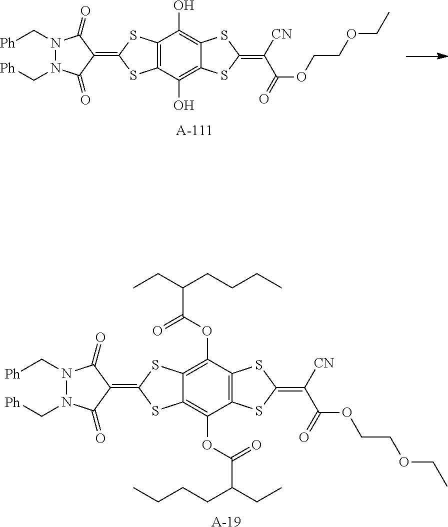

### (Synthesis Example 24) Synthesis of Compound A-25

0.28 g (yield: 74%) of a compound A-25 was obtained by the same method as in Synthesis Example 7, except that, in Synthesis Example 7, 2,2-dimethylbutyryl chloride was used instead of 2-ethylhexanoyl chloride.

¹H-NMR (CDCl₃): δ 7.27 (m, 6H), 7.10 (m, 4H), 4.76 (s, 4H), 4.19 (m, 2H), 1.89 to 1.69 (m, 3H), 1.44 to 1.25 (m, 20H), 1.10 (m, 6H), 0.94 (m, 6H)

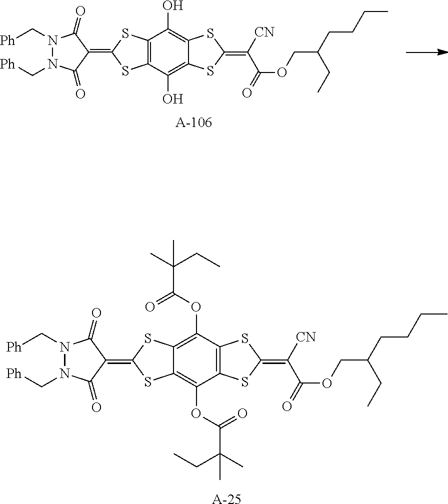

### (Synthesis Example 25) Synthesis of Compound A-112

0.31 g (yield: 39%) of a compound A-112 was obtained by the same method as in Synthesis Example 2, except that, in Synthesis Example 2, tert-butyl cyanoacetate was used instead of malononitrile.

¹H-NMR (dDMSO): δ 11.5 (s, 2H), 7.28 (m, 10H), 4.80 (s, 4H), 1.51 (t, 9H)

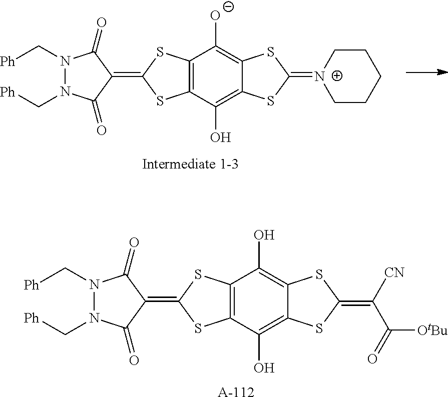

### (Synthesis Example 26) Synthesis of Compound A-20

0.28 g (yield: 65%) of a compound A-20 was obtained by the same method as in Synthesis Example 3, except that, in Synthesis Example 3, the compound A-112 was used instead of the compound A-104.

¹H-NMR (CDCl₃): δ 7.27 (m, 6H), 7.10 (m, 4H), 4.75 (s, 4H), 2.69 (m, 2H), 1.88 to 1.76 (m, 8H), 1.54 to 1.44 (m, 17H), 1.13 (m, 6H), 0.99 (m, 6H)

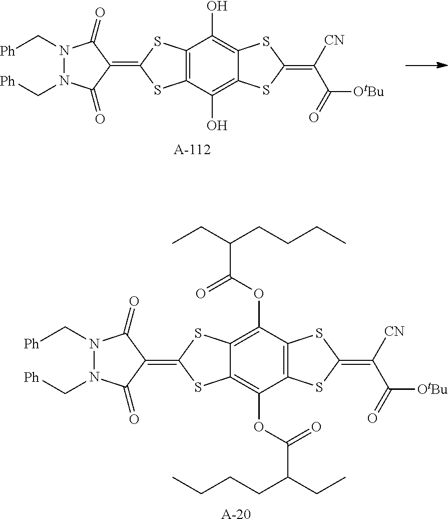

### (Synthesis Example 27) Synthesis of Compound A-128

0.24 g (yield: 48%) of a compound A-128 was obtained by the same method as in Synthesis Example 7, except that, in Synthesis Example 7, methacryloyl chloride was used instead of 2-ethylhexanoyl chloride.

¹H-NMR (CDCl₃): δ 7.27 (m, 6H), 7.10 (m, 4H), 6.52 (d, 2H), 6.00 (d, 2H), 4.76 (s, 4H), 4.19 (m, 2H), 2.13 (d, 6H), 1.69 (m, 1H), 1.44 to 1.25 (m, 6H), 0.94 (m, 6H)

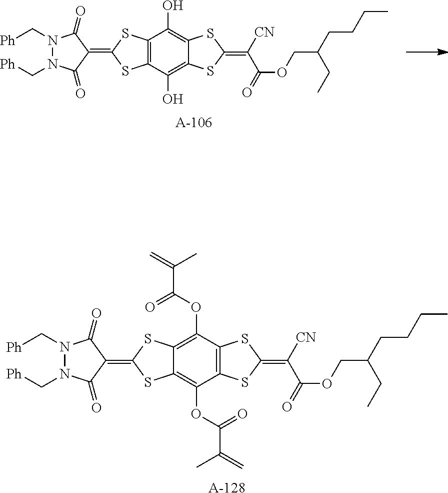

### (Synthesis Example 28) Synthesis of Compound A-196

0.35 g (yield: 81%) of a compound A-196 was obtained by the same method as in Synthesis Example 2, except that, in Synthesis Example 2, allyl cyanoacetate was used instead of malononitrile.

¹H-NMR (dDMSO): δ 11.5 (s, 2H), 7.28 (m, 10H), 5.96 (m, 1H), 5.32 (m, 2H), 4.80 (m, 6H)

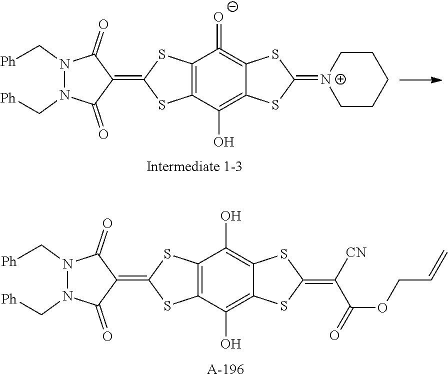

### (Synthesis Example 29) Synthesis of Compound A-141

0.30 g (yield: 74%) of a compound A-141 was obtained by the same method as in Synthesis Example 3, except that, in Synthesis Example 3, the compound A-196 was used instead of the compound A-104.

¹H-NMR (CDCl₃): δ 7.27 (m, 6H), 7.10 (m, 4H), 5.96 (m, 1H), 5.32 (m, 2H), 4.75 (m, 6H), 4.28 (t, 2H), 2.69 (m, 2H), 1.8 to 1.6 (m, 8H), 1.5 to 1.3 (m, 8H), 1.10 (m, 6H), and 0.94 (m, 6H)

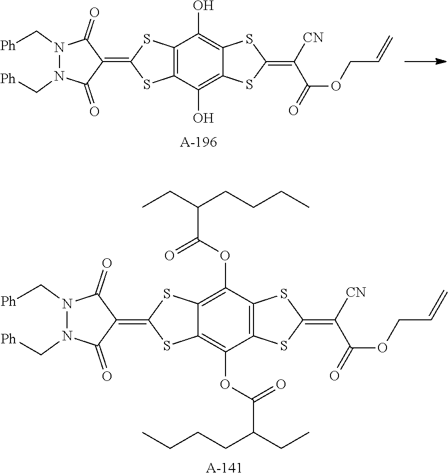

### (Synthesis Example 30) Synthesis of Compound A-197

0.35 g (yield: 93%) of a compound A-196 was obtained by the same method as in Synthesis Example 2, except that, in Synthesis Example 2, ethyl cyanoacetate methacrylate was used instead of malononitrile.

¹H-NMR (dDMSO): δ 11.5 (s, 2H), 7.28 (m, 10H), 6.05 (s, 1H), 5.70 (s, 1H), 4.80 (s, 4H), 4.50 (m, 2H), 4.38 (m, 2H), 1.88 (s, 3H)

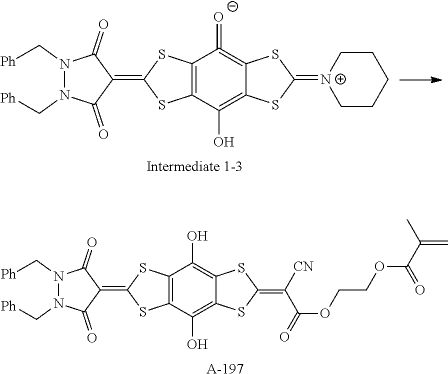

### (Synthesis Example 31) Synthesis of Compound A-143

0.20 g (yield: 45%) of a compound A-143 was obtained by the same method as in Synthesis Example 3, except that, in Synthesis Example 3, the compound A-197 was used instead of the compound A-104.

¹H-NMR (CDCl₃): δ 7.27 (m, 6H), 7.10 (m, 4H), 6.17 (s, 1H), 5.60 (s, 1H), 4.75 (s, 4H), 4.50 (m, 2H), 4.43 (m, 2H), 2.69 (m, 2H), 1.8 to 1.6 (m, 11H), 1.5 to 1.3 (m, 8H), 1.10 (m, 6H), 0.94 (m, 6H)

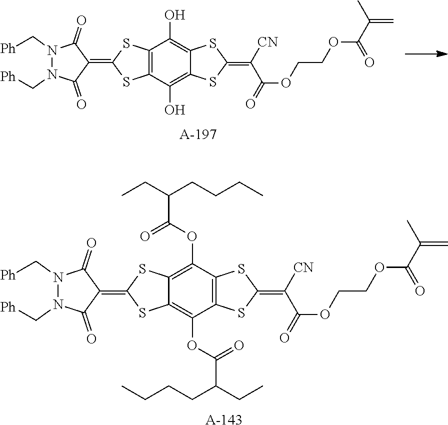

### (Synthesis Example 32) Synthesis of Compound A-142

2.1 g (yield: 60%) of a compound A-143 was obtained by the same method as in Synthesis Example 31, except that, in Synthesis Example 31, 3,5,5-trimethylhexanoyl chloride was used instead of 2-ethylhexanoyl chloride.

¹H-NMR (CDCl₃): δ 7.27 (m, 6H), 7.10 (m, 4H), 6.17 (s, 1H), 5.60 (s, 1H), 4.75 (s, 4H), 4.50 (m, 2H), 4.43 (m, 2H), 2.77 (m, 2H), 2.56 (m, 2H), 2.24 (m, 2H), 1.95 (s, 3H), 1.5 to 1.3 (m, 6H), 1.25 (m, 6H), 0.98 (s, 18H)

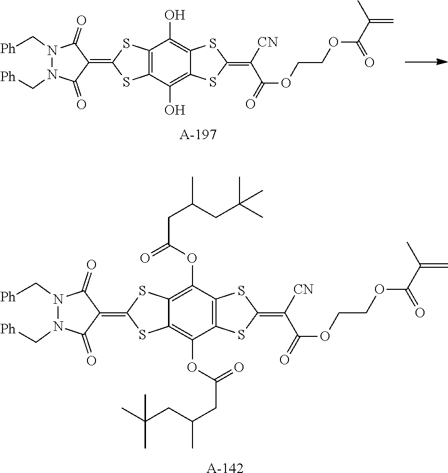

### (Synthesis Example 33) Synthesis of Compound A-245

3.0 g (yield: 45%) of a compound A-245 was obtained by the same method as in Synthesis Example 2, except that, in Synthesis Example 2, 2,6-bis(1,1-dimethylethyl)-4-methylcyclohexyl cyanoacetate was used instead of malononitrile.

¹H-NMR (dDMSO): δ 11.5 (s, 2H), 7.24 (m, 10H), 5.73 (s, 1H), 4.80 (s, 4H), 1.6 to 1.2 (m, 7H), 0.99 (m, 3H), 0.85 (s, 18H)

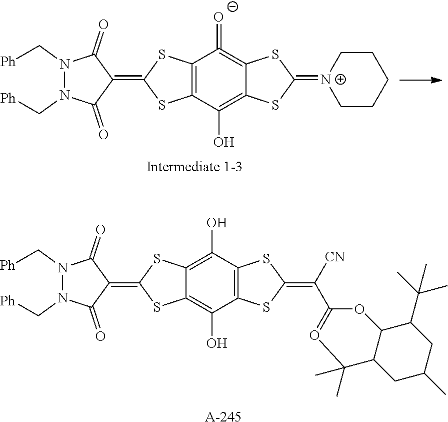

### (Synthesis Example 34) Synthesis of Compound A-201

2.1 g (yield: 53%) of a compound A-201 was obtained by the same method as in Synthesis Example 3, except that, in Synthesis Example 3, the compound A-245 was used instead of the compound A-104.

¹H-NMR (CDCl₃): δ 7.27 (m, 6H), 7.10 (m, 4H), 5.80 (s, 1H), 4.75 (s, 4H), 2.69 (m, 2H), 1.9 to 1.0 (m, 29H), 0.98 (m, 9H), 0.87 (s, 18H)

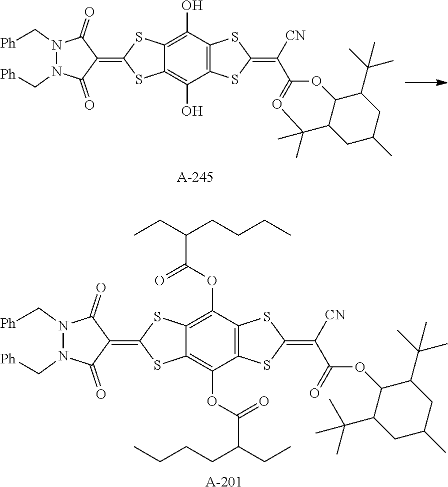

### (Synthesis Example 35) Synthesis of Compound A-251

An intermediate 3-3 was obtained by the same method as in Synthesis Example 1, except that, in Synthesis Example 1, 1,2-dibutylpyrazolidine-3,5-dione was used instead of 1,2-dibenzylpyrazolidine-3,5-dione.

Next, 1.20 g (yield: 91%) of a compound A-251 was obtained by the same method as in Synthesis Example 2, except that, in Synthesis Example 2, ethyl cyanoacetate methacrylate was used instead of malononitrile, and the intermediate 3-3 was used instead of the intermediate 1-3.

¹H-NMR (dDMSO): δ 11.4 (s, 2H), 6.05 (s, 1H), 5.70 (s, 1H), 4.80 (s, 4H), 4.50 (m, 2H), 4.38 (m, 2H), 3.62 (m, 4H), 1.88 (s, 3H), 1.44 (m, 4H), 1.23 (m, 4H), 0.87 (m, 6H)

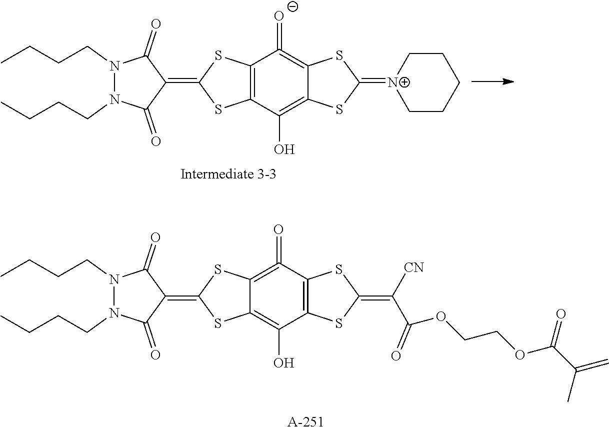

### (Synthesis Example 36) Synthesis of Compound A-232

0.70 g (yield: 68%) of a compound A-232 was obtained by the same method as in Synthesis Example 3, except that, in Synthesis Example 3, the compound A-251 was used instead of the compound A-104.

¹H-NMR (CDCl₃): 6.14 (s, 1H), 5.60 (s, 1H), 4.52 (m, 2H), 4.43 (m, 2H), 3.65 (m, 4H), 2.69 (m, 2H), 1.8 to 1.6 (m, 11H), 1.5 to 1.4 (m, 18H), 1.26 (m, 6H), 1.10 (m, 6H), 0.98 (m, 6H), 0.91 (m, 6H)

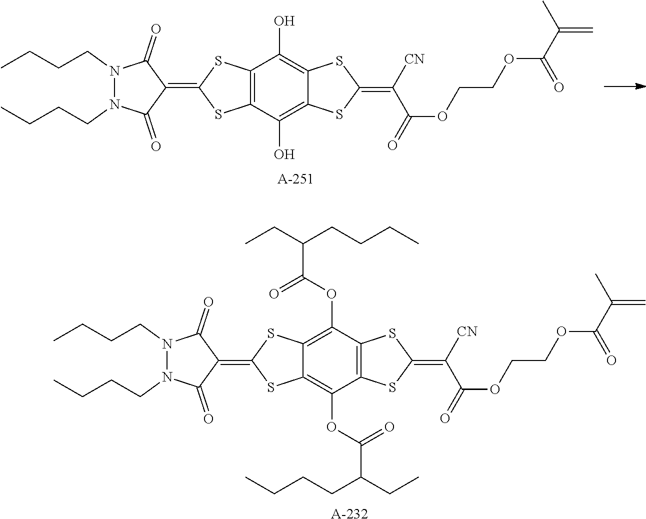

### Test Example 1

2 mg of the compound (exemplary compounds (1) to (44) and comparative compounds (1) to (3)) shown in the following tables was dissolved in 100 mL of ethyl acetate, and the solution was diluted with ethyl acetate such that an absorbance of the solution was in a range of 0.6 to 1.2, thereby preparing sample solutions 101 to 147.

An absorbance and a molar absorption coefficient of each sample solution were measured using a spectrophotometer (UV-1800PC, manufactured by Shimadzu Corporation) with a 1 cm quartz cell. A maximal absorption wavelength (λₘₐₓ) was measured from absorption spectrum of each sample solution, and absorption ability of UV-A was evaluated according to the following standard.

In addition, for each sample solution, a value of a ratio of the absorbance at a wavelength of 440 nm in a case where the absorbance at a wavelength of 400 nm was set to 1 (absorbance ratio A₄₄₀) was calculated, and colorability was evaluated according to the following standard. As the value of the absorbance ratio A₄₄₀ is smaller, the coloration less occurs.

The evaluation results are listed in the following tables. The numerical value in the column of absorption ability of UV-A is the value of λₘₐₓ, and the numerical value in the column of colorability is the value of the absorbance ratio A₄₄₀. In addition, the value of the molar absorption coefficient at the maximal absorption wavelength is shown in the column of molar absorption coefficient in the following tables.

### —Evaluation Standard for Absorption Ability of UV-A—

A: λₘₐₓ was 390 nm or more.
B: λₘₐₓ was 370 nm or more and less than 390 nm.
C: λₘₐₓ was less than 370 nm.

### —Evaluation Standard of Colorability—

A: absorbance ratio λ₄₄₀ was less than 0.02.
B: absorbance ratio λ₄₄₀ was 0.02 or more.

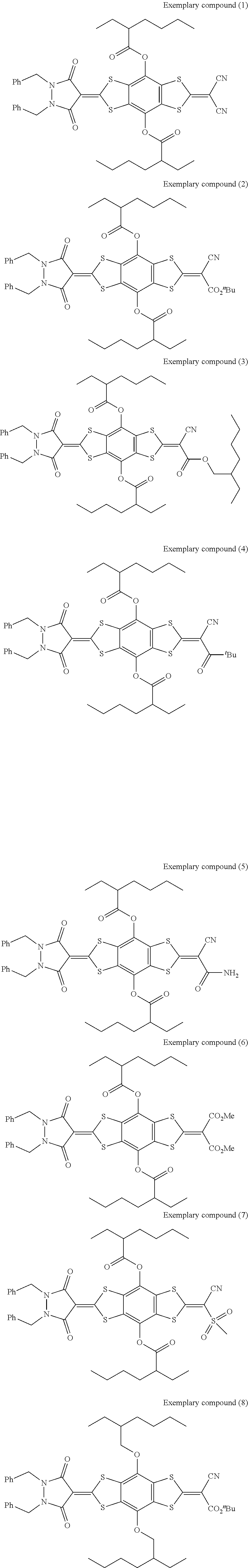

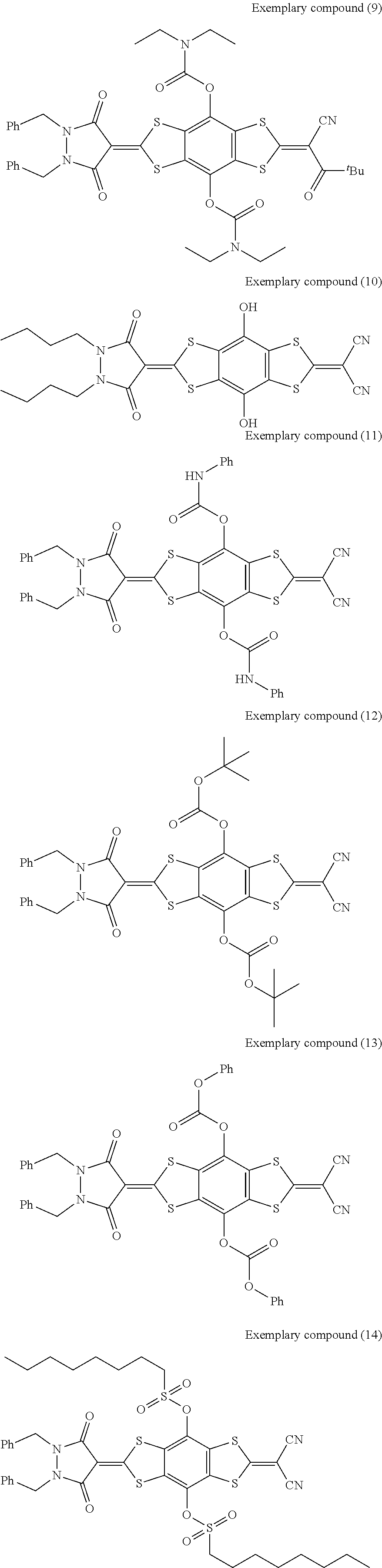

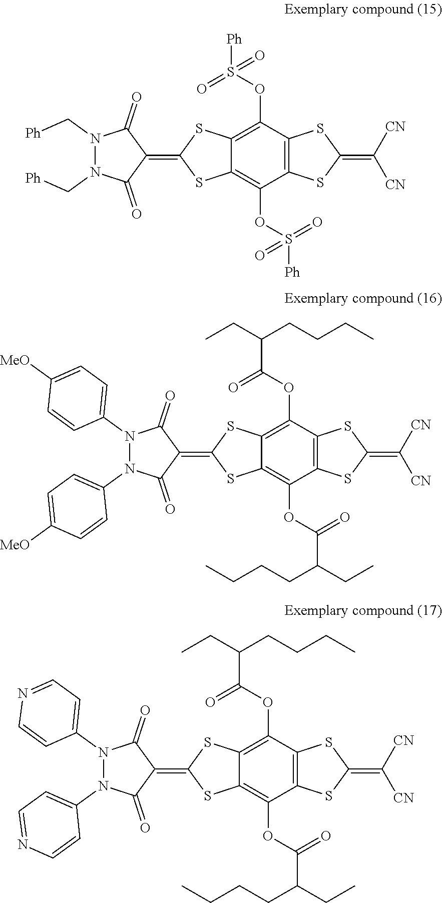 [SMILES: N#CC(C#N)=C1Sc2c(OS(=O)(=O)c3ccccc3)c3c(c(OS(=O)(=O)c4ccccc4)c2S1)SC(=C1C(=O)N(Cc2ccccc2)N(Cc2ccccc2)C1=O)S3 ; CCCCC(CC)C(=O)Oc1c2c(c(OC(=O)C(CC)CCCC)c3c1SC(=C1C(=O)N(c4ccc(OC)cc4)N(c4ccc(OC)cc4)C1=O)S3)SC(=C(C#N)C#N)S2 ; CCCCC(CC)C(=O)Oc1c2c(c(OC(=O)C(CC)CCCC)c3c1SC(=C1C(=O)N(c4ccncc4)N(c4ccncc4)C1=O)S3)SC(=C(C#N)C#N)S2]

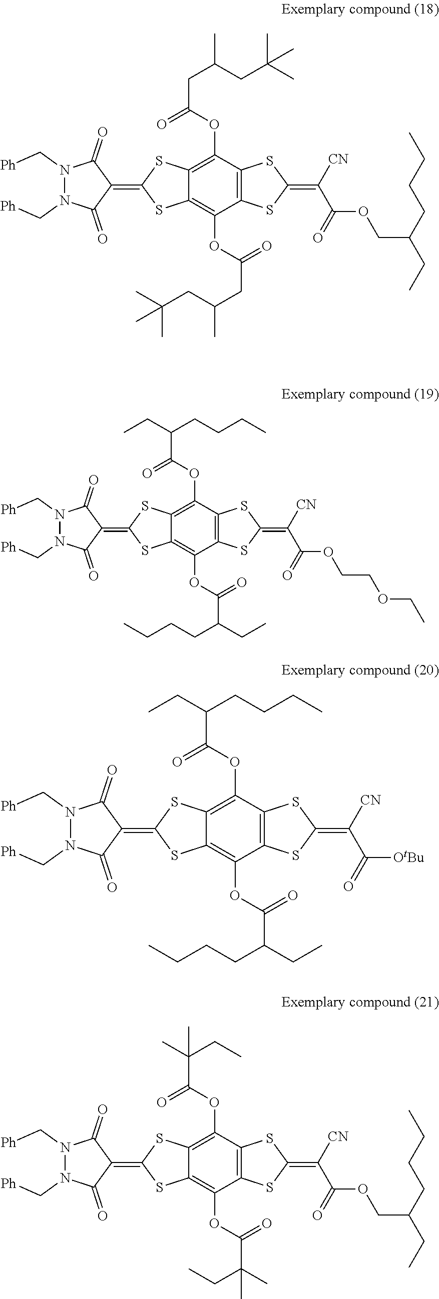 [SMILES: CCCCC(CC)COC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C#N)C(=O)OCCOCC)S2 ; CCCCC(CC)C(=O)Oc1c2c(c(OC(=O)C(CC)CCCC)c3c1SC(=C1C(=O)N(Cc4ccccc4)N(Cc4ccccc4)C1=O)S3)SC(=C(C#N)C(=O)OC(C)(C)C)S2 ; CCCCC(CC)COC(=O)C(C#N)=C1Sc2c(OC(=O)C(C)(C)CC)c3c(c(OC(=O)C(C)(C)CC)c2S1)SC(=C1C(=O)N(Cc2ccccc2)N(Cc2ccccc2)C1=O)S3]

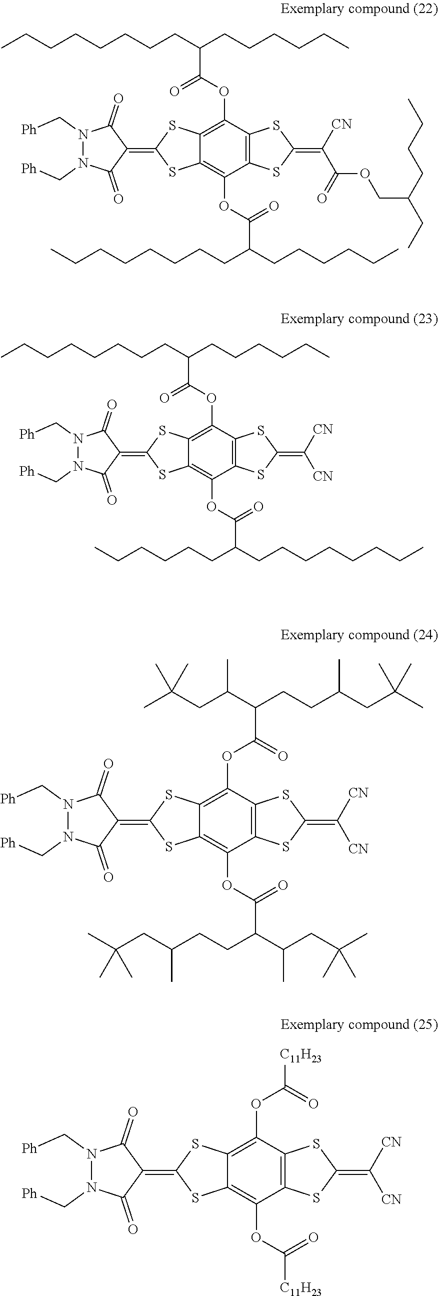

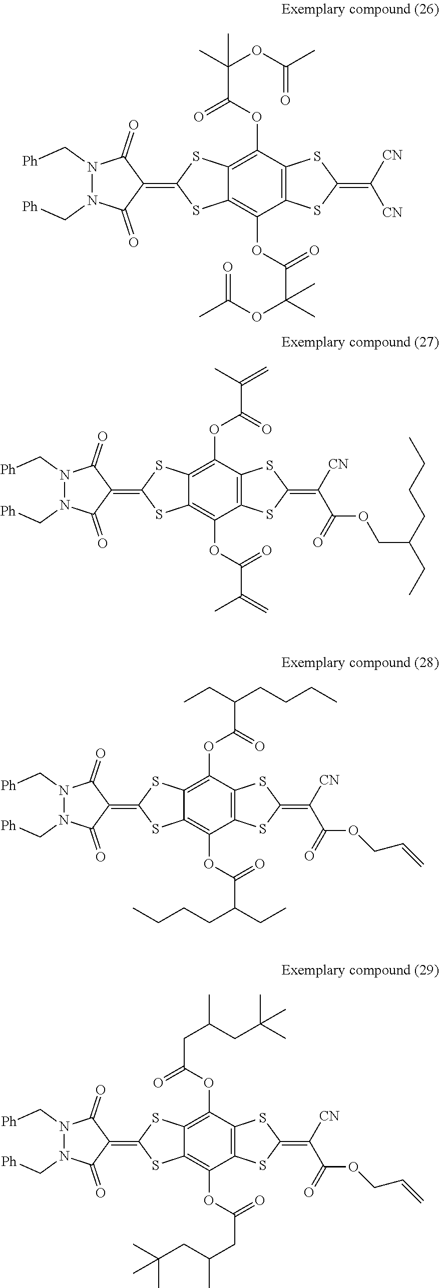 [SMILES: CC(=O)OC(C)(C)C(=O)Oc1c2c(c(OC(=O)C(C)(C)OC(C)=O)c3c1SC(=C1C(=O)N(Cc4ccccc4)N(Cc4ccccc4)C1=O)S3)SC(=C(C#N)C#N)S2 ; C=C(C)C(=O)Oc1c2c(c(OC(=O)C(=C)C)c3c1SC(=C1C(=O)N(Cc4ccccc4)N(Cc4ccccc4)C1=O)S3)SC(=C(C#N)C(=O)OCC(CC)CCCC)S2 ; C=CCOC(=O)C(C#N)=C1Sc2c(OC(=O)C(CC)CCCC)c3c(c(OC(=O)C(CC)CCCC)c2S1)SC(=C1C(=O)N(Cc2ccccc2)N(Cc2ccccc2)C1=O)S3 ; C=CCOC(=O)C(C#N)=C1Sc2c(OC(=O)CC(C)CC(C)(C)C)c3c(c(OC(=O)CC(C)CC(C)(C)C)c2S1)SC(=C1C(=O)N(Cc2ccccc2)N(Cc2ccccc2)C1=O)S3]

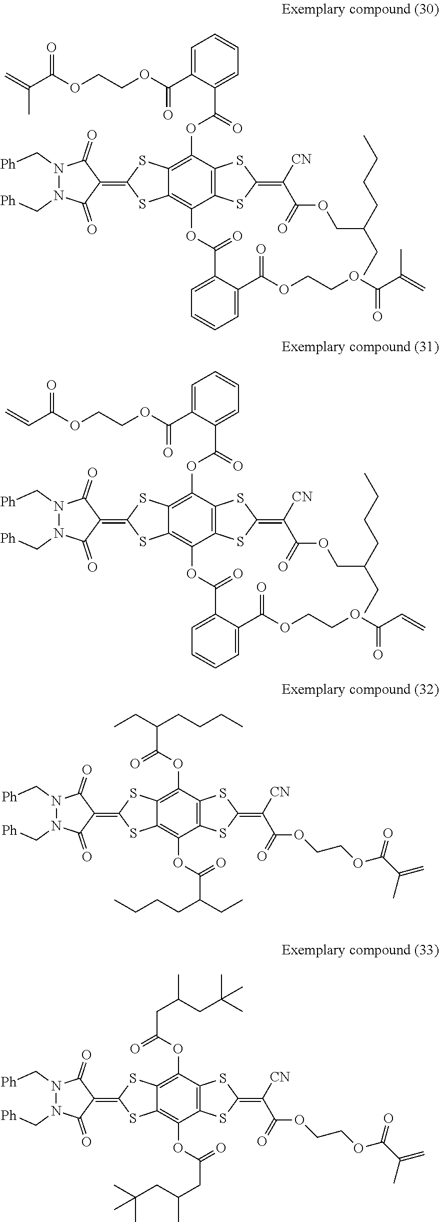

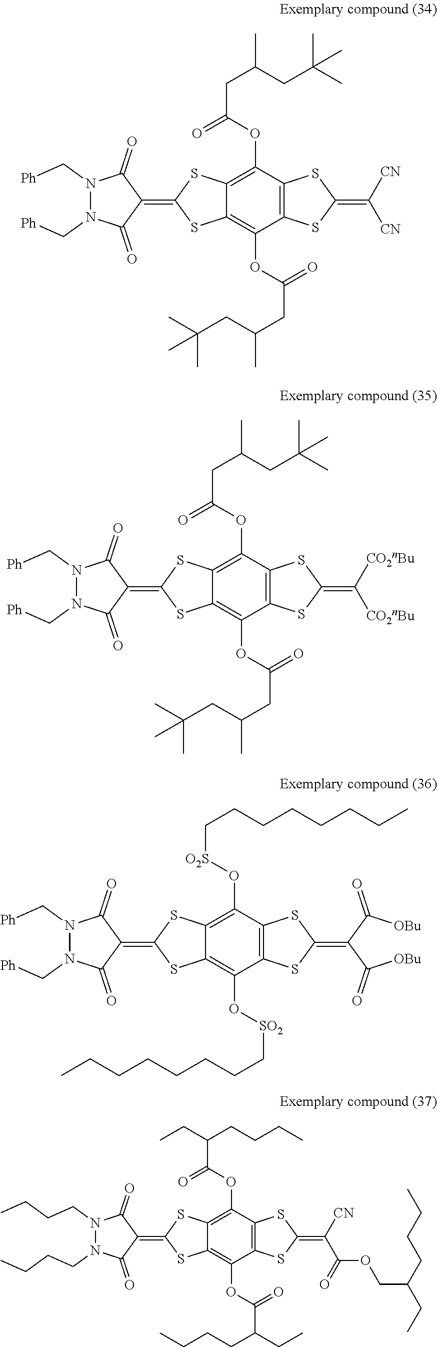

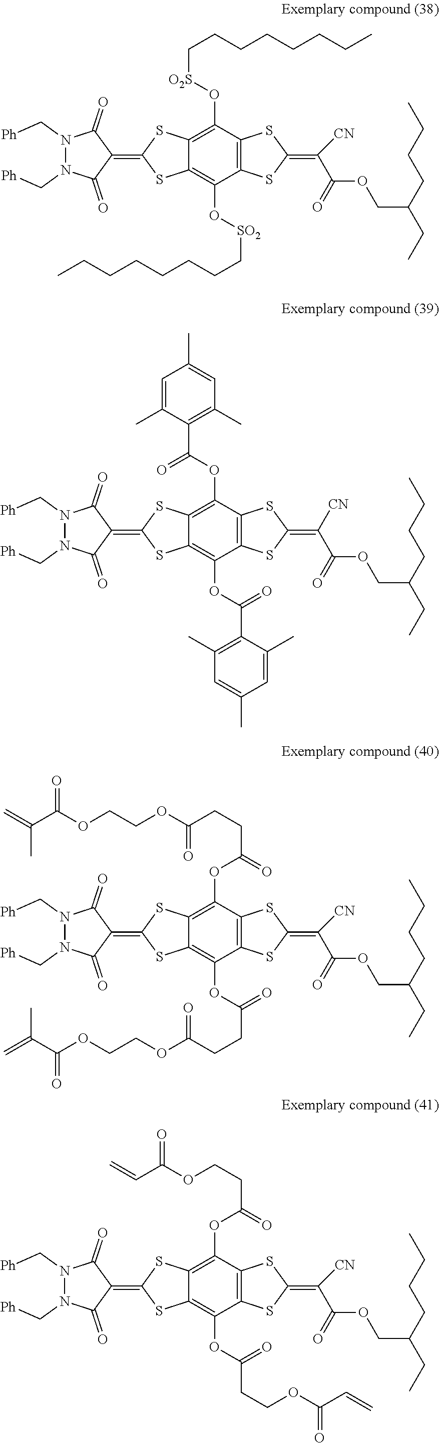

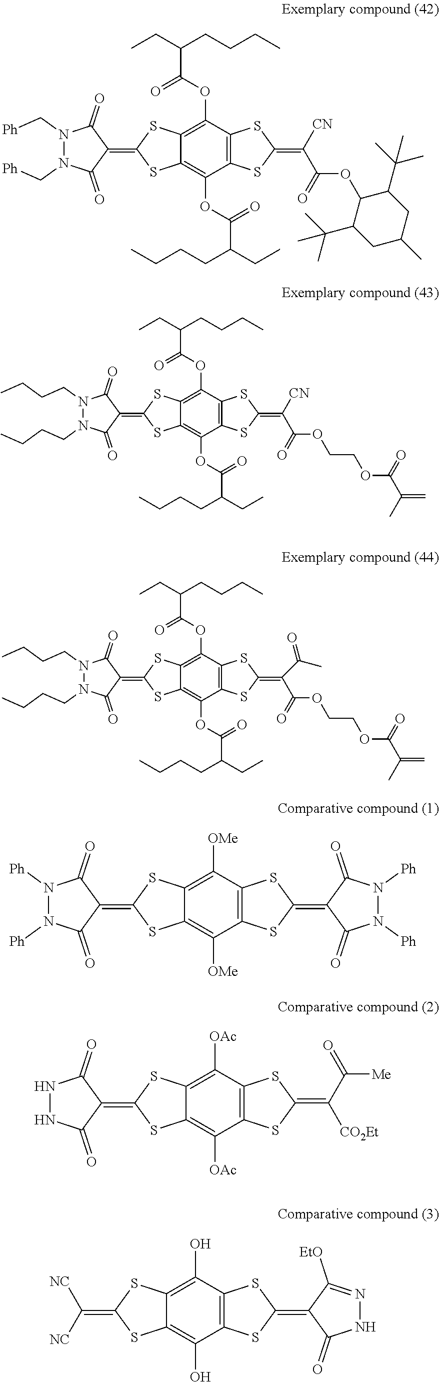

TABLE 1

| Sam- | | | Molar | Color- |
|---|---|---|---|---|
| ple | | Absorption | absorption | ability |
| solu- | | ability | coefficient | (absor- |
| tion | | of UV-A | (L/mol · | bance |
| No. | Type of compound | (λₘₐₓ (nm)) | cm) | ratio A₄₄₀) |
| 101 | Exemplary compound (1) | A(392) | 101600 | A(0.007) |
| 102 | Exemplary compound (2) | A(394) | 102000 | A(0.006) |
| 103 | Exemplary compound (3) | A(394) | 101800 | A(0.004) |
| 104 | Exemplary compound (4) | A(401) | 116300 | A(0.004) |
| 105 | Exemplary compound (5) | A(394) | 87400 | A(0.007) |
| 106 | Exemplary compound (6) | A(396) | 95000 | A(0.006) |
| 107 | Exemplary compound (7) | A(390) | 90900 | A(0.009) |
| 108 | Exemplary compound (8) | A(395) | 100000 | A(0.006) |
| 109 | Exemplary compound (9) | A(403) | 110500 | A(0.004) |
| 110 | Exemplary compound (10) | A(390) | 90200 | A(0.009) |
| 111 | Exemplary compound (11) | A(395) | 97000 | A(0.007) |
| 112 | Exemplary compound (12) | A(391) | 99000 | A(0.004) |
| 113 | Exemplary compound (13) | A(391) | 98000 | A(0.005) |
| 114 | Exemplary compound (14) | A(390) | 102000 | A(0.004) |
| 115 | Exemplary compound (15) | A(390) | 103100 | A(0.006) |
| 116 | Exemplary compound (16) | A(392) | 105000 | A(0.009) |
| 117 | Exemplary compound (17) | A(394) | 102000 | A(0.008) |
| 118 | Exemplary compound (18) | A(394) | 101400 | A(0.006) |
| 119 | Exemplary compound (19) | A(394) | 99900 | A(0.005) |
| 120 | Exemplary compound (20) | A(394) | 97400 | A(0.005) |
| 121 | Exemplary compound (21) | A(394) | 105000 | A(0.005) |
| 122 | Exemplary compound (22) | A(394) | 101800 | A(0.004) |
| 123 | Exemplary compound (23) | A(392) | 101200 | A(0.011) |
| 124 | Exemplary compound (24) | A(392) | 87300 | A(0.007) |
| 125 | Exemplary compound (25) | A(392) | 97000 | A(0.006) |
| 126 | Exemplary compound (26) | A(392) | 99700 | A(0.006) |
| 127 | Exemplary compound (27) | A(394) | 105000 | A(0.008) |
| 128 | Exemplary compound (28) | A(394) | 102000 | A(0.006) |
| 129 | Exemplary compound (29) | A(394) | 104000 | A(0.006) |
| 130 | Exemplary compound (30) | A(394) | 101000 | A(0.006) |

TABLE 2

| Sam- | | | Molar | Color- |
|---|---|---|---|---|
| ple | | Absorption | absorption | ability |
| solu- | | ability | coefficient | (absor- |
| tion | | of UV-A | (L/mol · | bance |
| No. | Type of compound | (λₘₐₓ (nm)) | cm) | ratio A₄₄₀) |
| 131 | Exemplary compound (31) | A(394) | 100000 | A(0.007) |
| 132 | Exemplary compound (32) | A(394) | 102000 | A(0.006) |
| 133 | Exemplary compound (33) | A(394) | 104000 | A(0.006) |
| 134 | Exemplary compound (34) | A(393) | 97000 | A(0.007) |
| 135 | Exemplary compound (35) | A(396) | 91000 | A(0.005) |
| 136 | Exemplary compound (36) | A(396) | 96000 | A(0.007) |
| 137 | Exemplary compound (37) | A(391) | 98000 | A(0.010) |
| 138 | Exemplary compound (38) | A(395) | 110000 | A(0.006) |
| 139 | Exemplary compound (39) | A(394) | 109000 | A(0.006) |
| 140 | Exemplary compound (40) | A(394) | 108000 | A(0.006) |
| 141 | Exemplary compound (41) | A(394) | 109000 | A(0.006) |
| 142 | Exemplary compound (42) | A(394) | 106000 | A(0.006) |
| 143 | Exemplary compound (43) | A(391) | 109000 | A(0.006) |
| 144 | Exemplary compound (44) | A(397) | 106000 | A(0.006) |
| 145 | Comparative compound (1) | A(415) | 102000 | B(0.050) |
| 146 | Comparative compound (2) | B(385) | 75400 | A(0.007) |
| 147 | Comparative compound (3) | C(360) | 65000 | B(0.084) |

As shown in the above tables, the sample solutions 101 to 144 using the exemplary compounds (1) to (44) were excellent in evaluations of absorption ability of WV-A and colorability.

### Test Example 2

A resin composition was prepared by dissolving 7.6 g of the compound shown in the following tables (exemplary compounds (1) to (44) and comparative compounds (1) to (3)) in 7.6 g of chloroform and 1.1 g of a (meth)acrylic resin (Dianal BR-80, manufactured by Mitsubishi Chemical Corporation, containing 60% by mass or more of methyl methacrylate as a monomer unit, Mw: 95,000). The obtained resin composition was spin-coated on a glass substrate to form a coating film, and the obtained coating film was dried at 110° C. for 2 minutes to produce resin films 201 to 251.

### (Evaluation of Spectral Characteristics)

With regard to the resin films 201 to 251, an absorbance was measured using a spectrophotometer (UV-1800PC, manufactured by Shimadzu Corporation). A maximal absorption wavelength (λₘₐₓ) was measured from a spectral chart obtained for each resin film, and absorption ability of UV-A was evaluated according to the same standard as in Test Example 1.

In addition, for each resin film, a value of a ratio of the absorbance at a wavelength of 440 nm in a case where the absorbance at a wavelength of 400 nm was set to 1 (absorbance ratio A₄₄₀) was calculated, and colorability was evaluated according to the same standard as in Test Example 1.

The evaluation results are listed in the following tables. The numerical value in the column of absorption ability of UV-A is the value of λₘₐₓ, and the numerical value in the column of colorability is the value of the absorbance ratio A₄₄₀.

### (Evaluation of Light Resistance)

With regard to the resin films 201 to 251, light resistance was evaluated by performing a light resistance test under the following condition 1, obtaining a rate of maintaining an absorbance at the maximal absorption wavelength (λₘₐₓ). Specifically, an absorbance of the resin film at λₘₐₓ was measured using a spectrophotometer (UV-1800PC, manufactured by Shimadzu Corporation), the resin film was subjected to a light resistance test for 3 weeks under the condition 1, and the absorbance of the resin film after the light resistance test at λₘₐₓ was measured. Next, an absorbance retention rate (%) was calculated from the values of the absorbance of the resin film before and after the light resistance test at λₘₐₓ using the following expression, and light resistance was evaluated according to the following standard. As the absorbance retention rate is higher, the light resistance is more excellent. The evaluation results are shown in the tables below. The numerical value in the column of light resistance is the value of the absorbance retention rate.

$$Absorbance retention rate ( \% ) = ( Absorbance of resin film after light resistance test at {λ}_{max} / Absorbance of resin film before light resistance test at {λ}_{max} ) \times 100$$

### —Evaluation Standard—

AA: absorbance retention rate was 90% or more.
A: absorbance retention rate was 85% or more.
B: absorbance retention rate was 80% or more and less than 85%.
C: absorbance retention rate was less than 80%.

### (Condition 1)

Equipment: low temperature cycle xenon weather meter (XL75, manufactured by Suga Test Instruments Co., Ltd.)
Illuminance: 90 klx (40 w/m²)
Time: 3 weeks
Environment: 23° C., relative humidity: 50%

TABLE 3

| | | | Absorp- | | |
|---|---|---|---|---|---|
| | Compound | | tion | | Light |
| | | Dis- | ability | Color- | resistance |
| Resin | | solved | of UV-A | ability | (absorbance |
| film | | amount | (λₘₐₓ | (absorbance | retention |
| No. | Type | (mg) | (nm)) | ratio A₄₄₀) | rate (%)) |
| 201 | Exemplary | 5.9 | A(397) | A(0.005) | AA(93) |
| | compound (1) | | | | |
| 202 | Exemplary | 6.4 | A(398) | A(0.006) | AA(91) |
| | compound (2) | | | | |
| 203 | Exemplary | 7.1 | A(398) | A(0.006) | AA(91) |
| | compound (3) | | | | |
| 204 | Exemplary | 5.5 | A(405) | A(0.007) | AA(91) |
| | compound (4) | | | | |
| 205 | Exemplary | 7.0 | A(398) | A(0.007) | AA(93) |
| | compound (5) | | | | |
| 206 | Exemplary | 6.8 | A(400) | A(0.008) | AA(94) |
| | compound (6) | | | | |
| 207 | Exemplary | 7.0 | A(395) | A(0.007) | AA(93) |
| | compound (7) | | | | |
| 208 | Exemplary | 6.4 | A(400) | A(0.006) | AA(92) |
| | compound (8) | | | | |
| 209 | Exemplary | 5.5 | A(407) | A(0.007) | AA(93) |
| | compound (9) | | | | |
| 210 | Exemplary | 4.6 | A(395) | A(0.010) | AA(95) |
| | compound (10) | | | | |
| 211 | Exemplary | 5.9 | A(400) | A(0.008) | AA(91) |
| | compound (11) | | | | |
| 212 | Exemplary | 5.7 | A(396) | A(0.005) | AA(92) |
| | compound (12) | | | | |
| 213 | Exemplary | 5.9 | A(396) | A(0.005) | AA(93) |
| | compound (13) | | | | |
| 214 | Exemplary | 6.3 | A(395) | A(0.004) | AA(95) |
| | compound (14) | | | | |
| 215 | Exemplary | 6.4 | A(395) | A(0.007) | AA(95) |
| | compound (15) | | | | |
| 216 | Exemplary | 5.9 | A(397) | A(0.009) | A(86) |
| | compound (16) | | | | |
| 217 | Exemplary | 5.9 | A(399) | A(0.010) | A(86) |
| | compound (17) | | | | |
| 218 | Exemplary | 7.0 | A(398) | A(0.006) | AA(92) |
| | compound (18) | | | | |
| 219 | Exemplary | 6.6 | A(398) | A(0.006) | AA(92) |
| | compound (19) | | | | |
| 220 | Exemplary | 6.7 | A(398) | A(0.006) | AA(89) |
| | compound (20) | | | | |
| 221 | Exemplary | 6.2 | A(398) | A(0.006) | AA(92) |
| | compound (21) | | | | |
| 222 | Exemplary | 8.6 | A(398) | A(0.006) | AA(91) |
| | compound (22) | | | | |
| 223 | Exemplary | 7.4 | A(397) | A(0.005) | AA(91) |
| | compound (23) | | | | |
| 224 | Exemplary | 8.5 | A(397) | A(0.006) | AA(92) |
| | compound (24) | | | | |
| 225 | Exemplary | 7.0 | A(397) | A(0.006) | AA(90) |
| | compound (25) | | | | |
| 226 | Exemplary | 6.0 | A(398) | A(0.005) | AA(93) |
| | compound (26) | | | | |
| 227 | Exemplary | 5.8 | A(399) | A(0.006) | AA(93) |
| | compound (27) | | | | |
| 228 | Exemplary | 6.3 | A(398) | A(0.005) | AA(93) |
| | compound (28) | | | | |
| 229 | Exemplary | 6.4 | A(398) | A(0.005) | AA(91) |
| | compound (29) | | | | |
| 230 | Exemplary | 7.2 | A(398) | A(0.006) | AA(90) |
| | compound (30) | | | | |

TABLE 4

| | | | Absorp- | | |
|---|---|---|---|---|---|
| | Compound | | tion | | Light |
| | | Dis- | ability | Color- | resistance |
| Resin | | solved | of UV-A | ability | (absorbance |
| film | | amount | (λₘₐₓ | (absorbance | retention |
| No. | Type | (mg) | (nm)) | ratio A₄₄₀) | rate (%)) |
| 231 | Exemplary | 7.5 | A(398) | A(0.005) | AA(91) |
| | compound (31) | | | | |
| 232 | Exemplary | 6.8 | A(398) | A(0.005) | AA(90) |
| | compound (32) | | | | |
| 233 | Exemplary | 7.1 | A(398) | A(0.005) | AA(90) |
| | compound (33) | | | | |
| 234 | Exemplary | 7.0 | A(397) | A(0.005) | AA(93) |
| | compound (34) | | | | |
| 235 | Exemplary | 8.9 | A(400) | A(0.008) | AA(89) |
| | compound (35) | | | | |
| 236 | Exemplary | 8.1 | A(399) | A(0.007) | AA(91) |
| | compound (36) | | | | |
| 237 | Exemplary | 6.8 | A(395) | A(0.007) | AA(90) |
| | compound (37) | | | | |
| 238 | Exemplary | 7.0 | A(398) | A(0.005) | AA(90) |
| | compound (38) | | | | |
| 239 | Exemplary | 6.6 | A(398) | A(0.006) | AA(90) |
| | compound (39) | | | | |
| 240 | Exemplary | 6.9 | A(398) | A(0.006) | AA(91) |
| | compound (40) | | | | |
| 241 | Exemplary | 6.8 | A(398) | A(0.006) | AA(90) |
| | compound (41) | | | | |
| 242 | Exemplary | 6.5 | A(398) | A(0.006) | AA(91) |
| | compound (42) | | | | |
| 243 | Exemplary | 7.2 | A(395) | A(0.006) | AA(92) |
| | compound (43) | | | | |
| 244 | Exemplary | 6.0 | A(400) | A(0.006) | AA(91) |
| | compound (44) | | | | |
| 245 | Exemplary | 23.6 | A(397) | A(0.005) | AA(94) |
| | compound (1) | | | | |
| 246 | Exemplary | 3.0 | A(397) | A(0.005) | AA(92) |
| | compound (1) | | | | |
| 247 | Exemplary | 28.0 | A(398) | A(0.006) | AA(93) |
| | compound (18) | | | | |
| 248 | Exemplary | 3.5 | A(398) | A(0.006) | AA(91) |
| | compound (18) | | | | |
| 249 | Comparative | 6.4 | A(420) | B(0.056) | A(86) |
| | compound (1) | | | | |
| 250 | Comparative | 7.1 | B(389) | A(0.008) | B(81) |
| | compound (2) | | | | |
| 251 | Comparative | 8.1 | C(364) | B(0.099) | C(51) |
| | compound (3) | | | | |

As shown in the above tables, the resin films 201 to 248 formed of the exemplary compounds (1) to (44) were excellent in the evaluations of absorption ability of UV-A, colorability, and light resistance.

### Test Example 3

The following components were mixed to prepare a resin composition (photopolymerizable composition).
Ultraviolet absorber (compound shown in the following table) . . . 0.6 parts by mass
Polymerizable compound (KAYARAD DPHA (manufactured by Nippon Kayaku Co., Ltd.), compound having two or more ethylenically unsaturated bond-containing groups) . . . 2.8 parts by mass
Resin (Dianal BR-80 (manufactured by Mitsubishi Chemical Corporation)) . . . 14.8 parts by mass
Photopolymerization initiator (compound shown in the following table) . . . 2.4 parts by mass
Solvent (propylene glycol monomethyl ether acetate) . . . 79.4 parts by mass

TABLE 5

| Resin | | Photopolymerization |
|---|---|---|
| composition No. | Ultraviolet absorber | initiator |
| 301 | Exemplary compound (1) | V-1 |
| 302 | Exemplary compound (1) | V-2 |
| 303 | Exemplary compound (1) | V-3 |
| 304 | Exemplary compound (18) | V-1 |
| 305 | Exemplary compound (18) | V-2 |
| 306 | Exemplary compound (18) | V-3 |
| 307 | Exemplary compound (28) | V-3 |
| 308 | Exemplary compound (29) | V-3 |
| 309 | Exemplary compound (30) | V-3 |
| 310 | Exemplary compound (32) | V-3 |
| 311 | Exemplary compound (33) | V-1 |
| 312 | Exemplary compound (33) | V-2 |
| 313 | Exemplary compound (33) | V-3 |
| 314 | Exemplary compound (40) | V-3 |
| 315 | Exemplary compound (41) | V-3 |
| 316 | Exemplary compound (42) | V-1 |
| 317 | Exemplary compound (42) | V-2 |
| 318 | Exemplary compound (42) | V-3 |
| 319 | Exemplary compound (43) | V-1 |
| 320 | Exemplary compound (43) | V-2 |
| 321 | Exemplary compound (43) | V-3 |
| 322 | Comparative compound (1) | V-1 |
| 323 | Comparative compound (1) | V-2 |
| 324 | Comparative compound (1) | V-3 |
| 325 | Comparative compound (2) | V-1 |
| 326 | Comparative compound (2) | V-2 |
| 327 | Comparative compound (2) | V-3 |
| 328 | Comparative compound (3) | V-1 |
| 329 | Comparative compound (3) | V-2 |
| 330 | Comparative compound (3) | V-3 |

In the above table, details of the raw materials indicated by abbreviations are as follows.

### (Ultraviolet Absorber)

Exemplary compounds (1), (18), (28), (29), (30), (32), (33), (40), (41), (42), (43), and comparative compounds (1) to (3): compounds having the above-described structures

### (Photopolymerization Initiator)

V-1: Irgacure OXE01 (manufactured by BASF SE, oxime compound, photoradical polymerization initiator)
V-2: Omnirad 2959 (manufactured by IGM Resins B. V., hydroxyacetophenone compound, photoradical polymerization initiator)
V-3: Omnirad TPO (manufactured by IGM Resins B. V., acylphosphine compound, photoradical polymerization initiator)

### <Production of Resin Film>

The above-described resin composition was spin-coated on a glass substrate (1737, manufactured by Corning) having a size of 50 mm×50 mm such that a film thickness after film formation was 1.5 μm, and dried at 120° C. for 5 minutes to form a resin composition layer. Thereafter, the resin composition layer was entirely exposed to an i-ray stepper exposure device (UX-1000SM-EH04, manufactured by USHIO INC.) with an exposure amount of 1,000 mJ/cm², thereby producing resin films 301 to 330.

For the resin compositions 301 to 327, a degree of change in transmittance (degree 1 of change in transmittance) at a maximal absorption wavelength (λₘₐₓ) of the resin composition layer before and after the exposure was 5% or less.

### (Evaluation of Light Resistance)

With regard to the resin films 301 to 330, a rate of maintaining an absorbance at the maximal absorption wavelength (λₘₐₓ) was obtained under the following condition 2, and light resistance was evaluated. Specifically, an absorbance of the resin film at λₘₐₓ was measured using a spectrophotometer (UV-1800PC, manufactured by Shimadzu Corporation), the resin film was subjected to a light resistance test for 3 days under the condition 2, and the absorbance of the resin film after the light resistance test at λₘₐₓ was measured. Next, an absorbance retention rate (%) was calculated from the values of the absorbance of the resin film before and after the light resistance test at λₘₐₓ using the following expression, and light resistance was evaluated according to the following standard. As the absorbance retention rate is higher, the light resistance is more excellent. The evaluation results are shown in the tables below. The numerical value in the column of light resistance is the value of the absorbance retention rate.

Absorbance retention rate (%)=(Absorbance of resin film after light resistance test at λₘₐₓ/Absorbance of resin film before light resistance test at λₘₐₓ)×100

### —Evaluation Standard—

AA: absorbance retention rate was 75% or more.
A: absorbance retention rate was 65% or more.
B: absorbance retention rate was 50% or more and less than 65%.
C: absorbance retention rate was less than 50%.

### (Condition 2)

Equipment: low temperature cycle xenon weather meter (XL75, manufactured by Suga Test Instruments Co., Ltd.)
Illuminance: 90 klx (40 w/m²)
Time: 3 days
Environment: 23° C., relative humidity: 50%

TABLE 6

| | | | Light |
|---|---|---|---|
| | | | resistance |
| Resin | | | (absorbance |
| film | Used resin | Type of ultraviolet | retention |
| No. | composition | absorber | rate (%)) |
| 301 | Resin composition 301 | Exemplary compound (1) | A(65) |
| 302 | Resin composition 302 | Exemplary compound (1) | AA(84) |
| 303 | Resin composition 303 | Exemplary compound (1) | AA(83) |
| 304 | Resin composition 304 | Exemplary compound (18) | A(66) |
| 305 | Resin composition 305 | Exemplary compound (18) | AA(86) |
| 306 | Resin composition 306 | Exemplary compound (18) | AA(86) |
| 307 | Resin composition 307 | Exemplary compound (28) | A(77) |
| 308 | Resin composition 308 | Exemplary compound (29) | A(80) |
| 309 | Resin composition 309 | Exemplary compound (30) | A(79) |
| 310 | Resin composition 310 | Exemplary compound (32) | A(80) |
| 311 | Resin composition 311 | Exemplary compound (33) | A(65) |
| 312 | Resin composition 312 | Exemplary compound (33) | AA(88) |
| 313 | Resin composition 313 | Exemplary compound (33) | AA(87) |
| 314 | Resin composition 314 | Exemplary compound (40) | A(82) |
| 315 | Resin composition 315 | Exemplary compound (41) | A(80) |
| 316 | Resin composition 316 | Exemplary compound (42) | A(66) |
| 317 | Resin composition 317 | Exemplary compound (42) | AA(87) |
| 318 | Resin composition 318 | Exemplary compound (42) | AA(86) |
| 319 | Resin composition 316 | Exemplary compound (43) | A(68) |
| 320 | Resin composition 317 | Exemplary compound (43) | AA(88) |
| 321 | Resin composition 318 | Exemplary compound (43) | AA(88) |
| 322 | Resin composition 319 | Comparative compound (1) | C(45) |
| 323 | Resin composition 320 | Comparative compound (1) | B(57) |
| 324 | Resin composition 321 | Comparative compound (1) | B(58) |
| 325 | Resin composition 322 | Comparative compound (2) | C(32) |
| 326 | Resin composition 323 | Comparative compound (2) | C(41) |
| 327 | Resin composition 324 | Comparative compound (2) | C(40) |
| 328 | Resin composition 325 | Comparative compound (3) | C(30) |
| 329 | Resin composition 326 | Comparative compound (3) | C(38) |
| 330 | Resin composition 327 | Comparative compound (3) | C(36) |

As shown in the above table, the resin films 301 to 321 formed of the exemplary compound (1), (18), (28), (29), (30), (32), (33), (40), (41), (42), or (43) had excellent light resistance. In addition, the resin films 301 to 321 had excellent absorption ability of ultraviolet rays in the vicinity of a wavelength of 400 nm and had little coloration.

The resin films 301 to 321 were stored for 1 week under conditions of 40° C. and a humidity of 50%, and then left to stand at room temperature for 1 day, and the presence or absence of bleed-out and precipitation was visually observed. No bleed-out and precipitation were confirmed in any of the resin films 301 to 321.

### Test Example 4

### (Preparation of Compositions 401 to 424 and 432 to 437)

The following components were mixed to prepare compositions (photopolymerizable compositions) 401 to 424 and 432 to 437.
Ultraviolet absorber (compound shown in the following table) . . . 2.0 parts by mass
Polymerizable compound (compound shown in the following table) . . . 2.6 parts by mass
Resin (resin shown in the following table) . . . 12.9 parts by mass
Photopolymerization initiator (compound shown in the following table) . . . 2.5 parts by mass
Solvent (propylene glycol monomethyl ether acetate) . . . 40.0 parts by mass
Solvent (cyclopentanone) . . . 40.0 parts by mass
Surfactant (KF-6001, manufactured by Shin-Etsu Chemical Co., Ltd., polydimethylsiloxane modified with carbinol at both ends, hydroxyl number: 62 mgKOH/g) . . . 0.02 parts by mass

TABLE 7

| Compo- | | Polym- | | Photopoly- |
|---|---|---|---|---|
| sition | | erizable | | merization |
| No. | Ultraviolet absorber | compound | Resin | initiator |
| 401 | Exemplary compound (1) | T-1 | U-2 | V-1 |
| 402 | Exemplary compound (1) | T-1 | U-2 | V-2 |
| 403 | Exemplary compound (1) | T-1 | U-2 | V-5 |
| 404 | Exemplary compound (18) | T-1 | U-2 | V-1 |
| 405 | Exemplary compound (18) | T-1 | U-1 | V-1 |
| 406 | Exemplary compound (18) | T-1 | U-2 | V-2 |
| 407 | Exemplary compound (18) | T-1 | U-2 | V-3 |
| 408 | Exemplary compound (18) | T-1 | U-2 | V-4 |
| 409 | Exemplary compound (18) | T-1 | U-2 | V-5 |
| 410 | Exemplary compound (18) | T-1 | U-2 | V-6 |
| 411 | Exemplary compound (18) | T-1 | U-2 | V-7 |
| 412 | Exemplary compound (18) | T-1 | U-2 | V-8 |
| 413 | Exemplary compound (18) | T-1 | U-2 | V-1 |
| 414 | Exemplary compound (18) | T-2 | U-2 | V-1 |
| 415 | Exemplary compound (18) | T-3 | U-2 | V-1 |
| 416 | Exemplary compound (18) | T-4 | U-2 | V-1 |
| 417 | Exemplary compound (18) | T-5 | U-2 | V-1 |
| 418 | Exemplary compound (18) | T-6 | U-2 | V-1 |
| 419 | Exemplary compound (33) | T-1 | U-2 | V-1 |
| 420 | Exemplary compound (33) | T-1 | U-2 | V-2 |
| 421 | Exemplary compound (33) | T-1 | U-2 | V-5 |
| 422 | Exemplary compound (42) | T-1 | U-2 | V-1 |
| 423 | Exemplary compound (42) | T-1 | U-2 | V-2 |
| 424 | Exemplary compound (42) | T-1 | U-2 | V-5 |
| 432 | Exemplary compound (43) | T-1 | U-2 | V-1 |
| 433 | Exemplary compound (43) | T-1 | U-2 | V-2 |
| 434 | Exemplary compound (43) | T-1 | U-2 | V-5 |
| 435 | Comparative compound (1) | T-1 | U-2 | V-1 |
| 436 | Comparative compound (2) | T-1 | U-2 | V-2 |
| 437 | Comparative compound (3) | T-1 | U-2 | V-5 |

### (Preparation of Composition 425)

The following components were mixed to prepare a composition (photopolymerizable composition) 425.
Ultraviolet absorber (exemplary compound (1)) . . . 2.0 parts by mass
Polymerizable compound T-2 . . . 0.5 parts by mass
Polymerizable compound T-4 . . . 1.5 parts by mass
Resin U-3 . . . 13.5 parts by mass
Photopolymerization initiator V-8 . . . 2.5 parts by mass
Solvent (toluene) . . . 80.0 parts by mass
Surfactant (KF-6001, manufactured by Shin-Etsu Chemical Co., Ltd., polydimethylsiloxane modified with carbinol at both ends, hydroxyl number: 62 mgKOH/g) . . . 0.02 parts by mass

### (Preparation of Composition 426)

The following components were mixed to prepare a composition (photopolymerizable composition) 426.
Ultraviolet absorber (exemplary compound (1)) . . . 2.0 parts by mass
Polymerizable compound T-6 . . . 10.5 parts by mass
Polymerizable compound T-1 . . . 5.0 parts by mass
Photopolymerization initiator V-1 . . . 2.2 parts by mass
Photopolymerization initiator V-5 . . . 0.3 parts by mass
Solvent (ethyl acetate) . . . 40.0 parts by mass
Solvent (cyclopentanone) . . . 40.0 parts by mass
Surfactant (KF-6001, manufactured by Shin-Etsu Chemical Co., Ltd., polydimethylsiloxane modified with carbinol at both ends, hydroxyl number: 62 mgKOH/g) . . . 0.02 parts by mass

### (Preparation of Composition 427)

The following components were mixed to prepare a composition (photopolymerizable composition) 427.
Ultraviolet absorber (exemplary compound (1)) . . . 2.0 parts by mass
Polymerizable compound T-5 . . . 5.0 parts by mass
Polymerizable compound T-4 . . . 4.0 parts by mass
Resin U-1 . . . 6.5 parts by mass
Photopolymerization initiator V-1 . . . 2.0 parts by mass
Photopolymerization initiator V-6 . . . 0.5 parts by mass
Solvent (ethyl acetate) . . . 40.0 parts by mass
Solvent (cyclopentanone) . . . 40.0 parts by mass
Surfactant (KF-6001, manufactured by Shin-Etsu Chemical Co., Ltd., polydimethylsiloxane modified with carbinol at both ends, hydroxyl number: 62 mgKOH/g) . . . 0.02 parts by mass

### (Preparation of Composition 428)

The following components were mixed to prepare a composition (photopolymerizable composition) 428.
Ultraviolet absorber (exemplary compound (1)) . . . 2.0 parts by mass
Polymerizable compound T-7 . . . 60 parts by mass
Polymerizable compound T-8 . . . 25 parts by mass
Polymerizable compound T-9 . . . 15 parts by mass
Photopolymerization initiator V-9 . . . 8.0 parts by mass

### (Preparation of Compositions 429 to 431)

Compositions 429 to 431 were prepared in the same manner as in the composition 428, except that, in the composition 428, the ultraviolet absorber was changed to the exemplary compound (18), the exemplary compound (33), or the exemplary compound (42) with the same amount.

Details of the raw materials indicated by abbreviations are as follows.

### (Ultraviolet Absorber)

Exemplary compounds (1), (18), (33), (42), (43), and comparative compounds (1), (2), (3): compounds having the above-described structures

### (Polymerizable Compound)

T-1: KAYARAD DPHA (manufactured by Nippon Kayaku Co., Ltd.; compound having two or more ethylenically unsaturated bond-containing groups)
T-2: NK ESTER A-DPH-12E (manufactured by Shin-Nakamura Chemical Co., Ltd., compound having two or more ethylenically unsaturated bond-containing groups)
T-3: Light Acrylate DCP-A (manufactured by Kyoeisha Chemical Co., Ltd., compound having two or more ethylenically unsaturated bond-containing groups)
T4: benzyl methacrylate
T-5: BEAMSET 577 (tri- to hexafunctional urethane acrylate, manufactured by Arakawa Chemical Industries, Ltd.)
T-6: FA-512M (dicyclopentenyloxyethyl methacrylate, manufactured by Showa Denko Materials Co., Ltd.)
T-7: CYCLOMER M100 (3,4-epoxycyclohexylmethyl methacrylate, manufactured by Daicel Corporation)
T-8: OXT-221: (3-ethyl-3-{[(3-ethyloxetane-3-yl)methoxy]methyl}oxetane, bifunctional oxetane, manufactured by TOAGOSEI CO., LTD.)
T-9: CELLOXIDE 2021P (3′,4′-epoxycyclohexylmethyl 3,4-epoxycyclohexane carboxylate, bifunctional epoxy, manufactured by Daicel Corporation)

### (Resin)

U-1: 40% by mass propylene glycol monomethyl ether acetate solution of a copolymer of benzyl methacrylate/methacrylic acid (75/25 [mass ratio])(weight-average molecular weight: 12,000)
U-2: Dianal BR-80 (manufactured by Mitsubishi Chemical Corporation)
U-3: ARTON RX4500 (manufactured by JSR Corporation, Tg: 140° C., cyclic polyolefin resin)

### (Photopolymerization Initiator)

V-1: Omnirad TPO (manufactured by IGM Resins B. V., photoradical polymerization initiator, acylphosphine compound)
V-2: Omnirad 2959 (manufactured by IGM Resins B. V., photoradical polymerization initiator, hydroxyacetophenone compound)
V-3: 4,4′-bis(diethylamino)benzophenone (photoradical polymerization initiator, benzophenone compound)
V4: 2,2′-bis(2-chlorophenyl)-4,4′,5,5′-tetraphenyl-1,2′-biimidazole (o-Cl-HABI) (photoradical polymerization initiator, hexaarylbiimidazole compound)
V-5: IRGACURE-OXE01 (manufactured by BASF SE, photoradical polymerization initiator, oxime compound)
V-6: Omnirad 907 (manufactured by IGM Resins B. V., photoradical polymerization initiator, aminoacetophenone compound)
V-7: Omnirad 369 (manufactured by IGM Resins B. V., photoradical polymerization initiator, aminoacetophenone compound)
V-8: Omnirad 819 (manufactured by IGM Resins B. V., photoradical polymerization initiator, acylphosphine compound)
V-9: CPI-210S (manufactured by San-Apro Ltd., photocationic polymerization initiator, sulfonium salt)

### (Production of Resin Film)

### [Production Example 4-1] Production of Resin Films Formed of Compositions 401 to 435

The compositions 401 to 435 were spin-coated on a glass substrate (1737, manufactured by Corning) having a size of 50 mm×50 mm such that a film thickness after film formation was 1.5 μm, and dried at 100° C. for 2 minutes to form a composition layer. Thereafter, the composition layer was entirely exposed to an i-ray stepper exposure device (UX-1000SM-EH04, manufactured by USHIO INC.) with an exposure amount of 1,000 mJ/cm². Next, the composition layer was heated (post-baked) using a hot plate at 200° C. for 8 minutes to produce resin films 401 to 432.

For the resin films 401 to 435 formed of the compositions 401 to 435, both the degree of change in transmittance (degree 1 of change in transmittance) of the composition layer before and after the exposure at the maximal absorption wavelength (λₘₐₓ) and a degree of change in transmittance (degree 2 of change in transmittance) of the composition layer before and after the post-baking at the maximal absorption wavelength (λₘₐₓ) were both 1% or less.

Degree 1 of change in transmittance=|Transmittance of composition layer before exposure at λₘₐₓ−Transmittance of composition layer after exposure at λₘₐₓ|

Degree 2 of change in transmittance=|Transmittance of composition layer before post-baking at λₘₐₓ−Transmittance of composition layer after post-baking at λₘₐₓ|

### [Production Example 4-2] Production of Resin Films Formed of Compositions 436 and 437

Resin films 436 and 437 were produced by the same method as in Production Example 4-1, except that the compositions 436 and 437 were used, and the composition layer were adjusted to have a film thickness such that the transmittance of the composition layer before the exposure at the maximal absorption wavelength (λₘₐₓ) was 5% to 20%.

### (Evaluation of Light Resistance)

A light resistance test was performed on the resin film obtained above under the following condition 3, and a degree of decrease in transmittance at a maximal absorption wavelength (λₘₐₓ) was calculated. Specifically, after measuring the transmittance of the resin film at the maximal absorption wavelength (λₘₐₓ), a light resistance test was performed on the resin film under the condition 3. A transmittance of the resin film after the light resistance test at the maximal absorption wavelength (λₘₐₓ) was measured, and the degree of decrease in transmittance was calculated from the following expression.

Degree of decrease in transmittance (%)=(Transmittance of resin film after light resistance test at λₘₐₓ)−(Transmittance of resin film before light resistance test at λₘₐₓ)

### (Condition 3)

Equipment: low temperature cycle xenon weather meter (XL75, manufactured by Suga Test Instruments Co., Ltd.)
Illuminance: 90 klx
Test period: 50 hours
Environment: 23° C., relative humidity: 50%

In addition, a degree of change in coloration of the resin film after the light resistance test was visually confirmed, and the presence or absence of the coloration was evaluated according to the following standard.
A: coloration was not recognized.
B: coloration was slightly recognized, but it was in a practical level.

TABLE 8

| | | Light resistance | |
|---|---|---|---|
| Resin | Used resin | Degree of decrease | Presence or absence |
| film No. | composition | in transmittance | of coloration |
| 401 | Composition 401 | 3 | A |
| 402 | Composition 402 | 2 | A |
| 403 | Composition 403 | 16 | A |
| 404 | Composition 404 | 3 | A |
| 405 | Composition 405 | 2 | A |
| 406 | Composition 406 | 2 | A |
| 407 | Composition 407 | 3 | A |
| 408 | Composition 408 | 3 | A |
| 409 | Composition 409 | 14 | A |
| 410 | Composition 410 | 2 | A |
| 411 | Composition 411 | 2 | A |
| 412 | Composition 412 | 3 | A |
| 413 | Composition 413 | 6 | A |
| 414 | Composition 414 | 5 | A |
| 415 | Composition 415 | 3 | A |
| 416 | Composition 416 | 2 | A |
| 417 | Composition 417 | 1 | A |
| 418 | Composition 418 | 1 | A |
| 419 | Composition 419 | 5 | A |
| 420 | Composition 420 | 6 | A |
| 421 | Composition 421 | 12 | A |
| 422 | Composition 422 | 2 | A |
| 423 | Composition 423 | 2 | A |
| 424 | Composition 424 | 10 | A |
| 425 | Composition 425 | 4 | A |
| 426 | Composition 426 | 6 | A |
| 427 | Composition 427 | 5 | A |
| 428 | Composition 428 | 8 | A |
| 429 | Composition 429 | 7 | A |
| 430 | Composition 430 | 4 | A |
| 431 | Composition 431 | 5 | A |
| 432 | Composition 432 | 4 | A |
| 433 | Composition 433 | 3 | A |
| 434 | Composition 434 | 4 | A |
| 435 | Composition 435 | ≥30 | B |
| 436 | Composition 436 | ≥50 | B |
| 437 | Composition 437 | ≥70 | B |

As shown in the above table, the resin films 401 to 434 formed of the exemplary compound (1), (18), (33), (42), or (43) had excellent light resistance. In addition, the resin films 401 to 434 had excellent absorption ability of ultraviolet rays in the vicinity of a wavelength of 400 nm and had little coloration.

### <Synthesis of Polymer>

### (Synthesis Example 101) Synthesis of Polymer P-1

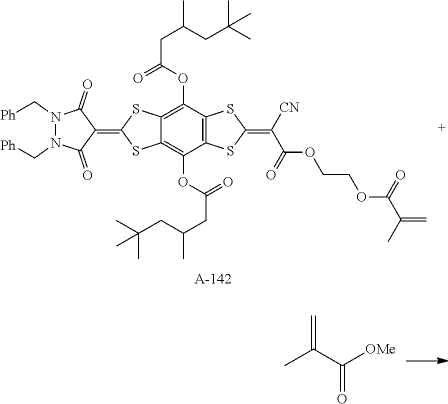

100 mg of the compound A-142 (maximal absorption wavelength (in ethyl acetate solution): 394 nm) obtained in Synthesis Example 32, 9.9 g of methyl methacrylate, and 40.0 g of propylene glycol monomethyl ether acetate were added to a 200 mL three-neck flask, and the mixture was stirred at 80° C. for 30 minutes under a nitrogen stream. 200 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 7.0 g of a target polymer P-1. A number-average molecular weight of the obtained polymer P-1 was 27,500 (in terms of polystyrene).

100 mg of the obtained polymer P-1 was dissolved in 100 mL of chloroform, and an absorption spectrum was measured. A maximal absorption wavelength of the polymer P-1 was 399 nm (absorbance: 1.61).

The polymer P-1 was able to sufficiently shield light having a wavelength in the vicinity of 400 nm. In addition, the polymer P-1 had a small coloration.

### (Synthesis Example 102) Synthesis of Polymer P-2

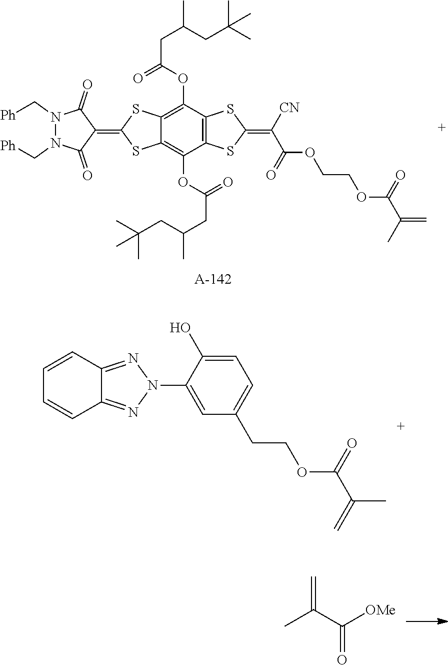

100 mg of the compound A-142 (maximal absorption wavelength (in ethyl acetate solution): 394 nm) obtained in Synthesis Example 32, 100 mg of 2-[2-hydroxy-5-(2-methacryloyloxyethyl)phenyl]2H-benzo[d][1,2,3]triazole (maximal absorption wavelength (in ethyl acetate solution): 338 nm) as an ultraviolet absorber, 9.8 g of methyl methacrylate, and 40.0 g of propylene glycol monomethyl ether acetate were added to a 200 mL three-neck flask, and the mixture was stirred at 80° C. for 6 hours under a nitrogen stream and cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 5.0 g of a target polymer P-2. A number-average molecular weight of the obtained polymer P-2 was 33,400 (in terms of polystyrene).

150 mg of the obtained polymer P-2 was dissolved in 100 mL of chloroform, and an absorption spectrum was measured. Maximal absorption wavelengths of the polymer P-2 were 399 nm (absorbance: 1.45) and 343 nm (absorbance: 0.83). The polymer P-2 was able to sufficiently shield light having a wavelength in the vicinity of 400 nm. Furthermore, shielding properties against light having a wavelength shorter than 350 nm were also excellent. In addition, the polymer P-2 had a small coloration.

### (Synthesis Example 103) Synthesis of Polymer P-3

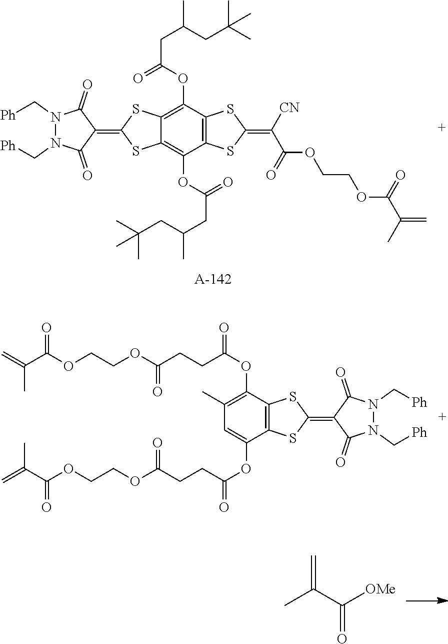

180 mg of the compound A-142 (maximal absorption wavelength (in ethyl acetate solution)): 394 nm) obtained in Synthesis Example 32, 20 mg of 2-(1,2-dibenzyl-3,5-dioxopyrrolidin-4-ylidene)-5-methylbenzo[d][1,3]dithiol-4,7-diyl bis((2-(methacryloyloxy)ethyl)succinate)(maximal absorption wavelength (in ethyl acetate solution)): 380 nm) as an ultraviolet absorber, 9.8 g of methyl methacrylate, and 40.0 g of propylene glycol monomethyl ether acetate were added to a 200 mL three-neck flask, and the mixture was stirred at 80° C. for 6 hours under a nitrogen stream and cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 6.1 g of a target polymer P-3. A number-average molecular weight of the obtained polymer P-3 was 38,200 (in terms of polystyrene).

150 mg of the obtained polymer P-3 was dissolved in 100 mL of chloroform, and an absorption spectrum was measured. Maximal absorption wavelengths of the polymer P-3 were 399 nm (absorbance: 2.12) and 383 nm (absorbance: 0.80). The polymer P-3 was able to sufficiently shield light having a wavelength in the vicinity of 400 nm. Furthermore, shielding properties against light having a wavelength shorter than 350 nm were also excellent. In addition, the polymer P-3 had a small coloration.

### (Synthesis Example 104) Synthesis of Polymer P-4

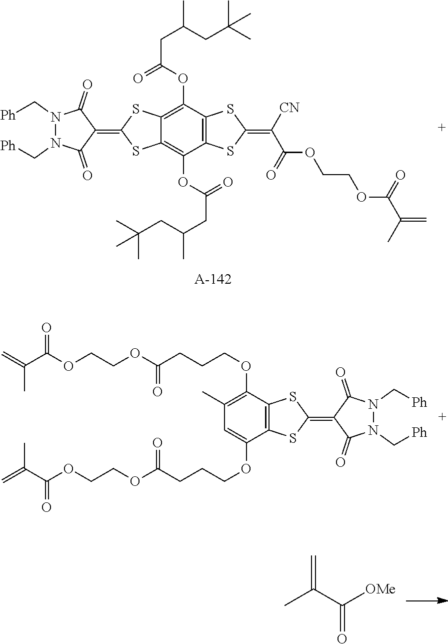

180 mg of the compound A-142 (maximal absorption wavelength (in ethyl acetate solution): 394 nm) obtained in Synthesis Example 32, 20 mg of bis(2-((methacryloyloxy)ethyl)4,4′-((2-(1,2-dibenzyl-3,5-dioxopyrazolidine-4-ylidene)-5-methylbenzo[d][1,3]dithiol-4,7-diyl)bis(oxy))dibutyrate (maximal absorption wavelength (in ethyl acetate solution): 387 nm) as an ultraviolet absorber, 9.8 g of methyl methacrylate, and 40.0 g of propylene glycol monomethyl ether acetate were added to a 200 mL three-neck flask, and the mixture was stirred at 80° C. for 6 hours under a nitrogen stream and cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 5.8 g of a target polymer PA. A number-average molecular weight of the obtained polymer P-4 was 31,900 (in terms of polystyrene).

150 mg of the obtained polymer P-4 was dissolved in 100 mL of chloroform, and an absorption spectrum was measured. Maximal absorption wavelengths of the polymer P-4 were 399 nm (absorbance: 2.31) and 390 nm (absorbance: 0.78). The polymer P-4 was able to sufficiently shield light having a wavelength in the vicinity of 400 nm. Furthermore, shielding properties against light having a wavelength shorter than 350 nm were also excellent. In addition, the polymer P-4 had a small coloration.

### (Synthesis Example 105) Synthesis of Polymer P-5

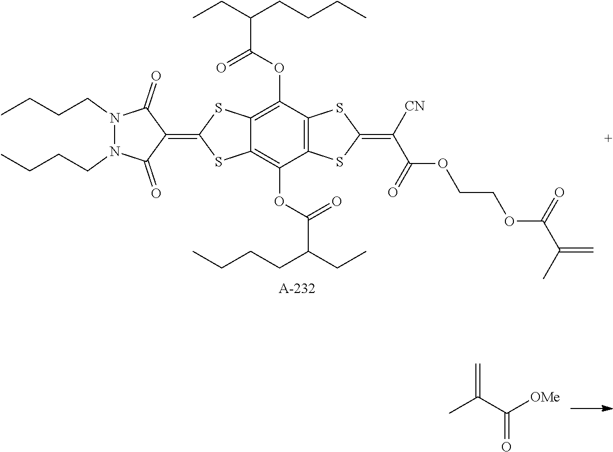

100 mg of the compound A-232 (maximal absorption wavelength (in ethyl acetate solution): 391 nm) obtained in Synthesis Example 36, 9.9 g of methyl methacrylate, and 40.0 g of propylene glycol monomethyl ether acetate were added to a 200 mL three-neck flask, and the mixture was stirred at 80° C. for 30 minutes under a nitrogen stream. 200 mg of dimethyl 2,2′-azobis(isobutyrate) (V-601, manufactured by FUJIFILM Wako Pure Chemical Corporation (hereinafter, referred to as V-601)) was added to the solution, and the mixture was stirred at 80° C. for 6 hours and then cooled to room temperature. The obtained reaction mixture was slowly added to a mixture of 140 mL of hexane and 60 mL of isopropyl alcohol, and the mixture was allowed to stand overnight. The precipitated precipitate was collected by filtration and washed with a mixture of hexane and isopropyl alcohol. 140 mL of hexane and 60 mL of isopropyl alcohol were added to the obtained powder, and the mixture was stirred at room temperature for 1 hour and allowed to stand at room temperature overnight. The precipitate was collected by filtration, washed with a mixture of hexane and isopropyl alcohol, and then dried at 50° C. to obtain 7.4 g of a target polymer P-5. A number-average molecular weight of the obtained polymer P-5 was 29,500 (in terms of polystyrene).

100 mg of the obtained polymer P-5 was dissolved in 100 mL of chloroform, and an absorption spectrum was measured. A maximal absorption wavelength of the polymer P-5 was 397 nm (absorbance: 1.57).

The polymer P-5 was able to sufficiently shield light having a wavelength in the vicinity of 400 nm. In addition, the polymer P-5 had a small coloration.

### (Comparative Synthesis Example 1) Synthesis of Polymer P-6

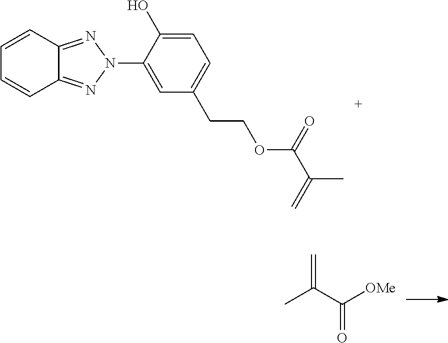

104 mg of 2-[2-hydroxy-5-(2-methacryloyloxyethyl)phenyl]2H-benzo[d][1,2,3]triazole, 9.9 g of methyl methacrylate, and 40.0 g of propylene glycol monomethyl ether acetate were added to a 200 mL three-neck flask, and the mixture was stirred at 80° C. for 30 minutes in a nitrogen stream. 135 mg of V-601 was added to the solution, and the mixture was stirred at 80° C. for 4 hours. Furthermore, 37 mg of V-601 was added thereto, and the mixture was stirred at 90° C. for 2 hours and cooled to room temperature. The obtained reaction mixture was slowly added to a mixture of 140 mL of hexane and 60 mL of isopropyl alcohol. The precipitated precipitate was collected by filtration and washed with a mixture of hexane and isopropyl alcohol. 140 mL of hexane and 60 mL of isopropyl alcohol were added to the obtained powder, the mixture was stirred at room temperature for 3 hours, and the precipitate was collected by filtration, washed with a mixture of hexane and isopropyl alcohol, and then dried at 50° C. to obtain 8.1 g of a target polymer P-6. A number-average molecular weight of the obtained polymer P-6 was 14,100 (in terms of polystyrene). 150 mg of the polymer P-6 was dissolved in 100 mL of chloroform, and an absorption spectrum was measured. A maximal absorption wavelength of the polymer P-6 was 339 nm (absorbance: 0.91). The polymer P-6 had low shielding properties against light having a wavelength of 380 to 400 nm.

### Test Example 5

### (Example 501) Production of Resin Film 501

500 mg of the polymer P-1, 7.6 g of chloroform, and 1.1 g of a poly(methyl methacrylate) resin (Dianal BR-80 containing 60% by mass or more of methyl methacrylate as a monomer unit, weight-average molecular weight: 95,000, acid value: 0 mgKOH/g, manufactured by Mitsubishi Chemical Corporation) were dissolved to prepare a resin composition (resin solution). The obtained resin composition was spin-coated on a glass substrate, and the coating film was dried at 60° C. for 2 minutes to form a resin film 501 having a thickness of approximately 10 μm, containing the polymer P-1. The resin film 501 had little coloration and had excellent shielding properties of light having a wavelength in the vicinity of 400 nm.

### (Example 502) Production of Resin Film 502

A resin film 502 was formed in the same manner as in Example 501, except that, in Example 501, 500 mg of the polymer P-1 was changed to 500 mg of the polymer P-2. The resin film 502 had little coloration and had excellent shielding properties of light having a wavelength in the vicinity of 400 nm. Furthermore, shielding properties against light having a wavelength shorter than 350 nm were also excellent.

### (Example 503) Production of Resin Film 503

A resin film 503 was formed in the same manner as in Example 501, except that, in Example 501, 500 mg of the polymer P-1 was changed to 500 mg of the polymer P-3. The resin film 503 had little coloration and had excellent shielding properties of light having a wavelength in the vicinity of 400 nm. Furthermore, shielding properties against light having a wavelength shorter than 350 nm were also excellent.

### (Example 504) Production of Resin Film 504

A resin film 504 was formed in the same manner as in Example 501, except that, in Example 501, 500 mg of the polymer P-1 was changed to 500 mg of the polymer P-4. The resin film 504 had little coloration and had excellent shielding properties of light having a wavelength in the vicinity of 400 nm. Furthermore, shielding properties against light having a wavelength shorter than 350 nm were also excellent.

### (Example 505) Production of Resin Film 505

A resin film 505 was formed in the same manner as in Example 501, except that, in Example 501, 500 mg of the polymer P-1 was changed to 500 mg of the polymer P-5. The resin film 505 had little coloration and had excellent shielding properties of light having a wavelength in the vicinity of 400 nm. Furthermore, shielding properties against light having a wavelength shorter than 350 nm were also excellent.

### (Comparative Example 501) Production of Resin Film 506

A resin film 506 was formed in the same manner as in Example 501, except that, in Example 501, 500 mg of the polymer P-1 was changed to 1063 mg of the polymer P-6. The resin film 506 had low shielding properties against light having a wavelength of 380 to 400 nm.

### (Evaluation of Light Resistance)

With regard to the resin films 501 to 506 formed in Examples 501 to 505 and Comparative Example 501, light resistance was evaluated by performing a light resistance test under the following condition 4, obtaining an absorbance retention rate at a maximal absorption wavelength (λₘₐₓ). Specifically, an absorbance of the resin film at λₘₐₓ was measured, the resin film was subjected to a light resistance test under the condition 4, and the absorbance of the resin film after the light resistance test at λₘₐₓ was measured. The absorbance retention rate (%) was calculated with the values of the absorbance at λₘₐₓ before and after the irradiation by the following expression. The absorbance retention rate is shown in the table below. The absorbance was obtained from the absorbance of the light using a spectrophotometer UV-1800PC (manufactured by Shimadzu Corporation).

$$Absorbance retention rate ( \% ) = ( Absorbance at {λ}_{max} after irradiation / Absorbance at {λ}_{max} before irradiation ) \times 100$$

As the absorbance retention rate is higher, the light resistance is more excellent.

### (Condition 4)

Equipment: low temperature cycle xenon weather meter (XL75, manufactured by Suga Test Instruments Co., Ltd.)
Illuminance: 90 klx (40 w/m²)
Time: 3 weeks
Environment: 23° C., relative humidity: 5%

TABLE 9

| Resin | Type of | λₘₐₓ | Light resistance |
|---|---|---|---|
| film No. | polymer | (nm) | (absorbance retention rate (%)) |
| 501 | P-1 | 399 | AA(93) |
| 502 | P-2 | 399 | AA(93) |
| 503 | P-3 | 399 | AA(95) |
| 504 | P-4 | 399 | AA(94) |
| 505 | P-5 | 397 | AA(97) |
| 506 | P-6 | 339 | A(89) |

The resin films 501 to 505 (Examples 501 to 505) had excellent light resistance. In addition, the resin films 501 to 505 had excellent absorption ability of ultraviolet rays in the vicinity of a wavelength of 400 nm and had little coloration.

The resin films 501 to 505 were stored for 1 week under conditions of 40° C. and a humidity of 50%, and then left to stand at room temperature for 1 day, and the presence or absence of bleed-out and precipitation was visually observed. No bleed-out and precipitation were confirmed in any of the resin films 501 to 505.

### Test Example 6

### (Production of Resin Film)

1 kg of a polycarbonate resin (manufactured by Sumika Polycarbonate Ltd., SD POLYCA 301-30, glass transition point: 145° C. to 150° C.) and 0.8 g of an ultraviolet absorber shown in the following table were stirred in a stainless steel tumbler for 1 hour to obtain a mixture. The obtained mixture was melted and kneaded at 280° C. to 320° C. (temperature on an inlet side of the raw material: 280° C., temperature on a discharge side of the kneaded material: 320° C.) for 1 minute using a twin screw kneading extruder (manufactured by Technovel Corporation, KZW15TW-45/60MG-NH) to obtain a pellet-like kneaded material. The obtained pellet-like kneaded material was dried at 80° C. for 3 hours, and then molded with a press machine to produce resin films (molded plates) 601 to 608 having a thickness of 0.15 mm.

### (Evaluation of Light Resistance)

With regard to the resin films 601 to 608, light resistance was evaluated by performing a light resistance test under the following condition 5, obtaining an absorbance retention rate at a maximal absorption wavelength (λₘₐₓ). Specifically, an absorbance of the resin film at λₘₐₓ was measured using a spectrophotometer (UV-1800PC, manufactured by Shimadzu Corporation), the resin film was subjected to a light resistance test under the condition 5, and the absorbance of the resin film after the light resistance test at λₘₐₓ was measured. Next, an absorbance retention rate (%) was calculated from the values of the absorbance of the resin film before and after the light resistance test at λₘₐₓ using the following expression, and light resistance was evaluated according to the following standard. As the absorbance retention rate is higher, the light resistance is more excellent. The evaluation results are shown in the tables below. The numerical value in the column of light resistance is the value of the absorbance retention rate.

$$Absorbance retention rate ( \% ) = ( Absorbance of resin film after light resistance test at {λ}_{max} / Absorbance of resin film before light resistance test at {λ}_{max} ) \times 100$$

### —Evaluation Standard—

AA: absorbance retention rate was 90% or more.

A: absorbance retention rate was 85% or more.

B: absorbance retention rate was 80% or more and less than 85%.

C: absorbance retention rate was less than 80%.

### (Condition 5)

Equipment: low temperature cycle xenon weather meter (XL75, manufactured by Suga Test Instruments Co., Ltd.)
Illuminance: 90 klx (40 w/m²)
Time: 24 hours
Environment: 23° C., relative humidity: 50%

TABLE 10

| | | Light resistance |
|---|---|---|
| Resin | | (absorbance retention |
| film No. | Type of ultraviolet absorber | rate (%)) |
| 601 | Exemplary compound (1) | AA(98) |
| 602 | Exemplary compound (18) | AA(95) |
| 603 | Exemplary compound (33) | AA(99) |
| 604 | Exemplary compound (42) | AA(96) |
| 605 | Exemplary compound (43) | AA(97) |
| 606 | Comparative compound (1) | A(85) |
| 607 | Comparative compound (2) | C(38) |
| 608 | Comparative compound (3) | C(41) |

As shown in the above table, the resin films 601 to 605 formed of the exemplary compound (1), (18), (33), (42), or (43) had excellent light resistance. In addition, the resin films 601 to 605 had excellent absorption ability of ultraviolet rays in the vicinity of a wavelength of 400 nm and had little coloration.

The resin films 601 to 605 were stored for 1 week under conditions of 40° C. and a humidity of 50%, and then left to stand at room temperature for 1 day, and the presence or absence of bleed-out and precipitation was visually observed. No bleed-out and precipitation were confirmed in any of the resin films 601 to 605.

### Test Example 7

### (Production of Resin Films 701 to 705)

1 kg of a poly(methyl methacrylate) resin (PMMA) and 0.8 g of an ultraviolet absorber shown in the following table were stirred in a stainless steel tumbler for 1 hour. This mixture was melted and mixed in a vent type extruder at 230° C. to 240° C. to produce pellets for molding using a method of the related art. The pellets were dried at 80° C. for 3 hours, and then molded with a press machine to produce a resin film having a thickness of 0.15 mm. The obtained resin film was evaluated for light resistance by the same method as in Test Example 6.

### (Production of Resin Films 706 to 710)

1 kg of pellets of polyethylene terephthalate (PET) dried at 130° C. for 6 hours and 0.8 g of an ultraviolet absorber shown in the following table were stirred in a stainless steel tumbler for 1 hour. The mixture was melted and mixed in a vent type extruder at 265° C. to 280° C. to produce pellets for molding using a method of the related art. The pellets were dried at 80° C. for 3 hours, and then molded with a press machine to produce a resin film having a thickness of 0.15 mm. The obtained resin film was evaluated for light resistance by the same method as in Test Example 6.

### (Production of Resin Films 711 to 715)

1 kg of pellets of a cycloolefin polymer (COP), dried at 100° C. for 6 hours, and 0.8 g of an ultraviolet absorber shown in the following table were stirred in a stainless steel tumbler for 1 hour. The mixture was melted and mixed in a vent type extruder at 260° C. to 290° C. to produce pellets for molding using a method of the related art. The pellets were dried at 80° C. for 3 hours, and then molded with a press machine to produce a resin film having a thickness of 0.15 mm. The obtained resin film was evaluated for light resistance by the same method as in Test Example 6.

### (Production of Resin Films 716 to 720)

1 kg of pellets of Nylon-66 (PA-66), dried at 80° C. for 16 hours, and 0.8 g of an ultraviolet absorber shown in the following table were stirred in a stainless steel tumbler for 1 hour. The mixture was melted and mixed in a vent type extruder at 270° C. to 290° C. to produce pellets for molding using a method of the related art. The pellets were dried at 80° C. for 3 hours, and then molded with a press machine to produce a resin film having a thickness of 0.15 mm. The obtained resin film was evaluated for light resistance by the same method as in Test Example 6.

### (Resin Films 721 to 725)

1 kg of pellets of polypropylene (PP) and 0.8 g of an ultraviolet absorber shown in the following table were stirred in a stainless steel tumbler for 1 hour. The mixture was melted and mixed in a vent type extruder at 230° C. to 250° C. to produce pellets for molding using a method of the related art. The pellets were dried at 80° C. for 3 hours, and then molded with a press machine to produce a resin film having a thickness of 0.15 mm. The obtained resin film was evaluated for light resistance by the same method as in Test Example 1.

TABLE 11

| | | | Light resistance |
|---|---|---|---|
| Resin | Type of ultraviolet | Type of | (absorbance retention |
| film No. | absorber | resin | rate (%)) |
| 701 | Exemplary compound (1) | PMMA | AA(99) |
| 702 | Exemplary compound (18) | PMMA | AA(98) |
| 703 | Exemplary compound (33) | PMMA | AA(100) |
| 704 | Exemplary compound (42) | PMMA | AA(98) |
| 705 | Exemplary compound (43) | PMMA | AA(98) |
| 706 | Exemplary compound (1) | PET | AA(99) |
| 707 | Exemplary compound (18) | PET | AA(97) |
| 708 | Exemplary compound (33) | PET | AA(99) |
| 709 | Exemplary compound (42) | PET | AA(98) |
| 710 | Exemplary compound (43) | PET | AA(98) |
| 711 | Exemplary compound (1) | COP | AA(99) |
| 712 | Exemplary compound (18) | COP | AA(99) |
| 713 | Exemplary compound (33) | COP | AA(100) |
| 714 | Exemplary compound (42) | COP | AA(98) |
| 715 | Exemplary compound (43) | COP | AA(99) |
| 716 | Exemplary compound (1) | PA-66 | AA(98) |
| 717 | Exemplary compound (18) | PA-66 | AA(96) |
| 718 | Exemplary compound (33) | PA-66 | AA(100) |
| 719 | Exemplary compound (42) | PA-66 | AA(98) |
| 720 | Exemplary compound (43) | PA-66 | AA(99) |
| 721 | Exemplary compound (1) | PP | AA(97) |
| 722 | Exemplary compound (18) | PP | AA(99) |
| 723 | Exemplary compound (33) | PP | AA(99) |
| 724 | Exemplary compound (42) | PP | AA(98) |
| 725 | Exemplary compound (43) | PP | AA(99) |

As shown in the above table, the resin films 701 to 725 formed of the exemplary compound (1), (18), (33), (42), or (43) had excellent light resistance. In addition, the resin films 701 to 725 had excellent absorption ability of ultraviolet rays in the vicinity of a wavelength of 400 nm and had little coloration.

The resin films 701 to 725 were stored for 1 week under conditions of 40° C. and a humidity of 50%, and then left to stand at room temperature for 1 day, and the presence or absence of bleed-out and precipitation was visually observed. No bleed-out and precipitation were confirmed in any of the resin films 701 to 725.

### Test Example 8

A resin composition was prepared by dissolving 7.6 g of the compound shown in the following tables (exemplary compounds (1), (18), (33), (42), and (43), and compounds C-1 to C-3) in 7.6 g of chloroform and 1.1 g of a (meth)acrylic resin (Dianal BR-80, manufactured by Mitsubishi Chemical Corporation, containing 60% by mass or more of methyl methacrylate as a monomer unit, weight-average molecular weight: 95,000). The obtained resin composition was spin-coated on a glass substrate to form a coating film, and the obtained coating film was dried at 110° C. for 2 minutes to produce resin films 801 to 815.

With regard to the resin films 801 to 815, light resistance was evaluated by performing a light resistance test under the following condition 6, obtaining an absorbance retention rate at a wavelength of 400 nm. Specifically, an absorbance of the resin film at λₘₐₓ was measured using a spectrophotometer (UV-1800PC, manufactured by Shimadzu Corporation), the resin film was subjected to alight resistance test for 3 weeks under the condition 6, and the absorbance of the resin film after the light resistance test at λₘₐₓ was measured. Next, an absorbance retention rate (%) was calculated from the values of the absorbance of the resin film before and after the light resistance test at λₘₐₓ using the following expression, and light resistance was evaluated according to the following standard. As the absorbance retention rate is higher, the light resistance is more excellent. The evaluation results are shown in the tables below. The numerical value in the column of light resistance is the value of the absorbance retention rate.

$$Absorbance retention rate ( \% ) = ( Absorbance of resin film after light resistance test at {λ}_{max} / Absorbance of resin film before light resistance test at {λ}_{max} ) \times 100$$

### —Evaluation Standard—

AA: absorbance retention rate was 90% or more.
A: absorbance retention rate was 85% or more.

B: absorbance retention rate was 80% or more and less than 85%.

C: absorbance retention rate was less than 80%.

### (Condition 6)

Equipment: low temperature cycle xenon weather meter (XL75, manufactured by Suga Test Instruments Co., Ltd.)
Illuminance: 90 klx (40 w/m²)
Time: 3 weeks
Environment: 23° C., relative humidity: 50%

TABLE 12

| | | | | | Light |
|---|---|---|---|---|---|
| | | | | | resistance |
| Resin | Compound | | | | (absorbance |
| film | | Dissolved | | Dissolved | retention |
| No. | Type | amount (mg) | Type | amount (mg) | rate (%)) |
| 801 | Exemplary | 5.9 | C-1 | 10.3 | AA(93) |
| | compound (1) | | | | |
| 802 | Exemplary | 5.9 | C-2 | 7.8 | AA(92) |
| | compound (1) | | | | |
| 803 | Exemplary | 5.9 | C-3 | 13.7 | AA(93) |
| | compound (1) | | | | |
| 804 | Exemplary | 7.0 | C-1 | 10.3 | AA(91) |
| | compound (18) | | | | |
| 805 | Exemplary | 7.0 | C-2 | 7.8 | AA(90) |
| | compound (18) | | | | |
| 806 | Exemplary | 7.0 | C-3 | 13.7 | AA(92) |
| | compound (18) | | | | |
| 807 | Exemplary | 7.1 | C-1 | 10.3 | AA(93) |
| | compound (33) | | | | |
| 808 | Exemplary | 7.1 | C-2 | 7.8 | AA(91) |
| | compound (33) | | | | |
| 809 | Exemplary | 7.1 | C-3 | 13.7 | AA(90) |
| | compound (33) | | | | |
| 810 | Exemplary | 6.5 | C-1 | 10.3 | AA(94) |
| | compound (42) | | | | |
| 811 | Exemplary | 6.5 | C-2 | 7.8 | AA(91) |
| | compound (42) | | | | |
| 812 | Exemplary | 6.5 | C-3 | 13.7 | AA(92) |
| | compound (42) | | | | |
| 813 | Exemplary | 6.5 | C-1 | 10.3 | AA(95) |
| | compound (43) | | | | |
| 814 | Exemplary | 6.5 | C-2 | 7.8 | AA(92) |
| | compound (43) | | | | |
| 815 | Exemplary | 6.5 | C-3 | 13.7 | AA(93) |
| | compound (43) | | | | |

The resin films 801 to 815 had a large absorption of light having a wavelength in the vicinity of 400 nm and excellent absorption of ultraviolet rays on the long wavelength side. In addition, the absorbance retention rate at 400 nm after the light resistance test was also favorable, and the light resistance was excellent.

The exemplary compounds (1), (18), (33), (42), and (43) in the above are compounds having the above-described structures. In addition, the compounds C-1 to C-3 are compounds having the following structures.

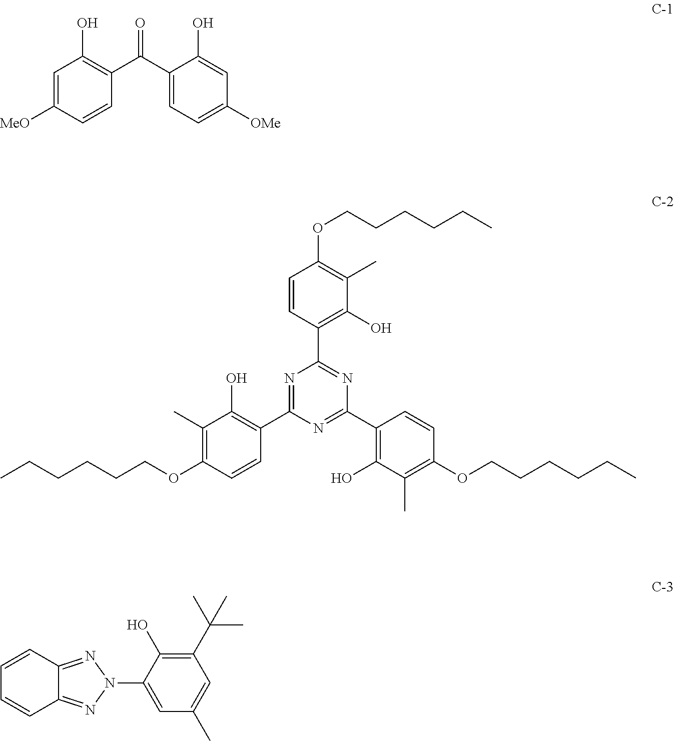 [SMILES: COc1ccc(C(=O)c2ccc(OC)cc2O)c(O)c1 ; CCCCCCOc1ccc(-c2nc(-c3ccc(OCCCCCC)c(C)c3O)nc(-c3ccc(OCCCCCC)c(C)c3O)n2)c(O)c1C ; Cc1cc(-n2nc3ccccc3n2)c(O)c(C(C)(C)C)c1]

What is claimed is:

1. A resin composition comprising:
a compound represented by Formula (1); and
a resin, 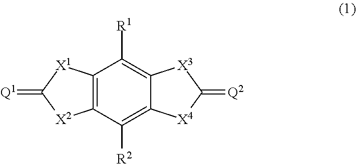
in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group, 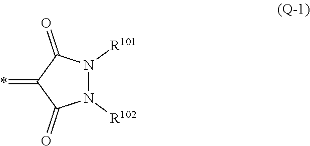
in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

2. The resin composition according to claim 1,
wherein R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
in a case where any one of R¹⁰¹ or R¹⁰² is a methyl group, the other is a hydrogen atom, an alkyl group having 2 or more carbon atoms, an aralkyl group, an aryl group, or a heterocyclic group, and
in a case where any one of R¹⁰¹ or R¹⁰² is a phenyl group, the other is a hydrogen atom, an alkyl group, an aralkyl group, an aryl group having a substituent, or a heterocyclic group.

3. The resin composition according to claim 1,
wherein the compound represented by Formula (1) is a compound represented by Formula (3), 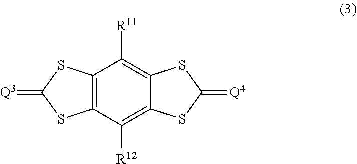
in Formula (3), Q³ represents the group represented by Formula (Q-1),
Q⁴ represents ═O, ═S, ═NR^{q11}, or ═CR^{q12}R^{q13}, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group.

4. The resin composition according to claim 3,
wherein R¹¹ and R¹² in Formula (3) each independently represent —OH, —O—Y¹¹, —OC(═O)—Y¹¹, —OC(═O)O—Y¹¹, —OC(═O)NR^{y11}—Y¹¹, or —OSO₂—Y¹¹, where R^{y11} represents a hydrogen atom, an alkyl group, an aralkyl group, or an aryl group, and Y¹¹ represents an alkyl group, an aralkyl group, or an aryl group.

5. The resin composition according to claim 3,
wherein at least one of R¹¹, R¹², Q³, or Q⁴ in Formula (3) includes a group including a polymerizable group having an ethylenically unsaturated bond.

6. The resin composition according to claim 1,
wherein the resin is at least one selected from a (meth)acrylic resin, a polystyrene resin, a polyester resin, a polyurethane resin, a thiourethane resin, a polyimide resin, a polyamide resin, an epoxy resin, a polycarbonate resin, a phthalate resin, a cellulose acylate resin, or a cyclic olefin resin.

7. A cured substance formed of the resin composition according to claim 1.

8. An optical member comprising:
the cured substance according to claim 7.

9. An ultraviolet absorber comprising:
a compound represented by Formula (1), 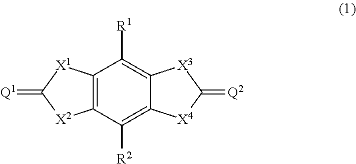
in Formula (1), Q¹ represents a group represented by Formula (Q-1),
Q² represents ═O, ═S, ═NR^{q1}, or ═CR^{q2}R^{q3}, R^{q1} to R^{q3} each independently represent a hydrogen atom or a substituent, and R^{q2} and R^{q3} may be bonded to each other to form a ring, provided that, in a case where R^{q2} and R^{q3} are bonded to each other to form a ring, ═CR^{q2}R^{q3} does not have the same structure as Q¹,
R¹ and R² each independently represent a hydrogen atom or a substituent, and
X¹ to X⁴ each independently represent —S—, —NR^{X1}—, or —SO₂—, and R^{X1} represents a hydrogen atom or an alkyl group, 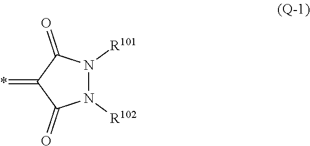
in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

10. The ultraviolet absorber according to claim 9,
wherein R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
in a case where any one of R¹⁰¹ or R¹⁰² is a methyl group, the other is a hydrogen atom, an alkyl group having 2 or more carbon atoms, an aralkyl group, an aryl group, or a heterocyclic group, and
in a case where any one of R¹⁰¹ or R¹⁰² is a phenyl group, the other is a hydrogen atom, an alkyl group, an aralkyl group, an aryl group having a substituent, or a heterocyclic group.

11. A compound represented by Formula (3), 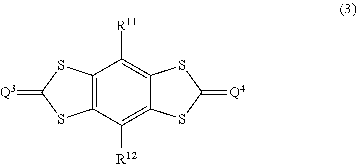
in Formula (3), Q³ represents a group represented by Formula (Q-1),
Q⁴ represents ═O, ═S, ═NR^{q11}, or ═CR^{q12}R^{q13}, R^{q11} to R^{q13} each independently represent a hydrogen atom or a substituent, and R^{q12} and R^{q13} may be bonded to each other to form a ring, provided that, in a case where R^{q12} and R^{q13} are bonded to each other to form a ring, ═CR^{q12}R^{q13} does not have the same structure as Q³, and
R¹¹ and R¹² each independently represent —OH, —O—Y¹¹, —OC(═O)—Y¹¹, —OC(═O)—Y¹¹, —OC(═O)NR^{y11}—Y¹¹, —OSO₂—Y¹¹, or a group including a polymerizable group having an ethylenically unsaturated bond, where R^{y11} represents a hydrogen atom, an alkyl group, an aralkyl group, or an aryl group, and Y¹¹ represents an alkyl group, an aralkyl group, or an aryl group, 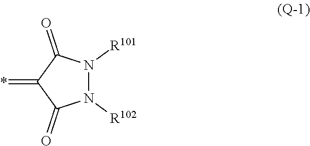
in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

12. The compound according to claim 11,
wherein R¹¹ and R¹² in Formula (3) each independently represent —OH, —O—Y¹¹, —OC(═O)—Y¹¹, —OC(═O)O—Y¹¹, —OC(═O)NR^{y11}—Y¹¹, or —OSO₂—Y¹¹, where R^{y11} represents a hydrogen atom, an alkyl group, an aralkyl group, or an aryl group, and Y¹¹ represents an alkyl group, an aralkyl group, or an aryl group, and
R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
in a case where any one of R¹⁰¹ or R¹⁰² is a methyl group, the other is a hydrogen atom, an alkyl group having 2 or more carbon atoms, an aralkyl group, an aryl group, or a heterocyclic group, and
in a case where any one of R¹⁰¹ or R¹⁰² is a phenyl group, the other is a hydrogen atom, an alkyl group, an aralkyl group, an aryl group having a substituent, or a heterocyclic group.

13. The compound according to claim 11,
wherein at least one of R^{ID}, R¹², Q³, or Q⁴ in Formula (3) includes a group including a polymerizable group having an ethylenically unsaturated bond.

14. A production method of a compound, comprising:
producing a compound represented by Formula (6) by reacting a compound represented by Formula (4) with a compound represented by Formula (5), 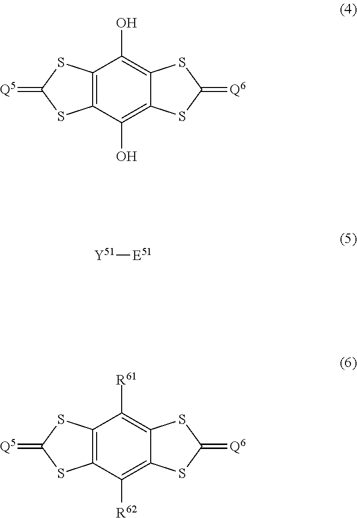
in Formula (4), Q⁵ represents a group represented by Formula (Q-1), Q⁶ represents ═O, ═S, ═NR^{q21}, or ═CR^{q22}R^{q23}, R^{q21} to R^{q23} each independently represent a hydrogen atom or a substituent, and R^{q22} and R^{q23} may be bonded to each other to form a ring, provided that, in a case where R^{q22} and R^{q23} are bonded to each other to form a ring, ═CR^{q22}R^{q23} does not have the same structure as Q⁵,
in Formula (5), E⁵¹ represents —COCl, —O(C═O)Cl, —NR^{e51}(C═O)Cl, —NCO, —Cl, —Br, —I, or —SO₂R^{e52}, R^{e51} represents a hydrogen atom, an alkyl group, an aralkyl group, or an aryl group, R^{e52} represents —Cl or an alkoxy group, and Y⁵¹ represents an alkyl group, an aralkyl group, or an aryl group,
in Formula (6), Q⁵ represents a group represented by Formula (Q-1), Q⁶ represents ═O, ═S, ═NR^{q21}, or ═CR^{q22}R^{q23}, R^{q21} to R^{q23} each independently represent a hydrogen atom or a substituent, R^{q21} and R^{q23} may be bonded to each other to form a ring, provided that, in a case where R^{q22} and R^{q23} are bonded to each other to form a ring, ═CR^{q22}R^{q23} does not have the same structure as Q⁵, and
R⁶¹ and R⁶² each independently represent —O—Y⁶¹, —OC(═O)—Y⁶¹, —OC(═O)O—Y⁶¹, —OC(═O)NR^{y61}—Y⁶¹, or —OSO₂—Y⁶¹, where R^{y61} represents a hydrogen atom, an alkyl group, an aralkyl group, or an aryl group, and Y⁶¹ represents an alkyl group, an aralkyl group, or an aryl group, 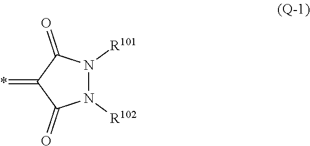
in Formula (Q-1), * represents a bonding site, and R¹⁰¹ and R¹⁰² each independently represent a hydrogen atom, an alkyl group, an aralkyl group, an aryl group, a heterocyclic group, or a group including a polymerizable group having an ethylenically unsaturated bond,
provided that, in a case where any one of R¹⁰¹ or R¹⁰² is a hydrogen atom, the other is an alkyl group, an aralkyl group, an aryl group, a heterocyclic group, or a group including a polymerizable group having an ethylenically unsaturated bond,
in a case where any one of R¹⁰¹ or R¹⁰² is a methyl group, the other is a hydrogen atom, an alkyl group having 2 or more carbon atoms, an aralkyl group, an aryl group, a heterocyclic group, or a group including a polymerizable group having an ethylenically unsaturated bond, and
in a case where any one of R¹⁰¹ or R¹⁰² is a phenyl group, the other is a hydrogen atom, an alkyl group, an aralkyl group, an aryl group having a substituent, a heterocyclic group, or a group including a polymerizable group having an ethylenically unsaturated bond.

15. The production method of a compound according to claim 14,
wherein R¹⁰¹ and R¹⁰² each independently represent a hydrogen atom, an alkyl group, an aralkyl group, an aryl group, or a heterocyclic group,
provided that, in a case where any one of R¹⁰¹ or R¹⁰² is a hydrogen atom, the other is an alkyl group, an aralkyl group, an aryl group, or a heterocyclic group,
in a case where any one of R¹⁰¹ or R¹⁰² is a methyl group, the other is a hydrogen atom, an alkyl group having 2 or more carbon atoms, an aralkyl group, an aryl group, or a heterocyclic group, and
in a case where any one of R¹⁰¹ or R¹⁰² is a phenyl group, the other is a hydrogen atom, an alkyl group, an aralkyl group, an aryl group having a substituent, or a heterocyclic group.

16. A polymer including a structure derived from the compound according to claim 13.
